SEQUENCE LISTING (11 sequences)

SEQ ID NO: 1 - DNA, 35931 bp, Homo sapiens
  LRRC8A
acttccggggcggaggaggctgagtggtgcagtgagggacaaacaaaaggaggcgccggagcagcgctgcggccggcggcgggacggagcggccggggcctggggctgcctgccgggcggccgggcgcggcgagcccaggtgagtggacggggtggggaaaggggcgcgagctgtcacctctcgaaacccacttacacacgccctcggccagctgcccggcccggggcgcccggggcatctggggctctgtctccgggccggccccctggggacctcccgcggtgttcggcctacctctgggctccccagtgcccggagtctccggggcaccaccttctcgccggctccgcggcgcgcgagccccctcccaggcacccctgtgcctccttgtgcttcagaaaccaggagtttccgcctcggctcccccatgtccccttgtcatcccctgggtccccccagatcctcacccctccacacacactctcccgttcctagaggttccacctctgggctctctccttgccatttctttttcaaagatttccttagctgtcttctcctgagttcctggtccccttctcttcccttgcacccctccttcctatcgttccgatgggggcagtgccctgacttgggggcaggatccccggctaggctcttggggcctttctgggatgggatatttgggaagaccggtccggaatctaagaacccagaccctgtcccagtcctgtgcattcaggtgggcccgagggcgaggcgagatccagtgaggtccaggcctggtgcagccctaggtgagctggatacccagcaaactgagctcccaggctgaaggggcatagctggggtcatggagttcatgatgattctgctgctttcgctgactgcttcttccccttgatctggaccggggtgaaccgaggtctgccggttctcagctgttgcgattgttgccttttttgggaaggagggatgggtcgggggttgtctggtagccgcctcctgatggagacttggctggaagggtgggcggttcctgggtgagccacacttccactgactcctcttcccctgccctacctcaggaagccttttgagaatgaggtgtggagggagagcacctggtaagaggaagggaactgggggagcccgaagttcccaaggacctcttccacctcttctgttagtagtactcttaacccctgccaaactgtattctgccctttggagcagacaacctggcagacagacaggctgtaagaagcgggaggagatcggctgttgctgatacttcctgtggaggggatgtgggagtgggagtttgttcagagagactgggcagtgctgattgctcagtctcactggtgttgcttccatgaaggtcaagtcttcctctggctcatgaagtgaggcaaggacagatgacctgtgctggaggaggctgttgtggacatgccagggatgagtgactgggtggctggtctctgcttccttgttttcctggtctcagggctttggcatggggtctgatgccctctgcgttctagggaccatagggtacagagcagggggtgcagggccgggcgtggtggctcacgcctgtaatcccagcacgttgggaggctgaagcgggcagatcacgaggtcaggagttcgagaccagcctggccaacatagtgaaaccttgtctctactaaaaatacaaaaaattagccaggcatggggctgcgcacccgtaatcccagctattcgggaggctgaggcaggagaattgcttgaacccagcaggcagaggttgcagtgagccaagatcgcgccattgcactccagcttgggcaagaagagcaaaattccatctcaaaaaaacaaaaaaaaaaacagagcgggggcacaggaaatgttcatgacttccccccagaacttgactagatgtggtggttctccatcccttaggcacacccaccaggaagccgtttcctttgcaaactcttgaatgacctttctggactgtagggaatcctggggatcactgccgtgggtcagaactcagggaagtggccggaccctgggttgctgcctggcttggcctggaccttgcctgtaggagttggagggaaatcagcctggttcctgactgcctcgtcttagagtagagctgattccaggttttcacatggagaaactgaggcacacactgatgggccaagacctcttggagattcaggtgggatggatgtggggctcttagagcccagcacagtgtctctccatgccttgactcattcattcattcattcattcattcagccattctttagaaagtgcctgtcttgggcctgggatggctgggaaaacatccctggcctcctgagagctgcagtctgggaggacaggcaaaccagaagacacataagttctgatatactgtggccaccatattaatagaaatattacagaggcagcccagaggaagaaggaattagctctgggtggttatgtagggggtggtcagggtatgctttctggaagaggagggactagaataattagtgacagggagtccaggggcccaagttctggacctggctctgccatgggcaggtgattggaccacccagcctcagtgggtgccccatccttgacaaggagggtagagtgaggtagaggttggcttggatggcctccgtgcctatatagctgtaggtactttactgcctagtcaccaccaccccgtatggaattgcagggttattgacctacctcccatgacggcggagaccatcttccatgtctgccctcccagcacagggcctggaacttagtcaatatagtaaatacccagtggtagaaatgatggaaaaacagccatccctcctagatcagtccaggtgtctttgtctacccccgccctcctccttcctgggtaactcctgctgatcctttggctctctctggtggctgggccactctcccagctcctggctccccgaaacccgtgtcgtagtgatcacagctttccctaccctgccactcccatgcacagttctgcacagtgtctggcgcatatggagtagatgctcaccagaggctggttgactgtcggagatgatgggtggatttggtgtttaagttggagtctgggcactggctggtctctgcctgacccagccaccccactgtggctcatctcttgccacttcccctctctggctctggatctcgccatgccgagcccgcaagccttctccagatgccatgtactctctcctccaggtaaacatgtgctgttcctcctgcctggaatgctctttttccagctcctactcttaggagtcaactcagacaactcctcctccagaaagccttccctgaccccactagtctgggtctagtgcttctcctggctcccccgcagcagagctctgagctctcagaccgtcgctactgacttggcacctcctcagtagaccaggttctggagggaggtgctctgtcagaacctcttttgtctgccaggcgcggtggctcatgcctgtaatcccaatactttgggaggccgaggcgggcagatccctggaggttaggagtttgagagcagcctggctaacatggggaaacgctgtctctactaaaaatacaaaaaattagccaggcttggtggtgcgcacctgaaatcccatctactcgagaggctgaggcaggagaatcacttgaatgcgggaggcggaggttgcagtgaaccgagattgcaccactatacaaaaaaaacctcttttgtcaccccagagcccagccaagggcctgctaccaagcaggctctcaggaagtgctggcggtgtggcaggtaactgcgacagtcacacatgtgcctgctgacggcagctctcgcttccttgtagggaggcagcgtccatggagcaaaaggaatgccaggatcctgcacaggcagacgcgggccagcctcagcaccgacagccgacgcgcagatagcagagccatccttggggtaaggactgggcccctctccttctcagccagctccagctccagtcaagtgcccttcccagcctggggagcaaagggtagcatctccatccccacagtcctttgcttttcttgctgtaggtccagggtagagtggcagacagcagaattactctcttcctccttctcccgctgagtaatgtgtgcttgccgcactcattgaatgctgagttgtttcattgcaaacctctgttttttctccttcaccccagatcttatcagtctgggtcttgctaatgcagcctcatgaccacatttcccaaaaagcgtggaccacgtttcccaagtgtgttccatgagacaccagtttcttcggtcacagtggtttgataattccaggaaacaaagacttcttgttatccttgctgcaggacttatcagagtcttgaatcagagccgtcatcaggattgagctgatcacgataagggagctagcgactcgtgtttccccacacttatttgatcgtgtagacatctttggggactgggattccttggcctgtgctttggaaaacagttcccagaggcaggagagtccctttagaacatttggaggattaaatatgttgacatagagcaggcagttggcagagtgcatgtcctagaaggtggtcccgagtcatagctggtgtgtgtatagacggctgtgtaatttgccagacatactcaggtcggtgatctcatttgaccatttgaggatgcttaggcagtggccattatctccagtttacagatgtggaaactgagaatcagagtgaggaagcagcttgcccagactcatctgtaaagaaggcaaggacatgcggcttgaatccagaccctggaatcctgggccagggtcttctgttgtctgccctgaggccttcctgcctttatctctcacctcagttttttgtgtgaaggaaggcctgagtctcccagcctggattttaaaagaaatagtaattttttttttttgtaattaccaacatagttgtatattcattatatggttttatactgtttggctatttttgggggacagggtctcactctgttgcccaggctggagtgcagtggcaccatcatgactcactgcagcctcgatctccaaggctccagcgatcctcctgcctcagcctcccaactagctgggactgcaggtgtgcactactgtgcccagctaattttatttatttatttattatggagatagagcctctctacattgcccaggctggtctcgaactcctgggttcaagagatgctcctgccttggcctcctaaagtgctgggattatgggtgtgagccaccatgccagacctgtttggatctttaatgtcatctaggaaatgagtctctgctgattctcatcttcagaggtgtgaatgggaaggggtatattcttccacatctattgttaccatttgcttatttttataaaaatctggtcatgtttgagttgctgattcacagtttgcttttgtttcatataatgatatcccccggacctcttttttttttgagatggagtctttgtcacccaggctggagtgcagtggcacaatctcagctcactgcaagctctgcctcccgggttcactccattctcctgcctcagcctcccgagtagctgggactacaggcgcccgccaccacgcccggctaattttttttgtatttttagtagagacggggtttcaccatgttagccaggatggtcttgatctcctgaccttgtgatccgccgccttggcctcccaaagtgctgggattataggcatgagccaccgcgcccggctcccctggacctctttcccatgcaagaggtagatagagctgtgtgtggtcctggcagaatgcacacccttgagcccccctggcccctccccctcacatgctgggagttttgagctgtttgggcatttacagcagcagagggaggtcggaagtgcctggtgtgtgattgtgtcgactctcctgtttatgggataaggtcagccaactcctggctctgccccataggagtttcctgttggttttgaggaactctgtcccctggagagaggggaggcctgagctgtagtctttcatgctgggcagcgctgagcaggaagcagcaggtcctgcttgtcagatcataattcctcctttttgccaaggatggcgtggaatgaaccgcatgggccggcctgaggggttttggcctccacccaccacctgggttcaggggacacacatgaatcaagctgagacaaattgagtctgagccattgttctcctgagcccagcccttcctggggataggaaggaacaggcagaccctgactcagtttacccttgctgtatctggaggaagtgttggttgtggtccttttggcatgctcctttcaagttttagttctgagagtgggcaggtgaggagcaggaggagggcccaccctgccacctgggtccaaagatgctctcttttggggtatagctgacctcaggaagggtttgggcaaggagagcatacaggcgtggcagcctcgttttgcggggctgcgagagcgtctggcaggatcgcgctgggagtgaggtctgtgccagagctcctgacatctcacctgtgtttttcaggctcactgtcctgtcgctttccctcccccacgtcacttcctccattcttccccaggagtctcttttctctcccatacactgcttgggagagagctgcagagctggcccctccacagatggtgctcagcgtctctggcttctccttcctgcagtagcaccttcatagtccgtggtcattctcagcaccatgtctcagtgctgagccacaggctgggatgctgcacgcatttgcccatcttaccttcgccacagccttgcgtgtgaggttgctttaccattttacagaccaagccccagagggatgaggtctctggcccaaagttatacagtgtacatgtggcaaagctaggacttgaacccaggttggtatggcttggcagctggggctctttctacttcaccctccattaccctcttttctcaggttcccaggcttttgggggccttcaaagtcacctggaagcttgtctaaaatgctgttcccctgggcccaccaccacagacctgcagagtctgccctggaaggttggccccaggaatctgaactttgaagatgcttccccagcagttgtgaggctggtgggctacaaacaatgctgagaaacactgaccggacgccattctttcagccttagtcccagtcgtctaccgcagaactcatgcccttggtgtggaagcagggctgagctccccacaggactgggactggacaggtttttttttttttgtttaattaattaattaattattttgagatggagtgttgctctgtcacccaggctggagtgcagtggcacaatcttgctcactgcaacctccacctcccaggttcaagctattctcctgcctcagcctcccgagtagctgggactacaggtgcccgccaccatgcccagctaatttttgtatttttagtagagacggggtgtcaccatattggccatggctggtctcgatctcctgaccttgtgatccacccaccttgttctccaaagttccaggactacagccgtgagccactgcgcctggccaatttttttaatttaattttacttttttgagatggaatcctgctctgttgcccagactggagtgcaatggtgccatcttggctcattgcagcctctgcctcctgggttcaagtgattctcctgtctcagcctcccaagtagctgggattacagatgtgcaccaccacgcctggctaatttttgtatttttagtagagacggggtttcaccttgttggtcaggctggtctcgaactcctgacctcaggtgatccacccacctcggcctcccaaagtgctgggattacaggtgtgagccaccgcacctggccagacagttgttttttagtggcttcattttgtgtgtgtgtgtgtgtgtgtgtgtgtgtgtgtgtgtgtgctgggaaggcgaggggcttaaccttccctcctcaaggacgtgtggggctgaggtttctagaagggcttttactcccagccacaaatgttccctgctgtcttccagatgcgtttctgttgcgtcttgcgtcttgcagcaagtttgtctacatctaaacgtctcttgagggcttaaaacctcaaaattaaattgcttgctgcaatctaagcctcacgcctagagcaatgttccagtggagtaaaagtggctgtaaaattccatagctttcacgtgccacctggacgccgtcagagtgccactgccaaacctgcgccctctgacgggcagcatgacaaacgcactgaccagcagcgggggcggtgggcgggaatcctggccctgaagggacacctcgcatctcccagctgactccatgagtcaggatccctgggggtagcccccttccatcccagagagtcgggtaccaggcagctcaccctctgtcagaccccccagtcctccccctggctcacctagggcttggccagtgcccactcccctgggatctcagcagaagatgtgtctgctgcctctctagggtactggtggccacatttcataacctgtctaagaaggcagcgctggtattcataacaggttaagacctgtaaaccgtgtgtggccgtgactccatgtgcaaatatgagccaacaaacgttggctggacgtggcggttcccaccagtaatcccagcactttgggaggctgaggtggaggattgcttgagcccaggagttccagaccagcctgggcaacatagtgacctgtctctacaaaaataaaaaaaaaattagtccaggcacagtggctcagcactttgggaggctgaggcaggtggatcacctgaggtcaggagttcaagaccagcctggccaacatggtgaaaccccgtttctactaaaaaatacaaaaatcagccgggtatggtgacatgcacctgtaatcccagctacttgggaggctgaggcaggagaatcacttgaacccgggaggcgaaggttgcagtgagccgagattgtgccactgcactttagcctggtgacagagtgagatcccgtctaaaaaaaaaaattagccaggtgtggtggcatgtgcatgtagttccagctacttagaggttgagactggaggactgcttgaacccaggagttccagaccagcctgggcaacataacaacctatctctacaaaaaatttttaaaaatagccaggtgtggtggcacatgcctgtagtttcagctactcggaggctgaggctggaggatcgcttgagccaaggagttcaaagttgcagtgagtcatgatcgcaccattgtgctccagcctcggcgacagcgcaagatcctgtctcaaaaaaaataaaacggcctgccttctaggcccttctgcccccagggcccagacccaacactgccttctggccctaaacccagggctaggctcccttccagagcttccttatagcagtacccgtgagaccagaggcatctgagtatggcctgatggggttccctgtagagccctggtttccatgtaccccctaaggaaaagagtatcttgtctcaagaagtatcttcaagggcctgggtcccaggtgggtccgggaggcctgcttgtctttgaggcaggttggcacaatggaaagtcgggcttggggtgcatcttcccagctcttcctagctgtgtggccttgggcaggttaccaaacttctctgggcctccgacatcacacccataaaaggactgatttaaactctaccataataataacagcagtgaacgttccagagccttactgtatggcaggcactagtccaaaagctttatatggactgtctccttctgtcctcatcacagccctccaagcaatagacttgtcaacccattttacagatgaggaaaccagagcccagcgaggtgaagggactttctcacggtctcgtggtgaggtgggtgccaccttgctgctatgcatgaaggttgggagatggggcaaggacatactgggcacttagttgacggtcagtaactgggcgctcctgccccagcctggcagggcgagggcaggtcctgaggctgcacctccagccccacctgctgcctctctctgcttggctccggagctgctgggggaagcccaggtgggtgtccctcacctgttgcctttcaccctagaaggcgggccacgcccttgcaagttggtgtgaggatgaacctgtttgggagcccagcccaggtgagagcagtttggaactgacccgtgctcccctccccctccagccctggcgtctcccctcttcctctccccaggaagtgagggtgtacccttctacacagggcgagtcccaagccaggcaacacccccagccagcagcctctgactcaccccagctgggtgtgggggcctccagggcagtgtccgccctggggagggtggaggctgtgtgtgccagacgaggctgagacctctcctcccactctggctctgggacaggggaagcacatgacccatggcccgtggtgggagcagatccacaggcttggaaccctcccgggacagggcctgttgagagcggccccttcgcgctccctgggagagcactggagggccagccttcccatctctggtacaggaggggtgccaacctcccgcctcccctcctgcttgggaagttttcctggctcagccttgtttgtgctttcacaaaaggctctctgtgccctgtttacccgcagcccgactgcctgccgcccaggtgtgggagccacgccaaatccctgtgggcccacatggggtctggtgcgtgctgggggggtctctctctgggacagagtgggcagcttgtcatttcttgactccctgtcttcgagaagcatccaggggatgtggactggggctgaacctaatgactggctctgagagcagatggccaactctgccacacggggtcctgagcctgggagggtctctgagtggtctgcgagttttcgaggttctgcgagtcctggggggtcctgtgggtctctgagtggtctggccctgctcagagacatggtgcttgaggctccgccctcacccaggtcagtccccactgaattctgggagggtcatctgcgatggcaggtgggaccgtggaacgacagcctgagtgtgcccggtgctgtattgacacatgatgccttacttttaattctcacgataacccatttcacagctataacccattttaggatgtaatatttccatcttgcagttgaggacacagaggctctgaggccccagtgaggcagtagtggggttggcgtttgagttgcccaactctggggtctgaactcctccctgctctgctcccaggaggtgaggaccgcaagctgctgagcgtgagtgttacgaggcagggcagggaggatgaggctgacctgtttctaatgtacggatggcagctacgcataaattcttttatcatcctgagaaaccacacagtgaggaaggtcttttgatttatttccaagcagtttttctactcataaacataccagatttaaataatggaagaatgtataatatagacagccctctacagtctcaccacgagtagacttaactatatatagttctgtgcatagctttctaaattcttttctgtacacagtaatgagtttgttattgatataattttattttttgagacagagtctcactctgtcacccaggcaggagtacagcagcacagattcggcccactgcaacctgcgcctcccgggttcaagtgattctcctgcctcagcctgctgagtagctgggactacagttgtgtgctaccacgcccagctaatttttttgtatttttagtagagacggggtttcgccatgttggccaggctggtctcaaactcctgacctcaagtaatccacccacctcggcctcccaaagtgctaggattacaggcgtgagccaccgtgcccagccatcattcttgttttaaaaaattgccaggcatggtggctcacacctgtaatcccagcactttgggaggctgaggcaggcagatcacaaggtcagtagatcgagatcatcctggttaacacgatgaaaccccgtctgtactaaaaatacaaaaaaatttgccgggcattgtggtgggtgcctatagtcccagctacttgggaggctgaggcaggagaatggcgtgaacctgggaggcagagcttgcagtgagcagagatcatgccactgcactccagcctgggcaacagagtgagactccatctcaaaaaaaaaaaaattataccagtaacagaccaggtacagtggctgacacctgtaatcccagcactttgggacaccaaggcaggtgaatcacttgaggtcaggagtttgagaccaccctggccaacatggcaaaaccccatctctagtaaaaatacaaaaattagccaggagtggtggcacacacctgtagtcccagctacttgggaggctgaggcaggagaatcgcttgaacccaggaggtagagtttgcagtgagccaagaccacaccactgtactccagccttggcaacagagtgagactctacctcaaaaaaaaaaaaaaaaagatatcatatttcctaatttttttcattgtggtaaaatacacatgacataaaatttaccaccttaaccatttgtaagtgtacacttcaatggtattaagtacattcatattgtgtatccatcactaccacccatctccagaactgttttcatcttgcaaaactgaaactctctgcacccattaaacgctcactccccatttcctgctcccccaggccctggtatcccttctgttttctgtctattcacttatgtgaataagtgaaatcaaagtatttgccttttatgactggcttctttcgcagataacacattttatatactgctcagcaaaattcttttatgcttaatatctcttagatgtctctggggtcactgcatacacatctacaaggaaggtggcgttattcctgatttaaagttgtagaagctcgggaggctgaggtaggagaatcacttgaacccgggaggtggaggttgcagtgagccaagaccgtgccactgcactccagcctgggccacggagcaaagcaagactgtctccaagataaaataaaataagttgtaaaagcagaggcttaggctaaacaatttgcctggggtcacagaattatttcttgctggagctggatttgaacctacatgtcccaaactccaagtccttgctctctctgctccctgagatccacctgtgcctgagggaaggctggggctctctgggttactcatttaacccccaggcacagagatgccctggccctggccaggaacctggtcccatttgtcctaggcacctgcttgagctgccttgtgggaagggcctgttctgcatgatgcccacttctacatctccctgggggcagggcaggaccaggtagggagtgcttacgagttctgcagggtcctcaggggctgaggagcagcagctggtaggaggagctgcctctgctgctccggtaacctggtaagatgcagtagctgctgctccccgctgagccttcgcctgtgggaggttggggagagcactggctatgcagggtaggaatgcatgagtgtttcagtgagttttgtgtgatgaaatggtttaagaaaagtataaaaactagaacagatacatccatatcaagaactcagctttgtcagacttcagtgttttgcagtaattgtttcagatacacacatgcacgcacgcacacacttgaatgtgcgcaggcacttaagtaaaatattccaagcacagttgaagtcccttccccattttcttccctccctagatacaatcattatcttggtttggagactttccatgcatgcttttgtatcttcggtatacttgggtatacaccttgtagaaaaccttatataacatatgcatcatctacaacttgttttttggatcaacattgcttttgaggtttatccacattgatacatgttgctacagttcattaactttaattgatttaaagcatttccattgtatgattagaacaacgtatcctttctcctgttgatgaacatgtgggtcatttctgattttttgctattacaaacaattcttcagtgagcatccttgtgtgtggtttctcacttatgtgtggagcagctttctaagatgcataaataatgaggagaggagttgctgggttgtagagtacgtgcatcctaccttctctaagagttcacagtttttgccaattgggggagtgaagtgatatgtgatcgttttaatttacattccttgattactagcaaggctaagaattatttcatacattggttccatttgcatttttcccctgtgaattgcctatgcgtatattttgccatttcccctcattggattgtttttgtcttatttatgcaggatttctgtatttattctggatactaatcttttcttttttctttctttctttatttttttctttgagccagagtcttgctctgtcacccatgctggagtgcagtggtacgatgtctgctcaccgcaacctccgccccctgagttcaagggattctcctgcctcagcctcctgagtagctgggactacaggcatgtgccaccaggcccagctaatttttttgtatttttagtagagatggggtttcagcatgttggccaggctggtctcaaactcctgacctcaaatgatccacctgcctcagcctccctaagttctgggattacaggcgtgagcagttgtgcccggctgtattctggatattaatcttttggtagttttaaactttggaaataactcttcctagtctgtggcttcttttcacttgtttattattatgtctttcgatgtatgctttaactttaatgtgatcaatacgttacgtttcctataaggatttatacattttgtggtaagatattcctatcttaatgacattaagacagtctctgatttttcttttctttttgagatggagtttcactcttgttgctcagactggagatcttggctcactgtagcctccacctccctggctcaagcaattctcctgcctcagcctcccgagtagctgggattacaggtgcctccaccacacctggctaattttttatatttttagtagagacaggattccgccatgttggccaggctggtctcaaactcctgaggtgatcagcccacctcagcctcctcaagtgctgggattacagacgtgagccacagcacacggcctgtcttagctattcttgatcctttgagatcgtgatttaaaaaaaaattttttttttgagacagttttattcttgttgcccaggctggggtgcaatggcgcaatctcggctcactgcagtctccgcctcctgggttcaagtgattgtcctgcctcagccttccaagtagctgggattacaggtgccctgcagcacgcctggctaattctttgtatttagtagaaatggggtttcaccatgttagtcaggctggtctcaaactcctgacctcaggtgatcctcccacctcggccttccaaagtgctgggattacaggcgtacacctccgtgcctggcaaaataatcttttttcttaattatgagtaacaaatggttataataaaaaaatagagggcaggggcagtggctcgcgacattgcactccagcttggatgacagagtgagactctgtctcaaaaaaaaaaaaaaaaagataagtttggggtgaaaaagggtaataaacagcagtctaacctcacttctaagattgttcagtgtgtgtgtgtgtgtgtgtgtgtgtgtgtgtgtgtgtgtgtgtgtgtaatttatggcagaggggttatgtactttttagatttaacatactttgaacatttcctcttgttatcaagtgttttgttctaatgcacttttgggattgcatagtattcccttatctgatcattgagtatgttttttgtttgtttgttttgtgagacggagtcttgctctgtcgcccaggctggagggcagtggtgcgatcttagctcactgcaacctccaccacctgggttcaagcaattctcctgcctcagcctcccaagtagttgatccagtagcttctttaaccagggtgtgcttattgtccacctactgcgtgccaggccccgcctgtctctttaccaggtgtcctggagccctctgatggttttccttcttccatttcccaacttaactctcgaatgcacttttcattcactcaccaggtgtctcctgaagtcagggttgctctggcagggctgggccaggcctattttgctctccacttcctgttcacatccacccgcaggctctggctggccttggcacactggatgtgttgaatgtttcaggccctggccttttagcagccctctaaggcagggcctatctttagccccattcaaagatggggtaactaaggctcagagaggtgaagtcacctgcccaacatcacaagtagcagagccagtctggccctggcccggcccttgcctccaagctcatgtaggcttgctctccactgacctgggctctcacacaggtagtttgggaagcctcatttctgtgatcttcatgatagtcctgagagcccacagggcatttgtcaccttccgccatgttaccatggggaaccaaggtgctgagaggggaaggcgtcctggctcctggatagcccctccctctctgccagctggaggccagaacagtcgggacaaggggtgtctgtgatggtggcggcagctctgcagtcacagcccagcagtctgacgcgggggtgggcagctgagggggaaggaggagtccaggctgggcagaggcctgggcgcacccaccccttggcccattttttacacaacatggacacatgtttaacaggtttgctgctcctttctatacagggaaactgaggccctgggagagaagctgttccacagctccgccaggacgcagtgccagctgagagggccgagcacagggaggataagcaagggcactttccgtaaggcagagatagcccagccacccccaccccacgcctcaaaggctcccttcccttccggggcatttctgaccacacccaggtgcaccccaagaagggctctcggtggggtctgctccatctgaatggcccaccctggaggtgctgcctgtctgggcccactgtaaatccaggtttgaacgccacctctgtcacttataggcggtatgaagttgggcagaagacttctgtctctgagccggtttccgtggttataaaatggacaccttaaaaaaaaaagccttaacaaaaggacattctcatatgtgttagaacgtggtgagccttgaggacattacgttaagtgatacagcccatcacaaaaggacaaataccttatgattccacttacatgaggtccctagagaagtcagattgacagagagaggaagtgggatggagtgcttggaggcggtgggaggaaggggtgtcgtgtttaaggggtgtgggtttcagtttaacgagatgaagagagttctgtgtatggtggtgatggttgcacaacagtgaatgtgcttcatgccactgaactacacccctgaacatgggtaagatggtacattttcagttacatgtgttttatgacagtctaaaaaaattgaggggaaaaaatggacctggaaacaatggggacctcctggctcagtgcaggggagaatctggccttggccttggttgtctgtttctgaactggaactgctctcttgtgggtatctagtcatctgcagtcctgagttcctgggcaggtccttgcactgggcccctccctgtggggattcctcagggctgggcagggaaaggaagggcccaggcttcctggctttagctgccttccctaccccagccccaacccacatccacctgaaactgctgctggcctctccaccacaccgggggcccacgccctgcagcaccccaccccaccttgtcccctttcaggtgctctctgggagagactctgagtgtatcgagtcctaacaattgactcttgtcctcaaagagaatctggcttcactaggtgctgaggaggtggaagaaagattataccagcctgggctgactgcgccccactctgcacaaagcactttaaaagcatttgggggctggacgcagtggctcatgcctgtaatcccagcactttgggaagctgaggtgggcagatcgcttgaggtcaggagtttaagaccagcctaaccaacatggtgaaaccctgtctctactaaaatacaaatattagccgggcatggtggtgggcgcctataatcctagctactcaggagactgagggaggagaatcgtttgaacccaggaggtacaggttgcagtgagctgagatcatgccactgctccagcctgagtgacagattgagactccgtctcaaaataaaaataaaaataaaagcatttagttcctaagacttgctgggtgcagtagctcatgcccgtagtcccagctacttaggaggctgaggtgggaggatcactcaagttcaggcgtttgaggccaaatgggcaacatagcaagaccccatctctcaaatttttattttaaattagtccagtgtgaggcccagcgcagtggctcacacctgtaatccccgcactgtgggaagctgaggtgggcagatcatgaggtcaggagctcgagaccagcctgaccaacatggtaaaaccctgtctctactaaaaatataaaaattatccgggcatggtggtgcacgcctgtaatcccagctactccagaggcagaggcaggagagttgattgaactcgggaggtggtggttgcattgagccgagatcgtgccactgcactccggcctgggcgatagagcaagactccatctcaaaaaaaacagaccgggcacagtggctcatgcctgtaatcccagcactttgggaggccaaggcgggtggatcatgaggtcaggagatggaaaccatcctggccaacatagtgaaaccccgtcttgactaaaatatgaaaaattagccggccgtggtggtggcgcgcctgtagtgccagctgcttgggaggctgagtcaggggaattattcgaacctgggaggcggagattgcagtgagcccagatcgtgccactgcaccccagcctggcaatagtgtgagactgcctcaaaaaaaaaatttttttttaccctagtgtggtgctacatgcctacagtccagctactctggaggccaaagcaggaggattgcttgaggccaggagtttgagaccagcctggacaacatagcaagaccccgtcccatttaattaaaataattccatggcctcctcccagcagccctgcgtggtatcatgcccaccatgtatgtgaagaaactgaggctgcaagcagtgaagagcttgtccaaggtcccacagagaggctaccagagtccaggcccacaccttggtactggcttcccctcttcatcctcggcaagagccgtgtgggagggagcctgcccctgtagggggcaggcaggtggctctgaccacccggctggctcctgttggggtggggctcctttccccacacccttgggaactcaaccctggatttggtgcctgcaccctctttcctgatctcccccgaagccccatcagagaggggcagtgacttttccagaatcacacagcagaggacaaggaaacgggtgcagctgggggtgacccctccagacaggttacccttaccaggggtaggcccaggcaccagccgggccaggcctgtgagagactccagtccaggcccttctcagctctgcagccagccccaggctctgccattggaaccccagggccctcctccactccccagacacactcaaactggagagaggaacctgaagcagaagtcccaccttgatcatttttttttacccccgctggacctagccaagggctgaacagagtccggatgttcccagaccaagcacggagttggggcgggtggtcagaatcagacctgattgggacggtgtttcctgccttgggcaggtggttttgcctctgtgagcctcagtttccctcacctgtgtgactgagctgggaacacttcctgcctcagaaggaggagtctgggtggtacagacaagccccaggtcggcggggctggggcggctcctcgcagaggaagcaggtgtttctgtgagcgaacacaccccactccctgaacacgagggaggtgttgcagtatcattgtcaccacatcctgcccgctcaaacagccgcgcccgccctggctgtctcattccagacttgggggcaggcaggctgctgcatgcaccgggctcctctcctccctcttctgttccccagtctttaggtcctggccctggatggggactccaggtgtccacatgtctcagtggaggcagctgtgtgattcagaagctctcccagtgccactttctttcccagcagtgtaggtggtgcgtggacccacctggtcattagagggcccgggccttcctgtggggcgggtgcttgccctggcccgtgagtcctggcatcactaggaggactgacttctggcctggagctgccagcccagggcgggcggtggtgtctgctggcccctttgtgacctgagcgctggtaatgctgaggggcggggaagctgcactcccagtcctgtttctcttggcaccgcctgggtgagaccttgggcaggcggagggcaagtcccacatggtggcgaggacctgagcccggtggtagagggacaggcctgctgggtttgttcttactctactctgccccttaatagcagggggatgttggcaggtgatgtcaccttcctgagccttagcacccccatctctgggatggggctatagaggcctggcctcttgggcatcaaggatgaccctaggagggcctgcaggggaggcctgcctggtgcctggcacacttccaggccctcagcactggctgtggccatcatcacccttgctgagtgccccattccacctgcctcacatggggtcctgctctgccttcctcctgtcctccccatccagcatagcctagaaagattggggctggggagtgtttggcatttgcagagaggtgctgttcttttttttttttttttttgagtcggcacctcgctctgtcacccaggctggagtgcagtggcgtgatctcggctcactgcaagctccacttcccgggttcacgccattctcctgcctcagccttccaagcagctgggactacaggcgcctgccactatgcccggctaatttttttgtatttttagtagagacggagtttcaccttgttagccaggatggtctcgatctcctgacctcgtgatccgcccgccttggcctcccaaagtgctgggattacaggcgtgagcctctgtgcctggcccgagaagtgctgttcttaaagagttcctacctaggccgggcacggtggctcacgcctgtaatcccagaactttgggaggccaaggcaggtggattggttgaggccaggaattcaagaccagcccggccaacatggcaaaacaccgtctctactaaaaatacaaaaattagctggatgtggtggcgggcgtctgtaatcccagccacttgggagattgaaacatgagaatcacttaaacccaggaggcagaggttgcagtgagtcaggatcatgccactgcaccccagcctgggtgacagagtgagactgtctcaaaaagggaaaaaaaaaaagtttgttactattctggttattaacattatcagtaatcaaagaaaggtggcccatcatctgggcacccctgttcctctcctaagagagacagacacatttgacacaggcatttaaaacttggtttagataaaaatctaggttctgggttacagtgtaaagtaaaaattgaatgtaaagtaaaagcacaatttggccaggcatggtgactcacacctataatcccagcactgtgggaggccgtggcaggtggatcacctcaggtcaggagttcgagaccagcctggccaacgtggcgaaattccgtttctactaaaaatacaaaaattagctgggcgtggtagcgtgtgcctgtaatccagctgcttgggaggcagaggcaggagaatcatgtgaacccaggaggcggaggctgtggtgagctgagattgtgccactgcactccagcctgggtgacagagcaatactctgtctctaaataaataaataaataaaagcacaatttattctaatacacagaaagggctgggtgcagtgcctcactcctgtaatgccaacactttgggaggctaaggccagaggattgcttgaggccaggagtttgaaaccaacttggccaacatagtgagatcctgtctctacaaaaaattttaaaaattagccgggcatgggccaggtatggtggttcacacctgtaatcccagtactttgggaggccaaggcgggcggatcacaaggtcaggagaacgagaccatcctggctaacacggtgaaaccccgtctctactaaaaatacaaaaaaaaattagccgggtgtggtgggcgggtgcctgtagtcccagctacttggaaggctgaggcaggagaatggcgtgaatctgggaggcggagcttgcagtgagccaagatcgcaccactgcactccagcctaggcaacagagcgagactccgtctcaaaaaaaaaaaaaaaaaaaaaaaaaattagccaggcatggtggcacacacctgtagccccagctacctgggggaggctaaggtaggaggattgcttgaccaggaggttgaggctgcagtgagccatgatcacgccaccgcactctagtttgggcaacagagcaagatccaatctcttacaagaaaaattataaaattataaaatacacagaaaggactggaaggttattattcagtgaaaagttaacagtgtgtaaccctggtggggattgtaaagcggattcttctttttttgctgctacgcttttttacaattttccgtatgttctccagggaacatctagtaccttatccctccgtgacagccatacgtctttcattgtaagagcaaccagccttggccgtgtgaccacgaatggccacagagaggcattctcacagagatcttgctgcgtgcctcctgtgtagcacagtctgggtttcctaatgtggcttctcagccagtgtttcccaagcttggccaatcattagagtcaccaaagttatgtgttaaaaatcaggttcccagccaggcacggtggctcactcctgtaatcccagcactttgggaggctgagacgggcagatcaggaggtaaagggatcgagaccatcctggtcaacatcgtgaaaccttgtctctactaaaaaacacaaaaattacctgggcatggtggtgcaggcctgtagtcccagctactcgggaggctgaggcaggagaatcacttgaaccctggaggcagaggttgcagtgagccgaaatgatgccactgcactccagcctggcgacagagcaagactccgtctcaaaaaaaaaagaaaaatcagatttccagcttctcgccatagtagtcaaactagactctctctgggagagcctgctgttgctgctgtttttaattcctttatacacacgcaaccagagatgattttacattaaggcctctctgtgatcacccagtttgtgagggaggagagccttattcttaatcccctctcttgaggccgctcccctaggagcctgtcattaagacccactatgtgacctttggtcttccgagtgttcccagaatcttgtacacacataactatacacacatgtggaattttgttgttgaagcagggtctctgtcacccaggctggagtacagcgacggggtcatagctcactgcagcttcaacctcctggactcaagcgatcctcccacttcatcctcccaaaatgctgggattacaggctaggattacaggcgtgagccaccatgccagcccccatgtggaatttttttttttttttttttttttgagttggagtttcactcttgtcgcccaggctggagtgcaatggcacaatctcagctcactgcaacctccacctcccgggttcaagtgattctcctgtctcagcctcccgaatagctgggattacaggcacccgccactacgccaggctaatttttgtatttttagtagagaccgagtttcaccatgttggccaggctggtcttgaacttctgacttcaggtgatccacccacctcggcctcccaaagtgctgggattacagacgtgaaccacgtggaatttttaaaccttgctctttgttttatggaagtgagttcatgttgtgccccctctgcatgttgctttctttcctcatattttgcgggaatccctccagccctttgctgcagctttacagcagctggttcccagtgaactgggagttttaaatgtgtccctctggtcaatctgttcattaaataggtggactttagaaaggagttcccgattgctcttactggaatgcaagggatggagcagagaaccctgaggctgcactggggatctgtctaatctcactgtgctcagtcttcctgtgtattaaatggggagcctgcctgcctcccccagggtggtttcccagccaagtggccacatgctttctgcagaagggctgataagtgggctgccccgtggagtaggcaggctttggctcccagctagatttgaggtggaattttggacaatggaagaattttcattgtcttcttcccctgacccctggtcctaggaaagccaggccaccctgtgctaacccccctcctatggctcccttttaggttgaaccatgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgtgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccgggtgagtggcccggccacagctctgcgtgtgggctggcgggtggcctggccagggcttgtggtggggcactcggcaggctcagtgtcctgggaccgtcgatgcccctgagtgtggagtcctcacctggggtttacagaaccatccaggatagaagctctgttcaagagacagactccttgggccccatcccaggcctgctgaatccgaatctctgggccccgggcctgtctttgctccaaaatgttgcagttctcctgcctggagagactgcatggccaggcaaggtctgtcccccaaggtgaccttgagccgggttcctcttcagcagggatgtgcttgggcaggaggtccaaacccgggggttctctcgcaaagccgggtggtgggagtcaagacttagcctggagtttgtgtcttcctcagcttgttcccagtcggacttggaggcaaggagccaatccgggtccacccaggacagaaactgtgtgggtggccagtgctggctgaggggaagccttggatggtctctgcagctcccacacacctttctccactgcatttggtggtgtcagaggcagagttgggcctagatggggctagtttgtttctgcctgcccttctgaaaaggaaattgtgcaaatcattgtcactgggagtttgggaggccacaccttcgacatactttcaagactgttggtggtctgtgggtgccagtctcttacctggctggggaagcccccatccaagggccccttggtggaggcaggcagtgtggatggagggctgggtactcctgtggctctgtggtctggcctccgaaatggaaacaaagccactgccattgtggggaacgtccctcttccacccgtcctggggacctgcaggttttcctgggcctcacatccaggtggagattcctgtagtgagacatgtggtggaggaagcctgaggccaggcgctgggagacaagggagagcccagccagccacccttctgcccccgacacagcagcagctgtcgtgccgggcccctgtgaacttcactagggatgccaccaggttcaggaggctgaagaagagactcggagttgaccaggcccagtgggtcaaacctgtaatccgagcacttagggaagccaaggtggggcggtcacttgagctcaggagttcgagaccagcctgggcaacatagggaaaccccatctctaccaaaaatacagaaatgatccaggcatggtggcatgtgcctgtagtcccagctatttgggaggctgaggcaggatcgcttcagcctaggagttcaagactgcagtgagctatggttgtaccactgtacttcagcctgggcaatagagtgagacccagtctcaaagtaaaatgaagagccctggagccggccattgaaacatggggtttatttgggggaacttacttacagggtggtccagcagcagtgagccaaataggaaaaccactcctgcttataaaaagcgtgcagctcatagagcattttcactaagcaccctcccctctgcagcctccgtgtggcaacccgcgtttcttagttcggttgttgctgttgggtgcatctgccatgcagcagccccacggtgcagtccctgttggtttcgtgggcacctgctctgtgttcgccctgtgccaggcatgatgcacctgtcctccttccaccaacctgctgagtcctcccaacagggtctgtctatagcatcctcaggcacggagggaggtggcccctggaatgcccggtctgtctatagtatcctcaggcacggagggaggtggcccctggaatgccctgtctgtcctcctgggccccaggacaggacacaccttctccagcatcactcagactagatgcttcaggccctccagatgcctgctggcttggaagccttcctcccctcttccccagggctcccttactgcagagcaacctgcaacccacatgtatcagggattttgacaagtccttatctccggccaccccatattatgacttgagcacagtgggagagtggaccagggcagggttttctcttgtagtttcgggacaggcccactgaataggaaaactggagtcccctggggagaagccacatttgggaagcacagaacaaacccagcccttctttaatctgtggagggggagaccctgtggcttccccatagcacagtgctgcccttgataagctgggacttcctcacagctcctgccattcctgccctgtcccgggtgctgtgctggggctgtcctcctggttgggggagggttcaagcctccagcctggctccccatcctgtcccccacccccagctcctcccgccaagcttgcctgttagtgtcatgcctgctggctgaggtcacccctcaggaaaggagctggcggcaagcacgctgtcccagaggaaggagaccctctggacaccctgtttagcacatggcttaatcacatcctgtccccaccccctggaacaccagagccttaatagggaaaacagttcacttgggccaaccccaggcaggcccctcattctgagtgggaggcctgagcccctcttcctcctcactcgggcttgatggctccttctcagccaccttggccagtggtgaccaggaggggtgggcgtggcctgcccacgcccttgcgtgctgtttctccagatttatactcttgctaatgaaagccttgacaagacagtattcattcgttcactcgtttactgatgtgtgcaaattgttatgttttgtttgtttgtttttttggtagagacagggtctcactgtgttttctaggctggtctttaaactcctggcctcaaagcctcccaccttggccgcccaaagtgctggtattacaggcatgagccacttcgcctggcctttgtgcagatgttttttgagcacttctagaatccagctatggagcaaggcgccggggacatggggatgaagaaggccaagtaacgaggcagttagcacagtgcgtaggcgctctgctggagcaccaggtccctgggaacacagaggaacaggcccctgaagaacgaggaggaggaagccaggtggaggggaaagggacagtccaggaggttctggtggggaggagcagtgagatgggctgcccaggaatgtgggcatcctggagagcagaggtggggctatgggggggggtgtggatggcaggggggtcctgtcctacacctcagtttcaattgtctcttcatttgtttaacagccgatcccttgccagcccataggctctgatgactaagacctagctcttttgttcaagcaagtgtttaccgaattcctagtgattgagtgggtgaaggaatgagctgacttgcagcgtgaggttcctcttgcggtagtgtgtggggtggggtgagtgagaggtgggaggtggaagagagctgttaagagaccacgattagccatcaggtgaggccacggggcctggcgtggccaaggcaggtagggatggagagacatggacggatctgggagacgctggagggggaattcaaagggacgggagttgatggtgggggtggtctgagtgacttgagcttttttttgtggattgttgagactggaggaagcacaggttttcagaggccaaggtgtgtctcatgtcaggtgtctggggctctgggatgtccaagggaccatcttgaatccacctgaagcccaaggagtcaccctttccaggctcatgggtgaagatgggtccttgacttgctcactgtggagagcagagagggagaaggacaggagtctgcagaagaggagggcgaggccagagcgtggggcacatggggcctggggagttggctggattaggaactggtgactttggccagagagttaagggtggacagggtcatggaggcagagtgaggagagagacagcggctttcctgcagttggtcaggaaggccagagagccaaggacaacgctggggccctggaagcctcctgagagagttgtccttttcaaagatcaggtcgagttgaggcctttcagtgccgatggggacccactgaagtctctggggaggtaggagagggtgcaattgggggattgggaaccaacttcaagcctccagggcacgggccggcgacaatccgacaatccagcagagaaggatcggtccagaggatgggtgtggctgtggtgggagccgagcaactgagagtacctccccttttctctctggggcctggagagggctccctggctcagagcagggcatgttctggggaggaggcctttcagggagggggctccagagccaggctccatatggacccgagctctgccactcgctgtccagagctttcccactgtccctgaccactgcatgtgtcagcagaggagatcacggggtgggagtggggtctttctgggaaggctctggcccctgggtgctccagagcccacttcctgtctaccctctgaccttcagcccctgctgacctcaaagccaaccccaaatgcattcctgatccttggtcacagtggtggcagtgtaggatacacaggttaggagcctttttgaaagcaccccctgtgcctgggctgggcatggtggctcacgcctgtaatcccaacactttgggaggccgaggcgggcagatcacctgaggtcaggagttcaagaccagcctggccgacataatgaaaccccgtctctgcaaacaatacaaaaattagccgggcatggtggtatgcggctgtaatcccagctactcgggaggctaaggcaggagaatcacttgaacctgggaggtggagattacagtgagccaagattgcgccactgcactctctgggtgacagagtgagactccgtcccccgcccccgccccccaaaaaaaaagaaaaagcactctgccgggccttggccatgcttatctccctgctgggaaagccacgttgctccccaggaactcctgggcccttctctaaacgcggctggagagagcaggtgcccgtgaggttgaggtccgagtctgcaaaacggtcctcagccacagagtgtccaagctcagtttctagtccaacctgtgccttttacaaatggggaaactgaggcccagagagggagggacttccagagttaccctgtaactgagtgccatatttggcagaagggaaatacagcatgggctctgagtgggaggaaataagacgtacatcctctcctgaccctttcggagaggctgcaacctcagacgagtggcgtgtcatctctctgggcctcagtctccttggcaggaaaatgggggcggtgatagagcaagctcagaagggctgctgtgccatctccccatcatcctcacttgggatcaaggcccagggctcctggctcctgccctcctgcccatcccatctgaaggaggcagcctcgggacgggcaggctcaactcatggccttacttacacgcagtaagaactcatggccccagctgattcctcgcagcagggcagggaagaagctggcatacgtgaggctcttcctgtgtgccaggagacagcacgaacccactgcagccccctgtttgctccttgctccgctaacagtgtggacggttcaccagagcatgggctgttgaaccgggttcaaatcctggctccatcacttaccagctgaacctctggacaagtcagcgtctttgggcctcagttaccccatctgtaaaattggaagagtattgacagaatatttataggtagtttgctggttaaataaattaatagtatcagtgctcacagtgagcctgccacacattaagctgcatacgggtgtcgtctgttaacagtaattgctccatatcctcagggaatgtggtccaaggtcccccgtggatgcctggcactgcaggtagtgcagaaccctatatgttctgttcgttgctattcacaggtacctatgataaagtttaatttataaattaagcatagtacacttaggctttgaggctattattatttttttgagatggagtctcactctgtcacccaggctggagtgcagtggcgcgatgtcggctctgcaagctccgcctcccgggttcacgccattctcctgcctcagcctcccgagtagctgggactacaggcgcccggcaccatgcccggctaatttttttgtatttttagtagagacggggtttcaccgtgttagccaggatagtctcgatctcctgacctcgtgatccgcccgcctcagcctcccaaagtgctgggattacaggcgtgagccaccgcacccggcctagggggtggattttctcagcatgctgttttgctttcataaattgttgttaagctgggcagaaccaacagcctggctcctggtgagccctgggatggaggtgggagcagctggccaatgcgatatacttcccagggccaggtgctggaagatagtggggatcgaggcaggcaggtctcatccttagagcccacatcaagggggtaacagatggaaggatgagcactgcgagtggggacatgggggccatgagacccaggagatcagggaaggcttcctggaggaagtgagcaagagctgagacctggtgcacaggcagatgttggccagagtttggcccagattgtgctgatgctgggggcctggggatcagaaaagatgctgagatcttggggagagagggaagggaagcccagaggccccgtccaagcccacacccacctcactctcacccctgcttttttccctccagatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgtggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtatttgtggcagttttagttttttgtttttttttttttaatcaaaaaacaatttttttaaaaaaaaagctttgaaaatggatggtttgggtattaaaaagaaaaaaaaaacttaaaaaaaaaaagacactaacggccagtgagttggagtctcagggcagggtggcagtttcccttgagcaaagcagccagacgttgaactgtgtttcctttccctgggcgcagggtgcagggtgtcttccggatctggtgtgaccttggtccaggagttctatttgttcctggggagggaggtttttttgtttgttttttgggtttttttggtgtcttgttttctttctcctccatgtgtcttggcaggcactcatttctgtggctgtcggccagagggaatgttctggagctgccaaggagggaggagactcgggttggctaatccccggatgaacggtgctccattcgcacctcccctcctcgtgcctgccctgcctctccacgcacagtgttaaggagccaagaggagccacttcgcccagactttgtttccccaccgcctgcggcatgggtgtgtccagtgccaccgctggcctccgctgcttccatcagccctgtcaccacctggtccttcatgaagagcagacacttagaggctggtcgggaatggggaggtcgcccctgggagggcaggcgttggttccaagccggttcccgtccctggcgcctggagtgcacacagcccagtcggcacctggtggctggaagccaccctgctttagatcactcgggtccccaccttagaagggtccccgccttagatcaatcacgtggacactaaggcacgttttagagtctcttgtcttaatgattatgtccatccgtctgtccgtccatttgtgttttctgcgtcgtgtcattggatataatcctcagaaataatgcacactagcctctgacaaccatgaagcaaaaatccgttacatgtgggtctgaacttgtagactcggtcacagtatcaaataaaatctataacagaaagtg

SEQ ID NO: 2 - DNA, 75235 bp, Homo sapiens
  LRRC8B
gatagcctccctcacaaattgcttacagtgaaatgccttgtgggtccctaaatctttcagaatgcatatccctcctataaatgagccctaaaacacagttctgttgaatctcactctgacaataacagctcatcttcatatgtatggtataaggacaagacttggaaatcatccctctgcccaccccaagacaaattcataattgactttccccctattttcacatttatcttatgtaaaatgtagatttactgagcatgagaggaacacataatcagtattcctctacccactcttcacacgcaaaatgtgaattcactgggcactaatcaaagcctcacaagaatgtaacttcttccctcattgcctaccttcccatttcccttttttttctttcttttctccttcccctcctgctggctctttcccctttaaatactgaagtcctcaaaaactctctttgcaaaaggcacagatcacagatgttcctgtatttgtgctttctcctggggaatgtccttaaccttggcaaaataaacctctaaaacgattgagacctgcctgtcatttttttggtgcttaaaatccatgatggaatttttaatcactattttagacttgggtaaatctggctgtgaaagtagtgacagagtcttttagaattttcttctaccaaaaataattaatcagaaagtccctgagagaatttcagatactcctgtaaggatcaaacctaaatttaaattagacttgtagatctgtgcatacccagtgtctcacacatataaactcaataaaaatttatcgaatgaccaagtgaagaaatgttctttcagcttttccttcaagactcagaaaaaagatatctgcagttccagaatggactgagtgaacaagaaaagaacagatcagtgacacaaagacagccttctttttccctttcagggtgactcaaattgtagtcctcatctgaacagtctgagacacaactcttgtctcatattaactcgatagggcatttgctatactcccatttacacaaactgctcagatgccccaaatgctgagtctgttcgtcttcattttattcctttgggagatgccggtacaaacttgttttttctttctttttttaaatggagatgtgaccaccatagcaactaaaagacataaatcttgcctaattgaatcagtgtgctcaagataatagccagggcagagactgagttaaaactggggtaagggtgagagtggggaaatggatgaggatgataaaaatatttatgtccctagaatttgtgggtttggattaattctgtttcgtaaggaatattgttggccttcaaatgcagtgccacttgggagagaagcaagcgggcagtagttgaggtgtctctgcagttgaatcgcagtgaaaggcaggttatgagtatagggaccgcccctcggccatgtggtcccctccctatcctgaggataacgacatttgagagaagccaatggtattggaaggccagaatcgaacttaagccgcttagttggtacaactgtaaaccctccctcacagaggctgggccctgcaatctttctaagcagggtaaagaaaagtaaggggacttccttttcgaagaggaggttcccgcgtgctgcacccgacctttccctcagggcccaacacgcgttatataaatgccaagttactcagggacgcacacgtgagaaggctggggcccgacacacgcggtgtagtgccgaagtcaccggtgtggcgcgcctgggggacacgccgaagcccatttccctcccgcttctggaaagcagtccccgctctttccagctgctccgcggggcgtatggctgtgcgctacctcctgcggaccccaggctgggcgagggggcagcgatggccacatggcgcgctcggggagatggcagctgcgctttgtttccagcagtgataggtgagtcactggctcccgcagccccagagctgggcaaacactcaggcccaggcagctgaaaaattcacagcaggagcagctaccgaagcgactgcagcctcttcctcctcttcctcctcctcctcctcctccgccactgctaccgccaccaccgtggtagccgcctccgcgccctgcctccgccccgtcccctcctcctccacccgcgaacttagtcagcggttcgcgccgccgagcgcccattggccagcggaacgcgtcgcgtcacaatggagccggtcggaggcggcgagccggacagcgccggggcttcccgctgagcccgcagcctccggcagccgggaaagtgcgggcgcggggccgcagcctgcccgcccggagcggcccaggagcacgccgcggggagcgcgggcgtccggggctggagtcgcgcaggtaggtcggagcgtgacaccccggttccaggagggcgagccgcgccgaccgaggtggtggaggaggaggaccggatacggggacgcagcaggtggggctggccgaacaggtgcggttacctgttcacgcctcacctgcgagccaggctgcgggccggtagcgcggctgcagagacgagtggccgcttcgttgtgctccctgccttcgggagagacggagtcccacgctgagaggacctctcctgtgtgctggggtcatttggagcaagaggtgtgggcggtcgcgccgagacggtcacctctctttcccccgggagtctcggcgttgcacctgagcgtgagtcctcggggcggggcgggaaaggggttccgctttcccaggctggcgtctcgctccagatgagcgcaactcccctgagggtgcccccatttggtcgctctgttttgccccctgcgctggcgctgttgggaactattttatgctcttgggaactattttatgctgttggatacgcgttggttgcctataatttggtggtggtgttgggcgttgatttgtttgagaaagaaagggcaagcgttaccaatttaagaagaagactcaagagtttcccctgagcttagttgagcaggtgctgagaggtcagaagggaggaggctcagtattggctcattaatctgtgtgctcaactgacttcctaaagtccatagattgcctttagttagatgacatttgtggaaaactttttttttctttgtggtcaaaaagacacccctctaaaagaacaaaaccgacatgtgaacttccttgaataaattcacttcgcctaaagacttcaaacaaacaaacaaaaaaacactccactggtaaaagataacagtatgaacattttaaattgtagatatatagccaaattaaaacaggcaatttgttcagcgtatattaagaagacattacgtagcatgatttttattaaaagacaacctatttgcaaatgatatagtagtctttgcagaacaccccagaaatcttaaaacagatttatctttatcagatggttctgtcccctaaacctaggtgtaggagtgaggatggaaaagaagtcaaactgcagtgtaattttttaaacaattccagtaccctctatgtgtacaggctacacctcctgagaaccccaaatgctctttgttttgtttctttgttttgttttgagacggagtctcactctgtcgcccaggctggagtgcagtagtgcgatctcggctcactgcaacctccacctcctgggttcaagtgattctcctgcctcagcctcccgagtagcggggactacaggggcccgccaccacgcccagctaatttttatatttttagtggtggcggggtttcaccatgttggccaggctggtctgaaactcctgacctcaagtgacccacccacctcggcctcccaaagtgctgggattacaggtgtgaccccaaatgctctctgaattgtgggtgccgaggtgactttctttttctcctggctttccttgtcagcagctctactcagagaatataaagtgacagccaacccccaaggtaattgggcctgtgtaattttagaatataagccctaacatgtctgatatttctgtaagctaaaaagaggtctcttgaatgggggtgatgttgtcattgagtgcacccttgggtcatggcactttccttatacttagtatccccaacaacatttttaaatctgcatgtcggaattaagtttaccattctttgaaatgtgttctctttttcctcaattggtaaggtgttccaacatgttcagcctattatgaatcttttcacattttgaatgatttcatttgactagttgagatttgcttttttaaacctcttctctaccactatcaccatttcccattgctattttaattttcagaatcctcagagtgaaaacaccaacagtaagtaaaatgaaaaggggcttaataaaacttaaaggatactaagccttccaagttttttagactaatccatgtattttcctgtagtggcctgaaatttgttattttttgttgttttctgaagtttaaggggaaaaatattttacaatttagaacatccttgttttagccaatgtttttcttataaacaagggtttttgtttttgtattttagttttcaaattcttctaatagtacctgagtattttatgttttctggactttttactattcatagcctgtttaacccaatcccggattttcatttggtgtctctaaagttgaattctctaattttactcttatttctccttcccagcatccttctaccacaccctgatattattagaaatgaatgcattctgattctgtcatgttattacacacttggtggcatgatttgcattgccttgcatggagcaggttctccactgctatcctgactcccagagcactgggtacatcagcaaacagttcagacctgtgaccattcaagaggaatttgggagacccagttatagtgtagaatttatttaaccatttgtaaaataccttctaatttgtgtcatatctgttttcatattgctgccttttattgctattgcctactttcattttattccccatctgtattttctaaacttcctacatgcctttattatgacttaatttctctggtgtttaaaataatgcctttggcagacataataacctatttttctttttctgatcattaaggttgatcttaagtaaaattaggcctagctccaatcactgatctctctcctaagttcttatcactgtggttcatttagaaattgaacaaagggtaacaataaactccctgttctatgactgcctttcctttaggattctactcaattgccacacaatttacatttactccttcagtgctgctgtgaacctgagtaaaggaaatgaataaaaatgagttgtcagggcttcaaccttgacctttcctttttgcctttctgacctttttgctgcttttgatatgggtcatgtgattctgcttgtttctttctagactgtatatatttaaacttcatctcttgctttttttaaatttcagaatccctttaaattctttcttctcatctgtcatcctgtgaccatttccagggctccattctgcaatgcctattcttcagctaacaagcacagcatatttcacagacgctgccagctgcacagcttctatttctcttccgtatccagctccatgcaggtgactgagatcttcctctgctcatctctcatggagaccacattctttattgttgagtagcatattccttttgatttcttctcaggtagcagatttaagagcatattcaaactttccttattttcttctgtcaaattccttttcagcattttgaaacttttctcttttcaaaatctcttgcaaattacaagcaagagttcttcaaggttttacctaagagaacaaacttcatagactgacaggcctgattctgaactctgtgccatcactttggatgagttgtggattagttaaaattagttgccagctacagagactgcaaagaacaatggcttattcaaaatgatgcttgtttctctttcacattaaacaaacttgcagagaggcattctatggctggtatgatggttctacagtcttctgagaactaggctgattctactttttggattatctaccatatataagatgaggccctcagcttcatgatcccaagatggagcttcagccatcacccccaagttccaagtagcagaaaggaggaaagaaggaaaaaggaaggaggggaaaggcaagagcccatggtctctgaaggaaaattcctggatgctgtcatatacttcttgttatatgtcattgcctagaacttagtaacatgacctcaggtagctggaatagagactgggaaatactatccttattccagacagtcttattcctagctaaataaaaaaaagtgggagactcaaatccttagaaaaaagtggggaattttggaggtatccaaacaccatagctatcctaaatctcttttttcttgactagtattgtctttctcacaggagtcttaggattaaaataaatgttatacattaacatatctaatatagtatctggcgtaaaacatgctttcagtaaatcctgtttatattcctccttacttcttttcctttaattgtctagtttccctgctacaacatcactggcccccaaatcctattaatattttaaatacttgggcatttaccccacttggtgttacgagactgctgtgttgtaccttgtcatgggaggagatgaggtttttgagtgtcagggagcaagacgttattactcaatgtcagcatttattcttagcctaaaaacatggcatgtgaaatcaaatagatctggacacaaatcctccctcagtatgcaaccatagtctctctctctctctctgtttttttacctgtaaactggataataactgaccattagaattgaaagaaaaaaagtatataaagtgcttaatactgtcctcagtaaagtggtagcttttcttttttaatttggcctaacttgtgtgagccttttcaaaaatcagtacttattctattttttagaaagttataaaattatctctaaaatcagtttatcacccaagtgacagcaaaattttttctatatattttgttataaaataacaaaatatggaatggttaatgatacagaatggttaagccttaaccttcctcagccttgaattatttttggctgacccactctcagaagctaaaccttaccaccatttaatttatttcacagtattttttttttcagtagctattaagcatatagtttgtgccaggcactgtactatattctggggatgcaatgatgatgagcaaaaatgcacatgttccccaccctcgtggagatctgggaccatactggggaggacaggtgaatcactcactcactgcaaacagatgagaagttgtcactgtgaggttgttataaaggagacatgggtcgggcatggtggctcacacctgtaatcccagcactttgggaggctgaggtgggtggatcacctgaggtcaggagttcgagaccatcctggctaacatggtgaaaccccgtctctacaaaaaatacaaaaaaaaaaaaaaattagccgggcatagtggtgggcgcctatagtcccagctactcgggaggctgaggcaggagaatggcgtgaacccgggaggcggagcttgcagtgagccgagattgtgccactgcactccagcctgggcgacagagcaagactctgtctccaaaaataaataaataaataaataataaataaataaataagagtatataataatataacttctcaatatgacactcaggaataatctaattcttttacagtttatttgggctatattagcaaactttaaatacagatgctgtcttgatacaacaagcagatgcatctgagacagggcactgtaaagtaggatatttttgtagagggccagagggttgatctgcatcatccttagcaaatggaatttgaaaccatatggctgaatgaagaaagactgttggcaaaaaatcagtagcgcataaagcagcagtggaaaatgaccacaggatgggaagtatgagcagtgtgtttcagagggttgggtgactggactatgcatgggatggccttctcaaagcgggtggacaaccccaacggccggtccctccgtacctttccattttgcacagacaggaaccagcatggcagggggtgctattgacaggtgctttgggaggtgccgtttctccttgccgtgtcctgctgagctattaggggagtttgtggggaaacaatgcctctctaatccctgtaacagccttggaggatggcagggagcactctacttccatgcatcttccacgtattgtggtattttccatgtattgtggtatggcatgactgagccttgggggttgagccatcagccattgcactgaagaatgtttcttgctctgagtggacaaatttaagataaattctgttcatctaaagattttaagttcatttcttttaacaaaactgagtttattcctggccctatttcaatgctaaaataagcagtcctaagctaagcaatgacttgtctttcagaagcatttctgaatcatgtagggaagtttaaatttttccctgaaggtttgataatttgagtctatgaaacaaagtgataatagcttcacaggaaaaaaagcctgataaattttattatgtgcacatgtatgcacaggagtcatataaaatattttaaaaactcaaagaaatggccagatggttaatgcttatataccatcttgaggtaacagaaagaacaggggctgagagcatggccaaaaacaggtaatggtggtaaatcaggttatggtggcaagacaggttatgggaaggagagaagaggaggcctggctagcaaaggtggtcttgttatctagatgatcctctctggtagcagccctcagatagaatagatggtaaatgtttctttcagacctttaaagctgtcagattctcagttaatctttcctagatccagacagacaaggtggggcctcagagaaagcctggctgcatcagtgcagatactctacagacacaaatctcccgaacaaatgacagctttactggcctacttctgttttgccctccgaacagccatctcaaaatatgtcaaagaagtatattttggggtgcaatattttggtttccttcaatcataatgcagtttctcagaggcctagtgttgtgtgtggtggtcaggttcgcaggcaatcccaagagagtttatgttggccagtggtgccctaaggtgccagagtctttcaccctcccttttcctggaatgaccttgtaacttggacttcctgcttcagatttgcctgggtatttgcagttcctccctcagtggcacaccaagctccttcctaccttaggggcttctcagttgctgttctttctgcctggaatgctcttcctttataaatttccatgactactttcttctcaagagtcagctcaaatgtgtcctcagaaatacgttccctggccaccttaggtaaagtacccataccccctacccatgttcccgtcatagtctttcaaaccttggtttattgtttgtttgcttgtttatgctctattcccctcacccccaccagaatataagttctgtgaaagcaagaactttgtctcccttgtttaactgctcttaagcctggtgtgtagtaaacgtgccaaaaccgctgcatgtgatagtgggtgaataatattacctcattgatatagtttggctgtgtccccacccaaatctcatcttgaattgtagttcacatagtccccacgtgtcgtgggagggactgggtggagataattgaatcatgggggcagtttcccccatcctgttcttgtgattgtgagctagttctcacaagatctgatggtgttataaggggcttccctcttcactgggcactcattctgtctcctggcaccctgtgaagagatgccttccatcatgattgtaagtctcctgaggcccacccaaagccatgcagaactgcgagtcatttaaacctctttcctttataaattacccagtcttgagtatgtcttcattagtgtgagaatggactaatacaccaattagtcataaaatggggataataatgtccatgccacctacctcacagaacaaatgagggcctggagttagtggattgaagatactttcctcagaatttgctgttcacctttagattctgtatttttgccaacagcaatactatttacccagttatgtatcccaagtttcttggaactcactctgggcttctccttgctttattctccctacagtcctcttaaaggattggtaaccaatttgtctagagtctatagccttcaggtccttctatcagtgcccttcctctacagcccctttcctgccgtgagtcttccaggaacactgccagttatcttctaactgataatgattttgtagtcttgagcatagtctatctgccagcatcacctacaaatttattgtccacactgcagaaagtaatctttctaaaagacaaatctcatcccataattgcctgggtttaataaccctttaatggctccctatgccccaggattaagtccaaacaccatggtgtggcatgtgagaaagtcttcctttgtctggcttctgcagctcttcagcttcatctcttgccactctgtcatctctgtgtccccagtgcatgtcccatggacacagtgtgcagtcatacccccatacaccagctgtcctaaactatttgcaatacagggaggaaggttctagatagccagatggcttcattccgtagctttgttccttccctctgtttgggatcctcttgacaagcacagaaccatctttctaggctccattaaagcataccttttctgaaagacccttcctgacaccttctgtctgcctctcacccccacagctcctcattccctcttctgtgctgcttctaagctatacctatcatagtacctaggctagagaacagcttggcctcctcctggatgcttcttcatgaaaaataatatattttgttcatttcctgggattcctgggccagacttcgtacatggcataccgtgggcatgcagtggatatccaggagagaattagggaatacaattagcataattattacctagtctagggtttcatttcaataacgtacttttgactggtatttgcataaaattttaaagaatatctccttgaaggttggaagtaacaaggtacattatacatgctgttgataggtatcagttgaatagctaaagtactttgtctatgagctagtgtttattgaacatttactgttttcagagttccagatttagcaacttgtaggggatgcttgaaatggcttttgtacttcaatgaaataatcctgactttggctttctattagaagtcaaaattttacgtggttaacacaggctcttcttggctttcaagtagtttataacagtatctctgaatgtttttgagtatcctctaaaacgattttgaaaaacgacttgttctcatatatttttaaagttaatgtctgaaatttttcatcataattttgaatagttgcagacagtcacatttctattataactattgggttttaaaaatcactcttttaaatgtacccaatggaagttaaattaaataccaaagtgatttgatatctcacatcatccgtttaagaaacgtataaataagctcttttagaaaagtcaggaattttacgtagatcttttttctcatggaactcattttattcctcttctgtaaaacttaatcccagtgtaacactgttatgattgaaagtttttcattatcatgtcttttgtgacatacacacacacacacacacacacacactcagtatacttgttttttaaaaagtgcttgtgacaataaggctctaaatgttgaaaaacatttcttttgaattgttattatgatttttcactagtacacgattgaccagaacaacacgtgtaatcaaagcaagtgtttctttttaaatggaagtgcttaagaaatcttgccatataaatatatgggaatgaagtcagtataattccattctatcttctaaacccctgagttatatgcccaaagatatctagtgatttatcacagaaacgatttttcaatgttctgttgcttgacatttgattaggtcttcctccagtgttgatctaagcaaagaaatagaaactttactctgactcccaaaaggatttttctcaggcctgaaagtctttcactttgtcagtatctgtaatcctatgggtatttcaaaatggccttctgcttggggcctggagatttttacaccccaaggcaatgcaccataagtggactcctgagaaataagatgtttgcctttcttttttgctgcagtaaaagggtgaatttgaaaaggggaaaaagaatatggctggaaagagccagcagaagggcagtatgtattttgaaggaggtcctcttaggcagggttaattgagttagaagtagattccaccacattgtcaattactcccaataagactcccaaaaccccataccccagcttgaagatcactggtttggatgctaaagaaaatttttcagtcaaattagctatcaactgttaggtccctaataggcccatttaaagattcccccttggtgtacacagggatatccgatagggaaagagcatggttagaacatatcactcttttggacccaggcatggtggctggagcctgtaatcccagctactcaggagactgaggtggaaggatgcagtgagttacgatagcaccactgcactccagcgaaggtgacaagcagcgaagagcaagaccctcttttataaaaacaacaataacaaaaaagaacatcacctttataaatatacctagtagaaactaatttgataccaaattgatttaattaccgtcatccattttaagaatgtacaaactcacctagaatagtaagaaattgtacacaattctttgatacccattattagactagatacaaaaagaacattagttaaagataaccagccgcttgactgacagtcatctgggtgagtgagtgactggtttctaagaacaagtcttaatcccactggaaaaaaaaatgcttaagcattttatcaagaaagctaatttcttggaagagtttgtgaattccagcatgcaagaaggtttggtcctaagctagatttgggattttttttttctttacaaataaatgcaattgagagggagaggacaacagtttggagttgggcctggccatcaccttactgagttcggagcctttcctggggccctctgggtttctggatgttctgttcctgtgtaagagggtactgtatagctgtccgctgctctcttgccttcatatagcaaaacaacttgtagcaaatctctgcagaaacccccacccaacacatcacttactatgatagacatttgcccatcaatgctaacatattaagtgtttatattcacatttattctcctgatgacctgcagtagaggggacaagcattatccccatgatacaaacatggaaactgagttcctcaggggatatgtggcttatgtaagtgaaccacaaagttaccagcagatatggcactcgaatcctgataaagcagctttttctataattattgcctggctccttttcacaggtgtattattttagaaaaatctctaaggattgggaaacttgccttaaagtgatagtatgaagattgagatagataatttcataaaatacctagtagagtgcatggctcttagcaggccctagagttttcattcaatacatttttttgagcagctaatatgtgtcaggcactgtttacaggcacagggaataaatccaacagagaaaacagaaaaaaccctttttgcatgaagcatatatgctagaggaggggaaataagcaaatgcaaggtgtaatgtcatgcaataaagtgctatgaagaacagtaaaacagagtgaaggactagagaatggttcttgttggggattaggtagtatacaatcagggacagcatttctggggtgggcacttttgataccgagtatgatgctcacctgagtctgagttcaaactttgagtttatttgagtaagctgattcgagtcatggtattccaagttgtgtgaaagaaacctcagagggtacttttaatttttaacttttttatattatagatatacttgaaggtctttcccagaatacacacatactcatgtgtgtacaatttcaacatgccttgaaatcatccttagaatcctcaagctcatacccctgttctatttcagtcattcttttaactggtaagatactaagattgagaaaggcaaagtaaaaattttccactgtgtcacaactaattagtgactgaatcaaggacctaagattatcacaaagtcatttattgctttttctattgaaatgtgtaagtaacaccaggtctggtaggggaggcttgaggcttgagttcaaggctgtggcactacgatcatgctataaatagccactgcatttcagcctgggcaacatagcaagaccctgtcgcttaagaacaaaatgtgcaagtagctagagtgtttctcctgattattttgtgtatgcattgctatggtctgagtgtcggtgttccagcagaattcaagtgttgaaacctaatcccaaggtgacagtgatgagaggtaggacatttggaagatgattggattgtaaaaacaaagccctcatgaatgggattggtgtgttttttgttttgttttgttttgttttaattttgagacagcgttttgctctgttgcccatggtggagtgcagtggcatgatctcggctcactgtaacctctacctcctgggttcaagcgattctcctgcctcagcctccctagtagctgggattataggcacacaccactacaccgggctaatttttgtatttttactagagacagggtttcaccatgttggccagcctggtctcgaactcctgaccttaagtgatccagccgcctcagcctcccagagtgctgggattacaggcatgagccaccacgcctatccaggattggtgttcttataaaggaggttggagggagcttgtttgcctctcccaccatgtgaggatacagcaatagggtaccatctataaggaactggacctcactggtcaccaagtctgctgacatcttgtgatctcagaattcctaacttctggaactgtgagaaataaatgtttgttgtttattagccaccctagttcatggtattttgttataggagcctgaatggattaagacatgcatcatttttatttacacagagtaaaacctccccaagtatgtggctggtagggctccttgtcctgtttactctgaaagaaacttcactctgaaagagcatggttagaacataccactcttttggacccaggcatggtggctggagcctgtaatcccagctactcaggagaccagtatcacaagataaagaacatttcaggtattccaaatgtcctagaaaattatgagcaaggagaactctggctggtggcaggagagaaaaaaagcaaaataagaaacagaggtcttgaagtgggaagatttctctttccttatagatgaaattttggcatggcaaagaacagttaggaactgatagtgcctttgaaggtctgaaccagtagacaggagagaatgttcctctttctttcatagaagcagtgactcccttagagtgaagtgactgtcatcccccaccccaaccccagtgatttcctttggggacattttatacagagacttggaagataaacttgtcacttttagggaaatactcattgtagtaagttatcttcccaaccacacttccttaacggatgtgtcaacttttagtactacaaagtaactaaatattttaagtagcagtcatccacactgcagcatgagtatgtcatttccaaaactagatgctgtttaaaggtttaaattagcatcttcgaggtttttttttgtttgtttgtttgttttttagacagagcctcactttgttgcccaagctggaatgcagtgacatgatctcagctcactgcaacctccacctaccgggttcaagcgattcccctacctcagcctcccaagtagctgggactacaggcacccaccaccacacccggctaatttacgtattttttaatagagatggggtttcgccatgttggccaggctggtctcaaactcctgacctcaggtgatccacccatcttggcctcccaaagtgctgggattacaggtgtgagccaccgcacccagccccttcaaggtttttaatacatctggctttaaaatctcagaagcctagcaagactacctcctagaaacattgatatttattgacattttagggtattttctattgttgctctcttaaaggcaaagaaaacctatttttctagttagaattaccccttaatgcattgacaactctgaaagaagaaattctagccttttctttcctaatcagtgaaaggccctttcccccatattgccctctccgccccccatatttgggtatttctagcctactttttcatctgtatgccctgttactatggtgacaggcaccatagaaatagagaacaaagcagatgtaataagcatctcctacatggtttacaatgctaaaaagagatctaggattttcaggcgttgctttttcttcactgatgatagaactggaccagcccaacattgtgggggtatagggactggggttatgggctgatttggaccctacaattcccatcgtcttttactttatcagtccttattctacccaggagtcatgaaggtgaagaaagactaatgggagtctgcatggaaagtgtaatatagaaattagggccacagaaacaacacattgcaaaggacagatacttctgctgaagcatattccattggagggaatcaagccagctgtaatgttcaccagtcattgatccttttgcttgattttgcattgttcaacttaacctttcaatagaaagatttatcagctaggtttctcccacttcaaggtacttagcctatgttagattttttttaaattaaataaaaataaatctggattactaataacttgcaatatttgctcctggccattggggccattgaacttttaaaaataagtagttgaacagctaatgcatgcttattttgtgcttgttttagaattataattaaatattcagcagatattgtgaaagttagctttccataagatccaggagaaattttccataaattgtttttaaccttctacatgcatgtctattattttatttattacatcttcacgtatcatttctggatatctttaatcaattacctaagattaaataaagtgttccagctactcaattccatcacctggacaattatttttaagtttattattttttaagttttatgttgcttatatttttatgtttagggacctgtatagttttaaaataatactatgtttagaatataagttgtgatacatgaatcaaatatttggagctatagaagctgaatgatccatgtgtccattacattgtagtatgagggcctgaaactccttctacctctaaattcttgtttctttgcctatgtggctcttttctttttacttgacgagcaaaagaagtatactttgccaattttgttatctgttgtaatggccctaacagtgatgtgaaaatatgtctgaatcaaccccacgctttgagttttaaaaaacccacacagtgtaaccttccccagtcatgtcagcttttcccctttgtgacacgaatgaaagaacaagagcattggagtgggagttggaacagccagtgctgaccgtggcacagcctctgccaggggtgagcttggagatacatttatatctctatacaaagcatacagcttcctcaggaagagcccaggagccccaaggaaggttttgagagattatatcagattagttttgggaagtaacactggcaactgtgtggagggtgtgctgaagatgagagagatcatattgcgagaccaagtagggcagtgggatgaagtagaggagactagtagaaaagttagtttagaaaaaagtcataggattaaatattctatttccttttatcagaagatcctatggtaattacttaagtcaaactgtgattactgacattttactagttttactatagcttctaactttttcagagaaggagtggattaagttttgaaccagatgatttggggtcaaattgtcaaatttcaggttaccagtgagtgtgctttatattagcacaactttttaaattatagcttctaaacatttatgtcttctgactggaaatgggaaaagatttgggggaagtactatttgttctatggaagaataatgacactttgcacaggagaggtttgctgaaaccaggtaatccctccaggcagcttagctcattgtcttccagaaagctaaaaaattatgggctcattgggtaaggtttcttggcctagagagaatagaggttgtatttgtgaggagcttattctttcatggattgctgttctctaaaacctcctgggcagagagagttcccatcacttttgatgttgtcatcactgtgaacatagaaagtagaccaggtagagttatttctatttttatcaattaaaaagctggctttctggccgggcgcggtggctcacgcctgtaatcccagcactttgggaggccgaggcgggcggatcacgaggtcaggagatcgagaccatcccggctaaaacggtgaaaccccgtctctactaaaaatacaaaacattagccgggcgtagtggcgggcgcctgtagtcccagctacttgggaggctgaggcaggagaatggcgtgaacccgggaggcggagcttgcagtgagccgagatcccgccactgcactccagcctgggcgacagagggagactccgtctcaaaaaaaaaaaaaaaaaaaaaaaaaaaaaaaaaaaaaaagctggctgtctgagagactccataacttggctaaagttaaggcagatggtaggcaggataagagttcagtctcctccatctttttgctgacttctactctaccacatgcctgaagtagccaccttcacacattcttcagaacagaaagcattgactataccaagaatgaaattttcctgataattttagtaactgttctcttgatataatgtacaaattatattatttaaatgactaattcacagattaaaattttttaatgtaatatatggcttttacctttaaaagttgtaaaatcctgggagcatttgaaacttgaacacaaacccaaggctactctcgtgtaccaaaaatatggaactagacagagacctcctggccttgaacatgtcttgctttcatgacatcttggttattatttatttctgcacctacctttagctaagggactagagaagagtgtcatctgctagggcaagcttttgagctctctggctcagttctcacagtggctcagtctcctatgagtttcagagtcttcaaggtggttcctctacctgccccacagcatctgaagcacttactgtgattataataaaataactgaggatttgtttaatctttgtctctgtcaccagactataaattccatgaggtagagatagcacctgtttgaggacatcatgggttttgtttattttgttgcttctccagggccatttaccacataacagccactaaatatttactgaatgactgagtgaatgaatgaatgaatgaatgaatccatgtttagcctgttgttcattccatggagctaagctactttttatttttctcagagatctctttgaagaatattcatattgatgctcgttttactattatgaatttactatttgtgcatgtttagataagtcacataactgtctcagttgccaatctgtagtggagaaaatctcatggggttgttgagagttttaaataatttatataagcacttataaaaatacttaacataaagttatctcatcatgagtattggcatccattatggttataatatacaagcatattcaactacttcaacctcacatcagctacttgttagggcctatgtgtcctcacactagacttgttttgtcttatgtactggggggacctattaaggcaagctccacctgatttatcttttgtaagttgtcagttcagttaactagacttaaatttactttttcctggtaatttttaaagtagggtcaaattacttaatgatgaaaaattagacttgcatagttgttgatctaataaaagcactattgacaaaatagaaaaaattatagctgtctcaggaccagatcacaaaagcccctaaattttcgttatagcctgaaacttgttcaatcttcacattttctgaaattcagccttattcagtaaatagacaagtaaaagaaaactatggcccttcacatttttaatgtatattttgagacatgtagactataaccttgaaaagtcttttatgctaattggtcatcatgggtaacaatactcaaagtattcaaagtaacataagtctaaggtataaattcatatttcatttgttaggagtctaaaacttctttgattctttttctgttgtgcatgaaggtaacaaacgccagtgagtggtagttttcttattataaaacaatctattatacaagaaaaaatttattctaatattatctaagttttgaaaaactgattttttttttttttaagttggagtcttgctctgtcacctgggctggagtgcagtggcacaatctcagctcactgcaacctctgcctcccgggttcaagtgattctcctgcctcagcctcctgagtagctgggagtacaggcacatgccagcagccctggctaatttttttttttttagtagagatgagattttgccacattggtcaggctggtctcaaactcctgacctcagatgatccacccaccttggcctcccaaagtgctgggattacagacatgagccaccatccctggccatttttttttgagttggggtcttgctctgttgcccaggctggagtccagtggcactgtcatggctcactccagccccgatctcccaggctcaggctatcctcctgcctcagcttcctgagtagctgggaccacagttgcatgccaccaagcctggctaatttttattattattaattatttgtagagatgaagtcttgctatgttgcccaggctggttttgaactcttggactcaagtgattctccctccttggcctcccaaagtgctgagattaccatcataagccactgcgcccagatgatttttaaaatctctataaatgtctgtttatgcagaacaaggtgtaagaatacatagttatttggtgtgtgcagtagggtttcatgagaagggaagggaaatgaaggaagctattgttcagtagtttacacttttgattcttattgttttcctctctagtaacatcccagagatgttacacatggtaaagtcgtagtaaaaaaaaattatttctaaacaaaatgtttctttccctaatgagattctggtttgggtgctttattgtcagtgtgctgggaattttggaatgttctctgtgtatttctatgattttgacctaataatctcatttggatttaccaggctcagcagctgttgtttttatcgactattgctaagtcatattaatgtccaaagttgaaaaatccttttcctatgttggcctagattattcagagttttacaaaatttaagccttttctccaatgcctgtaatttttatttgcttgtaagaaaccttataataataataatatcccagtgggtactttagatgggctaagcctatttgcagatctctcgcaggatggtagtgaagtttgcaggatggtttgtagaacagtgctttcttgataaatccatctatcctcttttctactggaggaagaaaaaacctcaattatattgactttaagtactgtttgatgtaacatgatagatctttgcagtttgatatgaatgattttcctttggaaaagcagaatgcaaaactgagaagtcaaagacattgagaatgtcctaggagagcataaactcaaaattataaccattttattggatacaattggaaagttttatttgaagaggaaatcagatgttgaattttgccttttaattcaatttttaacgtaataggaagttttcttacatagttgaaaacatatattgctttctttgtaatttctaatttgtttaattataaggcagtggttttgaggtctaaaaaagatactaggtatgctgttacattcctcccagggtgagttttcttgattcttaatcccatctgatctctgcactatcacattcccttttttcctgttgggagtttaaatttattctccttatacatttaggattcaaaccaatagctcttcttgttgtctagtgatatggtcaatgatttctgcatttacaggtaaagatgagcatcaacagtgtactttatcagttataaaatcatctttacataataatcacatttgattccgacaatttagagagggagagaggaagaatgactgaaattaattcctattcctcagagaagttacacaatatacccaaaatagcatgtaaacagaagggcaggacaagaaccaggccttttagctctgtgtccagtcttctttccaccctgtcatactgtgtctgcatgccctgtccaaggcatttaccaagacatttcccacatacagaatttggttaatatgctgaataaaattgttgtgtcaaattttaataaactctataaaaccaattggatatcctttggttctttccctgttaaacgtgaattcttgcactatatgtatggtgagagaggtttcctgctttcaaggccaatgacagtgagtgttcaacttaagagttaagaacaagaagacaacaataagacagatgtcccaagaaggcatatgactcacaagttcagtggaaacatcgtaggaggtgaggcttgcaggatgtgataagatatggaaagttgaagaagggggtggacgggaaatgtttgagcagaggtgtataggaaagaatgtatgcagtactgaaatcattgcggatgaaacgatacatccaggatttgcttcaagacacctggcatgggtgtgatatgaattgatcattgttggagctgggtaaatggtatatgggggacccttataatattattcatacttctatatacattagaaaatttccatagtaagaattttttttaataaaaatggtatattatgtgttttttgaacaataggttaggctagcctatcagaatagagagcatgttcaaaggcatagtaggaagggttggaaaagcggcctggtaccaaatttgatggtgggggaggatttgtgaatgtatgctgtggagccttggtgagtgctagtaggcactggttcattggttggacttgagttctgcttgactgtgagcccaaagtggagttgcagtccagtgttggcatcttatcaagaatggcagtaactccactttaccatctgcttcaggagtgtcagatctaatgtgccatattcagttctggaaggcacatttaaagaggactttttgcgaaccaagtttcaatggtgggatatatcaagcacggaagagtaactcacatcttcttagccatcttctttctgtcagaacactccatcagctcatggttgtctacctggaatagatgtattttcccttcttccaagtctctctactactccctttcaaactctttgtcttttgagagccaaagaataggcaataaaaagcaaattaaacacattgctgtgattccttagtgatatgaaggtacataatcacaaaggcatctgactcaacctcctggcaacagactaacagctgactgagctatacccagaaaggagccctcagtgtagctttctgaagcaaagttcctcctccttcttgctgaagctatttctctatagctaagtaaaaaagacaagagatgtatcagaaatttggttaattgataatattaaggaattgttaattatttgtatatgattcagtaatgattctagagctgacacacataagcttgtgaaaactgattgttgtgttataggaattttcaagctagttaaacatagtcattaaaaactaaattgtacaaacttaaagtactaaaagtaaaataaatactcaaaactctcacttcctaattattttgctataatttgttgttatctatgctcttaaggttatttatgtctaaggtatctgcagagtagaagtgctgtataatgatgtgtgacacatctcttctcaactccacattcagtgacatcatgttggtactttgaaattggccatggtgggaatcatggaaaatggcaaagaaccagggtcctttttttttttttctcagaaacctggttgttaaatatttaccagcatgccactggatattattatgatattatggttatatcttaaagactctttctgttttagagatccatactgaaataagacagaatatatctagaatctgtttcaaaataatccaatgagggtgagtattgtggtgttgaatcagagtggtgggcagatgggtttcattgcaccactctctcaacctttgtgtatgtttaaaattttccataataaaatgttaaaacagcaattatggaatgttacaatgaaaataaattgcctgtacctagtagacatagatagcaatccagtctggcacctgtctttccttcaaactttgatgccaacattcctttgataaaatcatattctcaaattttggagcacttattttgtaaatggttcacataggtcattgattttaaagatctgtttctcattaaataaaatcaagaaaccaccatgtgcagtttatcatgcccatcagggcaaaggaatccactagagccttcccaaggacagcaaaaagctttcttcatagtgggcggggaggggagggggtgcggggcattcatttgttggattgcaatcctgtttgcaaaaccagttgtacaaatagggaaaaatgaatcaagtaattgctcagaaggctacttgcagctacatgacagctgtgtaccctttagatttattttcagtctagctgatttgagtcattgcccaaaaaaggagtgctgtcaagtgaattgtcttttatgcagatgatgacagcctttctaaatattgcctggctgatataagtgatttgtttggaattattggcctatgaattaaattagtcctaatccaaatgattttaaaagttctcgcttagattatatattgattacatttcacaaagagcacaaaataactatatttttttctagcatatatttagacatcaaatggaaaagctatagcatgaattactcagaatacatcttttttttaagtttgaagttttaatttcattcttaatatcaatttggagtatggtaatagtttctccttggatttgattatgcaataatgcatacaaactaaatccagttgacatggaaaaatatttatgttgtgtaatgaatgcaagtggtcactatgattatcccatcaaacagagtagtgaggaaattattaggttttataatgaggacagagaaggacaatcagaaccttgagggggaaagtagtctattctgtcttataatattaagttaatgtgagtttctggtctgtgtagaaatacaggcttattctcttaaggtaacgacaaaaaaattcaaagcctcaagcaatgaaaacagcagatgcaaactcaggtttgttttgacccctcagttttgtttcaccctgggctgaaacagatactgcaaataggaattgagatcagggaagccagaagtcagaagagtgtgtgtaggagaaagatcattataacttctcctcataagacagagctggacagaaaaatcacctggagaaactgggagcctttgttgacactttgctgcagtttttcccatgttaattcctttgtaccaatacccttcagggctctgcagggtcccaccacaacatacattctgagagtttgtttgaggcgtataaatacaatagaagtcatagttccaaattaagttggagcatgaagattggggtgtgattgctaagaaaaaaggacatattaagttcctgaagtgagataatcacagacttgtgattattcactcaaaaactatttactgagcccctactatctgggctaggtgatgggataccacagtgagccctacaaatagaagccctgtgctaccagagcttactgcttactgtgcagagaaaagtgataaacaacataagtatatagtacattaaacggagacagataatgtaggaaaaacaaaaccagagcagttcttcaaaattaaattcccaaaggataacattttccaactctatcaaagctagcaaagagaggagattggtatttataaaaatacaacacataccacatcagaaattccttcatgtttctattaaccctgttgatatgctaaatatattcactatctctacaaccattagagaacaaagtaaagatgaagtagcttggaaagggagcagctctttaacactgaagtgaaatgaaaaatatgcccttatgttttgttagtgagttgacagttcatgatcaggaagacaatgagcacctgcaatagggaggctgcaagttatttttaaaaagaatgtctttgattgcttccaattctatttatctccagttggacaatcaagtagaagtcatttgaagagatgttttgtagttatgtggatgcggatttgcatatgtgggtgttttcgaacatataaacttttaatgaattcagtcatcttcataaaaaactaaattgtcccaactttaaatggaaatattctggaaggaaggtattaaatgagttcttttatatgggagatggactgaggctgcattgagttatttattttaaataacttttaaaattttaccaagtgcaggtctttaagtccaacattggcccaagtcttcagcaaaaacagcttaacaaactcatttatattttttcctctccctagggctccagtttgtgttttcagctgcctactagacctcactacctggatggcccacaggtactgcaaactcagtgttttaaataacacactcattagcccccttcatcattgaccctgtggccaaagctaggataagtcacttgtccttgagtcctgactctccttaacatctaatctgctaccaagtccttctaagggatacctaaggagccctagagtctatgcccataagctagttagcagagaacctgctcgaaagcacttaaaagtaatagtagtacttttgcttctatcccctcttctcttcacactcaggcacatcttagttcaagccacctttcctgtttatcatctggacaattgatctatcctgatctcctccctatgtgtatttctcctttttaacgagttgttcatgctgctaccaaagtggcaaattctaactttttaaatttaagatttgacgctgactggattaagtcactccctttcacaaatacctttttgggttcaatagaatggtacctgtgtctcattaggtctggacctaaatggccttttcatctcaccttcttcagctttcccgctaggttcacttgcgagaacagtactcagtcaaaccttagctatgccaaacctttaataaagcataaaagattaattatgttaattattagccttgctttgatatgaaagggcaaaattcaccagttagctccttggtcttccactgaatattggaatattaactagcactttataacagagcctttcaccagcccaactttactgctcttcccttttcccagctctctagtcagccagaacccacagttctgcctaaattccttcattcctggtcagtctcactgatactaccatggtcttcactctcactgcttcagcattgaatgactgcagtaggttcctgcctggtttccttaccttcatttcccccggcccccgatcctgccccattgtatctctcatgccttaccaggttagttttcctaaaatattgcttgcattctattttagaatctataattgttctcttttgtaagtcaattcaagccatactctcatattctggaatctccaaccgtaaatgtcctcactgcctcatttatttgtgaagtcgtccccctctggccgtgaagatcctctgcagcctcactgctcttcctcaggcaacacccctctgctctttccctccccactaccccgctggagatttttttcctgctccagcccctgcatatccctaacggctcagagataccttatctatgttgccttctccattccctgagctatacctgttcatcttacaagaggaagctttggatggttggaagagcaccatgtttggtgtcagttataggatcaagctcttgctcaccagccatgtgtcatcagcagctaattttttttctctctgagcttgtatccttatcctaaagtcaagtgtatgatttaaatgactatgaaaaaaatgctccataaacttttgagcattatatagatgttcagagttgttccactccactatttaatccttcatttttttcctgttcactttttgtacaaatttctcattttcctaactttataagtgttttaaggatagatagaaaatgaaatatactttcttatcttattcagcccataccacagcaaatgctcaatgaattctccctgtcaagagaattcaatgaatataaatataaaggatcaggctctgtggtgaaagtgcagtatttgataaagtgagtgagggagtaacaagaaaagagggtccccaaagaaaacgggactgactagcggtctggcctgcctcaatacttttggtgcgtccacagacaagctctgaatgagacccactagccaggctcttcaacctcttaacaccatggcaatagaattgactcagtttcttaagtgtgacactgttcttaaaacatagtaggctctcaagtgtttctagaattcactagccacatgatttgattattatatatatttatatacatgcatgtaccgtgctaatagatttgtctgtcactgcttatttattcatagttttttttccttttacatttcattcttatacagagcagcagtatgtctgctttgtctgcagagtagcactaatattcagatttgcattgtgtttgtgcagtgtctaataggggctacctgatcacttgctgaagtcataggtagttcaggaactaccttcttatttagattgctaggacattttcataagaatattgtactgtatataatactaagcttactcaaatcctgcttaaaattaaaaataagtataaagaataataattagtaaatgagaatatattctgtttttgcatggtgtcttctagccccttctcccacacccaatctccttggaataagagatatggtaatggagcccagaaattcatgatcctacttagtatccatcttagcagaaaaatgtaaaccatagtgagttttgtttgctaacactatccttagacaataagaataatcgttaggtacttataataactctcactatgtttaggttactgagtcagttaaaagcagactaatcagtaaaatacattttctggttttagagagcttataatttacaatagattaaagggaatgacagtaaccacagaaaaataattaagagaaatgtgatttacttgttacctaagaagcaaaagatacatttttctccaaagtaagtcattaaagattgcaatcgtttataccagagttgatttactagcatgaaaatgattgcttaaccctgctccctgtgttcaccaggcgggttggtgaaagcttgtgtttcagatgagccagcctggctacaggaaaagatatttgcatagaagactggggacactgccacacaagaaaatgaagaattatgcttttgaaattccttttattacttccttgattaaattgttgtttccaagggaattggattttcagatgaaattttaataatgcttcagataattccaattgtgttctttcattttactctgctttcacaatatgatcatatttggtttcacttcctggttagtgcacacaagcctgttgctgggaaatatttgaataaaatgtttatgagagaagactattgtttcaaagtagaggaaggcatttttgtttgatttagacttagttcatgtagtaatttttatagaaataaaatttctgacatgtaaattggttaaaaacactctttaaaaataatttaaaagacttatttcgcactgaaattaagttaccttgtggtgaaatcatctcttctacagaagtgtttgattaggtaccagttgtgcaacaaatgaaatgtttgtccataattagagtaattgtaacttgattttaaacagaggtttgattgcatggtgagtgtcataatgccaaatgatttttccattgattcagggcccagtgtattcttaagttgatgtttgatctcagcatgtttaacctggcccagaatatatctggcacccttttgtgtgtgtgttgtaaatgactgttcttctgtgttacagcttcatacaaaatctttgcctattctaaaagtggtatttttaccattaagtatttaattccaccagagtactcaggtattacctagtctaaaaaagtacaaatcaaatttatttctctttggaaaaagtcagttaagaatttcaggaaacgtctgcttccttaaaatctaattactttgtccatttgcatcagtttactggctaaatctgaaagtttaattttttaaagcgtaactatttttctactcagaattttaattttgaagtccatttatgttttatttctcaaggtacctagatatataccctaatataaaattaggcttcctgctgtcaatcttgaactctctccaatgaattctttctgatgctttcaaattaattttcttgatatataatagtatgtccctaaatgtatttttctgtcattcccttgctcagataccaccagtgacttcctactgcttatatcaagcctaaattcctctctccttgtaaaaagatttgaatattctaaaagtgacatatttaccccctaaagcaataatttctcaggtgcctgttatgttctgggcctgagttaaatcttgaaaattttttagtgtggaatttgagacttaattagcatttgatcctaatcacctttctactcttagccccatcatttctgtctcataaattccactccatctatgcatctgctcactaattcattaaattgaccttggctttcttttgccttctttatttgttgttccattcctccacataaaatattatgtgccctgttattaaccaaccccttttaccccttattccttcaccccctaactatataaatcttacgcatctttcttggtccaactcataaaccctgtacagtgactattgttcttctagcatgtattactttaagatgtaactgtataattcatttgtttgatctttcttgtacttatgccttctttcaccaatgcataagctccttttgagatttaggtctgagtgtgacacctttcgattgccttcattacctgggccagttctttgcacccagcagaaacttcatcatgattgtcgagtacacctagtgtcagagcatgtgcaagatgtaatatttccacttgctgttggtgacacaagttggtgttacataaggccttcttcatttctacctttcttcaactgagagtcacaaagttgttgccttccttaaatggataagactctccacacaataaaaacagatggtattgtcaaatataaataggtaaatattattttttgtgcatggatgagagagaaaagacaggaaaaaaactacacttgtagttttcttggcacagtctttttgcccttcaaatttggttgctaagaaataaccatagattcatctgctgtgtataaaggagaaacttagagactctgtgtgttgtgcagctgattttgtcactcagctatgacagagccccaccctgatttagaagtcatgtgtcaaacttccagatggggagtatatcttttgtctcctggaaaaccattgaaactctgtttccagagggcccatgtttctcctctttgctaagaccaagtgaagctgcagaatcctgctgagttatgaagcccgaagccagcctaatgtggagaaatgacctgtcaccttttttggaatgtaggcattctatgtgattcatattagttatttgtgcgttattttagaagaaccttactctatcacttcagagtgcccagagctgccctaacttattctgaatgctatggctgagctaaattcgtcttctctgctgatggggctcaaatggagtctttagtctctgctaaaggattcaggaagcctttcatgtcatgtagaatttgggatgtaaattgaaggtaagaaggtatgaagattgtaagatatttttgcagtttttgcttccaaacaaggaggtctaattaaaactcttgtatctgttcaattaatatttagtagcagcttctacaagctaagccctggggtgcattgataagcatgatacatgtagaccagacctcagagagaaataaacctttaatcaaacaattacttataggtttgtgataaatgttatgaatggaagggatagagaactacaggagcatgttgtgaagtgacctgtcatcatgtcaaggaggtgtcccttaggaaataactttcttagtcattgactcagggcatcagtgacttaaacgactgccttgttttggccttgaaagcaagcttgaccttttaagtgaactatctggcactgtttgaaattgcatcagccagttggaataagaattcgctctcctgtgagacttttcttatacagcatcatgttatgtgtgtaataaagtcatatttggcctttgcccccatgtcacatcacagagccccattcctgataagagtgtcctttgttattcatagcgagcccctgttaaccatacctaagtttgttctaatgtggtaactcactgtaggcacctgggtagcttcaggatggggcctggtcaccagaggatctaaccatgtgattccagcattggaacttttacgcccaccatcaacttctggaaaggggagagggactggagattttcagtcactaacggccaatgttatttaatcaatgatgcctacataatgaaattttcataaaaacccctaaacaatggagttcaggtagcttccaggttggtgaacacatcgaagtgctgggagggtggtgaggttggaaagagcagagaagctccatcctcccactgccctcaaataccttgccttttacatctcttccatttggctgttcctgagttgtatcctttataataaatcaataaaggtaagtaaagtgttttcctgagttctatgagttactctagtaaattatcaaatttgaagagagcgttatgggaacccccaaatttatagtcaagtcaaacagaagtacaggcagcctggattcctgggacttgtgattgagtttgaagcagaggcagtcttgtagaatggagcccttaaacctgtggagtctgatgctaactctgagtagttaatgtcagaataggattgaattttaggacacccagttggtgttacagggagtgatatcagaaaaaaagacatctcactctgcatttaccctaaatgtaaaccagacattctccctctccagctgaggagcaaatggcaaccagtgattttgactaccagcagccattaaccaagttacaaatatgaagcattaatttaacaccaaggtaaaaatggacattttacctgaatcataagtgacataagagcttgctctttcaaaacagactggtttctttctgaatgctcttgcccaatgcttcattccccacaccttcctccatggaaaataggagctggaacagataagcagtgttggctgactggaatcttttaaaaagccttctctgggttgggcagttaggcgtgtgcactccattggcacagtgagccagagattgtgcgcattctgtttttgtctttttccaaatccccagttgacagacattaagggatccattatcattccattattgcaaggcagatttggagaagaattttctactgagatggttgttttgtcacagtgaaacaaatgagtgacattctatgtttgggccacatagttcttctgaacccctgggcctcaatgccttttttgaagcgttctttttgacttcacagtaggacccaaatatgatgaaatgtcaatgtctttgcaacacttgctattttgtttccctggaaccacagtggcataaataaagctcacttcctgggttgtttttttcaaagataccatcccaaggccaaaagcatcctctttttaaatcactttgctagtaaaagaattaggtttctttataagagttctcttttgcatctgtttgctactgcaatgatttcagagtgcttccatgatatttattttaccactaatatcaattcttgagccagtgtgattatctgtattttaaagctgaggagatggaggcctgtatttagtgagtgatttaagcccatgcagatgatggtggaggcaggcctgggactcagtccagtgttcattttccacactgctgccatggaaaaccagcccctgcttctcactctcactagtgcgaggagcggagaaacaaaatgagactgtactcagtcagcagcagatacataaaagtttcctaatttttatttatatgtttttcctaatttttatttatgtgtttttatgttgtgtgttcccctgctacggcttggggaacattgaaattaggaaatttatacttacctaaaacagctctagaacatttacacagtaacagatcagcaagtagagcaattactctaacatgtgcagggttgactctcacagcagtgactgctgctgaccagtaagtcaagaaaacttctgtaaaaatgcctagtatctgtaagcatagacaccagtgaagtatagatagaaataaagccagagacttaaaaaaaaatcccttgccatcacagcagggttccaaactgattggaatccatgagacaggcagcatgataaagcaagaggtggccatagctaactgtgaggctgagtgagtgcccacagcgagtcacagggtgaagcatcctgttgtcatgtttgcactgttgtcaagtgatcagtgatcactgtgatcacagaattcataatccagtctgccctgcattttggggtcaaacttagcactcctgggaaggaaacagcaagagccaataaaacagaaagcaagagaagctggagatcaccactctttcccctcacttcccattggcaagcctccccaaccctccctgagacatcactttgtctttataataacaaagcaacaagtcaagaggtgacatgttttagaaagagctctctgactgcagtatggaaatgggactaaggagggcaagaaaggaggcaatttcagagtcgtccagcccaaagatgacatgggccccatccaagacagtggtagcaagattgtagaaggggctagtttaagagaccattggatttgagggaatagaggaagagaaagtttctgggttaggtgcctgccttagtctgtttgtgctgctgtaacaaaacacctaaaactgggtaatttataagtaacgtaaatgtattcctcacagctctggagactggaaggtccaagatcaagatactacagatttgatttctggacgttgaacatggtgtaggagtagaaaagcaacagggacggaaggagagaacttaccccttcaagcccttttataaggcactaaatcccatcattgagggcagagtcctcatagcctaatcacctcctaaatgctccatttcttaatattgttgcactgaggattaagcttcaacatgaattctgaagaggacacaaacatccaaaccatagcagtcaatgccttagcccttgatgttgctatcaacctgagattcggggatcaaggaaggacaggtatgatatctaggtgtgggaggaaacggtagatgcagtttgggacatgctgtttgagttgcttccacacatctcggtatatagccaggaagatgttggggagatgattctgcagcttggggttagggagctgggctcaccatatacacagaggtggcggatgaaaccttaagcataatgctcaggatgtcacccgagcatagcatatgtgtgagtagagaagctacaggcccaagactgaaccccaatgcttaagggataagctagtattaggaaccttccatggtcctaatacctttttatcttcagttaaaattagtacttcctgccgggtgcggtggctcatgcctgtaatcccagcactttgggaggccgaggtgggacaattgcttgtgtccaggagtttcagcaacattgtgagactctgtctctacaaaaaaattaaaaaatagctgggtatggtggcgtgtgcctgtagtcccagctactcgggaggctaaggtggggtgcattgagccatgaccgcaacactgcactcagctaaatgacagagtgagaccctgtctcaaaaaaaaaaaaatatatatatatatacacacacacacacacacacacatatatgtacacacatacacacacccacacacatttttgtgtatatgtgaggcagagtgagtgtccatagcaagccacatggtgaagcatcctacttatttgtaaacatataaataaattttaaaaattaatacttccattatatgcttggcttgatgaccttttcttgctataaccaatttttagtcgctttttcaaagagaccagtttatgagttgacaattgtagacattgtactccttttctaaggaaacctacaccattgtttttcctggtgttctaacttttaaaccccttctcaatcctcaccactccagaggagtctttcagactagatattccctagctccaatcacagttaataaactcatagcattgtgggaaactggaagtagagactctatcaatcatttgttctctctaacattacatgtaaggaaaattagattacgtgagatgacaaggccaacattatgccgtcagtaagatgtaaagtgaggagtagaacgtgggtttcctaacacctagttgggtattcatttgatcaggtcatgctgccttgtagtgttgatccttccctatatctcagtaatggttactttccttgatttgaacatctaatcacattactacattcccctcatccaccaatatagttctgtgcagaggcagagcagctgtttctctattattaactcatatataaggctttaatatatctatctcatttggtatatctttttttttttaatactttaagttttagggtaaatgtgcacattgtgcaggttagttacatatgtatacatgtgccatgctggtgtgctgcacccactaactcgtcatctagcattaggtatatctcccaatgctatccctccccccttcccccaccccaccacagtccccagagtgtgatattccccttcctgtgtccatgtgatctcattgttcaattcccacctatgagtgagaatatgtggtgtttggttttttgatcttgcgatagtttactgagaatgatgatttccaatttcatccatgtccctacaaaggacatgaactcatcattttttatggctgcatagtattccatggtgtatatgtgccacattttcttaatccagtctatcattgttggacatttggcttggttccaagtctttgctattgtgaataatgccgcaataaacatacgtgtgcatgtgtctttatagcagcatgatttatagtcatttgagtatatacccagtaatgggatggctgggtcaaatggtaattctagttctagatccctgaggaatcgccacactgacttccacaatggttgaactagtttacagtcccaccaacagtgtaaaagtgttcctatttctccacatcctctccagcacctgttgtttcctgactttttaatgatcgccattctaactggtgtgagatggtatctcatagtggttttgatttgcatttctctgatggccagtgatgatgagcattttttcatgtgttttttggctgcataaaggtcttcttttgagaagtgtctgttcatgtccttcgcccactttttgatggggttgtttgtttttttcttgtaaatttgtttgagttcactgtagattctggatattagccctttgtcagatgagtaggttgcgaaaattttctcccatgttgtaggttgcctgttcactctgatggtagtttcttttgctgtgcagaagctctttagtttaattagatcccatttgtcaattttgtcttttgttgccattgcttttggtgttttagacatgaagtccttgcccatgcctatgtcctgaatggtaatgcctaggttttcttctagggtttttatggttttaggtctaacgtttaaatctttaatccatcttgaattgatttttgtataaggtgtaaggaagggatccagtttcagcttcctacatatggctagccagttttcccagcaccatttattaaatagggaatcctttccccattgcttgtttttctcaggtttgtcaaagatcagatagttgtaggtatgcggcgttatttctgagggctctgttctgttccattgatctatatctctgttttggtaccagtaccatgctgttttggttactgtagccttgtagtatagtttgaagtcaggtagtgtgattcctccagctttgttcttttggcttaggattgacttggcaatgcgggctcttttttggttccatatgaactttatcttttaatatgtttgtctccttttatatgcctttatccactcgtgctttaggtgagcagttcgaccccacatatccagagactatggaagtaacaaggcaatcatgcaaaaatgagtttattacgaaagtctcagggatatgtggatggatggatggattgatctatttatttcacataggcacccagtaagtgtgtctgctatcactaaggccaaggattgctattactgtcttataaccacctcccaaaacacacacacacacacacacacacacacacaccctctgcattaccaccacccatcctcccactttcttatcttttccttttcttccctgcctttacctaccctattattgccgtccttgatgccttctggtggattgacctcttaacagaagattaacaggaggatcaaatactttgcatatccattaaattattagtgaattttgtaaacatgtgggagcttcaattgttattttaggaaaaaaaatctcccatattcagttaccttgatagtcaccagaagattttttaaatttcttaacttcatatattgctatttatccttttttttttctttccgagacttggtctcactctgtcacccaggctggagctcagtggtgtgcagtctcggctcactgcagcctcgacctcctgaactcaagcagtcctgtctcatccccctgagtagctgggagtacaggcaggcgccactatgcccagctaatttttaaatttgttgtagagacatgatcttgccttgttgcccaagctagtctcaaactcctgggctcaagtatcttcccatcttgagctcctaaagtgctgggattacaggcatgagccaccttgcctgaccatattgctatttagtaaagaagacctaaacattttctagacaaatttgggggatcacattctctctcccacccaaatcaggatgttttcccacccagggaaggatgtacccttcctctggctatctagcatgtcaaccttaagtatttagcaggcaggcaggatggaagaattcaaagtaacaaacaaaattgataattaaatatttagtagttattatggtaacctatgaattatgaacaaagcaaattcaacacattaaaattatattttttgtgttggtatgttttctagtatattgagaccaacaatatatttaatttgtgtaactcatttatattcattttaaagcaataagaacagttttcctgttttacatgagtatagattttttgtggttttatatatatatatgtaacaaaatttactttttaaactattttaagtaatttttccttttttaagtattttaactatttttaattctgtggcattaagttcatttatattgttgtacaaccatgattatagattttaagtcgtgaccctctgcttgggtgacagtcatatcagacctgtctgcagcataaatcttgactgggattatagacattgcatcaaaaagtagaggttaccggcacaggttaactttttaaaaacattgcatcaaggtatattaaaggcttcacaacactttgccttccttatgggccacacaataaattattaaggaaagttacatttcttttcacgacactttctagtaatgagtcttgtgatatatggtgagaactggcatggaaatcacaacttaggtggaaaaccaaacctcctaatatgtaatctggtatttgtttataggatcatactttaaaggcaacctttaaaggattaattttagataaagttactactatgttaataaataatcatgtttagtgcttacattgtacctgctgctgtgctaagcatttgaattgtagttgtctcaattaattcttaaaacaactccattaggttgaaaccattattatggttattatccctattttacagatgagaaaagggatactcagcttatctaagatcaagcagatggtaaacaacaaagtcaaaactcaatcttaactattctgcttccttcccttccaagtctctcacattttcatcagttttaggataagaactaggcctaacaaagtaacacattgaaaacatgtgatactcctttttaaagtctttcaaaaagaaggtcatatatgggtaatatggttttgatttattttaaccacattgtaatcggtatgtctggaagaaaagccatgtaaaacactgttatttaactacagattaaacttgagttattcatcttgttattaaattgttggtaaatgtttcttcataagaggctgtgcaagcaggatgtacccttgctctggctatctatcatgtcgactttaattatttaataggcagccagggtggaggaagcacttgttggttctgtttcactagatggaatactgagcaatgagctaggaaaacaaaatagatctcatttttaaattgtctcttttagtttttctaaaagatagattgtagctttgaaaaaaggagagagctgtcacattctatataaaactcctttttatatagctccttggagaggctgtgtagcagatggctcttagtcagctctgttgtgaatgctgggtctgcttatgaacttgagcaagtttgtttctctgaacctcaactttttcacgtttagagttggctaataatatctacttcaccaggtacacagtaagtgttatccttcccctaactctatcactcctttccattcaacagataattattgagactgttagtgtcaggttcttcctgcacacccgactcttctgagagctcaaaggctgaggcaggaaggcatcaaatcagatattggtgatacagtttaatggaagcataatagaactatcagtattccctaaatgctaaagaagcatgaaggactaactacttgtttagggggacaagtagggtagcagtgtttgttttaaacatcttttgtgtttaatagtcacttaaatactgaacattaaatgtttgtatttggtggtcagaaattagctcaagcactaggctgatttgcagtgcatgacaatttgattttgtctttgggttaagagtgtgttatagcagatgtagcattatttacggtgatcattatcagacgttattggttttatgagactaaagctatctaaggacgtgcaatattgtcagctcaatcatagctcacagcattgtggaattagaacatttatgaagcacctaatatgtggtaggctctcaggagactagaaaggtctttgccctccagagttcaggacaagtgggcacagaatggtttgatgtgcagcatgcttgtttggatgataccttgataatcccattggtaccagtatagttcttgttgcattatactaaggttaacttgtggaaaagggaatgattgtgaaatcataatttgataagatattaaggtcattttggaatgctgtggcatttatccagtgtccaacactgattcaaaatgactctcagtcattataatgcaccagtcatgctgaatttgacagcagttccccattcagatttgtaaaacatcccatttggtgagtaagttggcaaatataaatttttctcctttctccctgaaggatgaagacttagcctctgcttctgaatttctgcccctgccatggggtgatggaaatgcctgaggctctgtcgctgtcatttgctttgccacccttccatacccaccatgtgtttgcaccagtggtgttcacagatctttctgaaagccaggccttctcctctgtacccttggttcctaaatgggtggtgaaacctgatctgctgtccttcatttactcaacaatgatttactaaatattcatggcacagagagggacaagactgacacgggagtgggctcatcaccgatcactctgagctctgggcaggctaaggatttgatgccacttcacactggtattcactaaatatttgttcgccgtgtttttaagtggagtagtgaagcactgtatcgaaaaagagtagcaaaagagccctctgggtccgccttttgccagtttctgacccgaaggaaatagactcatgatattttcttccatccacattcaataccatccttgagaaaggttcagtaggtactatttttgtttaatctagttaagtttccagagatctaaataccccattgatgaatccatacgtagtagctgctataatatataggaccttaagctaacccatgaaagagacattgggatacatcctgggatgccaataaatgagatactgccccagccctcaaagaatcgcaggcaatagcttcacatatattaataaacaatgtaaaagaaatattagtaaatgacttcctggaacgcagatgaaggagcaacaaattcagcctgtgctggtctggatggctcatagaggtgattccacttgatccagaatgggacagatgatgcagtgggatgttctttttttaaatccatctgttatttatgattttgaatttttcaaactgatgaaatgggagactttaattttacatagtaaagccttaaacttatacaattgcccgaggcagggtgaatgagtatgatattccagtgattcctgccatagctgtcctgccaaacaagatcccagtgactatgcctttaacccagttcaagtcctttgaccagagattcttgatatcttgctagatctttcctaagatttaactatccctacaagacaaatatacccaaatggaaaaccaggctcaaattccaaaattgtataatagttctttgtgcactacaaaaaagtttgttttgtcaaatccaccatctctcctgacaagaaagaaacaagaacatcaagagtaagatcctttggggctttttgtttttctatcagaaagctgattgctgctgtgttgttccttaatcctcagtcacttgggagccgttccttatatgtccttggttgggagccttcctttcaggaggaagctcttatttgagaaagaagagaattctcagtgttacacttatctccaaatcttccagaatacccttttatttttaggttatcttggaattgactttttaagttaaaatacacccgtcttgtgtccttcctctggagagagaatgtttttgaaggggtagattagctagttgtgtaacactcattagcatagcaaactggtggacgtgctgacgcaggccgcctgcctcagcagctgggtcggtcagaattgctttaacaatctttgtcctctttttatcctgcctaatatggactatttatgctattctaagaagcattgtcatttagtatttctcatttcattcttccaacttggtacttttatgttttgctaaggaggaagaaaggtgataagtttagttacagtttctaaaattggtttgaatgatttggttttatcctgaataaagaaactccgctatattttgttatttactttacttgacagcagtggcttcaccctaagatgacaaattgtcattatgagagagctgtcatttatatcttctcagcaaaatactcctctggtttaggtaaatgttaaataggagagaggaatgaaatgcattagtaaatatgctgaaaaatgttcaattcaactcaaagaaatgccaaataaagcaacaataagatcatttttcacatatggattagcaatttttttaaataatgatttttaaatctggtgaggtgctactcatacattgttgacaggaaagaaaattggtataaactttctgagaaataatttgatgctgtatatcagagggctcaaaatgtttatgctcttttgacctaattccttcatttttgaagtatatgcaaaaaaaacatatgaagagtactgagatgtttacaacattattagtaataaaaaaaggaaaattgaaaatttagtaggggaatgacttcataaactaggacatatccatgtgtgaactatatgtttccatcaaaaatgatgcttaaagaaacttcatagcagtatcaaaaaagcctgtgatatataagtggcaaaaaaaggtctcagtattgtgccattgaattttacaatggcataattgtataaaacgagtaggtcagcaaaaattaaaaactggacaaagaaaactgtcccaaatgttaagtctttcacgtttcccctatttaagaagcatagatactttcagtttgtttttccgtcttacatttgtaaaatcagtggtactcatatataatgctatcttattctccattaaaaattcaatgtttatgttttgtgccttataagctactgttgtgatgaagtaaaatgtgtagtttgtgacagtgatttgtttctctccctaggtaatagttaacctcttctgtgagaagtcagaaggtgatctctttaatgctttctttttaagtaagttatcttttgtatgtttcataaaattattacatatgttggaaattttagaaccatatcagtggataaattatcttgcagtcctatgatgcgtaaaatataataatatgcttgtgatttctccttaggaatttttcaaattgagactaattgcagaggttccagttgaccagcattcatagggtaagattaacatcctgaattgtaaacacatggtaaagataattacaattaagttggtagcttggggcaaataaatactgaatacttagtatgtaaaagagattactgttcttacagatgttttgagtaaaatctttctgaggtttttaaactagttaataaacagaaaattacatctgtataccttttagtaaaaatgtcatcttcctactttttgtggattttgtatgcaggaaaatatatgtgtatatgatatttgtcattttttaacagtaaaatctaaggaatataataaatggccatggcagacttggagtccatgtctgtatttttctgatttttgtttaaggtaggatttttaatgctaccatggatttggttttaatgctaccatagatttggtttgcatacttctattaagtaaaagatggccatactttctttgagtaggcaagccagaaataaacaagccctatactctgttgctttctgcaaacatcattcttcttttgtaattgcagaagattaattttaagttagattgtagcagtgcttttagctttgacacaatgaatgaagaatttgtacactgtattaaatagcacaactctctgatgttctgaatacagcttaaaagtgtcacccagctctgaacatatagtctaggtgaatcagtggtgaagacagggggtctgtcacctactacattctaaacatcctttttttacagatgaaggtgaagatcctttgacttcttaggtatattcacatttccattctgactctttgagttaactataaattttttttaattttataatttcaacttttattttagatttggatatacatgatcagatttgttataagggtatattgcatgacactgatgttgggagtaaggatgatcctgtcacccaggtagtgagcatagtgtacaataggaaaactttcagcccttgcctccctcccttcctttctgcttttggagtccccagtgtctgttgttcccatctttatatctatgtgtacccaatgctttgctcccacttataagtgaaaaaatgtatttagttttctgtttccgctttagctcacataggctaagggcctccagcttcatccaggttgctgcaaagaacatgatttcattattttttatggctatgtagtattccatggtgtatacgtatcatattttctatgtttttctttatcttttttttaacttttaagttcggaggtacatgtgcaagtttgctacataggtatatttgtgtcatgggggtttgtggtacatattattttatcaaccagggattaagctgagttcccattagttattttccctggtcctctttcttctcccaccctccaccctttgataggccccagtatgtgttgttaccctctatgtgtccatgtgttctcatcatttagcccctacttataagtaagaacatgtggcttgcagaacatttggttttctgttcctgcattagtttgctaaggataatggcctctagctccatccatgtcccttcaaaggacatgataccattcttttttatggctgcatggtattccatggtgtatatgtaccacattttctttatccagtctatcactgatgggcatttaggttgattccatgtctttgctattgtgaataatgctgcaatgaatatacacatgcatgtgcctttataataaaatgatttatatgcctttgggtatgtgcccagtaatgcgatggctgggttgaatggcgtttctgtctttaggtctttgaggtattgccacattgtcttctacaatggttgaactaatttacactctcaccaacagtgtataagcattcctttttctccacaacctcgccagcatctcttttttgactttttaatagtatcctttctggctggtgtgagatggtgtctcattgtggttttgatttgcatttctctaatgatcagtgatgctgatcattttttcatatgcttcttggccatgtgtatgtcttcttttgagtaatgtctgttcatgtcctttgcccacttttttatggggttgttttttcttgtaaatttgtttaagttccttaagatgctggatattagaccttagtcagatgcatagtttgcaaaatttttttcgcattctgtaggttgtctgtttactctgttgatagcttcttttgctgtgcagaagctctttagtttaattagatccatttgtcaatttttgcttttgttccaattgcttttggcatcttcattatgaaatctttgcccatgcttattgtcctgaatggtattgcctaggttgtcttccagggtttttataatttggggtttttataatttggcctttaatccattttgagttcgtttttgtatatggtgtgaggaaggggtccagttttaatcttctgtatatggctagccagttatccgagcaccatttatcaaatagggaatccttttcccattgcttgtttctgtgttgttgaagatcagatagttgtaggtatgcagtcttatttctgggttctctattctgtgccattggtctatgtgtccgctttgtaccagtaccatgctatttttggttattgtagccctgtagtatagtttgaagtttagcaatgtgatgcctccagctttgttccttttgcttagcattgccttggctgtgcaggttctttttttggttccatgtgagttttaaaatagttttttctagttctgtgaagaatttcaatggtagtttaataggaatagcattgaatctatacatatattgctttgagcaatatggacatttttaatgatacagattcttcttatccatgaccatggaatgtttttccatttcttttgtgtcatctccaatttctttgagcaatggtttgtagttctccctagttatctgtattcctaggtattttattctttttgtgccagttgtaaatgggagtttgtccctgatttggctctcaacgtgactgttgttggtgtataggaatgctagtgattttcatacattgatttcgtatctggagactttgctgaagttaccagcttaagaagcttttgggctgagactgtggggttttctagatataaaaccacgacatcacaaacagggatactttgatttcctcttttcctatttgggtgcactttctttctcttgcctgattgcccggcaaagacttcaatactatgttgaacaggagtggtgagagaggggattattgttgtgtgctggttttctttctttcttttttttttcttttttgctaaggaatgttttacttccaattatgtgatcaattttagagtttgtaccatgtggcaatggcattgagaagaatgtatattctgttgttttggttggatagttctgtagatatctatcaggtccatttgattcagtgctgagtttaggtcctgaatatctttgtcaattttctgtctcaatgatctgtctaatattgtcagtggggtgttaaagtctcctactatttttgattgggagtctaagtctctttaaggtctctaagaacttgctttatgaatctgggtgttcctatgcttatgaagcttcatttggccagatatgaaattctgggttaaaatttcttttctttaagaatgttgaatattgacccctaatgtcttctggctggtagggtttcagctgagaggttcgctattagtctgaagggcttttcattgtaggcgacctgacctttctgtctagctgcctatttaacattttgtctttcatttcaaccttagataatctgttgattatgtgtcttggggatgatctccttgtgaagtatcttactgaggttctctgcacttcctgaatttgaatgttggcctctctagctagattggggaagttttcatggatgataccctgaaatatgttttccaggttggttccattctccccatctctttcaggtaaaccaatcagctatagattcagtctttttatataatcccatatttcttggaggctttgttcattcagaaaggccatcttccaactttgagattctttcgtccacttggtctctgctgtctgcctgtcatttgaaaggtgagagtgtgattcagaagggactacatagggagtgggttttgttctacgtattatggcttgcttactgtggtgttggggggtggtcaggaaaatagtcttataaacagcaaaaggtgatggagaaggatttgaggttgccttctatttacaccttctctgtaagaattatccagcaactagtggcttatgcttgtgttaatattacaagaatatggctgcaacttcttttattttaaatagattttagaaaatatggtagcttactaaaaacttcaaagaattgagagatcttttcctaaagagaaaaatattatctacattaaatatatgatgtattctataactgccagaaataagtaaaaatgaagcaatattccttaaagtttaggataactcaaatgtcggccaggcacggtggttcacgcctgtaatcccagcactttgggaggctgaggtgggcggatcacgaggtcaggagatcgagaccatcctagctaacatggtgaaaacccatctctactaaaaatacaaaaaattagccgggcgtggtggcaggcacctgtagtcccagctactcaggaggctgaggcaggagaagggtgtgaacctgggaggcggagcttgcagtgaaccaagatcgcgccactgcactccagcctgagcacagagcaagactccgtctcaaaaaaagaaaaaaaaaaaaagaacaactcagatgtcattaggatttctagaatttttccatggttttaattcactcagtaacttttttatttaatttattacttactatttataattatttattttatttcatttattcaataactttctccaagggcttctgatgactagggtgattttcctaatgtttagagtattcatcaaattgtttcccagacccattatcttactacagttttgggtcagaagctctggaatggcagttgtatgtctctcttctcctttggtttctgtgtattattttatgatgatgtcagcttctttccttgaattctgatttttgtgttccagaaatactgtgcttctcctttacctcactgatcctgcttctctatcctttttcaatcttccttatctaataaaccttccttatctattttatagaggggacagcaattctgtccttcactcactaaattctttctctcagataattcatttctgtggcactgaccaccacttaagtgttcatgacttcctacggtgggctaaacatggctgctaaggcctagcaactctgcctatcaagaggtggagttgatatccccacccttggaatctggctggctgtggtaggttttttgatcaatagaatgtggcagaaatggtgtggagtaatttccaaggctgggccttaagagacttgtagcatctgcttttgtgtgcttgaaacattcctcttagatcttagtctcttgctatgaggaagcccaattagccatatagaggactggcaaggcccctggtcgacagccgaagctgggctcccagccagcaatcagtagaagcaccagtgttgtgacttgaggcactttgggccttctgagtattctagcattccagccctcaccatgtgaactgccagatcatcccatagaatcatgagaaattatgaattgttactttagccactaagttttgatgtgacttgttacacagaataaagaatcaaaacaaaaattggtacctagaagtgagatgctggcataacaaaaatctggcattttggctttgagaccaggtgtcagtgaaggcctgaagggcagttagaatactattgctattggaggatgtcaaatggtacccttgttttatattgagaaaaaagtcagcaagaccatgacctacagtaacatggaacatagaaaatgaacctaatgaacttgtggatctagctcaggagattttccagcaaaatatcagaagctctgagtgacccctttaactccttttttaactcccctttttttttgatactgtgatcaaagtatttcaagaaagagactaattaaagaagaaactattttaattttaagcagactttagaggaagtatttccaatcctgaattttctcagattgtaaatccgactgtttctcatccccagctcctctacccagcaaaagaccagttcctctacccagcaaaagatactcaaagaaagaagtggcctcaggtccaaagatcagatccagaacattactgccagtaaaacttgggctcaaagtgaaaataaaagcaaaggcatagctgtaagacccttcgttaaaacgtcaaaaagattgaaggcagtgcctcatagaacctctcagctagacggaagggcttctaataatttcaagggtcttgtcccacagcattctggaactgatttagatgacaagatcctggacttcaagcctgaatttgatgccataaggggatgaagtttttggggggcttgggaaagggtttagtgtgctttccatgtaagaggaatgtaaatcatttctggccagagagtagactgtggtggactaagcatggctgcagactctttgccactcctcctattgaaagatagagtccacttcccttccccttgaatctggactggccttgtgactcctttggccagtagattttggtggatgtgtcactgtgtagcttcaagcctatgccttaaaagagctgtagctttcacttttatgcttggaatgttgtcttagaacccacccaccatgctatgtgaggacactcgaagagccacatggagtctcacatgaaggagttaccagccagcagttagcaccaactaccagcctaaggaatgaggacattttgtaccttccaaccatctggaaggtgtgccagccaacactatgaagtaaaaaaacctcccacagaatctcaagaaataaaaccttttcttaagccattaaggcttggaatatgcagaacagttagcaaaaactcttccatctctattgttgctgagttcttgcctcttcctaggaaaggctctatcaccctgaatacaccctaaaagtttgaatggttaggggatctttttcatttttgtacatttaattattacataaatatttattgtgccccatgtgctggaaactatgtaagatactatcatatcataggatcaaaatctgaatccacttgactcctgcctctccgtggacctctttatctgatgttgctaggtggtactgaattttaatttcccttgactattttattctttgctttctttttaaacatattgtaaactttagccttattattctattataaagtattgagtttaagagaaaatgtattcccattcccgtgcctgacaaagacatgttttaaagtacacattaaacaaaagaaaactgcttttcaattactaaatgcagtgctcagtaggacagaaaacgtactgcttctcttaaatgctagacctttaaaacatttgctggatgacagcactattgaaatagcctttcagctgctccctttgtttctaatccctctcccataatccatcctgcatctctccatagctcagtgattcactgttctactgcttcacgttcctgagcctctaaaggattcatgtggagatggtagctgttactttgtagtctgtagaggggagagagcatcctagaagtctagtgcaacttccaagaagacttaaaagacaggcacttttctcagacagtttattgtctatcttgtacgctttctcctcctggggagatttgggggaaaaaaaaaatctggcctctcctagaccaattgacttgctctttgggagtaaggcctgggtttacgtttcttttaaaagtgccccaagaggttctaaggtgaagctgagattgagaatcactgtcttaggtttgtgattcttgaaaagtggtccttggaccagcagcatcaacatgaactaggaatttgttagcaatgcaattcttgagacctacttcctaccttacaaattggcaactctgagggatggactaatcacttcatgttttaataagctcttggtgatttttatgtacattaatgttagagaaccactattagaaaaatctttctttctaggtctcaaaactacgactgagtttgggaagcatgtttgttatttgaaaatcactgtactaacaagatttgaaagaactacaaagggacataaggaaactagtatatcaaatgttattgccggtgatcatttaagcacttgaaatatgtttaatgcttgtcattaaatctctcaagaatggtgaaatgagttacttagcataaagaaaaggggcatattaatcagagatactgatgcattgtttcctagagagagaactcaacaaaaggcgaatttaaatttcagttagatatatgttagtcccaagaaacatcttaatgggaagtagcactaaacattttgaagggatataaggtgatttttaaatcaattttcaatgagagtagtgtagagaaccgctatttggaattgttcggctataatccctgcctggggtcagagacagatctttgaagtagcgtcctcttctcagtatataatgacactaaataccatctgatcatcatttgtttattttttcacttgttattttagaaatgctttcaaagtaaaataaggactactgggaagtaaagatgcctaacttgaatgttgatgttacagtcttagagactttggagtaagattgtcaagtgaattcatgtgttttgaaaaagcttttatgacttcttaatgaggtttaaaagctgaaataatgggattagaaccgtaaaatccattactcttaacatttctttggattgatttagtgtgtttgtatattagaaatagatattttatttggattgaatcatcattgtttactaaaatatggtctggaaacaatttcctggacagacagtattaaatggcataatgacaaaagatagctatgcttaaactctctagttaagcacttcaaataggaaaattatcatcattttatacataggaatgctgagatgttttatatatacttaaatccagatgctatatatttaaatattaagttttagttcagtagaaaatatttagtaggaaaatgtttagctgagaaagtagattatatgtaacggaagacattacagaaaactacatttgcatccttattcaaacccagttataatgcagttataacccagttataatgcagatattggatgagttgggaaagagcgctaattagctttttcttcagcttttgagagacctaagactgtggcgtagttaattggattgactgaggccatgtggtctggtgtgcatgaggttgggatttgagtatgaggacctaagttccagttgcaactttatttcttagaggttttgtcaccttgggcaaggttaattctttgggactgccttaatttctatgcatataaacataaagagtaatactatttatttcaatggcttgttttaagtataaaacaaattaatgtaaatgagacatgctttgaaggtagaaaaacagatgttaaattattaataaagtagaaagtatatttctaatatttaaggagaactgtataagaagaatcatggctgagtaacatttttgaattatgcaataaattcttttctggcatgttacaaatgcagccgctatggaagaatatgtaaaatgtttcacacatcagatgtctttatcatgctcagtgcacataaatgcagtcatgtgatatatgttgtaagacttttataaaggcttgtgaattgtgaatgagaaatcttactccagttgtgccctgtagttatgaattagtgtacagctaggttcttatacatactaactgtgctttctaacctgcaaaacaatttgatgctggatggtgactaattgtaaaataggctttgcacatgacttggtaaacccaaagtaagttttggacatgcccaaatttttgatatttgaaatgacaagcttaaaagattattgctaaaagtatcttaaatacaatttggggtcagtactcttaggaagatttgaagtactttttaaaaaggattgtataatacaaaatctccctagaaactttttttttgaggtttcataggcactaaaagatataatttttactttcaattaaaaacaaattctagcattttacaattttctcacagttcaaattatcaatatatatgtaatttaactatcacaaattattgaactataaatgttacaaatggcttttattaaactctaaattaaacaaatctaatataaagcctaagtatgtttaataataacataatttcaagtaatttcagccatatttctgagctgtatctgaaaaagtatattaacttatcactttttctaatgagattggtaggctaaattttaggagcactgagacttatttggatagatgtttggttttaaatatgggtacagaaatcacttggtgtgacaagctgtctgaaaaatgcaaataagaacgactgtgtttgaggaatgagtgagagaaattaacatatgtcgactacttagaaaagctgtgttcatctaagtgttgtagacacagaattttaaaatatgttgtgtttaaattgctgtgggaactaacctgtaattgaaaggaagttcagggagtgagtatggtttaatgaatgtgagtggggccctgtagatacacctttgccagaatgtgccccagtgtgaaaagtaaactgacctatgaaaatctaagccaggcacagtggctcacacctgtaatcccagcatttgagaggccaaggtggaaggattgcttgaggcccagagtgtgaaaatctatttgccatgtcatgtcttatgactgagagggcttctggagacagtatctccagtttgtttgtttgaatgactgtagctatttcaagatatctatgttttctcatagaatgaagacaaacacagagatggtgtgtctaagaaacttcaaaaggtgtagacctcctgactgaagcatattggatttatttaatttttttcactgtagtaagtataaatatttaaaacactttttttggtcaatgttgacttgtatttttattgaataaatattcactctgtcaaatacccttatcattgttagagctattgttgttttcattcttctacaatttcagttgcatatcttaaatgaatttaattctcaggacctttgagaggacaaatgaaacatgaatttagagctgaaagaaccctttgaaattatctgcttcactctttttatccattacaagaaattcctttgcaactctcctgatggatggtcattttatttatcaagattctgctcaggacatagagctgaactgtttcacagagcagctagttcgatgactttccaccacagattgaatcaaaatgtgattttctaaagcttccatctacctggttctaccttgggaacaaatccactattctttcacctgtgaggttgttgcgcaaaaatgtaaagataaattctcgggctcacctagtcatctattatttcagctgaatatcatcgattgcccgtgttcctctgtaggaaacggtttagaaacaacagctcctggttggttttttgcccattttatctttaacctgtaactgagcattgggtttcagtattgtgatctcacgaggcaggtacagaaggactgcagctcctttatctgggtttacctattaatatggccagatgttataatcagtatttcagcgtaacagctgtctcatgactagctgaaagtatgcatacatgtcaactaaaatcctaaactgtttttcacatgttctttctttcatccagcagtgttttctaccatccctcacacccctggccacatcatatttatagtattttgaggtctaagcccaggactttctctttgcttcatgtattcctctcattttagtttctgtcttacctcgacacacaattatgtaaggcagagaggtgaggcagaatgagaagagttgagagttagaaatagatccactaaactggagtgaaggagagaagccctgaagagctatataatccacctgtaggtcctaggaggaaggtgaatccatataatattatgtgatacccagagaggacacaagcagaaagtagtgctccagtccagcagttaagctcagaaattgtaaaatgccttaacaggtacaagagcatgaagctgtagggttcagggtcagatggtcaggaatatgctgaggatgagacttattaacagggagcagggactctgtaatgggactctaaggttcaaggcaggataccagaccccagaaagagggcaaatgagagtaaaaaagtcttcctggccaggcacagtggctcaggcctataatctcagctctttgagaggccaaggtgggcagatcatgaggtcaggagttcgagaccagcctggccaacatggtgaaaccccatctcctgtctctactaaaaagacacaaaaattagctgggcgtgatgccatgtgcctgtagtcccagctgctcgggaggctgaggcgggaggattgtttggacccaggaggtggaggtttcagtgagctgagatcacgccactagactccgtctcaaaaaaaaaaaaaaaaaaaaaaaaggcttccttggggatgctactaatactagagtgtctagagagtaactctggcccagaataaatgataagacttcagactcaagaaaatccaagagaaacccttgaaaatagattagagctggactaccagcaagggggcaccatgctaccattattatttttcttctttttcttatcagatattttctaagacccacatatttccttttactttactctgtgccatcttatagatcatctggattttactgattagcaatttttgaaacaattgatgtatgtaagactcaacatagaaattctcaggaatgttgtctgaatacttttcagtatgaaaatacaagataatcttgttttcctgaaggcaaaagtaatttaataatggaatatgcctccatcgtttatccttttagtactttgccatcacaacattaaacatgagagggagtccccatgatagaatattctagcagagcttttctttcttattcttttttaagccataacttttaaatacttcccaacttttaaatactctaagttttttcatactcatgtacttaaattacaaagcgaaagcttgtgtggaaaggagataatgcattcagtgacaggttgttctgggcatagaattgtagaccccactaagcctaaggggtaatttcacaaagcagagcttgcttaaaagaggctccttgtggtgactaaaacaactcttgggctgtaagtgatgcttcagacatgggacaaggtgtggaaaggagcttactcagacattttggaaggtaaaatgtgaccactggaaattaggtggctggctgtggtgactcatagctgtaattccagcactttcagaggccaagcagggaggatcacttgaggccaagagtctgaggcctgtgagaccctgtctcaaaaaaaaaaggaagaaaaaaagaaagaaagaaagaaaaaaatattagtagcataggaacaagtttttcaaatgtcagttgaagaaaaattggctcctggtttttagtagcctagcctggagttgagatacaagaggtaacgagataattacctcaagggctacccctaaatttctttctagtcagtatcactaaatggtcccccatctaagagactatggttcagagttggatggcctatagtttcaaatgaaaccagaagacttatgtgccccttaatcagctcttccttttaggttttaccccagagctaaatcctacttgggaaggagggcatgtgtgtatgaatattagagtaaaatgcttatttgagtacttgcttcagtagtgtttcttttatttccctcttccagtttctgtcctcctacaagggaaagtcatgattacactaactgagctaaaatgcttagcagatgcccagtcatcttatcacatcttaaaaccatggtgggacgtcttctggtattacatcacactgatcatgctgctggtggccgtgctggccggagctctccagctgacgcagagcagggttctgtgctgtcttccatgcaaagtggaatttgacaatcactgtgccgtgccttgggacatcctgaaagccagcatgaacacatcctctaatcctgggacaccgcttccgctccccctccgaattcagaatgacctccaccgacagcagtactcctatattgatgccgtctgttacgagaaacagctccattggtttgcaaagtttttcccctatctggtgctcttgcacacgctcatctttgcagcctgcagcaacttttggcttcactaccccagtaccagttccaggctcgagcattttgtggccatccttcacaagtgcttcgattctccatggaccacccgcgccctttcagaaacagtggctgagcagtcagtgaggcctctgaaactctccaagtccaagattttgctttcgtcctcagggtgttcagctgacatagattccggcaaacagtcattgccctacccacagccaggtttggagtcagctggcatagaaagcccaacttccagtgtcctggacaagaaggagggtgaacaggccaaagccatctttgaaaaagtgaaaagattccgcatgcatgtggagcagaaggacatcatttatagagtatatctgaaacagataatagtcaaagtcattttgtttgtgctcatcataacttatgttccatattttttaacccacatcactcttgaaatcgactgttcagttgatgtgcaggcttttacaggatataagcgctaccagtgtgtctattccttggcagaaatctttaaggtcctggcttcattttatgtcattttggttatactttatggtctgacctcttcctacagcctgtggtggatgctgaggagttccctgaagcaatattcctttgaggcgttaagagaaaaaagcaactacagtgacatccctgatgtcaagaatgactttgccttcatccttcatctggctgatcagtatgatcctctttattccaaacgcttctccatattcctatcagaggtcagtgagaacaaactgaaacagatcaacctcaataatgaatggacagttgagaaactgaaaagtaagcttgtgaaaaatgcccaggacaagatagaactgcatctttttatgctcaacggtcttccagacaatgtctttgagttaactgaaatggaagtgctaagcctggagcttatcccagaggtgaagctgccctctgcagtctcacagctggtcaacctcaaggagcttcgtgtgtaccattcatctctggtcgtagaccatcctgcactggcctttctagaggagaatttaaaaatcctccgcctgaaatttactgaaatgggaaaaatcccacgctgggtatttcacctcaagaatctcaaggaactttatctttcgggctgtgttctccctgaacagttgagtactatgcagttggagggctttcaggacttaaaaaatctaaggaccctgtacttgaagagcagcctctcccggatcccacaagttgttacagacctcctgccttcattgcagaaactgtcccttgataatgagggaagcaaactggttgtgttgaacaacttgaaaaagatggtcaatctgaaaagcctagaactgatcagctgtgacctggaacgcatcccacattccattttcagcctgaataatttgcatgagttagacctaagggaaaataaccttaaaactgtggaagagatcattagctttcagcatcttcagaatctttcctgcttaaagttgtggcacaataacattgcttatattcctgcacagattggggcattatctaacctagagcagctctctttggaccataataatattgagaatctgcccttgcagcttttcctatgcactaaactacattatttggatctaagctataaccacttgaccttcattccagaagaaatccagtatctgagtaatttgcagtactttgctgtgaccaacaacaatgtaagtaaatccattctttcttttattcagtatctgccgtacttatagtgcattacatagggaaagaaaacacacttcaaatcatactgtgtgttctcaagccaagcccaatccgatcttgcataaattaccaaaacactgagcatccatttttaacctaaattacaagtcatcgtggagattttatgaaaaaacataaaagcatttggcacagttcctggcacataatagttgctcagcaaatgtttttctctttgtttttttcccagttttctacataaacatcacaaagtattttaaactatttaattcattatggtagagcttactttttgttttataaaggttttaggtatttagggagtttggcaaaacttttcagctttccaaatctgtcataataaaataacttcttgttctaaaatgctgggattatgaattattcataaatcctttttgtgatgtggtgaaaaagatttgaaatctgcatgcatgtctagaaagatgattcaaaatgaaaggcctgcctctataatgcaacactacagcttcttagaagctttttccaggatccatgctttagaatgaaccaaatttgcaatagtaaaaccaaattaaagaaaacttaactttaccaaaccatgaaaaaacaaaataaatgagtagtttcctaggttaagatatggaggaggaggaaaaaggaaaaattcactgtagtctaaaggcaagagagtgaggctaatgtccatttgggaagaaagagggaaaacctggtcagaaatcaaatctaccaagggatgaaactgacattcctctacttgaaaatgggtacagctattccaccacacttcaaaatatattatccaaaaattagctgcgcgtggtggcacggacctgaagtcccagctactcaggagcctgaggcaggagaatcgcttgaacccgggaggtggagttgcagtgagccgagattccaccactcaagcactcaagcctggtaacagagcgagactctgtctcaaaaaaaaaagaaagaaagaaagaaagaaaatgtattatcttttagactttttgtagttcatatgtcatggtaaatatagctatgagcatggtgttggctaataaacatattaatcatacattatttttgaaaaacatgggtgttagataaataccagaaaattgggggatgagagcaaaattcatgctgatttttagaactctcagactttattcatatgtaaaaggagagagtccattgttagataaatgagacatcttcagatcttaaaggtttatgagattttaaaactacattaggattgacctagactaacagtgtgcatcagaattacccaaaaggcttgctgaaacacagatggctgagccccgctcccagggtttccgattcagtaggccagacgtaggacccaagagtttgcatttctaagaagtccccaggtggagctgatgatgctggcctagtgactgcactctgaaaaatcacaagtctagaagtgtatcagtgccatttaattatgcaggtgattaatattaatattttgatcacattttttccattgttaaatttataataaaaatatcaataattacttttaaaggcattggttccactaagcagttatacacagttaaatcactttcaaaaatggatttttgaaagtcggagcttattcaattgttagctggccatgcattatttaaatgtatgagcaggtgaatgtttccaccaagttaatgtaatgaagaaaccttgagaccattagtacttttgacaccaagtttatggaatcctggaccatatctgaaataataagtgtgaatatcaggcacatgctttcttgaataagaaagaatactttgtagcacaggaaaagtcttacgctgaaaatactttataaccaaaatagaatggtatctttttgagtgggttgtcccagaaatctgagaaaaagtagttgctgatcatctttggagccaaaatgatgaacatgacatctttgaattggtgaacttagggtcaaagaagcactgttcagagactatgtaacaacaatataaataaactaaaactcatcagaggacagccaccaggaagatgacaactctgatggaatactttagcctcatgatgggaactataggtggcacgggagaattgtctacaatattggtcttcagatatttgaaaaatttacatggagaacaaaacttgctgggaatagctacagtgttagaacttagggcaagcggagaaaactaggaccatcaggggaagatttctcatctttcccatatagcattcaaagattgaatgagcacttgaggagggaaaagtcattttcttattaataaagctctccagacagggactgccagatactcagtaggaaggctgttaaagggcatcaggaattcatgtttcatgtggggatttaactaattttaagattatttacaaccttcagagtctcatgattctgagttagatcaaaatattgaatcacgaagtcaggagtttgagaccagcctggccaatatggtgaaaccccatctctactaataataaaaaaattagccgggcatggtggcacgcgcctgtagtcccagctacttaggaagctgaggcagaagaattgcttgaacccgggagacagaggtgcagtgagccaagatcttgccactgcactctagcctgagcgacagagcaagactccatctcaaaaaaggaaaataagaaaataccacattggctgcatacatagttagacatgcatatacaaagaaattcaagccaattaatccatgtggaacggcagttaataaagattcctataagaggcattttaaaagtctgatctcaagacttgggcagcgtatcaggtttatagatggagtcaacaaatactacctcttaagcagaaaagacattagttgaaaggatattaggtatctcatattaccactgaaaagattgcaaaaccaagcttggaaaacaatcaggcacaaagtaagtcaggcagcagtgaggactgtagccagaatcatagcggaaaagcacaatggtgaggatacactgacgaggaccttaccagctcagagcactgcttctgttcttgaaaacttgatgttgctgctgttgtgaccctaggcctcccacagaatagattcttcatggcttccacttctttgtaatgttagccccggttcaaagtccgagtctgagcaactgattggcatagctaatgaatgcccacattgtggctataggagagctgagaacacaagaatctggtgtcagtagtttctctagtagaaagtgagctctactacccaccaagactcataaggtagggaattccctcgacatgggaacgtgctgggcagtccctgggtggcggatattcactgagcaggcacttcagggtgtcttccataggacggtaataggtgtcaccacaaaaaacattaaatgtttcttttgatcatatttttgctgcaggattcttcagagcctttaacatatgaatatgcattgtgcttctccaaggagagatggtatgcaatattcatcaaatgtgtttgagcaagacacctttttttatagagtgactcttgtgttctgtgaagcataccctaagaacactggattagggaagcgttaaaaaatagaggtcatccacagaggactgaaaaaaacataaccaaatataataagagatgagaccatttagtaaggtgggtcagaatggagagtgcctggaagtctgccagtcaaattcatgtttgattcattggatagtgactagggtgagacttgagcttgaaagtgctagttagcctgaactggtaccttcaaaggaggctctgattgtagcatataaaaggcttaggagaggacagccaagaaacaaaaccccatggaaaggttgaatcagttatgatttagaggtaggagtatagatttaaaacaagagacaaagactctcatacctgaaaggaacactgagtccgcagactgcagcctgggggtcaagtctacccactgcctgtttttatacagcctaggctaggaaaggattttacatttttagatggttgggggaagaagatcaaaagaatgttttgtgacccgtaaaaattatatgaaactcaaatttcaatttctacaaatagattccatcatttttattagtgggcctgtattacaacaaaattatattcaattgttatattttgaatttcattacttaaaaatatatggaatattttttctattatataaggacctacataatattttcacttttgcctcttggcccagaaagcctaaaatacttattcttctggtcctttggagaaaaactttaccagcccctactctagtccactgcccttattttactgaggctcagagcaattcacctgaggtctgaagtttcctggcaagggggctaggatggaacccaaacattctgatttctactctactattcttctactgtgtctcctatgcatcaatccaagtgattccacatggggtacagtggtatatgtgggtggacatggaaggtgggagaaatggaggcaagatccatccctatcacagtgccaaagagtaattggattatggtggcattgattgtaatggagagaagcacaaggcatgggaaaggattgagctaggtttgctgaaatcttagaccagtccaatttacacctaggtcttcttacttccacatttgtgcttttttttttttttcttatattagtttgaagacttctagccagaaggtgaataatgtagaaattccttctgaagatcatctatgtgacactgggttttgttatttttggcatcactacttagaaacagaaatattcagctgtgtttcttgaaatgattggactttagggtttggtaagcatttcctgagatggcatgtgtcagtggccagaaaaaaaaaaaaaaaaggaaatctggtattgagaaggattctaaagtaagatctcagcctctgggaaaaagccaggggtatttaattaacaatgtcagccatagatgtaaaagaaaaccatcctgttctcggggacttcaattcattttgctgaagacatttggttgtcagagaattctaggatgtagagagataatatagccattagtttttgttgttgtcatctattttgtagtttatcatatagcttatgtatgtgtgtattatatgttgtagtttattccataaaactattttattgttgaagtatgtcatccccatagtagtctgtaagctttcccattggtttttttagaagaaatcagtgatccactaaggtaatttttggctctctggctcatgtgtcatacagtctaagaaagggcatgctaaataaattagttttaaataattgttttctaacattttttatagaatttgttgacccaggcaaaattggagttcttgtaagcttcaaggcaaactatttttaaagctgtaagtagatttttcaggataaaattgctgaattgaaggaaagtcttgagtctttaacaaaactccccaggctcctttagaaaaattgaatgagagtaggtgaggtaagatcctgtgccttagctgggcagagagctggagagaaagccccagagatggactctcgcacatagccagcgcagcccttgactcactttcacagcagcaatgctaaggggctggccctgaggcccactcccactaccttgggaaatccctctcttctagtggagcctaagaagttcactaaaatcacagatgtagagatttggaaggagactggtgggaatgtggtagtgctgatagccagacaagagagtgagggcagtggcttcagggtaatcacccagtaggaaatcagtccagttaaacgaggtacttgctgggaatctgaagctgactgaagaagggaaatacggaatgcctaatgcagctcaggttagggcacttttcttcataccactcattacagacaattgtgtaatcgtttcatgtttctctcttccgtgagacttgtgcttctcaactgggaatgattttgcctcccaagagacatttgtctggaaacatttttggttgtcacaactgggaaggggagtggcactggtaactggtgggtagaggctgaggatgctgctaaacatcctacactgcacaggacagccccacagcaagaatttttccacccacaatgtcaacagtgctgaggctgagatgtccatggtgacctgcattaggctaaagttttcaaaggcagtctttctaactcagtacttgatcccagcagctagctgggagcctggcacattagaaggcactcagcacgtgctcactgaatataaatgagccaagcctaatttcttttttagccagtttgcctgagtcatttcataggcaccaacaagcactcttaataatgcacactgcaaaggactgattttttccttcacttggacatcactgatcctacttctgagctgcaggtatctctgtgcagaaggggttgccttagaaagaggttgcatttcttctcatctctctgcctcatctcccaatgtaagtctgcatatgtagggtttgcatctgctcttgggaaggaaatgaaagtaaagggtagatatttttccctttgtttatatctaatattttggtgtacaatgatgatctatatggaatgttgttcttagcagtgacaatcattagatttatttttaaggatatacagcactgaacgtgtgttagagggaggggtgatgtaggagaatggagtttacaacatcattaacatttttaatggaatttggtcattttttagtttatataaaagctatttatccttttgaggagagacagttctggatggccctagaaacttgtctcccagccggtctgcattagcccgtcagaggatataggccaagtccctgagtgccctttttatccctgtttctaacaaagccagttttggaaccaaggaacctacattatttcatagtttttgtaggtggcccctgtccccagccaatatgtagagtattatggggggtagggagcggtggtatgtgcagtctatctatgctgaggccagacttatccctcagtgaagtgatgagttgaagggattgtgtatgtgctcaacttcttctctggaggtctttcctcaataaaccctatttcttctatgtactgtggcgcccttagaaattgacatgcaacctatctaccatatagggcagcagtcaacgggagcagctttaggagttgacctaatttgcataatatttattatatttaaaagtatctgtgttataaattatttagttgaaaccatgccagagggaaggaacaagagtaaatattcaggtgtatgtgtatgtagaggggaatgaaatgtgcatgtatatataatcatatttttttgctctgcttctgtaggcttttttaaaaaagagaataaggttttcagtctgctgtcagattcttaggatgtttttaagggtattattcattacattacacagtaaactttttatgaagactcttgagggtaatttatttctgatcaaggactcaagagtaattatgatttttcttactggtttgtttttatcttaacgacaatgtgtgtataactacattttatttaagaattgcttgttagagtgtttaaaaccaaattcgttgcccacccatatacagcaaggatgtaggtgtccagggtagccacagtcctcactcctcattgtactcctgtcttcattttccctctatcttttcatctacttcaaatttatcatcattgtagtctgcatcacttataaacctccagaaatgttttgtttttttttttttggaaaaaaggtaaatgtcttatcataagccaccaaaaacctattttaccagcttcttttttcttccctttccagattgagatgctaccagatgggctgtttcagtgcaaaaagctgcagtgtttacttttggggaaaaatagcttgatgaatttgtcccctcatgtgggtgagctgtcaaaccttactcatctggagctcattggtaattacctggaaacacttcctcctgaactagaaggatgtcagtccctaaaacggaactgtctgattgttgaggagaacttgctcaatactcttcctctccctgtaacagaacgtttacagacgtgcttagacaaatgttgacttaaagaaaagagacccgtgtttcaaaatcatttttaaaagtatgctcggccgggcgtggtggctcatgcctataatcccagcactttgggaggccaagatgggcggattgcttgaggtcaggagttcgagaccagtctggccaacctggtgaaaccccatctctgctaaaactacaaaaaaattagccaggcgtggtggcgtgcgcctgtaatcccagctacttgggaggctgacgcaggggaattgcttgaaccagggaggtggaggttgcagtgagccgagattgtgccactgtacaccagcctgggtgacagagcaagactcttatctcaaaaaaaaaaaaaaaatgctccagggctttaaatgagaagtaaaattttctaagttaataaagatgaagaatgggtgactattatgatgaaccataactaaatgtcttattaaagcaactgagtgtctagccctaaattaaccaggtaaaaactgttaacactaacctgaagttttgtgaataactgttctttaacttattgagatgttgcaagaaatgcacatccagggtggactgggagctatgaaatgactaaattcctccttgcagtgtttaccttcaagattgtataggtattctctcctcttcttcccccagtccccattacttatttgcacacttgttttaactgacttcctgttttgatatttatcaccaagaccataaactcatcaatatgaattttcttgatgtatactcacgactgtctttgatttatgttcctaaatttttttagatagggtatgttgtgcttggcagaattcttttttggcttaatttttacttcctttcccagcttgcttgagtcttccttaacctggttttctcttaacaccaatgatgaaccctgagaaaatgcctgcatttggcctttgcctagaacaggagctgagtatttaagatgtattatcctcggggcatcctgaaaattaatgtattccctaaaatttcttcttgtctatcatcaaggcctttttttatttgttaagaatttttatattaaatgtaaaacatataaatattttctattgtataatcttggattcaacatctgtgaggatcatttttgtaagatacatagaatttgtgcatttctttatgaacttactgttagtgttcttggtttcggctgtttatgactcatgtttatgtacattctgctccaaaatgtttttgtactctgcaactaattacttttggataacaaaagccttgtgtttaatgccctaaagtatttgggggtttccttgtgagagggtggccgaatcatctacctctctcttccatagttgctgtgcaatcgtacgggtaaaattcttaattacttacaaaaactgtgatagaggggagaggtacagatttggtttttaaattgattttaaaatactttatagaaatttagtaagagttaaagcaaaagactttttttccctccatctatgtaatctctagctatgattataatgtaaaacagcctctattcagtgtctaaaatgagttctcaaaacccacaaagaaatagatccatttaactgaaattcatctcatgcctttatctaactcttctggaaaatacattttgctcctttttataaagagctttgtgcttggccttttattatgcatatttttaatgaataaaatcaactgactcatttttgggaaatgtacatctttaatatgtttttccctaagggtctaggcattatgatgccacctcttcattttggatataactatgaagaaatctattaactgcacctacacaaaactggaaaacactataggtaaacagtactgtgtatatggaattgtggtgcgtggtatgtgttttctcttgctttgttccagaaagatgaaagtgagtctttaaagcatagcaaatagagcttcccctcacctgccacacactcctgcactaaagttcaaagaaaagaattattcaaatcaaatgaaatttgaataatattagctatcaggaatataggacaccaaacacactgcctcacacattattgtttattctgaacggaaaggaacagtgtaaaaataaactacttaccaaaacttgcctttagtcagaacattcagctctcagggaagcaggtataactgatgaaacttctaggacttcagaaaacagattctaattatatccatgtttttcgttgttattttcattttttaaagtgtgagttgatgtttattttagtgtttcacaatagaattatcatgtatactgattccagtgtggcatgtgattaagttttggacatgaatatgccaaaagtgcttaaatgtttttggccacataactagctagaatttcaaaacaaaattcagatttgctaccattcacctgacgaacttctccatggctcgtcactggtttggttcaactgcagaagtcattggctaaattttccttatcacctacgtaaatttctagaagaatttactagcaggattaataatgaatattataaactgctttccacacactagggtataattattggtgcaatctatgttgttcttgatgccatggttttacatcttttaatttaaaccttatacaccagtgtttaggtcgcttttgagacaaggtagccctgatgcaaggaaaaatgaggctactacttcacataaaaaatagggtgttaataccacctttactgtactgttggagcagatctttattttacaagtgttcatattcacataagagaattttaaatgaagaaaattaagtttgttaaattataattatgagactttcgttggcatttgatttcaagaatacatatgcattttccaaaaaataaatgtggaggttattcagaacaaaattcatagcttacggtagtaatggcatggattaattttatttgcatgcacacacagacacacatacttcatatatgcaaattatagcattagtttttgatatatctagtggtttttttttcaaaattgcaggaggttgtagattaaattaagtatgatagcacgttttttaattcattaatcatgaagtgctctgttccctaagttatgacatttagaagaaatcacatgctcaaccttaatctgagaacctaagtgtttttgagcattccagtaaaggagtagtgttcttttcctaacttacatcagggtacatctgtgtggcacacagatactatcttttcattcttcactcccagtcttaatttctttcccctgtatgtcactgtattttatgtcacttcacattgtgttaaacagggtaaatagagatcctcagtatctcagtgctgtggcatagctccaataggtgtgctgtggtgcactgcagtgatgtatttgcataacattgtttgatagtgaaaaattttttttcggttcaagtgttttgtgattaaaattttatgtttaattaaaatattgcataatattgatgggttcaaaaaccattaaaataatcaaacaattattctgtgcccttaacattattttcttgtgttttcaataaaattttcttttcatctttccatcttcacattctcttagcatcatcctatgcacataaggtatgttgttttccttctccactcacctaccgcactaaatttgatcagcaattgtacagtaacagaatatatttgtgccagaagatttgcaatgatatttgagcatgtatttatttaggaggaagccagtgcctgtaattatgtctcattgagtagttttactttgcccatcctgcagtatttagagctgatctgtacccaaagcaggacaatgtaactgtagatctgctttgttttcagtaactgtacattgcatcagaatctgtttatttctatctggaaacagacagaatggtgggggagggaaggagttattttgcatcctagtttgtattatgaagtcatcatatatatatttgaaatagtataagctaattactgttgcagaatgccctcgatataattatttcttttcagctggatgtgaaaagctgaagcagtgaaaacctgcagtcttatttcactggtttccttctgtttcttgtccatcacttctgtatccacagggagctatctactacatacctttggtaacaggctacattctcttcagattttttatatgaataatttcatcagcactaaagcattaacatagaataatgagccaaagtactgagtcgagatggctttcagttgagtttaatttcatatttaacttttgcatttaacttgtataatacactactgctgagaaaaacaattatgaatgccttctgcatgttgtacattatctctaacagagatggtgcaattttaagaatacaaaggggtatagagttagaatggatgtgttcgtgcatatatatgttgtatttaaatattaatgtttacaagaagattttttttaaattcttcaaatacttcacttagtgaaataatgggccaatctggaatagagacattttatttggttagtgcaagagtgaaagctagcatttgttaaccttcacatatttatctgtgtacaattgtttttgcttctggtaatgaattattagaaaatggaagattttgttcaatgtttttggccctcaggtttactgtgtaaatctgcatttttggtggtaaatccctttgccacagattcagtagcttttggtaaacttcactgtttttaaagtaccttttgtgtaaaataaagatccaatttttataacaaatggcaaataaaaacagtaaatgctggaa

SEQ ID NO: 3 - DNA, 103726 bp, Homo sapiens
  LRRC8C
tgaatgtaccatatgtctctatctccaggttcccatacaaattaggaaactaaatgccagatgattaaaacagttttctgcttgtttccatgggaaatcaactgctgggagccctcaggaaatgagaggaagaatgtgttcagctgaaggtgtttgctagtgagaacatgcactctgcttgcaacccaggtaccagtccacagactgttcaacattttggcccaggtgagacaccatcctaagctgccggccctgctggtggccaagtgagaccatggagatcattgtgccctgtcctactcccttgtgcctccaccactagggggctgggcactatatgccaggtccttcatgttaggtcccagccaatggtgccaggaagatttattaatgacaaacagacagtaatgcaggggccctagcctcatcaacccagtgtgagttcagcatattttgagcccactagcaggtcttctggaagacaggggactatcaaggattcttgctagaagggtcacttctggccatctacaactagacagagttaattcaggggcgtgggaacagcattctgtaaaacacagccaagtgtgcccgaggcaactgtgccaaggatatctctaggaggctgtgattattacttgttcctgcttgccccagaattagctccaatttgcctgaaagagataacattgaagaaggaggttgcaaagagatttcattattgtcttgacagtgaaaatctctgatttgtctggggaaaattggttcaggagctgactctagtctaacttgagtgtcgataaatctcatcccacctcttgcaaatttagggggaaaagacaacctttcttggctttattaaaccagagtgtgacttgggcatgccataaggaggctcatagatggaaactaggcagtaaaggagagcataaagtttaaatttggactcagacaaccctgaatgctaatgcttgttctttaacttactatgagatctcagggaattctgagccactatgagtgcctacaaagtgaggatatgaatacctactcctcagggaggttatgtcttcataacttatgctatatgtgtcttcataagcttatgttaaataagctaacgtaagtaaaagcatttgttctgagcctgctaggcagtggatactcaatgccttgtgccttctctttcctttccctctgttgctatgttttactgccaaaattggaaacagatagcttactgagcaataaataggcagggccctccctctcccatacaccactatcaccagtaccagcaacttgatttttctcagaattactatctaggatatagacagagcagagaaatccaaatcttcacacataacatacccacttacttatatcttatcatctaggcttgggtttttttgcctaggtagaatatttttcagagaaactaagtgatgaaggacactcatgttgtcttctgcctaaataaaccattagcaggaccctgtgaaattaaaaccacggttcacaattcagtccaattgccttgccctaagctagtcaaaactatgttgactcctcctgtcttcagatgattcatgggcatgattccttagcatgaggctatcctaacatgttctaggtaagccagattagaggaagcagtttaatcattttgtggtcctgggcaggccattagtaaatcagtttttccattgcagctatgctatacaatcatgctccagtggcttgatctacttccatggattccagtaatagctatttaatactgtctgtgttctgtttccacctcaactgaattctgacactaactaccagaattggcacagattaagggacagtgtccttcatgagactgccctcacttaggatgccagtcatgagtctcaggaggtcccaaggccactcacacttctgcccaactggcttcaaatttaatgactcctatgaccctctcaggttcagtcactcactagaacaactcacagaactcaagaaagcacagatttgattacagactttttataaagactgtatctcaagatgagccaaatgaagacatacatagggcaaggtcagggagagaccatacagcttccatactctcaccccatggacccaacgcacacactctcttggtataactatatgttcaccaatcaggaagctcccctgagccttagagtccacagtttttattgggctttcattatatatatatgactgattaaattattggtcaaatgattaaactcagtctccagcccccttcccctcctgactagttgagtttttaactcaggtcaagctggcccaaagtttgagcacgctaatcccatggttggtctttctggtaaccagcctctgtcctgaaactacataggaggctatggtcagacacgtcatttgcataacaaagagacctctatcacttagggaatgcagagggttttaaaagctttggagtaggaaccagggaaaaagaccaatatactctttattataccacagtctcctactgagatttacaaaccatgcaggattcgagtttaagtttttccctttggacagtggggagttacttaaagcaaggggaaattgttattgagcagctactctgggccagccattcattttatatgtgttgaaagcagcactttgggaagataaatcagatgatttcatggagattgaactggataaaactaaaatgggaagactgggaaggagacagtggccactaggtaggcataaagtgatgagagcaaaactagaggagagacgtggaaatgcaaagggaggaaaggaaggggttgagagatattttgaagtaagatttcatcttatctaaagttttgtcaccattacaaaatactacacaatggttagagtagttaaaactgcttgaagagttaaaagtactttctaaagatagccaagtaatatagataaagtggctcctctgatttacgagaccttagtattttatctcaaacaaacttcaggacctgtgtcttaatttttggtggcatactcccattggtttctggacagaaatattgacacctcttgttttcaaatggctgaactgtttctgttttaaaattaatattgcccaattttacatttcaaacagccttttgaagttggtttgctttagagtacgaggtcctccctactctgtagatcacaaatttatgagggatttgaagagatcttgagatgtatccctttatagatcatgtaaaaaagaacacataacactggtgtgttaaaactagctatttcttcctgcatgcgatttcagtaaacaatgaaagtcttttttcaaaattattggtgtgtagccatttttctatatcagtaactaggacattgactcattcatatccacagaagttctacaatatgacagatactttagataagaatttctcaaaaatacaatttcctgtgtttaaatttggggtgggggaaagaaatgatgattttagtgtatcatgggtgatatattaaattgaaataaaaatgtagaaacattttaagttatattagttttatatttatataaaatttatcaattttaataaaaataacaatttatttacataaatgaatacaaatttaaataatcaaataaactttttatataataaatacataattatatgtatattatatgtaatattaattcaggtaataagtattcattatgttcactttttaaacattttattataactaagctaacttataagaaaactgtcaattagtaactctttacaatataattaaaaatttgaagaaagattccctttggcattatactatacataattaaatcaaatcaacaaattttaacctctaccatatggaaaatagaaaaaaatggaaaactttaaaataaggatgcaattagctgttaaatattaataaaaattaatgtttgtcacatgcttacaatatgtcaggctctgttctaatattttatatgtattaaataatctaaccctcacaaccagactataaagtagacactataattacccccatttatttctcaggaaactgaggctcagagaagttaagcagcttatttaggttctcataggtatggaaggtaagagtgatgattaatgactatagagtagtttagtccctttggactgctgtgaaaaaataccttagactgaataatttataaacatttattgccttagttttggaggctgagaagttcgggtcaagtcaccagcagatttggtggctggtaagggcttctctgcttcatagatggtacctttttgctgtctctttgctgcatcttcatatggcaaaagggataagctccctaaagcctctatcatgagggcattaatcccattcatgatgctggaaatctcatagcctaatcacttcccaaaggccccacttcttaatactatcacactgggaattaggtttcaacatatgaattttgaggggacacaaacattcaaaccataggaaagcatttacagaaaagaaagtgtgagaaaaactttatgaagatagaatttgaagtgaacatttaaagactgatggaatttggagagaaagagagtatagtgataaggacaaaaaggaagtaagaaattagaagatggtataaagacacatgcaaaatcagggaggcagtaaagaactataaacccatcagagccatgtgcaaatttgattaaagaactgcaaagttcgggaagatgatttttcaaaaaaaaaaaaaaaaaaataggcgatttcctactgggtctacagaaattcagtcatcatatatgttataaaaaatgccaatgcaggtttaaatccaggtaaattcaaatacttccagttaaaaaatgtcaatacttccttttagctacttgagatttcacaagatcaggagattcatacttgcttctcttaaacttcttaattcactctagataacagaaacagaaagtaagttccactcaactctgttcttcagggtcttaacagaaagacagaaaaatatccaccaatcttcacaaaagtttttcagcaaagaccatgacgactcctcattttcttgtgaaaatggttgcatgggatgctagaagtgaaacttgcttttgcaactgaaatgtgtatcatactatttgtcttcggtagccaggacactgagtagatgtaacgttgcagagaattttgcatgaagcctttttattgctattttttagaaatcattttggagttttgagaggctaggcagcaatctataaaggttgcttattaaatgatttcctcatcatcctaagaaataatcataaaagagtttaaaaattccttttggccaggcgcggtggctcacgcctgtaatcccaacactttgggaggccaaggcgggcagatcatgaggtcaggagatcgagatcatcctggctaacatggtgaaaccccgtctctactaaaaaaaaaaaaaaaaaaaaaaattagccaggcgtggtggcaggcgcctgtagtcccagctactcaggaggctgaggcaggagaatggcgtgaacccaggaggcggagcttgcagcgagccgagatcgccactgcactccagcctgggcgacagagcgagacaccatctcaaaaaaaaattcctttccccttaaagcactcagaagtcttgcaccactcctcacctcaccctccttgggaaactggagaggctgagctgccagtctgatctgaggaaaagtattgtgatgtgacttgtagcctaggcagcatcacaatgtgcccacctggggaacctggaaattgggcagtttggggctggcagtaatagtctttataatcatgaaaaatggaaacatctccaggtagagaattagtaacttttaaaatattgttgctctttcctccctctttggatatagacttaaagacactcaacatctcttggtatttgttttaggttactaaatgatttttcaaatgcatctatgttaggagtctgggatacagcaaaaccttatgtgaaacatttccaagtggcataactagaatgtctccctactcaaaatgggctatacggtatctttttttctcagtcaacatgggctgatgcataccaattcacccttaatggtcctgaaatatattcactgtagagaaatgaagatcaaaaacatcgtatatgtgaagataattaggtatgaaactccagataagcaagactgtttatttttgtttagcactctagggggaaaaaatgacattttccagcagtttaaaatactgtgatgttttaagatagaaagggcgtgataagagtgtgggctttcaagtcaagctgtccgggttatactcctggctgtgccacttactagcagcatgaggctaggcaagtgacattagctgtatgtgcctcagactccctatctgtgaaatgggaatagtaagattacctgttatcctgttgttacatggatgaaatgaaatcatacatgtaaaatttttttttttttttgagatggagtcccgctctgtcgcccaggctggagtgcagtggcgcgatctcggctcactgcaagctccgcctcctgggttcacgccattctcctgcctcagcctcccgagtagctgggactacaggcgcccgccaacactcctggctaatttttttgtatttttagtagagacggggtttcactgtgttagccaggatagtctcgatctcctgaccttgcgatccgcccgcctcggcctcccaaagtgctgggattacaggggtgagccaccgcgcctggcctacatgtgaaatctttaacatcatgcctgacgtagagtaaatcctcagcaaatgttaatcagctaccttgtctgtatcattcaaatcactgcagcaagttgtcagcctacaacttgcttgtcatcttacaaatgtgcagacctggttgctcttaaatcaatactgttatactttaaagtctctgataatttttcaatcagaaagtgtttgtttcaaagcaaatgatcagatatattattatagaaggtataattttgaattttggttaagccaataaaatgaacaattaatataaaatggtatcagatcagatataaatagctattatctggagcattttacaagctaaagaatattcgagacacttaagagatcagagatgttttcagtctcctaaatgagaccataagctgttttcatgttgtggttaaatgctcctccttttggtcactggaagcagttcatcttatctgaaccctagtgttttcattttggaagctatcaacgcaactaactcagacacacacacacacacacacacacacacacatgcgcacacacacacacattctttaatgtaacatggtaattccttttcactgagaattgatttttaaataatttttcaaaataaaatagaatataaaatattgtctcatcattgtaatgaaatgttatcatgctggactttaaattaatggtgtcttttctttgaaattccttaatcagagtacatcacttctaatagctctctattgaatcttcctcagaaacaaggatggaaggctatcataaatgccacaagagaggctgggcacagtggctcacgcctgtaatcccagcactttgggaggctgaagtgggcagatcacaaggtcaggagatcaagaccatcctggctaacacagtgaaatcccgtctctactaaaaatacaaaaaattagccgggcatggtggcatgtgcctgtagtcccagctactgggaaggctgaggcaagagaattgcttgaacctaggaggcagaggttacagtgagccgagatcgtgccactgcactccagcctgggcaacagagctagactccatctcaaaaaaaaaagaaaaatgccacaacaggcctagaaaagcattcataggagagaatattcattaggccaggaatgattcagagagggagggtagataagtatatcaagttatcccatcaatttccacctagtagagaggaaaaagctagtcgttgtaaagaaatgtggagggtctgaaattttcttatccttggaaactaaaatactagcctgctacactttctgggatggtggtataagacgtgaaaagtcctgggtcagagacaaaagaaagtttactactcacttcaatagcagtatccaaagtatcagcatttttatatcagttcctggagacccacttcccacagggagatgcaaagagagcgagaaagcacctaactccacagtggattgtgttataggagagaaatcctgagctagggaatatcaaccttttatgagcagtaagtatgcttcccgtttgctttggagaaaaacattatctttattatactggacagtaaacatgtctggcttttgctccagagaaagacattatctctatctttcaaggctgttcactgtacaaatatccttgaacatttattttggaagaaagagtacaaaaacactagagacccatagagaactgtcttccaatagtcaccaataatagactattctatgtcatcttttttgtgctcagaatatgtgcgtgttctatatcagtttcagctatttctgaaaggatgtcatggaacccagaggctaacaagagtatgtaacagccagggaactattcctcagtgtaacagaacaaagctgcactattaccaatgagaatggatactctttgtcctattaaagatgtcaggcactctattatgactgatgtttgtaaggaaaatagctgaagaaaacatctaccaagagggcttatcttgacagatattcattaaatgagttatagagcccacaattcccttctctttccaacaagaaaagtcttatgaaggctctgcagtctttaactcaaaaataacaaaatattctattctaaggaattttattcttttggaatgttgataggtgtaataaaggaaatagctccacggccaagtgaagttaaagaaatgctaggttcagcaaagtttaacaagcatttttaatacaggacttatcagagccctaagcatattaatacgcattgtgaatcttcaaggaagatatactactttccaaacaataggaaccatagattcctatttctcaggatatctcaaggggtagagttcaatggacagctgcttagaaatgcctcattagaggaacggctctctcagtgggatggatggtggaagaccaaggtacttcgggaagatactggctgactggtctgccctgcctgtagacagaattcctgtagatagcagctagcagtgatggatcacttcctgcttccttgattaaatgctggtggtaatatccattactgactgaaatctaaggaggataaacatgtataattttaatatctatgtataaaattctcacaccaaacaagccctaagaagacatctttcccaatcctctcattttcaaatgaggactgaggatgtaaagatattaagagatttctcccaagtcacaaagttggtcaatgaaagaaatatgactggaatccaggtctcatcaattccagcttatgttcttttcactaacagcaaaagtgccattaatacatcactgatactgagaagggaaatcaggctttgttgttgctgatgttgtttcctaaagagacaaaaatatgagtggattcaggaaactaagaagctccaaaaatgctaaaaaaaaatgcatcatacctcaggatgggtggtggctttcacggacagagcatgtgcgcatggctacggcatttcctaaacatacaaagcaccgtctacaataattttccaaagagagaatcatgtttatgtgagtaacaagagcatcttcaatttcactaccgtgataaggtttcctgagccatggatcttcatgcctcaaagagaggacagcttctcattcaaaacaaagaaacaattgcttaggcttaaaattagatgcttcgtcagggctgatgccactgaaatttgaggcaattcaatgttaagttgtgatctgacaggctctatggcatattgtgtatgcataataaaatatggtgttatgaaaagactgaaaaatttgagccctggaaaaaaatcagtcacattctttaggagatattattaatgtatttttaatcaaaaaatttctaaaccatttaaatgttccattgtctttagaataactcacttcatgggtacagccagtcatttgtagatttaattaataggttaacacatgcaaactttttagaacagtagctgcaggtgccaaatacataaaagcaagcaactattccttagtaacgcagttatagcagtgatatttaggtgcaaagaagtcactcctgacaatacaaaactttttactttaaacttacactacatatgtatatagcaaaaaactcctatatgctactactcacatcaatacttttagccaccaaatgtgtgggtttctcctcacaataattcagttctccaatggacactgactggtatcctataatttaactcagttctgatattacctgaagatagcattagaccccacaggctaagggctcagtcccacaagactgcttccccatcccccaatttcacacgccaatcataagtggcaggttcccatgttatattaggatatataccacttttgtccaacttgtctacaaatcaaaggttcccataactcctcctcaggtttaatcatttactagaatggcctacagagctaggaaaagtttgctttctagattactagtttattacaaaggatatattaaaagatgcaaataaatagccagatgaagagatatatagggtaagatccagggtcttgagtatagaagggtcttgagtatagaagcttctgtatcagtggagttttggggtacacctacccctacagcaggtgaatgtgttcttgttcatcaacatggaaggtctctgaactccatcctttttggtttctataaaggcatcattacataggcccgattgattaaatcattagcgttggtaattaagtcagtttccagttcctctacactcccaaaaggtcaaaggtccaggtggggctgaaaattccaaccctctagtctagacacacacacacacacacacacacacacacacacacacacacacacacgtttaagtttccatagcaaccagctcccatcctgcagttatctaggggctttccaaaaattgcttcattaacataaacccaggtgtggttgaagaagacatcttatgagtaataaaagacacttttttcacctttatctggagctatttcaggaactaaagacaaaacccaaatataatcaaaaatgatcctatagctctaatcactgaggaaattacaagagttttagaagctgtaagccaggaaccatagacaatgaccaatatatatatatatttcttattatatcataatataataatatttctcatgtgaaagttacttatctatcgacccatctgaacaataattctaagaatgagactctctgtctggttatagttcatgttgccttaactcggagctacggacacattatgatctttgattaagaagtttgctttcagttgtaaagtgcttttcaattaaagagtaagctgtaaaaccagctcatgaaggcacatatgggatatgagtacactagtggaaaaagcacaaatcaatgcaattttgaaatactgaaaaagtacattattaaccttactgaaataactgatgggttccatttaaaaataaatactcctttcctataggattaagtagtgagtaagcatgagaaccctgaaattgacttgcctaggcccaaatcccaactcttctactcactgattatgtgaccttcagctagctactggattacaatgggcctctgttttctcatctgtcaatatgagaaaataatactgcctgtgtaatggatagctatggtgaataaatgagacaatgcatataaagctagcacacattgagcatcccaaatctgaggtgctccaaattttctgaacactgacataaggcttataggaaattctcattggagcattttagacttttgaatttggtatgctcaactggtaagtatagtgcaaatattcaaaatctgaaatccagaacacttctggtcccaaggaatttgggtaacagatattcaacctgcatttagcacataatgtgtacctcataagtgtttattctttactatttccaaagaaaatttcacaagttagtgggatcaaacaattctgtcttggggtggtaaaataaataaggtttgttttagaagaatctgattcaaagttttatagaatcctatttttaggtccttggtactgtagggagcaaaaaggggtgattcctttccttcctatcataacagttgacacctctctaacaaaggcaaattaacaagagaaaagcataacaaatttatttgatcgtagttttgtgtgacacaggagccttcagaatgaagaccccaagatataggggcaactattcctttattattatttttatttttgatttagggtctcactctgtagatcaggctggagtgcaatgtcctgatcatagctcactgtaacctgtaacttctgggcccaagtgatgttgcacctcggcctcccagaatgctggggttataggtatgagccaccttctcttggcccatttttatgcttgggttcaatgaagtatggaaagctgtgtagaaatatgattggacaaaaggggtatgatccagcaaggggaatggggaaatccagcaaggcctgtctgttcagatttcttcttggcctttcgatataacattccttcctcccaggtatggggtaggaccccctggaatgagggtcttaatttctttatggccagcagttagcaagaaagagaaagggaagggagggagaagattagagtaaaatgtttaggctctaaggctggttttggggaaaaggttctggtttatatgacccacctcagggaagaaggattccagtttctatggcttgcttcaagggagaataagagagtgagagacaggagggcaggagaaagctagacagagactttgcttctgaggctgctttttattccttcattttggggtatcatttttagccccaatagaccaccatctcctctggtacaaattcaactgccattttgggatcaagtccacatttgacattacctctcaggcaactttctgggcaaaagaattttctagggaaggttttatttttccctgtctagcatttttaatggcacagagtcaaggatttcaaaactgttgctaaaggtagcatttatcttgcaggtattaattcagaaagtgctaatggttgcaacagtctaaacaactgaaacttaaaacaacaacgttttcttttagctcagcctcttcccacaaagctcagaaatggtatggggagcagatgtgcacagggtggatagagaatgcagcgtaaaatcctgctggtggttttggtttagaatgtagattttacttgcactcttaccaggtaggatcaactgaaatcaaatgcaaaacaaaacaagaatagcaggttgatctgaaagaacaatcccagcattgacatcatcaaggtttaagcagatacatatacataatacataaatttagtatgaattatttatgagctaaagggttcaggttttagagcctgtagtataataagagttatactgaaagtcagaaccaatacaagaaactgaaacacttaaaaactcattatttcaatatttaatatcaggtcctgtgcagggtattgataatggacataaaagtgaatcaagatacagccctgccctggagaggctcccagactctgtagtgaagaggcatgtaaagagacaaatgagagcatgccatggtaagtgctgtggtcacacaaggaaggaagagttatacttgcctagaacctgtggaggcttcatagagaaggtgacttttaaattatgccttcaggccaggcgtggtggctcatgcctataatcccagcactttgggaggccaaggcgggtggatcacctgaggtcaggagtttgagaccagcctggccaacatggtgaaaacccatctctactaaaaaaaataaataaatacaaaaattagctgggcgtggtggcgggcacctgtaatcccagctagtcaggaggatgaggcaaggagaattgcttgaacctggaaggcggaagttgcagtcagcgagatcacacagcatgagactctgtctcagaaaaaattaattaattacgccttcaaggaacaactcagcccagcctgagttttcttacagctaacaacaaaacaacaatgtgacacaattgctgttaccatacaacccactagaggggttacagatattctaagtctcaccacaaccctaaaaggtagttatattccaatagactgatgaggaaactgaagttcatagctagagatacaagatttaagcccagacctatttcattccaaatcccttaccatagccctgcatagcccagaaatatctacttctgcttctatcaccaacctgtacatggttaacttgtaattacacagtctttagtgtgagaactactaactctggaagtccaattagaatgtcaaaatttagcctaaagaattgggtataaagtgaaaggtcaagacagagtctgtgcacccagttcatctagctcacagtaccctctccttcctggagatgtgctgaacttcaagcacatttgcttcaaaatggtttattctggtttgtgtgtatgcatgattcccatgaactatgaacaccctgaagtcggagaataagccttgtacttctttaagaccctcactcaagttgactgattgctaattctaccagtcctttacagagagcttgctaagtgtggatgttaatgtgaattaggtggactgaggtaagatcactagatttaaggtttacatgctacaaaataagaagaggtgaactaaccagtgtagcatagtttttagactagtggctttcaagcattaatgtgcatgtgaatcacctgcctacacgtgaagaactacccagttgatccaattagaaggcagacacaagagtagccaaaagtattgcaataaagcaacttggggtggactcaacttgcaggggattcagatcccaggtttccaaacctttttgctattcctggttcttttgctttcagtgaagagtagttaagtacaatagtagctctcatttactgactacttacaaagtgattggcactgttcttggtgctttgagcatattagctaattctcacagcaccctgtagtactatacctatagggatgataaggggatgtgggtggtggggagggggcagataaaaagaacagtaaatttcctaagtttaaactggaagaatagggatttcatcctggttcacctgagtacaaagtctaaggttcaatgattccttgacactgggtagcaagggagcatttcagggatgcctaagcttgtggtcaggtgccaacggtacaaagatagaagtatatcagagagcccttgcagaataaggccctccacaggagggttgtgaccttggtacttgcctcactgtgtttaaattcaggtaaggtaagagaattgaagaggtaaagctaccttagagagtattgctgcatctaccctggtgacttacaaggtacttgaatttttttttttctagcaaactgctaaagttagcctttgcctttttcccagagactctctaggaatttttcccatctgctgactaggaggtaagcccggacctaaatttttgtcttgttgtaggttaattaagactggtgttagattcagctcctaaggcctaagagaatttaaggttgtcttctgggaaggacacccaacagcagcattggaattggaaaaaaagtgtaggattttgctgagtgagcagggggaaagcaatgtaaagcccaagagaatatcttttgaacaaacgtccttttcattttggtttagaaatagaaacgtggtaaaaggaggcgactgttttctgaaggcttgtagcacgaaaacaggccaactggccagcaactagttgtctatcagtttctctactcacatacatctgccttttccagactgccagcgcggagcactagtttgactttgggtgctcttcaaatagctggcattgataaggcattcccagaagactgtgttacttagtaatcttgtcggtacattttttcactgggcagagattttccaaacagagagctgccagctttcaagacgtcagagtgctttttcatcctgaaattgggtgggaaaatggggagaagaaaagcacggttcagatcaatcaatgagagagaacccctccctccatactcgttaaacaacaggtggttttgctgtgcaccgtcaactcctttatgcgggagctgtaggcggggcattccaactgagtctccctggaatctgccccttagcccgtattgcaggggcggtgcagttggggattccggcctgaaaggagacttggttgtcaaggggctcaagggcttcatgatccaaggatattgggttttgcttttctccttctccatcttcctcattctatcccaccgtcccctccccacccttctcttaggaggccttcgcttctcctgtggaggggttatcttcatcctgaccctggccttcggaggagttcccagaagggctatttttagtggtggctgctcccctctaacttacccaggggaggagctgcggtggctgagccgacgctgcgcgcacagccgcctgtggttttccgcgcattgtgagggatgaggggtggaggtggtattagacgcagccgaatcctccctcagagtccgccaggtgggcgtctcaggggtgggagtggccgcgtcgtgaagcggagagaggatttctctcctggtcctggagaaggcccccggcggccggcggcatccctcgctggcgagtcccgggagcgaggtggtctctgcaggggaggaagttcccgggcggcgcggcctgcgtcacagcggggctggcaccgcggagtcgactcggcgggcggtggcgaccccggggccgggcgctgggcggcgctggctgccgctagccgggctgcgtgcggggcggcgctggaggcggctccgtccgcggccggggaggcggaggggaggagggcttgcggggctggggcgttagctgcgtcccggctggaagcccgcgaagagaagtaggaggaaggcgccgccgtggccgcggccgcagtgctcgggcgcgacaagccatgagcagcgaccccgcgggcgacgccgccgtccctggcagccgctgagcccgacccccaggcagctcgccgctccctagcaccttctccagtcgacactctcgcgcccggaggtaagggcgggggatccgcggagggatggagaggggcagcccgcagagggccacccgctcggtccgctgtgcgcgccggaccgggactcgccccgcctcactcgcgtccttgcttccccccggctctcagacaggcagaatgtggttgcccgcccggcgtgtggtgctgggaaccttccgctactgcggctcaggagcccgccaacccctaccccacccaatccgcgctagcccgagcggccgcgcgaggatctcagcctggcgcccaacgccgcgccgcagcggcaggtcctgcagactgggcgcgggggtgggggggcggtccgcgaggggaaatgcgtccgtggccggggcgatcgttgttctttcctacctcgaaaatctctgagaccccggggccagtcctttccggtctatcacccccctgccacctccgaacccttgaagttagcacttttgggcagggaaaggaattggaaggtttctcgaagaaacgtgagagcaccgtgtgctcacctgcaggacaggagggaaggggttcagcttgatcgggcgggccccagaagccaggtgtttaggccagcggttgaggccaaaaccgctgccttcatcccacgcctaggctcgctgatctttccctttccagctggttcccaccaatgagatgattagggatggagttagcaactcaggagccttagatgtgttctgtggacacgaatgcctttcttttctccctcagccggttcttccctccccaacccccacgcctgcccggtatacagcaaagtccaaacagtttcttccaaacctcgcatcctccgtagttttgcaacagccggacacctagcccgcatcctaatgcggatatttcagagtattacggctaaacttcccttggtttcaaattccttaaacatgttaaagcctcagcaaacgttaggaaacattttcaggttcccatcctcaaagtaaaaaataagaaccaatccatctttgttggagtgccggtgtttggcacggggaggcttgaattacctccatttaaagcagcctttgaaggaaggaataggtaaaggaaatcgcagccgtttgtaagtcacatacttctttcgcggtagtgtgagaaacgtagccttaaccaacagtcacatttcccgacaggtatgtgatggtttcccaaagcatcgatgaggcttcagcgtgggcaggtcattaagatgccatttagacgaaaaaggagtaaaagtggagtagtcctcttggttagcacaatgctgcaaaatcgccagagccaataatttttatacatttataaattctgtttatttttaaataatttgttgaaaacccaaaacgtaatctgagctgcataaaactcgctcaattcataaacaatgtgttgagtgtgtaaacaaacattaaatgtatacagctctagtctaggatatttgcatttcttgtgacttgctttaacagggaaggccaaggctataattggtttgctttactctggagtgattaagttctgtcttggggtcagtttgtttcctttgagggggatgtagggcagttttgcaaagatggatatttgccatggttcacataatagattgggcacagacaggacccatcaacccaacaattgtatcagagataccaagtatatttaacaattttcttattaataaaatcaagcctttattaatagtactaatctattatttctcagattaagcactttaatgaaatgtatttttaaaagggaatgcttttagtgaaaataatgcattaggagctgtaagataaatgaatgaaaatgttgcactttatttccatgaagtttatcttttaaaaaacaatacaaagttagaaatatgtttaactcccaagtgttcaatttttaccaaattgttatgatttttaatactctatagtattattaagttatagtcacctatgacattataatagagcgagcctaagtttaaaatgaaatgaggtggaaactatatttattttgccaagaattgtgttaatgatttaatatggtacattgctataacataatctagctgcatgctagatacctaatacacattatctcaggttcttacaatgaacccctaaggtaaatggtgccatccacattttacataagaaggaagttcagagtttctgatttggctgaagactatgcagctaagttagtgcctgaatgaagccaagcctccttagtcttaatgatatacttttttttattatccaaagctgcttatttattttttctatataaatttgttgttaactcaccatagtgactatatgaggaaacagagattgtaaagttttctgcccagcgttttccatataacagaattccaattttgaatcaaggccatctgtttctaaaacccatgttcttgccactgatttctcttaaatgactgatccagagcttaaccttatggacaatttactcctgcccatcagctattatcactgacatgacaatgatggtctcatgggctgtaacacactcccaagattttatacttcatttctggagtgttgactgtatctccagtttcagatcactcataaaagcatattacaactaatgagggttacataaccatggaggtatccttgaatctacttgattccttttgaactataaatcattctggtttggcatggggtaaagagaatggcctccagaaaaagctgctgtcaaattcggactctgccgtgcagtaggagtgcaaatgaggtcagattattcaacttctttgaaccctggtttcctcttgtataatgggattaataaagcctccctagctggatcactgtgcagattacttgtaatagtgcatgtaaaggacagctcttgtcacctagtaggtgctcagtaaggattaacaccagtgtatcctagcactggtagaaacttctgctgtatttcactttatgtatattttacatgcacgtagtgattagtcattgctatcagtgattcactctttatgtaattatacaaaggtctaatctgattcttagcatctttaacttttgaatcacatccttttttgttgtcttgagtcagaaacatgtactcattctatagtttttcctttgctcttactcttttattttatttttactttactaacaatatgttgatggatcggaatttgtattttttggcaaacatactttccttttttctgcctctcctgatctcttagccttgtatatcactcagggtaccatgacaatgccagtctctccccaccacacacctcacccttgaccaagcctccaccagctgaaattctgcatctcctacaaggctcagcataaaagctacctccttcatgagcctgcctgtttccctccagatccttgtcagttcacttataattcagacaacacgtacccctccaaaagtgctgagattacaggcatgaggcacagcacccggcccactcctaatgctattttaaaatttttttaaaattcttatttttccaggtaatatatttgaaagactataaactctttgacgtcagggatttttaaaaaatttctttatatttttgcttgggataatacacagatggtttctatttcctgaatgagagacaaatcggatattctgagatccttacaataattaggaaagacacccaaattgttggtggcaccttctttgttatcttgagacttgttcaaccgaggcttctcgactggtttaactggtattacatttgctggaaagctgtcattcttaaacaaagccttggacattaggtaccatgtaagatagctatgtttcattaataaattgcttccatggtgatatagtaacctcattaggttgcaaatataccgagcatgtcaaccgtatttgagtagtttggtagtttagatgggtaatggttcaaatgtattctttaataatagaatttactagagaggaaggaacacctacctcagagaagaggttgagataatgagcaaggaggatttcagactgctttcgttgaaaataaaactcatcatggcacagtgtggaggctacaaccagaagtggacttcagagagagaattagcacaagcagaagacccactataaatatgactcactataaatatgaggggtagaaaaataaaatggaagaggtaaaataaaggaagcataactataataccttatgactgaggcctgctagatattggtcattcttggactgtaaaagtaggaaagagactgtggggtcaagatgggcaagaaagagaaactaatgtcaccttggacatctagcacagcaatagacttccacttggtccagggccagcacagagcaacaagagtgtaatctcagaactttgtttgaaatacatagtttggcaggaatatattgaaaatttcctaatgattacaagatagaaaatgtctaagctcttttccaatagcaggcataagacaaacaaaaccttagactctctacttaggagtgtaacataccatcacaaatttgaggagtgaatttaggatcattttctggtattcgatgggtgttcttttaaaatacggttctaagtctaagtctaacattcggtgtatctaaccgaatgttaattgatggagacaaggtgatacgggttcagaaaatagaattcagaaaagaaaaggaagaattggcaaaattcagaaatcaattttaagaaaaatgttaaagcttagggaagtaactgaaaatgttccagatgaccaaagtattttgagtcaaagaaatccaggagagctatatgttctttaaagaacctttctaaggactcaggagaataaaagctctttccataccagaagaggttgtgaaggtaaaacaagaaggaaattagtttgtctaaaacaaaaatccatccaagggccggaaacactagagagaacacagtggagctaatttcatttctcaacctttttttgattattaacctcaatctgtgaaccttttttccataaattgtaataacacagatacactgtgtatctgtttatgtactgtgactcttcagtggatcacaaaccattatttaaggttttgttaccccgccttcacctcaatcaatttttcctaggggcaagattgtcccccgcttgagaacacatggtgtagcatgattctatatcatgtttaaaatcacaaaagtgccatataaaacttaaagacatgaaaaatagtcaacatatctataaatttttgtatgaatatatacaataccttaatttacacatcctctggaattaaattactttttttgcaaataaaacatactttattgacccttcagctgaaacttacatgtaaaaatgtaaggtttctagtcccaattttacattgacaaaattattaaattcataaagttgtctctcaaaagcctcccctacctgtatccttttatttcctggaagggaagccctccgcaaacttcagacattcaacatcctttatatatttctgttatgctggaatgctgtggtattattcattcttttgactaatcaattgtatgagtataaattggtatgtctctattagagaatttatgttttaaactataattttttattttacttattattttattttattttattttattttatttgagacagagttttactctgtctctgtctccaggctggagtgcagtgacgcgatcttggctcactgcaacctctgcctcctgggtccaggcgattctcctgcctcagcctctggagtagctgggactacaggcacaagccaccacgtctggctaattttttgtctttttagtagagatagggtttcaccatgttggccaggatggtctcgatctcttgatgtcatgatccacctgcctcagcctccccaagtactgggattacaggcgtgagccaccacgcctggcctacttatttataattatgaaacactgcgttccataactaagagatttcagttcacaacaagtagatctgggaggaggagagggcctctgaagaaattcttggctttctcatgtttagtgccttgcttgggttctttaggtagcatgagcctatcatggcagctgtgaggactagaggagcttgtctcctttatatattggctgctttcagcatctggagcctggcatgctcccttcccccccaacaactcatccctcatctcctccagcttttatttaaaccatattcttttcctttaccctcaaacctccagaaatatatttgtttttccacatattttctctgtttgatttttggagctacactgaaactgccacaagggcagaggccatatctgtcttgttcactgccatatatctacagcacttgcaaagtgtctggggcatagctgctcaataaataatcatgttataaatatttaatgacataatttacagtaacatttcttacctatttggagtatatccattgtcttttctactattttagattctgatgatgaaatttgaaagtatacttagaagtcatacttcttaatagggacattaagagacatgtaaatatttctatatgtgattgaaaagcaggagaaagtatataaataattataaattaggagtaagcatataaaacatgagttaagacatcaaagtaaatcctaggtaacagtcactaaataaactaaaaatgtgaaaactcatacatcttgtcagttctccttatgtatttttcttttttgagatggaatcttgctgtgttgcccaggctagagttcagtggcacaatcttagctcactgcaaccgctgcctcctgggttcaagtgattctcctgcctcagcctcccaagtagctgggattacaggcgcctgccgccgcacccagccaatttttgtatttttagtagagacacggtttcaccatcttggccaggctaagcttgaactcctgaccccatgatccacccacctcagcttcccaaagtgctgggattacaggcatgagccactgcacccagcctctccttatgtattttaagaacttatcaaaatcaacaaaaattactaactagtctttttcaaatatcttagagatttatgaaaactagaaactggctcaaatatctttttaatatgaaagatatatcctataaatgtaacttctgggaaaatactgtagagaaattactatgagaatggcttcattagcaaaacattgtcaggctaatttatattatctcttgatattagcaaggttcagtataatatgcttatcagtaaagttaggagatactgtccagcacacataactttctagttagtgcactgctatgtggacaactgtgaccagaaagtgattatcaactgaggaagtagcagctggaggtatgtccagggtttggatctgagttgggtgttttagaattttatctgaggatctggatggcataatagaaaacatgcaggtgatatttatataagtgactttgtggtgtatagagtgccttgattttgaagcaagaatttgaaaagagtttggcaaaataaaatatataaaattaaagtgaaaggaaataatcaaagataagcaattaaattcatttaaggaaagcacagggtggtacattgaataaaaccaagcaaatgacataaatatactacatttgtatttgttttgttaaatttgtttctgtagtttccagtatttagaatagtatatgacatgaagcaaacatttaatacacatttgctgaataaatatatgaataaaatataagatggaggaagacaacatgtgattagagcaaaattaagattattcctaaggggatggaaatgcgtaagtactaaactgttagaaaaactttttggacaaataatttctgagcatgtgttaaaattttatttgtcttaggtgtaaaatgtttctggtagatggattcaaaaattcattcttagggacatttggtaagaaaagtgaatggtcagatatagctaacctagatttaaaaaatgtgggttcaaggtcactagacttttcactgacacctaacaatttaactggatggctaggtagctggtttctaagaacaagtgtttccctattaaacttaatcagaaaattgcttaattattaggaatatgtgtttcttcatagaaggagattgtgaattctacttcacaaataggttctgttgctgatcaagttagaaacttttttttttaatagtctctcaatttagacagtctttgcaggtctgtcttggtcaaggaagtctatccatctcattacatttttgcattagggtcattgaatttgttacctactcattaacttttaggtttctttatttacactttgcatccagagtcacagttacaagcactatttatataaacactgataatactttgaactggttttctataccagggaagattttcaaagcataatctaaactaacatagtttcagtgttatctactttatataatttatggtcctgggcttctatatttctgtgttctgttctacttagcatgctttgttttaaaaggtaccgcaagaggagcttattaatccgtttaaacgatctttttatgtgatcaaggcattcgctgccatatgatattgcacaaccagtaaggccaatgagttaacatctttgtggaagctttgtgaaatccttgaagttatgtttttattgcttctttcacactagaagtaatttttatagtactttcctctcagcttgtctactccaaggccagaactcagggagtatctaatttatctccatcagattttgcctctagtacttgtaactgtggtaaccctattttcttgaggacgcttttttcctcctcagattcttatcctgctagatagttatattccgtactgcctaaaaagttgagattcttttgaccatagagtagataccagactagtatccagggaggatgttctgtgcattgctacaatttatgtagtgtagcattagtaccttaataggattttcatgtcaaactaaatttatctctaaatgtgacactttgggcacagcatgttagaaattaggtttattcatggctgggtgcagtggctcctgcctgtaatcccagcactttgggaggcctaggtaagcggatcacctgaggtcaggagttcgagaccagcctggccaacatggtgaaatcccacctctactaaaaatacaaaaattggccaggcacagtggctcacgcctgtaatcccagcactttgagaggccgaggtgggtggatcacgaggtcaggagttcaagaccagcctgcccaagatagtgaaactccatctctactaaaaatacaaaaattagccaggcatagtggcacgcacctgtaatcccggctacgcgggaggctgaggcagagaattgcttaaacccgggaggtggaggttgcagtgagctgagatcatgccactgcactccagcctgggcgacagagcaagactccatctcaaaaaaaaaaaaaatacaaaaattaactgggtgtgttggtgcgcacctgtaatcccagctactcaggaggctgagacatgaaaatcacttgaaccttggaggtggaggttgcagtgagccgagatcacgccactgcactctaacctgggtaagagagtgagactgtgtctcaaaaaaaaaaaaaaaaaaaaataggtttattcaattatataccagtaggaaggaggatatattctattcttttttgttgtttgtttgtttttgagacagggccttgctctgtcatcctggccagagtgcaatggcacaatcttggctcactacaacctctacctctgagactcaagccatccttctacctcaccgtccagagtagctggaagcacaggcacacgccaccacacccagctattttttgtggagacagggtttcatcatgttgcccaagctgttctcaaactccagagctcaagtgatccacctgccctgcctcacaaagtgctgaagttacaggcataagccaccatgtccagctgggaggacatattcttaatgaacctatacaaatagctacattaccaggagcagtaaagtgtctcaatgtgtatagtaatgactaccattctgcatggttttgtggactaggcaaacctatttatgtcacattttatagagaattgtagattccatgatagtggagttatatacaaaaagttgtgttgcttttaagtatgataagaataagtaaagtttatgtaaatgatcattaacaaaaatgagttgtgtttttgtaagtttacctctttaaaattcaattttgaaaggatttttaggttctcctgggtattcactagtttattgtcacagcttttcctgttatttcatacaggatgttttccttcatagtgatagaatactagacattttgtgagttccagaattctaaagcatgtgctcatgctgtgtagtattctgaagaatatagtccctcaccctctaaaaccgggcttctgacatacacggtactatttgataacataacaatcaaacatttgaattttgatatagttataggcataaccaataatcagaggtgtttaagttacaatttctttgtttcaagtttttaaaaaagataacctacaaatgcactgaaataacctactaaattaagggttagagataataatctaagaagaaaagcaggggcttatcaaatacctttcaaaaaataaaacatcaatggtaggatagctttctatcaattaactcttgagtttataacaattataaactgaccaaaatataaaaccccactattttttatttagtttatttatttagttgagacaaggtctcgctctattgcccaggctggagtgcaggggcacaatctcggcccactgcaaccttcacctcccaggctcaggcagttctcccacctcagcttcctgagcagctgggactacaggcacgtgccaccgcacccggctaatgttttgtatatttagtagagacagggttttaccatgttgcctacacttgctgcccgctgtggcctcccaaagtgctgagattacaggcgtgagccacagtgcccagacgaaaccccactatttaaaggcatgggaatgcaaccaaagcaagtagaaatggaggggaactgatccttgacagaaagagactccactggatgagatttgaattatgacttttctcctgaaaccagtacccagtccatatggtctacagctactaactctcatacctgacactgcagttatacatagtccaaaggtagagtttgaaccttgctctgtggttggaaagtgaaggggaaatcctagggggaaaaaagtcacataaaagagattgccctcaaatccgcatataaactcttaccaaatctttgcctgaccccagaagtgctcatgagaggagttaggctccatgtaaacctaaacccctgagcagcaggaagcctaaaatgcttgagcagacattttagctgctgcccaccttagagaagacagtttagtttcagtataaccgagtcaatggcctgctaaagctaaaaatcagtattttccaaagggaaataatagactacagagcttccataatatgtcatccagaatatccaggataaaatcagactagtaggcatgaaaatgcacaggaaatataactcattatcaagacaaaatacagtcaatagaaattaactccaagatgatccaaatgttaaagttagcaaataataaattttaaaactgctattatagtattgtgaaaagaaaatatagtcataatgaattaacagatgagaactctcagcaaagaaatggaaattataaaaaacagtaaaatagaaattccagaatgaaaaatccactggatgagcttaacaacaattggaggtagcaaagtaaagagtagagaatttgaacatgtattgatagaaataatataatctgaacaatagagagaaaatgattgaaaaaaaattgcacagagccataatggcatgtgggaaaatgtcaagataagaatgtcctggaaagagaagagaggaaaaaatggaggaaaaaagagagaaaaaaagtgaagaaagaatggccatacacttcgcaattatggtgaaaacacatcaacttaccaatctgaaaagcacattataccacaagcaggttaagtacaacaataccattcctgggtatattaccacagtctaactactgaaaaccaaagataaggagaaaactatagaaacagctagagaaaattaaaatacatgtcatagagtataacaatatagatgatgagtgagttttttctcaaaaaaggacaaaacattattattgaaatattagagtgttttctctgaacccaatagaattaaattagaaaataggaacaataagatgtttagaagagcctcaaatatttgtaaataaaataagaaacttctacatatcttatgggccaaagaaaaattcaaaaaggttaaaaatatttcaaactgaatgataaaaatatagcattgaaattaatgggaaaaaataatgagatagctaaagcagggcataggtagtttggtaggaagatgatcagatagatagatagatagatagatagatatggttacttagaacttgatattctatattatgaaacttttaatattaatcatcttaatttccaccacaagaagctggattagattcccaaaagcatagaaagaaatactagatttaagaacagaataaatgaaatcgaaatcagatgagtgacagaaaaatattaaataagacaaatgttggtactttgaaaacattaatgaatctagccaaaccattaaagaggtgagggggagtaggggatggagaacccaaattaccaatatcaggaatggaagaggggtatcactacagaccctactgacgttaaaaggatagcaaggaaatattgtgaaaactatatgccaatacatttgacaacttaaatgggaaaaagtttcttgaaaagcacaattacaaagcctgacagaaattttatttataaccaaaaactttcccataaggaaaacttcaaaggggatttaatcccaagaatgcaaggttggtttaacattcaaaaatcaatcactgtaattcatcatattaaatgatcatatggtttttctcctttattatttcagtagatgcattcaatattttcagcatctagtaatgataaagattttcaaagattttctatcaatctcatacacattttattttaaaataacctatagctagcatcatacttaatggtgaagttttgaatgccttccccttgagatcagaactaagcaattatatcttctctcatcacttccattcagcattatacaggaggctggccagtgtgataaggatgaaaaaaattaaagattaaaagaaagaagtagaactacttttctttataagctttgtggttagaaattgggattccatattctaaggaatctacaaagcaacaacgaaaaataataagtgccacactaacttgtaatcgacatgaagatatgtaaaatttaccaaaaaatcttaccagtaaatctgttgacaatcaccatgtcaatcagcccttgtatatgaacatgaatatgattttacttactctgaaagggtatttaataacataacattaataatattttacctgttaatttatctttacctgacagctttccccataatgtgagtgcaataaacagaattgcctactgttgacctaattaaagattaaggaacaaatcaattaaaagtaattatttctagcctctctttcttgtaagtaccatatcctggattataggagacaaagcaaatcttcggtgctacctagatgctattatgtatcaaacatggattgggttatcaaagtgcctcctcctttttatatagctccaaattaggaacatgatcttaaaggaaggcagcattttaacatgttgtcagataagaatgcagatgcccagggaattgagaaggtcatagtattgcctagcttatttactaataactgtatcagaaagtacataagaaatgttagaattttaatttctctagaggccaggaatctttattattcttgtttcatttcggtattttgaaatactaaaaagcagcacaaaagcatgaaattgacttgccgcttctgaaagaacacatttgggttgggtgagaaaaaatatataacttttactctctgggtacaatatacggtaagtaaataactttacagatttatttctagaaaaggccatctcgaaaataatttttcattttgagttaccagttatctatcctacttagctacttcaaaaaacttaattttatctcaaaatgtgttatttttcatacaaatcacacaggccaaacaatggtgtgtgacaatttttagtgttaaatgttagtttttaaaataaaattactaaagtgggaggatcacttgagcgcaggagttcaaaaccagcctgggcaacatggtgagagccatctctacaaaaaataaaaattaaaaaaattagccgggcatggtggctcatgcctatggttccagctactctggaggctgaggcgggaggatggcttgagcccaggagactgaggctgcagtgaactatgtttgcaacactgcattccaacctgggtgacagagcaagaccctgtctcaaaaataaataaaattaattttataaagcatgttatgaaataagattatagtcttatttgaatattatgaagaaaaaaatatgtttataaatcgaatacataggttaaaactattgtataagtattttgccaaagattattcattgaattcctaattttaatattagcccctgcttaaaattaattatctaaaattgcaatttaaattaccatttaaaataattactgttcgtattcaaagcctataagagaaatacaacatcccatgatttaacattactgcaacattgcctaaaatttacaaccattaaagaacagacactagttataattcattgttgttgggtacatctacagtcttaaatttttagtataaataataaccaaccatctgacctatgagacaaatataaggaagttaatatcttctagtttaatgcacattgtatcagagaacagacagggtgaaccaaaacacatgaatgaattgcacaaactaaaatacatttttaacagctttattgagatataatatacaaaccacatatttgtttacagtatacaattcaatagcttttatcataattatagaattgtgtaactatcaccataatctaattttaaaacatttttatcaccccaaaaagtaacatctatgcattagctattacccctgctccccatcctctacttctcccacagccattactctatttttgtcacaatagatttgcctattctggacagctcatgtaagtggtatcaaaaatacgtgttttttgtgactaacttctttcacttagcatatatttaaggttcattaatgttgtggcatatatcaatgccccacttctttgtcaaataatatttcatagtctcaacataccacattttgtttatccatccatcagttaatgggcattttggttgttttcaccttttagcttttgtaaatcattcttttatgaacattcatgtatatgtatttgagtacctgttttaaattcctgtggatattcccctaggaatagaatttctgggtaatatgataactctgtgtttgacattgtgatgtaaatgaggtataatttttgcatcctatccctaactaaaataaaatccatgctaaggcatttaactgtctttttaatgacgtttaacaaactaatttgtctaagcattaatcattactggaatatcaattgaacttcaaattttatataattaattcatagttgtttgcaaagtactattaaagcactagtaaagctctttacattttggatttaaagttctagttaaatatggaaagagctgctgaaagctgacagggtagctagttgcatttttgttgttcttgttccttatccctagtgatctgtacgtcatctgttaactccaaacattccttctgtgcatttttcccctctttttgttgtgtgtccagaaaagattttttttaagattttcttattctagatataaataactgctattagtttctgtctagaaaatgtgaactccatctatttgaaaaacttcacagtcatgctaatttctctctggaaggaagtaaagttattttacctcctggaggtaaataaaacacctttccttgaaacattctttcaggcactattcctttgcccagggtggtggaggaactatcttgacaggctgtagattctatctgttttggtctatttagagatgctcttttaattcactgtcacccagacttggcagaaatttcctttaactctttcttatcacacacctgtgaaatcacccaggcagatttcccctcctctctttgtttcaaagtgaattcatttgtttaatgagatgttcttcaaaacacaggacaaacttttgtgacccctttaactttcattcttagaggcattctaagcaaatgagaagctctcattctagtagttgagtcctaggtattcaaaggactcactgaccaaaaatatagaacactgtaagtaactgtttacagacacagacaattgtacaggaaacctctgaacccattcctataggatacaattttaactgttcaacaaatgtaaatactgatacaaatatatacacaaacccactgcaggcaaaacacaaaataacttaagatttctgtgaatgttgtcataggataattttcaaaaagttaaacatgactcttaaatatagaatgaacagtcaaagatttatttaactcataaatgagggagccagcaggatggcaaaactggtgcaaagagtgttcaaggaattatttttgaaaggaatgttagaataacattgctagatataaaactaggtaatgtccttcaggaaaataaaacccataatgtttttagttttcttttctaccagccaaaaatctacaagactggaatttaatcagtagaaattaggaagtaaaaaaaaaaaggtatatttccccaagtaatccatgggtgggccaattaaataattattcagtattacattgaaaggatatattgctctacctttccctcacttccaagttttgaatactttttgaaacaatgtacacttgcttatcctaaatgttctttttaagggctttggttgtttcttagtaatatagtaaactttaagccatagaatgtgtcaaatacaaaacactggtggtggaaatcttcaaacttagaacatgggttctaggaacagaagttttgggttctacacttccacacttacatccctccaatatcaaatactgctctgatttcaccctgggggaggagtcctgttttaactccaaccccaagttcaaccacttcttagagctcaaacagctctccaagctctcaacttagatggcaagtcattttaagcccagtagaatgggagtcccttcagatggccattagacattgacattcaagtgctcaaattgccatatctcagaaaaaaaataatccagtgatcaagaagcataacacagtgggtaacaatagcatatatgccatgaagtaccttgatccagaggctgacaataactgtttctcttgaggtgagcaggtctccaagggagttctgagaaccattgcaacaggaattgacaagctgtggcccatgcattttgagttagtgcaaactgttttggtatggcccacttgctgagattggcttttctatttttaaaggattgtttaaattttttaaaaagaatatgcaacataggccaggcatagtggctcacgcctgtaatcccaacactttgggaggccaaggtgggcagatcacgaggtcaggagatcgagaccatcctggctaacacggtgaaaccctgtctctgctgaaagtacaaaaaattagccgggcatggtggcgggcgcctctactcccagctactcgggaggctgaggcaggagaatggcatgaacctgggaggtggagcttacagtgagctgagatcgcgccactgcactccagtctcggtgacagagcgagactctgtctcaaaaaaaaaaaaaaaaaaaaaaagcaacgtagaccacatatgatttataaaccctaagtatttgctttccctccctttataggaaaaagtttatggatccttgcattttaaaaaagattgacaatttacttgttagtgtatgttaaagctttgtaataaacttgctgtaagggaggagtttgttagtaggctaattcagagatttgctcttgatagggacattttgtagcaaaagagaattgttagaaaaattactagatttgacatcaaaataaaacaatattttgttaaaggtcactaaattcttggttgacagctggtcaactgcatgggtcatatctaatttcccaaaataagtcctagtgtcattaaagttcatgagaatcaattacagaagcattttgtcaggaacactcatttctttctgagaggaggttgtaagtaatatcacacaaacaggtttgggactatttttctttttcaaggcaatgtagtatatttttaaaagcatacactggagtgctggggtgcagtaaaagaaaagtaaaactgtaggaatcttttgatgccacactaactcataccttgttggaatttagatacacaatgaaatatgaagataaaacaatgggaaaagaatcagaagaccatcacagagtgggttagagcattgaaaacaggatctatgtgggaaagatgaaaggaattgagatattgctgctaagatggggtccaggaaaaagttagtaattatttccaggggtatgaaaaggtctctgaatctgaggagcaattttgcatctcttctgaaaagagataagagtttaacctgctgcacaaagattttggtggttaagaataataattttgaagcttcaagtaatcaaccttttcacaaatttgcatagtggctgggtctctgggatatcttaaaaatagaacagagttggggctctctgtcatgggtttagtgtggtcttgcctgaaggtttaatcagataaccccttatgatcagtcttgcattatcatttaagaccacactaggtacgaggctctcagcggtggatcaaagggaaaataaaagaggaccacactgggtgcagatcaccagatccactggctttctaggggattaaaaagaaagccactgtcccagaggacttactctatgacagaaggatagggtaaacaaatatacaagtggcaagttacggggtcgcagtatcacaggagaagggggaatcgttgcaatatataaacatctgaaaagggacacaattaaaaatacagctcataaggcaaagatctggaggaggtggggagacacagatatctatggaggagaggtctgagaataggggacagcaaatataaatatgttcccctggcattatggaggaacagcaagaaggccagtgtgggtgcacagctctgaacaatagggagagtggtcgttaagagagggtgctgtaggctcatgaagtgtcttgtccacctctactctgaatgagatgaggaaccattagagagttcttaatctgacttgcgttttcaaaggatcactctggctgtggtatagagcaatcattagccaaccttttggtctcagggcatttgtactcaaattattgaaaattccaaagagctctaacccctaagagctcagaaatgtaaatctgattatttattaattttaaaataacaataatgccactgaatgttaacacaagtaacatatgttttgaaattatattttccaaaacaaaagaacaaaaagatgtcagtgtttaagtttttgcaaatgtctttaatgtctggcaatcagtctgttcactgtcatgtatcatgtagcctctagaaagcttcactgtgcagtcacaagacaatgaatttaaaaggccaataatgtttagtattcttgtgaaaatagttttgacctcactgattccccaaaaaggcacactgtgagaaacaatggtaaagagtagactaactgtaagagacaaggatggaacctattataataaaacaaatgagacatgctggtgccttgggttatagcagaaaaggtagggagaactggttagattctagatagattttaaagattgagcctgcaggttttgctaacagaatagacctgaatgtgagagaaaaaaattaaaaattgactccaaggttttgacccagaggacctgcaaagatggatttcctatgtactgaaatatggatggttgtggaaatagcagatttggagtgaaattaacaattattttggactcgttaatttcaatccagcagtatcactgctaggtatctacccaaaggaaaataaatcattatatcaaaaagctacccgcacttgtatgtttattgcagcatcattcgtaacagcaaagatatggaatcaatctaagtgtccatcaatggatgactggataaagaaaatgtggtatatatacacaatggaatattattcaatatatatacacaatggaatattattcatccatggaaaaggatgaaatcatgtcttttgatggatagacatgatagaacatggatagaactggaggccattttcttaagtgaaacaactcagaaacaaatagtgtatgttttcattgagaagtaggagctaaataatgtgaacacatggacatagagtggaataatagacattggagacttggaagggtagaagtgaggaggcagctaagggatgagaaattgcttaataagtacaatgaacattattcaggtgatggttacactaaaatcccagacttcaccactacccagtatagccatgtagcaaaattgcacgtgtaccctttaaatttatacaaaaaaaagagaaatgtctactaaacttccagttggaaatgtcaagttggcagctggatatttaagtctcaattcaggggatgggtgtcttactccctttatattcctcaatactttttaaggagagaatacataaaaataaattttctgtgtccttctatgtctgaaatacactatttattctactctcaaatttgaaagttagtttggttgggtactaaactctacattgacaaccactttcactaagaatttgaaggctttttacaactattttctggcatacaatattgttggtgaaaagtgtaatactgatctgacttttgtttctttgtatgcacccttttaccctctctatttgctgcctcctccaagcttttaggatcttctcattatccccaatgttcatgacagtatgggtctttattaattcattgtgtgctagcactttagtaagcctttttttttttttttttgagacaaggtcttgctatgttgcccagactggatttgaactcctaggcccaagtgatcctccttcttcagcctcttgagtagctgggattacaggcatgagccaccatgcccagctagtcagcagcctttttaatatgaagtcccatgtcctacagtttgggaaatttttcttgcactctcttcttgatacctctgtgtcattttctcctttcactctttagttaatttccattattcaaacattgaatctggtgtctgtatattatttttccccgtctgttttttttctactttaagatgaatttcatcaacattatctaaccttatgctgattttttcattatcttatttttaatttccaagttcttttcggtgtgtttattttataaatacagtatctcttgctagctctctgagaatattaattatagttgggaagtttttaagtgtgaactcctgctctctgctttactttcggttttagtgtgtctttatttgttcctttgattgtgtatttgtcttatttatatctgtctttcagttggaggcattcctcaaatgactgatgatctatatctgtgtcttcaaatgtcttagtttgttttgtgctgctataacagaataccaagactggataattcataatgaatataaaattttgggcttacagttctgaaggctgggatcccaatatcaaggtgcaggcatctggtgagggccttcttgtaacattatcatatggcagaagccatcacatggtggaatggcaaagagctagagagagcaagagccagagggggcaaatcgactcccaagataacactaatgcatgcatgagggcagaaccctcaagacctaaacaactcttaaagatctccttactacccttgctaacatgtatcccttacttgtctgttgaagatgattaatactaagcctgcatcccatctcagaggtggggaggaaggtatatggagggctcctttaaggacacaactccaagttgagttcatcttttctgtttatatgccattggctaggactaagtcccacaccatacctgccttcaagggggctggaacatgtactttattattacgtaaaaaggagaggctaaatactggaaaacaagcagctgtgtctcgactgtgatgctgccatcttacagcgagaggtacaaagagcacagggacattgtagtagctcacaacaaagattcaacatgagggctctttcagtgaactttcctgaagattgtataactcaccttccaagaactctcccttgaggggacatgtttatctcacagattgcagctagtttcctcagcctcagctgctaaccatcctgtagaccctataaaggcagctgtgtttctggatcgcagtggtcagaggaacagcccttccataagctggcaatctccacatcatcgggtctaactgcctgtcaggtgtggggtggggatgggcccttgctcttaagcacctgttcattaactttatcattattggtattagctatcgaggctttcttaaaacttcaggtcatgctctagagatgctaggtactgagtaagctcaaagagtcacatagtcagtggggaatctagtagaaatgcacatttgagacctctgggtttccaggcatgtaattcaagaattggggcagtttggcatttcacagagtttggtttctatatatccatgcaacaagcataggtaacacatggagttacagataattgattgggaggaattggccttgaggcaattcttgaggcaatggaaaaaatagcaaatgttttcaagaggacaccacaatttgggggtactgttcggcactttccatgaacagcagatatagtggaacatacagtaaattggattttttagaatccagaagattcccaagagagaagcctagctattttttttattcaaagtttattaaacaatacaaacattgttctaaaaagcaagaagtctggaaatattttcttaattaaataatgtagttgacaattttcacatttactgccggctacttatccaagtgtgcatgattcaaaggggttaaataacctgcccaaagtcactagctagtagccatagagctagatttcaaacctacgtctttctggctataaaacatacattgtaactatttcattatattatacaaaatggtgagtagagatggcttccttttatttttattttttagattaatgtgaccgcttatgactaaacttagaattctgaaaatgcagatgtccaatgaggtagtagtatatacatctaaggcaataacaggagtcttttttgtttgttttactgttgacatatattgccttggactaaggacttcatatcaccctaagtgtacaaaactgtaatcagttcataggaagagaatttgtacaaaattaaaattttgtattaaagtgctattactatattgttggtatcctaaatagcaacatatcctgtctttatgaatggtttgcctaacgtggtagtgggcccaggaagtagtgaccattgtggtttagaagatgcttagtatggctgagcatggtgtctcacacctgtatacccaacactttgggaggccgaggcaggcagatcatgaggtcaggagttcgagaccagcctggccaatatggtgaaaccccgtctctactaaaaatacaaaaattatctgggcgtggtagcacacgcctatagtcccagctacttgggagactgaggcaggagaattgctcgaacccgggaggcagaggttgcagtgagctcagattgcgccacttacttcagcctgggtgacagagtgagactctgtatcaaaaaaaaaaaaaaaaaaaaagatgcttagtataatgaaaagttactaagaatttctgaaaaattttcaggggacctattcttcaaaagaagggaaaaattttaaacctgaactggagtcatgtacatattagaatattttataaacaaaaacatagttgataagacgtgtcattaatgttaagcagcagatttacttccagaggatctcaggaatgaagtgtatgactgtgtgtaactttacatttactcagcttgactatgatttgtttttttattgaaatataccatgctttcaaaaagcccccttgtgccacatccaagagcagaataggttctttatataaggaaaccacaaatcttgaaaacagcagtttcccttgatgttctttggccacactactgtttggtttaactagcgtttctggtattggagttgtttgagtctcagaatgactttgcttagaatttttatatttctgcccgacctcttcctgttgacatatttgaactgaatcaaaattatttgagtgatcatataagcatgttaaatatagcatttgaaaacagcctaagggtctgaaatattaacttattcgggtgtgtttgcttggaaacctggaatgaagactgtttgaaaatatatttgtttttagaatataatctaaaaaaggctttaaaaattctgaggctcagtaaagttttaaaatagtaattattcatatctcaacatctcagacgccaaggcattttatgtagtggaaatggggcaggtggtagaatcactacattttaaaaatatgcagctatgtcctcattttaaggaagattatacctaaataccatttctatttttatatcaggatcacctatgaacaggatttaggaaatcctcattatctttccaaaactacacaattgatataaaatttaacatttgtgtcattatttttcattctgagtgataaactataaaatgtttcttgaaaatatgtatgtaccatatgctgaaaacaggcacatttctgacagggaaatgaactacctgtactctcatttagctattgggtgaagtgtagattccactgtttaaattctttaaagtttataagcgcctgaccaagaggctatagagctaaaagatttatagccaaagaattaggagccactgctaatggaaaagcaaacaaatttttaaaaaaagtatctgagtatgtgtatattacactctaagtatgatatatatttaaaagtgtctctgatattataatgagagttaataggaaattttaggttgtgtcttctccaggttttttttttttttgtgtgtgtgtgtgtgtgtgtgtgtgtgtgtgtgtgtgtgtgtgtgtctgtgatgaagtcttactatgttacccagtctggtctcaaactctggggctcaagcagtcctcctgctttagcctcccaagtagctagactgtgtcagcccagctatcccagtttttatgaaacacttctgtaaaggaaaataccctttcctcctaactaattcctatattcattcaagaaggaattcaactcagttttcaattgttttgttctgttacggaatgttaggtttagtgacttagattaaccagttctaaaaacagattacctgggttgtatgcaggcccctctgcttactgctctgtaaccccaggcaagatattgaccttcctaagcctccatttcttgagtaagagtaggcttatagtacctggagcccagagatatttgggcgttaagtaatgtaatatgtatgatgctcctgacacttctaagtacacagtaaaccttagctcgtgttatgatggaggtggggttttcctttatacataaagagaacttcattttacagtaacagtaattaattgttactgttaataaaactgtcaataaaatggtaatgaactgttactctaaaataaaatgcttataaatatggaccccatcagaactacagttttaatgttcaaaggatcatctacattgagaaactgttttgagaggtaaatcattgctgtacatgtggactagctctgtgtcatgcaaaagtgttactgctcagacaactaggtcaggggctgggcatgccaaaatgagcaacctggtctctgcccttacagaatttacagtgtgggaatggagatagttgagaatataaataacccaggggagtgaagaggtgaagagagcaagtaggaaagtgttgatcttggactgcttatgaaaatactcagccacgggcttccaaaataggacactgcagctgtggtctgagaccccttcaaatattatccaagcagcgtgctcttcttgagagagggtgggctgtgtggggaagcctgacagtcctgatgtggtgatctgggaatgggtggtgtccaggtaagtattaaccaaagatgtgatattaaaatataaattcatggagtactgaaatcattttaagtgagtaaaccctgcctttgataggcccagataaagctctggtttttccctaagacctcacttagtgcgtagctgtactttttatcacaaacagaagaaacagatgttttggggagaataggtctcaacctcacctacatgttggaatcacttgaggacctctaaaacaaaacaaaacaaaaacctcatgcctgggccccaccccagcggttctgatttaactgcacccaggcatcagtgtttttaaaactctccagatgattccaatgtgcagccaaggttgagagccactcttcctccctatcctaggttttatttccccccagatgttccctaataaatttcaggagactagctcagccaaattctgtcaacaagttttccaatcagaagttattaatttaataccagtccatgtgttttccctttgaaaacatgtgcactttctgcatttcagtaagtaatgttggctcctggagaaatactcatcttaagctcctagtggagcaaaagaacctttctcatccagcaccctgcctccagacagcaagctcaccatcaaaacatgattcactgtcctatttttcacatctccttggcatgaccaaccctcagatttcgtcctgtaaccatttatttggtcatttaacaatgggttgtttcttcctaaatctctcatgcagcagtttaaagcctgtaatgtgcaaagttgaaacattttgtatttggcactgaagtctatccatggacatccaggctctggactcctctggtagccttttgtattcctttgcttgtctcaatttacatagtgtttctttttgttttgttttgttttccaagtttcagttatttactaatcagtgcaatcgccaatagattacatcaacatgatttcatgcatttagaggagaagtatttcctggttaagcggaaaattgtgtggatggcttctggaagaccttcattctaaagcagctttatagtgaaacatttgttagaaatctggaccttctttcttcagtttgctgtaatccacaattactgagtagaacttgtattgatcattgggagccagtttgttccagggctctggattattctttctgtcccaactaacatctggattgaacaatgccagacacgagatacagtgctgctccagtacctccagctccaatgaatacaaagagggagatcaagcttagatgcttcttggactgagtgatgatgtggtggagcatgtttgctgaagaggcctccgattggaaaggagagaaccagcctagccaccaggctctggcctaagtagtatctccatagtgtttcttgataggttctgtttctaccactttactcttctaattaacagatatctggtgaatgacaaatatgggccagacactcttctgggcattagggacaaaacattttgcaaattgggcaaaaatttctgccctcctgggacttgcattcaagtgggaggagagagaaaaggaaatgcataattagctaaaatatatatagtatgttcaaaggtgttaagtgctatggagaaaaataaggcagggcaggaggaaagggagtgctatggctgagatatggtgtgatattaaattggctttgcagtattatctaaacaagaggagggtgaaggcttggaccagaggagtagtgatagaaatggtgaaaagtggttgcaatctgggtaaatttcagcaaggcctaaagaaggtgaaggattgagccctgcagataccaggaagatgagaattccagacagagctgttgcaaatgcaaaagcaggagtgaacctggggtgccaagaaaccactctaacaggggcatccgatcttttggcctccctgggacacacttgaagaagaagaattgtcttgggccacacataaaatacattaatgatagctgatgagctaaaagaaaaaaaatcacaaaatctcataatgttttaggaaaggaaagcttgcaaatttctgttgggtcatattcaaagccatcctgggccacaagtggtctgcaggccacagattggacaagcttgtgctagaaggcccagtgtggctggagcaaagcgagtgtgcaggagagggctatgagactggtgtggggtgggagtgagattgcagagggccttttaggacttttgtaaagattttcgctttttcacagagtgagatggagaattattagaggattttgaatggagaagaaacccaggatcattctggttgctcacttgagatgactctctaggggtcattggtagaagtggagagactaatcaggaagatattgcaataatcttgcaataatctaattgagagatgatggtggcttggatcggggtagcagctaagatgggtcagaaatcgttggaatctgaatatatttttgtaggtaaaactttttgcccttatgtctatgtaatattttttgatattcctcttaaaatagagccctaaactccagtaagggacctgaataatgctagttatagcaaaaggttaactgtctgactcttgaataccacaaatttagtattgtgttttcagcccccgccatggggtagatattattttcctatgtttcttttttttaattattatactttaagttctgggatacgtgtgcagaacctgcaggtttgttacataggtatacacgtgccatggtgctttgctgtgcccatcaacccgtcgtctacattgggtatttctcctaatgctatcccttccccagcctcccatcccctgacaggtcccagtgtgtgatgttcccctccctgtgtccatgtgttctcattgttcaactcccacttatgagtgagaacatgcagtgtttggttttctgttcttgtgttagtttgctgagaattatggtttctagcttcatccatgtccctgcaaaggacatgaactcagccttttttatggctacatagtattccatggtgtatatgtgccacattttctttatccagtctatcattgatgggcatttgggttggttctaagtctttgctattgtgaagagtgctgcaataaacacacatgtggccggacgcggtggctcatgcctgtaatcccagcactttgggaggcagaggcgggcggatcatgaggtcaggagatcgagaccatcctggctaacacagtgaaaccccacctctactaaaaatacaaaaaattagccgggcttggtggccagcacctgtagtcctagctactcgggaggctgaggcaggagaatggtgtgaacccgggaggcagagcttgcagtgagccgagattgcgccactgcactccagcctgggcgacagagcgagactctgtctcaaaaaaaaaaaaaaaaatacatgtgcatgtgtctttatagtagaatgatttataatcctttgggcatagacccagtaatgggattgctgggtcaaatggtgtttccagttccagatccttgaggaattgtcacactgtcttccacaatggttgaactaatttacactcccaccaacagtgtaaaagcgttcctgtttctccacatcctctccagcatctgttgtttcctgacttttaatgatcgccattctaactggtgtgagatggtatctcattgtggtttttatttgcatttctctgatgaccagtgatgatgagctttttttcatgtttgttggctgcataaatgtcttcgtttaagaagtgtctgttcatatcctctgcccactttttgatggtgttgatttttttcttgtaaacttgtttaagttctttgtagattctggatattagccctttgtcagatgaatagattgcaaaaaatttctcccattctgtaggttgcctgttcaccctgataacagttttgtttgtttgtttgtttgttttttgctgtgcaaaagctctttagtttaattatatcccatttgtcaattttggcttttgttgccattgcttttggtgttttagtcatgaagtctttgcccatgcctatgtcctgaatggtattgtctagattttcttctagggtttttatggttttatgtcttacagttaagtctttaatctatcttgagttaatttttgtataatgtgtaaggaaggggtgcagtttcagttttctgcatacggctagccagttttcccaacatcatttactaaatagggaaccctttccccatttcttatttttgtcaggtttgtcaaagatcagatggttgtagaggtgtggtggtatttctgaggcctctattctgttccattggtctatatttgtgttttggtaccagtaccatgctgttttgattactgtagccttgtagtatagtttgaagtcaggtagtgtgatgcctccagctttgttctttttgctaaggattgtcttggctatgtgagctcttttttggttccatatgaaatttaaagcagttttttctaattacgtgaagaaagtcaatggtagcttgacagtgatagcattgaatctataaaattactttgggcagtatggccattttcacgatattgattctttctatccatgagcatggaatgtttttccatttgtttgtgttctctcttatttccttgagcagtggtttgtagttctccttgaagagatccttcacatcccttgcaagttgtgttcctaggtattttattctctttgtagtaattgtgaatgggatttcactcatgatttggctgtttgtctattattgctgtataggaatgcttgtgatttttacacattgattttgtatcctgagactttgctgaagttgcttatcagcttaaggagatgatgatggggttttctaaatatacaatcatgtcatctgcaaacagagacaatttgacttcctctcttcctatttgaatatcctttatttctttctcttgcctgattgccctggccagaacttccgatactacgttgaataggagtggtgagagagggtatccttgtcttgtgccagttttcaaagggaatgcttccagcttttgcccgttcattatgatattggctgtgggtttgtcataaatagctcttattattttgagatacattccattaatacctagtttattgagagtttttagcatgaaagggtgttgaattttgttgaaggccttttctgcatctattgagataatcatgtggtttttgtcattggttctgtttatgtgatggattatgtttattgatttgtgtatgtgtaggcctaggttaatatacatgttggcgtcttcgtttttagttaaaaaatttaaaaagcaaaatataataatttaatagcaaaaaacttattgaataaggaataaaatatatttttgcgcagctataaaatgtgtttgtgttttaagccaaatgttattatgaaagcatcaaaaagttataaagaatgtttaaagtttaaaaagcaaaaaagttgcagtaacctacagcttattgttgaagaaagaaaaattttttttataaatttagtgtagcctaggtgtacagtgtttattataaagtctacggcagtgcaaagtaacatcctaggccctcacattcactcatcactcacttgctgacccatccagagcaacttccagtcctgcaaactccattcatggtaagtaccctatacaggtgtactatttttaatcttttatacatttttattttacctttcctatgtttcgatacacaaatacttactattatgttgcagttacctacaggattcagtacaggaaaatgctgtacagatttatagcctaggagcaagaggccaaaccctatagccaaagtgcgtagtaggctataccgtctaggattgtataagtacactctatgacatccacacgacaaaattgcctaatgatgcgtttctcagaacgtatccccattattaagcaacatatgactgcattttgacccaaatgatcaatgattgctattgcactgctgttgcttaataaacatgtacattcccttcttgcagtctttaccttattccatggtaggtccctggaagttgacagaggccggcaatgtgagtagaggaaaaaacatgatactgttaataatataaaggagacgagttaacgggtgcagcacaccaacatggcacacgtatacatatgtaacaaacctgcaccttgtgcacatgtacactagaactttaacaaaaatatatatatataaagaagcccatgattcaaaacaaatgaggcccaggacagttcttgtcccactcctctcctttgttccttctcccaactctctaaataagatagaattgtccttttccagctccatgcagatctctcaggtggcctctttgacttctttgcttcctctttgcccctaaacagggtgcaggtatcatgagaaaatgcagagtagtacaaaagaaatctggtgttctcttccccaaaaccaagtgcaccttactctaatttctgttagtcacataaacatttctgttattcattttaataactggaagaatttcaggttaagtagatgctcatttctcatttcattggttcagcaattacttattgagtaccttttatgtgccagatactgttctaggctctgggagacaggagtgaacaaaacaaagtccccatttcttagggagcttaccttatgatgcagcagagagatagaagtataactaaatgagtaatacccgtatagtacatctgataaagataatgctattgaggaaagcaacttagggaatgaaaaagggagtgctgggatggcatgctcttgcttttggtgagttggtattaatattttcaatagggtagccagaaaaggtgtctccaataatgatatatgtgcagagacctgaaggaagtgagaaaacaatattcatgccaatatctggtggcagaatgctccttgctgcaggaagtccttagaccgttttctcctctctatatgctgcctaggtggtttcatcctatcactttttataaccctaatgactcccaaacttctatctctatcccagatctttcctatctctagtccagtgtatccagctgtttgtggagcccctccacttgggtgtctagtagacatctcagatttcagatgtctctatccaaactccttattccccactccctaaacctactttctgtagcctctcactccctttataaaatggcaactccattatttcagcttctcaacttaaaaatattggactcataatgattcttctctctcaaacctcacacccaatccatcagctagagcacgagttacttatattgggaaaaactaaaaaaggagtaaacctagggcagtgggaggaagaaattaaggtaaaatacaaagcagagcagtaatagagctactttctgggcagtgtttgatgggatgaggaaaatctaagctgaaggcatttgagccttggaactcaggttgctgggaaatcacatcctaagccacattcccttaaaagagctaaaactttaaagtggtttgctatgttttgatatgtcttaacatcaagagtgagttagttggcaaaactcaagctgtcaacccatgggttagaaagagactttaggtacccgtgagaatgttttacataaagtttacctaaagtattttccatttcctgcttctggaaacattgtgggacctgaatggagaagccagaagtgcaaagtcataataataattttaaaaaacagtgagcatttgaaccagtcttagaaaaatgaatgttctttgttggcatatctacaataaagcaaaacacttagctgaaacatcagggaaaggaaatgataaatgtcatggggtaaaagatggttaaatacttaagtatcttgattatattaatttgaaccatgaaaaggaagtaagtactcactgtccttctgttttgatttggtatacatttcccagcattcaaattctcagcacagaaggttcttgctgttttgctgcaccaagttcttggaaatcacatacagatccccctaaaggagattggaacagtgcaatatttggggactgcattctccacccaaggatttagcatcaaatgaaaaacaggtataaccaagagcataacacaaaagagcttcagtggctctggtaaaattttatttttttagctatttattatagaaggataagtttcaccctagtgactccttcatggacagaagcggaatcctttataacttctaaaaagatcattcctcagactggatttctctcagtgtcagattttaggctacagtgttcaagtcacaaatatgattgccctgaatgcaatcctgaaagaggtgaatttccaaataggtccttttgccaatggatatcaagtctgaatttcccgtaaaggagattgttagagactaaaataatttccctctttaaatgaaatgagcttggacattggaatctgaaagccctgaggtggaatttttgctatgcccttcacaggctttccaatcttggagaaattatttaatctctctttgtgtgtttcctcttctgtacagaaaagataatacctacctcgtggatttaatatggaggttcaacagataacatgtttaatgtctggcagttagtaagcatgataacattatatgtcaattatataccttttatttcctatttaaccaacacaatttttttgtttcttctgtttttataaataattctattttagtgtggtacctcaaattaggacagaagtctccaaacaccgataaattcttattctcaaaagtaaacttcattattcacaataaccaagatgtaaaaataaccaaaatgtccattgacagatgaatagaaaaagaaaatgtagtatatacacacaatggaatactactcagcctttaaaaaacaggaaattctgcaatatgtgacaacatggatgaatgaggacgttctgataagtgaaataagccagtcacagaaagatatgtgattccatatgtgaagtatctaaagtagtcaaattaataaaatcaaagagaacactggtggttgccaggggctggggggaggggaaatggcgagctgttaatactaatcaatgggcataaagtttctgtttgcaacatgagtaagctttaaagatctgctgtacaacattgtacctatagttacccaaactgtatggtatgcttaaaattttaagcgggtagatcttatattgtgctcttaccaaaataacatgtttaaaaatcataaagtcattatcatagctatcaaatttgattgtcatctctattgctaatattagtagctgtcatctattgagtgctaattcctgcccagtaccatgattagtgtttgccactcttacatgcttccccagcaatcctgtgaagcaagatactgttcttttccccattctcaagatgagaaaacagaaggattaagtaagtttcttgtggtcacccagcttacaagtgacagagccgggatacagactcaggcaccctgacttcagtgccatgctcccaaccactatgattctgaagtattatttttatattctagtactatattaatatgattgtctgcccttgagaggagtcatttattgccttaataaccaatgacagatctcctccaaaaaagtagagattggacatggaaaatgtaacctaggaggatgtttctatttcttatgtttgtttcttttttcttttgccttttcctataataaaaccagatatttgagtggagattctatgtcatttgcagaaaagtgagtgggtggaaataatccataatgtttaacttttttaattatttaagattatatatcactgaagaaaatctcatttgatccctgcaaaccctttcccattccccttatttaattctgtgagtcaaagaaaagaaaggtccagaatagttattatatcagcttagttcttattattaataccatgctttcactcccttggatattttctgtatttaatccgtttacatgctttttgcgtgttgccatagtaaagctcattccgtgatgtgagagaccctagtgctgaagaaggttttcttagcgttaaacctcgcattttcatttccgcattttatcttggtgttaccagggcaacccaaataatttcattttctcttcgaagttgatgcccttagcctgttataccctcatgctgactcattcatgttcttgccattttttttctagtgtaattctggtaactactttctttacaacatatgttttctgcttttctttctataatcatagcagctaatgcattgttggagtagtggttaaatatttggagggattacaaatcccactgaagattggaaaagagctgtggactttctcttcagaaaaatgtcaatttaaaaaataatgaatgaaaatattgaatatatagaagtttattcaagtatctctttacaaatctatttggttcttagcttttgaatagcaagttgcaagagtttatacagcaaggaatttatcaaaaaaactatctgaatctattccatctttaaatttagattgtgaaagtaaaggtacctgtttatttctgttttcatatcttcatcccagggcagtcccatgcttggttttataccctgtttgtctctggcatgtctcttggcaaaccacctgcaggagttaaaaagctagttggtatgcatcttttccagtgctggcctggcttatgcaagccccttgggtttgaatcagtataaagagctgaagagcaaagaaaggggataatacattttttggtttccttgtttatagatctgttactcacatatgctcttttttttctttctttttatttttttgagatggggtctggctctgtcacccaggctggagtgcagtggctattcatgggtaccatcatagctcactgcatccttaaactcctggtctcaaatgattctcccacctcagccaccccagtagtcacatattctactatagctttttatttatcttctatccccagtgcttggcacttagtagttaggtaaaatgtgttgaaaaaggttagggagggaagaaggttagagaggaggaaaggagaaaaggaaagaggaagggcatactcaaggaagggaagaaaaagagaaaagtgaaaaacagatcaaaggagactatcctctgagcaaagacaatctctgtgcatagctcagaagtcagttcctaaggaggaattctaggtggatttcgagtgaggggccctcatggcaggctgacacacatttggataaatgcaggagagtaaagacatggttgacccacatgttgctgcgtgtgttcagtctcaggagattgttgagcagcccatgaccggtgatgaccatcccactgagattctaacggccttcagtgcctgtcatgagaattttgtgaacttggtacaaacataggctattgagccagattttaacatggaattaatgtggaggcattaatgatgtttacgctgaattaatgtggaggcattgtgtggaggatgaccaagtataggtcagagtcagtttgcatgttctagaaggcagttttctccatttaacagccacatgcaaggtgaagctgtgaagctttacatgaatctgcagaaagcatgggtagattttcacacataccgtacaaaacaacttttggtgaatgagtcttgtgactttttgccagttccatctaagagcatgtcatgtgttgccctccttgtctcagatcatctggactggctcaggctacccctatccttatgtctctacagatagaaaatgacagggcccaaccttattcaaaataaacagaaacaaaacatttatgagtaatggatagcaaaatgaaacattttaagaggggaaaaacaaacaacaaaaaaagccattacgttgcagacccttcactctgtctgaaatgttctcctggccttggcctccaattttacctagctaactgttactacttatactatacttcatacttccatgcaagcatcacttcctaaacccccagtagttcaggtcctaacatatactgtcagagtgacctacacttctacttcatcatgcttgtcacaattgtgattaaatggttgtgtgtcattgtttactgtctgtctttccaccataatgcatgattcatgaagacggggattatgtctctcttactcatcactttattcacattaacgagctcaataccttgcagatagagctattctaatacttatagaaagaataaataaagttccaggcatcgtggtaagtgctaggaatataaagctttcaaagacaccgcatctgtcctttggaaacgttgtactccagtagctatctccattgataatctattcagcctctagaaacagaaagaccaaggatacattaagatcctagaactttctccacccccattcccctacatcccatgtctttctcacgataatcccaagactggccgtgcttgtgtccaaggatgtggaatagaaatgaaccaacttatctttccaccaatttccatttcttcgttgactggatatttatttatttatttatttatttatttttatagaaacaattttaatttatatctgtgttacctaatgttttaggtttctaaaacattaggctgtgttaagtttttaagatgggtcattggaaaagaagatcaaacattacaagaacaagtaaaaacaaacatccccacagttgtttcataaaccctgtcctttcccttggccctaagcctactttccactgcccaaaacatcatctacttcattcctaagtccttcttcagaacttccaaagatctctcttctttgaagtgttggacttttaaaagtgttttttcttctttcttccatacttttttctacttgtctaaagttttaattgaattcctatgacctttctatgctattttttattttttagcatttgtgattaatatatttaaacctcccaatttaacaacttttgagccagttttgctcttttcaagcacccgcaatccgcctttttaatcctcctgatgaagttgctgagaaaaatcagttctgaaatcagctttagatgtcaccttgcaagttgatacattactagacttccataggttttcaaccgtttaaaaaaatatatctgcaaggtctagatatctttgagatggttttctcaggaagacctcatctggctccaagccagctttcttactaataatagcaactccaagtgatttcaaaatactgtaccgtttgtcaatgaagatggcactgtttttggtcttgttccaagttagctgatgagctttatatttcttcctctacttttttttctgacataggatgcaaattaataaatccaagatattacacttagaccattcttgtaactttctttttaattgctcatctatttacccaataaatatatattgagcacctactatatgccaaatactgtgaagtgttgggaaaacagtggtgaacaaaacaaattatttctgccttcatggaccttatagcctagggaagaccaatgtaaatccaagagcacaaatgaatggaaagtactctctgacagaggtcatcagggggtatcctcaatgagagcctgaaactctggatgtgacggagtcagagagattggggacagcttccctgagcaagtggtgctagaacctgggtctgcggtatgcataggagttaacaaagcaaggtgggtgggtattgaaagaacgttccagttgtaggattagtaccaaaaggcccctgccactattatttatgtcttctttgtgatgacttttcaggatctacatagactgttggcctttgctatcatgcaatgtatgttacctagtttcactatttctttttgagggaggataggttttttatctttttaaattaaagaacatatctatcttcagaaacaatgtattttaattaaatgattcatattttccgagaatatttttcgtatagatgttcggcacaatgtagatttgagagaccagtaacatttggctatttccacctagagccttcctataaagaaacttttcatgccctttctcagcttctagaatgtgtgactctatgagcatcaaatgtgtttgcattagatatttagctctgctgtgaataaagaaaacttagaagagggaactcataaaatgtctaacttggaaaagatggccattaaatcagtatgttaaatatagagccaaacccagcaattatttctcctttagatacaaagccttgcattaaatattcattagctggacagacaaaacctaacattctgatttttaggtacattcttatcagttttaatgctcctgaagggccatttttcctggaggctggaggacctgaaatttttccttccatcacaaactttactgagctcatccaacaggaaagaccaatcaacagctggcatgagatggagggcagccttcttgaaaagctccaaagataattagtcaaccgttagtgtttttctgcaattatcaaactttcatggtccctgattctagatggtacattttaaaggtagattcctgtaaagattagcttaactgaaaaggaagataaaaatgatcatactctaaacccattagtctttcagtctctcactttaaacatcagtctcttggtttctgtgcagtgttactttgtttcctagttttttatgtttaacctagctggaaattttaaattttacccctatttaacaaaaacaaaaacaagtgaaaaagtgtttgacaaaaagttgctttagcttccttgtcacactttttggttgacagtttgtataagtaccagtctaccatctatttttatggaaaggtatttgaaaaacaagctacatttcttgtcagaacagaaaagacaactttctccctatgaaatcattaagagttcaactcaaaaaaacaaaaaaacaaaaacaaaaacaaaaaacctttttttgagtattctaggccccagagcaaggaatataaaataaaaggtgaccccttgccttttcagaaaactgtaaattccagaagttacaaaataaacaagaaactttagttttaatgctcttgagggcaggagtcttcttgccttccgcaccctcctctccttgctgcccttcatcagaaagaaagcagcattccccgcacatggctttgccagctcctgtcatgcctgggttttctgggctgcgctgtgtgttcatggtgtgcctgctgcttagaaccctgtgccctctttgttcaaggggtaaaactggattggaggtaaacctcagtctctttctgtacattgaaggaagagatcaatgatcttcaaaccaatcattacagatttccccagttgaatttgacttagcttttggatgttgagactgataaggtgattgaaaacctggccttaaccacagccaatgatattgagagttcaaggagaaacacacagccttttcaagggtttagaatccgattgggattgggaggaggtgtgtatccacgctacctcatggaagagtgtttctagtttaaaggagacagaaaagaaaaaaaagaaactggagtgaagaggtgactaagaaggccagtaagatgcagagagctgagccatttctgagagagactataggtatactcagaataatagatatatggaaagaaatgactgacatttgaggataatcgtttatgaacagtttttgaaggactattagatctcagcttagcctcatttcagtaaggatttctgaatttaaatgtacttgaattgtgcggaataggcattaaataattattagtatcatatacatatagctgaaactatttgacaaaaatcccaaagaaattaaagctttcttttaagatctcagtgagatttagaaaagcaatattccaaaccattattttgttaataatactttaatgtttagatttgttttctatcacctgtttcttaataggttcatgttctgacaaggtagtcacaacatttggccattttcttgggtacaagctaagacctcagaagacattactgatcaacacatcctgtttagcagtagtgacagaacattgtgcattaaaaattcaatgatatttaactttgaaattataaattattcctgacttctttcctcatctcaaacattctcttcagtagtggatgttctttgaataatcattatttacaacaaggagctatttggtaatagtctctctcttatttattgcaatgtcagggtctatctaaggggataataacgtaatgaaagagttggtccttactacaaaatgctcgattgggtgtgtctttatctgtgtgggtgataagcatggataagtcgattttagtgtaaccaatttgtattgagggctgcttctaagacactgtgctactttcttgaagatacagagatgagatataaattctatccttggtaaaataaaaatactgtaacaacgtctttaaatgcaccattggcacaggaaacaataatcaaccccatttacaaaggagtttcaattcatatttttataagaatttcttcaatgaatattctcccgtgagtgtatatgtgggtgagggattctctcagcacctagcacagagcctgacatacaattccctctcataatttatagtaagcacatgagtgaatgaaagtgaattatgcttgtgagagggaaaaagcagctactgatcatagatttttagtgaattcaaaggcctgtggctacaataatataggtaactgtggaaactgtcatgtaaagcaaaagccttgaaatgtacagcctgaagcacacacaattaaaaggatttttctttaaatatgtgaagagcataatcagaaacgaataatatcgtcattgtttgagagaactaaaattgctaggtagaagtacttttgccacacccaccaacacccttagaaaaataggttaagctcaacttgaagactttccaaataatgtaattggtccattaattgaacatacaatatcatggagatatccaaggaaaagatggaagtattcattgggttgcaagttccgcttacctcttaagattcctttgagtcaaaagttctatcattttatgatgctatgaggacagtatagcttaagaatcatttaaatgtgttaatgttcgtttattttttcttttatattgtcaaataagaaacatgaaaagagaaatttgagtacaggaaaagttctatttaaactctttgtttcttagcacagtttaacttctttcagttacagtcagcatcatgtggtattagcaaagagctctagatggaaagaaaaagatttggttaaaagttatagttcaccattagctgtgttccttgggcaagtcacctaatctctcttggctcaggattttcacccataaagtaaggggtctggactggatgatctctttgacctttttcaggtcctaaaattttataacccatttatgcttttctcatttaattaaatacatttctacagaaccatataattatgttctgcttcagtggatttttccagaaatattttgtcatgaatgccaaaactgtacaataaaacacaatctacagtcatgctctcccaagggagaaaggtataatttttatgtaattgggaaattttgcctttttgctcctgctttttatttccatcttttcactgccaaaagaaaagcaatttgttttacaactgatgttttcagcatattctgatgactttttgatttgtggagactttgggtacaactgaaaagaactcataaatttaaaatcatggcatttctgttcgttacctcttttgcccttgttcacactggctttagctatgctaatgtcctggccctgtgtgtgggatcttcctaccatgacagtgtcagacatatactttgcttttagcttcagtaataaaaattgcaaaagcaattatatcccactttcttaactaaaattactatgtattcacccatttattcatcaaacattagttgagtatctaatacatgccaggcattattttctaggtacttgtaacacatcgatgaacacaatagaactgaaaaattaagttctgccatcatgaaacttgcattcttgtggaagtagagatgcaatacatcaagaaacacaaggccgggcatggtggcccctgcctctaatcccagtactttgggaggccaaggccggtggatcacctgaggtcaggagttcaagaccagcctgaccatcatggagaaaccccgtctctactaaaaatacaaaattagctgggcatggtggtgcatacctgtaatcccagctactcgggagactgaggcaggagaatcgcatgaacctgggaggcagatattgcagtgagccaagatcgcgccattgcactccagcctgggcaacaagggagaaattctgtctaaaaaaaaaaaaaaacgaaaggaagaaacacaataaatgagggttttttgacagactgcacgtgattttatgagacaggagtcaaggatgactccaagatttgtggcctaagcaactggaaagatagacttgctatttactgaataggaaagctgtaagtggagaagggtttgctgtggggatggggaataggggactgggacaggagtaggttttggacaggccaagtttgatgtgtctagtggatatccaagaggagatgtcgagtacgtagtgggaactctgagtctggatggagatatgaatttgtgaagcgtcaagtatagttggtattgaaacattgaggccagatgcaatcacaaggggagtgaatgcattagggaagacaagaggacctaagagtaagcattgggatgttccaaacaagggggtcggggagaagaggaggaaacaacaaaggaatagccagggaagtagaaggaaaaccaaaaggctgtggtgtcctttaaaagccaagggagaaatggtcaaattacatgaagtgctgctcataaataagatgaggaatgaaaggctaggtgcagtggctcatgcctgtaatcccagcagtttgggaggccaaggtgggtggatcacttgagactaggagtttgagaccagcctggccaacatggtgaaaccccttctctacttaaaatacaaaaattagccgggcgtggtggtacatacctgtagtcccagctacccaggaggctgagacatgagaatcacttgaactcgggaggtggagattacagtgagccaagatcacgccactgcactccagccagggtgacagagccagaccctgtctccaaaaaaacaacaaaagaaaagataaggaattagaactgaccattggatttaacaacatagatgagcaatacacatgggtttcttttctgttgaattcctatttcatgtgctcaggaactattaaattgttttgtattcgttttgatcagggccattcactgggttactgacaggctacctaccatcatttgaaattggttcttgttttcttgaatgatatatgattgtgctaaaggtgatactatttacccactgtcagaaaaatgtggaataaaacaaatgttaactctacccacctgatagccacaaaacagtgttagcaatcgtgttggaatcacactagaataagtttcatattctttaggtcatggattgataaatagagcagaatacattctgggatattaattctaggcaagaaacacaaaaattatcctggggaggaggatgggaacatattattttaggaagagccatataaataaaataaactctatgtttgtacctaactttaccaatattcttttttttttttttgagatggagtctcgctctgtcacccaggctggatggaatgcagtggcgcaatctcagctcactgcaagctccacctcctgggttcacaccattctcctgtctcagcctcccgagtagctgggactacaggtactcaccactacgcccggctaattttttgtatttttagtagagacggggtttcaccgtgttagccaggatggtctcgatctcctgaccttgtgatccgcccacctcggcctcccaaagtgctgggattacaggcgtgagccaccgtgcccggccaactttaccaatattctttaccaaaatatatatctttgagaagtaaagttatgtggaatatggatctactaaaatagttttttgtaatagttttttttttttctggtctggagatggccacaggatatttgaattggtagttgataagtgccaggtgtttcatgctttcatgcttttttttttttttttttttttctgttttgagatggagcctcactctgtctcccaggctggagtgcagtagtatgatcccagctcgctgcaacctctgactcccgggttcaagcaattcttctgcctcagccacatgagtagctgtgattataggtacacaccaccacatctggctaagttttgtatttttagtagagatgggattttaccatgttggccaggctggtcttaaacctctgatctcaagtgatccacccacctcggcctcccaaaatgctgggattacaggcatgagccactgcacccagcctacaataagtattttaaataaggaaatgaatctgaatgtctgcaaaaggttaccgaagcagagatgtaataattttcatttttaaaattaggataccagaatctcttgatttaattaaaaatattacacttccaaagacattactaataagataaattaaaatcctttgtagcaagttgaaaatcaaggttggaggaaatataaaatagaaaaatgtttaactagaaataaaattatgctgattgatctttatctccctatttaaacccagtagtttattcagaagtggctcccttagcttgaaatgataactcttgaaattgtcgcaaatcacttagaaatgacagcagcaatgaaagagcaattgatcaagtgtggctgtcactttttcagaggaactgaaagttgtgaaagagaagtacaaaaaaagttttcattgagcaagcaaaaggtctatctaccttctctttagatttgtagttttttattcaagtcttcccttactcacctttttgcatcaaaaacattaaggattaatgctgtgttagatattggtttggggccttgtaatatgttaattcaataaaatctggtacatctgtgtattagtctgtcctcacgctgctaataaagacatgcctgagactgggtcatttataaagaaaaaggtttaatggactcacagttccacatggcctcacaatcatggtggaaggtgaaggaggggcaaaggcacatcttacatggtggcaggcaagagagtgtgtgcaggagaactgccctttataaaaccaccagatctcatgagacattcactttcatgagaacagcatgggaaaaacctgcccccatgatttaattacctcccaccaggtccctccgatgacacagggggattatgggagctacagttcacatgagatttgtgtggggacacagccaaaccatatcaatctgtatatatatttatatgtaggatacatgtacatatgtagacatgtagacatacatgcacatacgcatttactcaaaaaaatttaaatgcggctgggcacgcctgtaatcccagcactttgggaggccgaggtgagcagatcacgaggtcaggagatcgagaccatcctggctaacacggtgaaacctcatttctactaaaaatacaaaaaattagccaggcgtggtggcaggcgcctgcagtcccagctgctcaggaggctgaggcaggaaaatggcgtgaacccgggaggcggagcttgcagtgagccgagatcgcgccattgcactccagcctgggcgacagagcgagactctgtctcaaaaaaaagaatttaaatgcaataatagttgtgttgtagcactaaactttgccatcttgaccagctgattccctatcaagtcttatattctatcttatctagactgtgccaatattaagaagcagtagataatacttatatttcttagatgccttagtttgactttatttcccatattaaaagcatgaggtctgtgaagcaactcagacaaggacccagagaacctcacctgttgtgagtattcaggtctttgttttttcaaaggaagacttagataaaggaacagtgaccctctacctcggttctcaaagtatggttcatggaagcctggcaacctagacctttttaagggaccccatgaagtcaaaactgttttgtaatgctaagatgttttttgcctatttcactgatggtgcaaaaaccatggttagtaaggcaccttcacattactcaagatagaggcactagactgtgctgaagggattgtgttcttcactggtacacacttgaaattaaaaaaccaggttcacttgctgttcttattgaggcagtaaaaattactgttttaataactttcaacctttgactgcatgttttattaatacactatgtgatgaaatgggaagaatacacaaagcacttctgctgcattccaaagtacaatggttgtctgaagagaaagctcttgtgtaatggagttgaaagtagaatgagctactttatcacatcatactacttttacttgaataaaacaactgattattgagacttgagtatgtggcatatatatcctcaaaatgaatgaagtgatcctgacacttcagggagaacaactaaagtatttgttgacagcaataaaattcaaactttcaagtgataattagaattttggaaaacttgtgatcaccaccatgagcttgacagcttcccaagacttaaacatttttctgatgatagcagtggtgatattaatgaatgtgatttttaaatattattactaaaatgtgtcttaatattttattaggatgaaatgtgtcaacatttggaaggtctggataactcagtgatccaatgttttccaaattaccaatgcatgatgttacaaaatcatgcgttacaaaatcatgcatatgttacaaaatcatgcatgtgtaaaaagatccattcaaagtacaagatagactaatttttttttttttttaagatggagtctcgctctccaccaggctggagtgcagtggtgcgatctcggctcactgcaaccttcgcctcccaagttgaagcgattctcctgcctcagcctcccaagtagctgggattacaagtgcatgccaccacgcccagctaatttttgtatttttagtagagttggggtttcaccatgttggccaggatggtcttgatctcctgacctcatgatccaccaaccttggcctctcaaagtgctgggattacaggtgagccactgagcctggccactaattaattttaatataatgaagtttaaaaagttgtttgatagagtttccagtttccatatcacaactaagttagtcaaggtttagtatagtatcaaaaataaatatctacaatgatataaaaaggatactcgtcccttttccaaccacatgtctttgtgaggccatgtttggtccatacatttcagacaaaacaacatatcatatcacaatagattgaaatttcaggcaaaacaacatattatatcataatagattgcagaagcagatatatcttctattcagctggcttttatattaagccaggcattaaagggatttgcaaaaatttagaacaatttcattgttctactaattttgttttggaaaatatggttatttttcataatgatgtatcttgatggatgagtttactttttaataaattatatatatattacatttattggtttttcacttctagtatggtaaatatgtatagatgtaacccatgtaaatacttcagggttctcctcattgttttagagcatgaggggttcctgagaccaaaaagttagagagccactgctctatattggtgcattttatttctgaaattgttgctcacttttataaataagtccaatttcctctacgatgtaaagaaacctatggaggagacaacaaccaaatctatcaaggaacaatagactataacacaggttgtttgctaattatccctgccgctgcagctgattaggatcaccccaaatctgagttggatgagcattgcttcacgaataagcatcagaatttattcaacagacagttgttacgagcttcttgtttttcaggcagtatggcaagtaatggggatgcaaaatagagtaagacagtaccttctgaagtggaacttggaagtcaagatgaatttgtgccatctgttattgccactgagacaactgtgactatcaaacatttggggcagtgggactgtaataatttactgagaaaaaaagaggccataaagtgtcaggaaaatcaaagtttggactagtctgaaattttaatcacactagaaatatacaaagtaatgtattgagcttttgtgcagtttgcctgcaatacatgttttaaggagacacataggcagaacatgtagattctaccctgaagaaacttgtagcctagtgggtggtaacagaaaaaagttaagaaaagttgtgtttcagtaaaatgaggaaattgtattctgcatagatgtcaagtaggtaggtcagagaggagaacatcgccaggccgttgaaattttcaacactgtgttaacagattcaaaaagaagacaaattattgtataagaaagtcacaatgcagcaagacggaatagataacattgtaattgttgccaaagctgtaatcaaagaagaggctttgcttttatgtgggatgtttattgtctatctagttctttttttttttttttttttttttttgagacggagtctcactctgtcgcccagcaggccggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcacgttagccaggatggtcttgatctcctgacctcatgatccacccgcctcggcctcccaaagtgctgggattacaggcgtgagccaccgcgcccggccattgtctatctagttcttaattcagtattagggaaggaaggtgaaatgtttttggccctccttggccacccttattccttgccataagataaagaaaggatatttcttctcacagtaggaggatgattgccataagatacagaaagaatatttctcctcacagtagcaggatgattaacctcaagtcctgggtcacactaaaggcagtaagaatgagtaagaaaggaggaggcacatcaagaaaagaatgtacctctttttcccttttcccagagccgtgggtgcaccctattcatttatcttctgtcatttggttaaagataggagtttccagactgaactgttgtctcagcggagaaaattaggataagtttggggttcacagtgaaatgtgagaagtacaggaaacagagaaaaaaatcaagatacaagcatagagacaaaaaggaaacaaggaagaaaaattgaggcctcctcaatccaccaccatcaataagacctttcttcttaaaagacaaggaccacgtggaaacactgttttcctttcccatggaacaagcaaaatgctttataaatagggctcagtaaagaattagcacaaaagaatgctaaaattacacttaaaaatggtttaaagcaaaaaaaaaaaaaaagcttaaagcaataacccaagatggtaagataaacagcaaaaacatttcaaaggatgagctctagtctttctggcatcttcatgttctgtttaagagaaatgagggtccaggaaataactgggacttggaaagtgtctggccatgttctttctgtcccacctggagcacaaagggaaccttgtcattggacaagaaaaggcaagtacttgccttttccatgtgggtctccttatttcctcaactggtgattttattcagtggctcatcaaaacattgatgtgtcttgacagctcttgaactaattcagacctctgctgtgaggagttggagccacatttggtaacaatgcagtatgttgtactggaagataaattgatttacaagtaatctctcctttctcagaaacatgattcccgtgacagaattccggcagttctctgagcagcagcctgccttccgagtgctgaagccatggtgggatgtgtttaccgattacctctcagtagccatgctcatgatcggcgtgtttggatgtactttacaggtaggtgcctagatccctggcaaaatgggggccagagcaaagcacaagtcattgaaacctctttagggagctggctgtgtgatttttaaccatgtaagtattagtcactgttctctgtggatgtatttgtaatcatagcagagaacctgaaacacctatttactgggatttcctaatacggtaaatatggtacttgaaagatttcaatgagcccaaagagcaattgactgtagtcaagtaatgtctgatgtcactaacatcaaagggggccccttatctttccaaattggcttcacagtgaaatgtaacatatgtccagcaccattgatactgccaaagtggaactttttgcttttattcatcctttacccttacccctaataagattttctggaagaactaaaattttgatgagatatcattattttactgaatgtttagttctgaatttgaaaagaacccaataaaaatttatgtagatgccatgtatttatgtattgaatccaggtattttttacatagctgctatgttccaggtaccatactttgaggacatggatacatataagtagttcatttctaggggagaaataaataaactagcaatcaaaataacttgtgttatactttgatagaggtgtccataaggcactaagggaatgctgaagagaaaccctcattttggtttgggaataagagaaaaggtgatggttgaattaaaacttggagaaccagctaaagttagtcaagaacctctccttagctgaaagtaagaggacaccaagaggagaaaagaaagctctgagtcatggatgtgagaaagaacatgacatctttgggaaactgtcagtagaactggcacatatagggtgttgatataagagctgaagccatgcttgcaaatggactcatccagggagaatgtgatgaggaagatagtgtcagtaataacattaaagagtaaatggagaaggaagtggggcccttgaagaagactgagaaggggcagccataaggacagatgaaaaccaggagagaggcataggtcagaagccaaaggaagccatggacaatgatggcagccaacacaactaactcatggactaagaagaggaaagtagcaactacgtcattagaaatcttaggtcagtggttggaaaactgaatggaaatcaacgtattatagaagctatggggtagatgtgatttttcgggtagatcagctggaaaagaaggtatagggagaaagagaaatcactagaagtggtacagagcgaaaataaagtacttttaaaagttggccttaaaaatagtgaacacatactgcttcctatgtgtcaggaactcttttgaacactttacatgtattttaacaaatgtcatcctcatgacaactcttatatataggttctattactagcctcacttacaaatgagacacctgaggcacagagaggttaacgatcctcccaaggtgagtattgaagcctttattcaatccaggtgatttggttccaaagttcctgctcttagtccctcacaccatgctgtcttttatttggtcactgaaaatatctttccccctcccctccaacaaagggtgccagtgagaagttattgtctaaaaaaaggggcataaattcattcataaatgaaatagttgttgaagaacctactgcgtatcaggcactgttctactaagaattatcaacgaaggagaaaaaagacaaaaatcccagctttatggactttaaattctatgggaaaaataagcaaaataagcaaaatcaattagtaaatatgttagaagataaaggcaatgggaaaaaataaagcatgccaagatggtagaaggttcctgcaggtaagatagaagctgcaatcttcaaataggatggcctgggaaggcctcattggaaaggtgacttttggccaaggaattaaaagacgtccaggagcaggccaggcagatgtcctggaaggaggcttccaggcagagggaacagcaagagcaagaggcacatagtgaaaggaaggcacaggtcccttccttttctgcagaagaattggattttatgactttctcagttttctagtttggtttccccggagacctgataaaggtcaaatgttaactgtgttactaggcaacattatggcataaatgactgctgattggtcaactgtttctgggctggaagagtgataaatgcctggtaggaagctgtatcattggacttctgtgactgtagtgaaacttgtgattcagcatgctccttgacacatcaaagattgatctattgagttactagagatattggttcctttgaggcacaaggcttttaagccagggtaagttctgaacctacctaaagtagatcactttccttgcactggagatgatttggggaggagaggctcaggaggacagcagtgtggacggctacatggacagttgtgaagattttcctaaaggggaaaggctgatgttgccttgctggtgactctaatctgtgtatttctgaagtgattctggtgccaaatgaagtttatgataatcttataagtctgcatgttaaggtgaacctagaaagtcccctggacatgttacaggcctgaagcagaattacctctgtaatgttgaagaaaagccatgaggccagtgtgccagaaaaacgtgagtgaggggaagaacagtaaaaggtgaggcccgagacacagtgggtgccagatcacgtagggccttctaggccactggagggcttggctttgttctgagtgagttagaacatgtttgaaggatttatgaccagaagattagcataatttatgttttaacaagaccactacagctgttgagaatatactgtagggggtgaggatggaagtgagggtggtatttggaaggctagtgcaataatccaggcaagaaatgaattaaatggtttgagtagggtgaaagcagttgttctgcatgtattttgaaggaaaacccaccaagatttgctgaaagattaaatgtgaaatacaagagaaaaagagaaatcggccgggcatggtggctcacacctgtaatcccaacactttgggagaccgaggtgggtggctcacctggggtcaggagttcgagactagcctggccaacatggtgaaaccccatctctactaacaatacaaaaattagccgggcatgttggcatgcacctgtaatcccagctactcgggaggctggggcaggagaatcgcctgaacccgggaggcaaaggttgcagtgagccaagatcgcaccattgcactgcagcctgggtgacaaagcaagactccgtctcaaaaaaaaaaagagagaaatcaaggatgactaagatttttagacctcagcaactagaaaagtttgtttgtaaacatgttaggtttgtgatatatgttaaacatcaacgtggagatattaagtaatcagttggatgtaaaacccaggagtttggggttagtgatggtcattttcaggaggaacgaggtgaagatccaggatctaggtggaaggattatccctgatttgggtggaggaggcatcatccaccagaatgggaatgatagctacagatacatttttatttggtagggagacagaaagacaggcgtctgtgccttatggcttgttgtttctctgtgatggagaaaacaacatttactgagaggttgtgagggtgaggaatgcagtaggaattggcaataaggaaggtagtagagcaggggtctcaaggagaaggttagatttggggtggctagcttgggaaatagtagaggaaggtgacatagattagcgggagcactatcaacctgtctttattactgtgccatggctcagagcgctgctgcaggatctgcctaacaagcacacggaattgtttctcttgtttacacattccatggcaggatgaaaagagaaagaacacagaaaatcttttagaataaatagttaacttgcaatgaaatgaatgaaaataagagtgtacctatgaaaatgtctataatgagcatcatttaggagatgtgcctgcggtaagttcaggagggaattcaagcacaagctaaacctcttttcctgtgtgctggtcagaatcatcttcctgcagcacagccccacagtgtctctctacagtccagaaggcttttgggctccccgctgaccagccttctcagcaccaccactcttacctcgcacaatcagtggctcctgctcagacaggttaactaacctgggcccttcacacataggcctttttcatctcttcagccgttaaaaagaaatctgatgccagttgactgggtacaagaagtgttgtctgagatgacaaggttctgagaaaaagtgagatgccaaaagaaatattgcttcagaatatgaaggctgcagggcacagaattcatacggggtggactactcaattcaacaaacaatcctgtgcatcagcaatatatcagacactctgtggggtgttagatgctaaaagaaaaacagcacagcccatgccttcagattgctaattgtctgaagaccatagaaacacataagcaatgttttccaatatgtaaatgccttaatggtgcctcaagagcatcaaatatgatgttgtcatctcagtcactggaagtgagggactcaagaagataattgagtgaagtctcaaagaatgaatatacaaaaccaaaatatatattttaataagacgcatgaaggggaaaactcagcattaacagaatatttagtgtgatcattttacaaagccctcatggattggcacagtgaccggagggcccagatggcatttttcggagagggtggggtcagggatattaaacatcctgcaatcggtgggacattcccacacagcagaattgttttatccagaatgctaatagcacatccatggagtaacactggcaagagaacctgtgctagaatctgtatgtactttatttagttcccagaacaaacctgtgaaagtagattagtcccatttttcagatgagaattccaaagtgtagaaggttagataacttgccccaaagccaagagggatgcagctgagattccaatcttaggctgattctccatgaggcgtctgtcacccctgtgcacactgctatcaaatattatggaaccatcacaattgcacagattgtgagggcaaggtttggcttataaaacaggattttcagtgtaaaagcaagtacaaaatgataaaaagagaaaatcgttacaaaaagtatcaattttatgaattataaattaaaattttaaggggcattcactatatacctgctatttaaaaagaaaaatttgcactctatagtgagcattaacaaaacaagaaagaatgcatggtacaaattatcaaagtaaaactacctaggatccatataacaaattaatgttccataggggaagaatttaagctcttattttagctctgctatgataatccactaaaaatggcttaatgactgataactttataaacaaaggattctctgtgaagctcaagttcctttctcattcccaaggcgatgttaaaattgttaaaatatatttttatgtcttgattttccacttgtaaattgaaagtgtatcattctttctgatcttatttacaaagcaagttaactcctatattggaatacatgacaaggcaaaatctaaggtgaagttagaagaacactctttgatgataaaatagtaatccaacagcttttagagtaactattttgtaagttgcttttttcatgtcaagttaacagttgatatctgtttactcctaaaagattgtattcatataggttcaacactactcagcttggaaaatactgggggattaaggagattattttctatcaaggggatgttgattctttcttgcacttatgtaaaaggattagaaccagcacttttgccttacttgtctgacaaatatttattcatttctgagcaagtagctcttttcaaggctgccatgaaataagagcatgtataaagagcattccttagattacatcaatccacataagaacaaatataaactcactataagccaggagagtgtaggagtatatgtgtctgtcctattcctgtacagtcatacaccctcaaggaccaatagtgtaaacacccaactttgtgctataaacaacactattcaaaaactcaacacattcaacaaaatagaccatatcctgaaaggtcaacacgttgcttcctggaaaaataagattgctttagatctctgctcagtgactccaggttatcttattatgaccaaatccaaaacagcccatgggcagaccgttaccaaatatagaattagcagttagaaaccagaaacaggaactggaagatgcaagccccctggaagccctaggggacctgccttgtgattgtgacatagaaaagacatttcctgctttctgaatttggaggggaaaaaagaattatttcaatgacaaagccacctacaaaaaaatcctggaagatttaacccaatcactaggagcgaagtggccagcagggtcacactatcaaaaaggttaggacatgtgagttttttggttagagctgatccccgctttgatttttagggcatactgaaattaatttctacttctctgatttagagccatcaatgtcagctccatgacaaatgaccttcaggtgaaaatgctcataggcagttcactggaaagtacaggcaatcagaattatagattccagaatgatatcagaagacttcattttgacaatgtgccctgaattcttgcttctactccccccatgcagtcatctgctgtatactgtctgggatttctagtagatctttttctttggccaaatttgaattttatagatagaagcccagaaattcttacttagaatcagtaatactgattacatcagaatactgatgaaatcacagatagaagtgaaggcttaaaatcaagacctcatctcagatatgttccttttcttttatcttttcttcctttttttttttttttttttttttttttttgagacagagtctcactctgtcacccaggctggagtgcagaggcgcggtctcggctcactgcaacctctgcctcccagttcaagcaattctcatacctcagccacctgagtagctgggtttacaagcatgcaccaccacacccagctaatttttgtatttttagtggagacagagtttaaccattttggccaggttggtcttgaactcctggcctagagtgatccacccacctcagccttccaaagtgctgggattacaggtgtgagctactgcaccggcccagttgtataaagtttccatgtacccaccttacatctccgttctactagggtctttggggaatataaaacaaaaattatttagatagtgaaaataattgtaaagagcaacctttgtcatctctgtggggatggtagtgaatggtgtgaaagaaacactatttcaaaacctctgttacatctagtatcaaaaatatgccttagttgggaccttctttttatggtggtttatttgaactgacacttttgatcttccctagaattttacttcttgtcatctatcaccttgacaattttatgtggactgaattccaattttatgtggactgtatacagggtggttataggaatacaggaataaattatattccagggagaaaaaagtgatatggtgtattacagaagataagacttgtggggcatggtaacattctacagatgtccacttgggggtttctagcaaacaaggactttcagctctgcttcattttgtttcgtttgttttgttattttggagacgagttctcactctgttgcgcaggctgctttgaaactcctgggctctagtgatcttcctgcctcagcttcctgagtagctgggattacaggtgcgtgacaccatgcccagcttcttttttttttttttttttttttgagatggagtctcactctgtcacccaggctggaatgtgtgcattggcatgatctcactgcaactccacttcccgggttcaagtggttctcctgcctcagcttccctagtagttgagattacatccatgcaccaccacacccagctaatttttgtatttttagtagagacgggctttcaccatgttggccagaatggtctcaaacttctgacctcaagtgatccacccgcctcagcctcccaaagtgctgggagcccagcccatgcccagcttttttgttataatagcacttcctgtttctaataaaagtataaagactttttttttttttttttgatacagagtctcactgtcgcccaggctggagtgcagtggctcaatcttggctcactgcaacctccacctcctgggttcaagcaattcacctgcctcagcctcccgagtaactgggactacagacgtgagccaccatgcccagctaatttttgtatttttagtagagatggggtttcaccatgttggccaggctggtctcaaactcctgacctcaggtgatctgcccccctcgggctcccaaagtgctgggattacaggcatgaaccaccgcgcccggccctatataaaggcttttgaacatacatagtgacatttttcatcatgtaatgacaagttcaaaatgttaagtttcctaagaagtgtttgaactactggcagtaatagggtcagctatcttaaaatagaattggcatctccctactcaagttattttagttcttaaaagctggtgagattctttgcactgatatccaaagctggggctatgtgaccttaagttcaatatgttaacttactgtttaggatgttgatgttaaatgttataggtgatagatattggactcagaaaatgcttaatgcaagctgagacagtgttgtagtgatattcacttgtagaacacttgtatgcatgattatatcagttagtagtcacttaattttatatttttagaattctgcaaaaatcttttgtctaataaataggccatcaactatctcatcattttaccttgtaagaaaccttatatgctcactggaaatagaaaacctggatttaaaaaaaataataatttcagctagatcaagttaatttaaagagaaaaaacaaaatctttccccaaaacttcaaacagaaagcatgggctctctcatatgctcttagtatgagtttaggtgaaacacatggcttttctgaggcccagttttataaaatgagcaggataggcatcacttccattttacagttttattgtgaaaaaaatggtgataaaatagaaatgtattgtttttagtctctttagaaactgtagacctcaaaatgtcttctctgggcaattactctgatcctccccaattgaaaaacaaaacaaaaaactgccttctcttcatttggggcatcttcatatagtcataagtgtgattagttgagtcttgacaaacagagattgagagaggtcttgaatccaggaccaaatgccctaattctgatagctacctacatctgtctcccagaagactttgcaggagctatacagacatagtggacaagatcaggcagaagaacatttaattaaagctaaggttctggccctaaaccacattatcagtttcctggtgagctaggccacacttcagataagtctcaagcactttctttgcctaagctctgaaaaatctacgtacctgcaaaaaaagtgggggtgtggtagggagttctgaaaaaaactccttagcttctttttctacaccttaatctctgctgtgctcaggtcaaagaaaggtaatctgagtaaagaacagacagaggggagaggggaaggggaaggggaagagagaagagggaggaagagagaggggagagagatattgatttattattgattcttcctaaaggtcatctgaacaatcctcttcaggccctcctaggctaccatcaacttcccagagagaggcagcttcaaacctgttgtgtcacatcagttttaaggaaaaagggagacttccaaatgatgactccctttagaagctctaaagagagagtcaaaacagactggtcccaaggagaaacaaccccttgtgacaccagagttcagtccttttccttaaaggtcaccatctctatcaagccctctttcacctagaaagaagtaaggaggagcaaagaagagtcttttctccccagagatcagccattatctggatgattcagaaaaatggaaaagtgcgtagatgaaattctccctctataggtaaaagacctgtttgtctgaaatgtgtggaacctgtcttatccaaattaatatttatggaatccaaataatcttaacttctaaaaattataataatgattgaaaaaaaaatcacttgccatccagcacacataaatgactaacaagtgccatgactcttgctttaactgcaagccttctatgatgccataacaaattatggatattatcttttaaggatgacactatctattccttcatgtagattggaccacaacctggcagttgatatcatctcacttggggagatatttttatttgctaaaaataaatagcaaattattttccaattattatagtaaatttctaaaataatcatttaaaaatatccttttagaacagttttatttgcatgcttcccatatttagaattctgtatctttgtcctgttcatacatcgaagataatctggatgatttatgtttctctttgaactctcccaatttcttttattctactcctgctcacagatttatcagtaaactgcagggtttctagagctaagtcagctgagtacagaactgaggtttcagtccctgggcattgcttcaaaaagagtttttcctgtattattttaagagcataattacagataaatatttcaaattcttcaaacaaccgaaacaaatagtctgttataaatcaaaacatgttctgtgttatgtatccattgaattttctcacatggttgttactttgaaatggatgctttccacatagatcttaaattgccatggatcaaataattcactggtctttatggcttatggaaaaatgccatgaagcagaatgagaagaagctgcttcaaatctcacccacccccttcatttctgaaaaattttttttgtaattccttgtttggcctagagaagctttcttcaaaagaagaggggaaaaaaaacaatgtggatacaaccaagatagaggtaataaaagcatccttgatggctggaaaccttcagtttaatgaagggttaaaaaaatgaacattaatagtatgtattgtcattcccaccctcagaaaagttagtatgtcaatatttacagtgataggagaatcgtatttactaagcaaaatttcaaaatgtagacatgattctaatgtggatatgatggtttcttttttacctaggaataaacagaccatgtgtcccaaatctggtactctctttgtgtctcttaaatgagttagcacttaactctggctttaggaatcactagactgatatcacttggcagttaggagagtgtgcaaaacgaattgtttcatgtcagaagaaggtagctagaggtcaaatatttccagatgttctctggcatctgaatatatgaatgcatatgcttgccttagctgtggtttgttcctccttagggatatgggggacagtgtttgttttatttattcacttcatatttattgcgtacctactttgtgccaggcaatgaaaactaagcatggtgtctatctttaaagagcttactggtccttatactgttatttgttctgtataatgatttaagatatgctgtagcttttttagtgcttaatctttctatcagtgacatttaatgttcctcaggcatttctcacggtgcataactcaaggagtttcacttgatactagcctttaattatgtattaataaaacctgtataaaagtaccattatactttaccactgagaaaatccataagttcagaaatctaagttttaatgcacttatctcttcaatcccttggatttttttccccaagcactccatgcctcaaatgtgccaaggtatgatttttttttccctttcttattgctgttggagaaataggcaactaataaaaacagaccttttactctgctcttgtaaataccttagactttggcagtttgccttagactggtttttctctcactttactatatgggtgtaaacttcctccctgtacagtgtgttatttcctggccatgatataaatgttaagcatcatcatcattaaaaataagggagattcagacaagtcccgaaggtggaaaaaaaaagaaattaaaaaagagagagagagagatgatccaaacaatggaatgctatatagtgctaaaaataaaaaataaaaaataaacggtcaagctatgaaaagacatggaggaacattaaatgtacattgctaagtgaaagaagctaatcttaaaaggatacatgctgtatgattccaactgtattatattctggaaaaggcaaaactacagacagtaaaatatcagttgttgcttagaaaggacaaaggaggggagggataagtaggtatagctcagaagacttttagggcagtgaaatgactctgtaggatattacagtggatacatgttgttatacatttgtccaaatctttatccaacaccaagagtgaaccccaaggtaactgtagactttggatgacagtgatgtgtcaatgtaggttcatcgattgtaacaaatgtaccactgtggttgagcttggtgataatggggagggctatacatggatggaggtagggagcatttgggaaatctctgtaccttcctttcaattttgctgtgaacctaaagctgctctaagaaaatttaaaaagtctttttaaaaagtagaagaaaagaagagagcatgtgcacagcaaacaggaaatgatgccacatacttgcagttttgcttttccatttctgttggagacaaggacggtgttcgtcctgatgatagtgatgcagcatgtgtggttctcatagatgtctatttctgtgctcctgtggcgattgcccccaaatatgcttatagtcaacataatgcacagcaagagttgtgaataagacatttgctaagaatgaaagcatttaaatattcactccttcagagccatctactctgccttttcagttccttttcattctcattctcaccaacattttttgtacttaaaatattttcaatagttcagccttgaaacacagtctacattttcagagttaattctatcactgatgaggggaatcaatctcatgcgtttggacaacgagtgctttctttgtcctctagcatcttgtatcttggggaaagtgcttagtcaagtttgtacattaactgataacttagtgacatgaaaattaattcctttcccttctatttctatccctttgcccaggagaaatgggtctacctcagaaaagaagagtgacactccatcttttcagtctttctccctctctctccccaaatgcatccttttgctcctgcctctggaaccacaggctctaacctagtagcttcaagtcccaagtgaagttccttctactccgccaagcctaccctgcttactccaagcctgagctcttactgtagatattgccatatttgtgctctcgtttgatttttcatgtggctgtttatgtccctctagcaagattgcaagtgcttttagaacagcaaatactggctatgcctctttggttttcctatgacgcttagcatgtattacgttggcacaaaactaattgtgttttttgccattactttcaacggcaaaaaccacaattacttttatgccaacctaatagtagatagtaggtattcaatatgtttctgttggtaaaggatagcctgctcttatctctgaacaatccctggagaatagatctttgggatgtgattttgctatatccttcttaggtcaacagggaaaacttggtttgttccgtcagactgggggcaagtctagatgatcaacttcagggtagaaatctttggtactacctatatttgaatcaatgaggtggagggaggatgaagcataaacttaactgccttatattggccacttgcctttttgtggctccactctacctgtttggatcaaatctgtcataaccaagttaaagtactgtctttttaaagaataagacacaatctaaagactacatctggatcacaagttaacatattggaatggtaccacctattttatcatacactgcaaattttcttacttcctcttcctataacaatataatcatagctgtgttttaaagtccaagctggtagctcatgcctgtaatcccagcattttgggggtccaaggcaggaggatcacttgagcccaggagttcaagactagcctaggcaatacagtgagaacccatctctataaaaaataaaaatagaaaacaggttcaagctggagaaataatttggggattatagttcatagaaaaagaacaatggggccaggcgcggtggctcatgcctgtaatcccagcactttgggaggccgaggcaggcggatcacgaggtcaggagattgagaccatcctggctaacacggtgaaaccctgtctctactaaaactacaaaaaattagccgggcgtggtggtgggcacctgtagtcccagctacttgggagactgaggcaggagaatggcgtgaacctgggaggcggaacttgcagtgagccaagatcgcaccactgcactccactccagcctgggtggcagagtgagactccatctcaaaaaaaaaaaaagaaagaaagaaaaagaacaatggactttattgtcgaggatataggaaagtaagtattttcatatgctgttggtggaacatgtgtaaagaaattcggcctttctggtggggagggaatttgacatataagtttaccttgtgtggcactacccaagcaacacaatttttcataataatttgtctcgactaatttttgtattatgatacacacatctgcgaaagatggtaaaacaggtgttacaattcaaaacagtcctgtgttcaatgtgtatagtcatgatagatataaaaagataacaaaaatgcatacacagaccccaaagcaagatgagcgtttctgtggagcacaaatggaacgctgaatgggtctgaatcaattgctgggctcttatccaaaactaagcagtagcagccaggagctcagctcctgctagtaaagggaaccataaatgcaccaagcaagataagagactggagcctggtcactgtttcaagccaaaagtcctcactcccaaaaagaagctgaaaaaatatagcgattaactggatgctggagccagtacttcagaggaaagtgtgcatctgaggtgcctgccgaccagaaactgagtcagcctgtccactgtcttgcagactgactctgtgaggcctgacatcacagtggaacatccaaataccatgataagacctattggtggactctggggggctgggggctgggtgcagaaaaccacaaaagtacactcaagagaagcagtggaaaacatacacctactcaggatgcgtctaccacccagaacacatgaaaaaaactatgttaagaaagactattaacaaaattaacagttggaacacagattcatgcaagatgaaattaagttttaaccagatgacaatgaatttaaaataaatatacagagagtggttaaaaagatcaagacataacatttaaaaagtaggggccgggtaccgtggtgaacacctgtaatccctgcactttgggaggccgaggcaggcggatcacttgagcccaggagttcaaaaccagcctgggcaacatggcaagaccctatctctaaaaaaataaaaattaaaaaataaaaaataaatttttttttcattagccaggcatggtggcacacacctgtagtcccagctacatggcaggctgaggtgggaggatcacttgatcccaggatttttgaagctactgtgagccatgatcatgctactgcactccagcctgggcaacacagcaagaccctgtctcaatcaatcaatcaataaagtaggtagatatgacttaagagctgatataacaaatttagaaatcttggaaataaagtcaatgaattataaaatatcaacagatagaataactggactgtactcagtagcataaagaagtaatgaattaaacaagagttctcagtttacccagagcccagcacagagaaagacaggaatcaaaacacacgctcatgaaagacagactgagagacactgacatacatctcagaggagttgtagaagaaaatggaagaaaggatggagcaatatttgaagaaatatttgctttaatttcccaaaattaaagaaagtcaagtccttagctccagatatggagtaggattaatccacatacattcaaaattaaagtgaacctgcagaaaatcctgagactaaaggtaaaatcttaaaagctaccttaaagatagattgcgcactaaataacacaaactgacagcaagcttctcatcagaaaaaatataagccagggaaaatggaataatatttttaaatgctgagaggaaattactatcaaaacagaattgtataacttaccaaatagttactgaagaatgaaagcaatataggaattgtcagtcttacaggatatatgagtttcctatccacagacttttattaagagttattaaggtgtatacttcaaccaaaaggaaaatgaacccagggagaagttataggatctaagaaaactagtgactcataatttaataaatatataaacaaacacaatcaagtacagattgtaaaaaaaaaaaaaaaagtttcaaaagaaagcaaaaacaaactctgggaacaacaaattggtatagttcagtgggtagttacaacatattcttggaagtggtgtggagatactaaaaattttaactgtacattttaagaaattatagataacatctagaagaacagaaatacagtcgctatcttcctaaccagctgagaaggggttgggtgggaaataggagatagagaaaaaatgagcagtgcagcagaaggcaggaaggagaagggaaaagtataaatgaaaatgaaaaatctagaaataagtccaaatatccaaatatgagtgagcaaaataatggattaagttcacttataaaaagaaaagggtgattagtttggatttttaaaaattcaaaaggcagaaacctaaaaaaattataagagtaaaaatagagattaaaagataatataacacaaatacccaaaaaagctggtagcgtaatgttaatattagatagcatttagccctcccatcttcaagaatacactctttatctaaacaaggtatatatttgagttttctaatatgcacaggttcttgtaaccaccactacaaacaggatacagaagaatctcaacaccccaaagaattcctggtgctgacctttgcaatgaacccgccaacctcagcctttaacctctgatctgttctatgtcctcatctctctgtcctatttttcagaatgccatgtaaaatcagatagtatgtaaccttttgagactaacacactttttagtagcaaaactccctacatgcatgtcatcatgggaatgtcttcagtaaattatagcacatccacaatatgtaattccatggcacttagaatgaagtagatctccaggtgccaatctattaacatctccaagacataccatgaaagagaaaaactcaaggtgcaggaacaatacatatagtaagatcttatttgtttatgtattatatacatgtaaatgcacagatacagatctggaatggtatacaccaaacttatcagggctcatgaaggaatggcaatggggataggaaaagggaaagggaggactctaccttctgtgttaaaaataaatgttttgcacatgaaaaaatgctcaccatcactggccatcagagaaatgcaaatcaaaaccacaatgagataccatctcacaccagttagaatggcgatcattaaaaagtcaggaaacaacaggtactggagaggatgtggagaaacaggaacacttttatgctattggtgggactgtaaactagttcaaccattgtggaagtcagtgtggcgattcctcagggatctagaactagaaataccatttgacccaaccatcccattactgggtatatacccaaaggactataaatcatgctgctataaagacacatgcacacgtatgtttattgtggcactattcacaatagcaaagacttggaaccaacccaaatgtccaacaatgatagactggattaagaaaatgtggcacatatacaccatggaatactatgcagccatgaaaaatgatgagttcatgtcctttatagggacatggatgaaattggaaatcatcattctcagtaaactatcacaagaacaaaaaaccaaacaccgcatattctcactcataggtgggaattgaacagtgagaacacatggacacaggaaggggaacatcacactctggggactgttgtggggtggggggaggggggagggatagcattgagagatatacctaatactagatgacgagttagtgggtgcagcgcaccagcatggcacatgtatacatatgtaactaacctgcacattgtgcacatgtaccctaaaacttaaagtataataataataataaagaaatttagggattaaccaaaaataaaataaaataaaataaaataaataaataaatgttttggccgggtgcagtggctcatgcctgtaatcccagcactttgggagactgaggtgggtggatcgttttgagcacaggagttcgagaccagcctgggcaacatggtaaaaccccatctctacaaaaaaatacaaaaattagccaggcatggtggtgcaggcccgtggtcccagccactcaggaggctgaggctggaaaattctttaacccgggaggtggaggttgcagtgagccaagatcacgccactgcaccccagcctggcaacagagcaagaccctgtctcaaaaataaataaataaatggtttttttaaaagacagatttatggtagcaaaaaaataaaatacatagacagacataacttaataatgcacaaaatctatgtgagaaaactgtaaaacactcttgaaaggcacaaaattagatgcaaacaagtgaaaacacacaccataaggaggaagaatcaacatcatgtcagttcttaacttataaagtgatcccaataaaaatactactagggttgtttttcctgtatctacgtaatctgatgataaagtttatcgtgaaagacaagcacaaatttgagcacttatcacactaattactgatttatatgtctcattcctgtgcttgactttgaagggctttttgttcttgttggtatcccaaatataacaactgacatatagtagatactactgcagaatgacttttcctgaggcacactcagaattcagcatttatgttctacatgtctggcatgtaatagataagctgccttcgtttatatgttctttgttatttcaggcagtttctctgaagctaagtggttttattttgttttaacctcagccctttggtgtctcattcatgcaaatgtggtaccttttcaactagttatcacaggtgggttctgctttttcacttccatacttttctggcaactgcctaaataagtgtcatcctgatgtgtcttaatgattctaacaaaggactaagacttaaataacatacatcatacaaaaatgtagccataaatattatgacttaaaatagtgaaaatttctattaaaaggcccgaatttcacgttgaccattcaaggtatttcagtatgcaatagagcatgcacatactccacttacagtgcatctctaaatctgattgttaccatttaaaacatttgatttctcttcctagattcgaacagcattttcaggcaggtttggatgggttgtttcttttttaaggaagcttgaattttggcctatgcttcccttgaaacccttcccctcccccatagaaagcaaaagcaagaaattcagcttatttcattgaccttagaattgaggaaggatcttcaagatcctcttttatcttttcaaaaatagctgttgtgttttctcaacatgatgatttgcagatcagaattgaagaatccaaattttagtgaatccatttaaccaattattgcaacccttgggtttcttttccataagtactgcatagatattatcacagttgtggaacaagtttatgtgtacatttttttctccaaggactttaattgggaagccttgaggagtattttatactcctggatgtcagtcgttcctaaagatccagaggataggccaggcgctgtggctcatgcctgtaatcccagcactttgggaggccgaggcgggtggatcacttgaggtcaggagttcaagaccagcctggccaatatagtgaaaccgcatctctactaaaaatacaaaaattagtcgggtgtggtggcacacacctgtagtcccagctacttgggaggctgaggcagaagaatcacttgaacccgggaggcggaggttgcagtgagctgagattgcgccactgcactccagcctggacaacagagcaagactccatctaaaaaaaacaaaaacaaaaaaacagatccagaggatattgggtcacaccccagtctaatgcccccagcaggttcctggaaggattaggttcccatggtcccagtttctgcttccatggaaatctttagattacagatttttacctagcccaggtaaactctgtacagctttgatcccagcatgaaatttgctgtgatttctacaaaatgcattcataaaaggtgtggctggtgtgtgtgtgtgtgagtgtgtgtgtgtgtgtgtgtgtgtagcagattttactaaagatttgagaaagattgaaaggtatttctatctcaaaatagaaataatgagcatatactccagccacctgctgtatggcttgacctaattttcttcttcccttctaacctgtccgtgctctcctgattcacctcctcctacttccaagcctgttcactccaatgtaaagtaggcagtttagctgtctctttgtaccttggcttaatctgacccttgctctcagtgtctcattcttctgtgtccctctcctcctccttcacttagacattttcctttccataaatgatttcattctaagaattctgagaattcttatagcagcttttagaatattaacttttctcacttttatgcattttggatgaggttctcttgttcattttgatttttttcttcacttctccccccagtttcttccccctttttctctccctcttcctacataaaggcaccacgtaactccattctcaagcctttttcctccaaaggcagctgagagttgtccttgaaagaaaatggcggaattgctccatgtgataatgtgtgtggatgagctgctggtgatgcactgaacctcaaggctttgtagaaggggatgcggttgggcactttctccctgcccctactccaagcactctgtcttagtcctgtaccaacagcctggaaccctacaactattcttttgagatttgttttgcagcctttctttaaggcttgttttgcaccctgatctttacattactgggtaaagaaaaagaaacgtgaagtataggtctgattaggcagacttctcaaaaggataggacagataaaacaaaaatttcctaattctcacaggctgctgttacaaagatatggcaaaaacactgtttaaccttttgaagaagaagaagaagatgacgaagaagaagacaaagaagaagaagctgcctgccttattttttttcttatttttctctttgaaaatactgatccttttcccagttcttccccacattgcagtgttcagaaatacagtctctatttaacatggagggtaacttcctctctcttatagctattgcactgtttaaatcttcatactgtttactgttggacctcctaacctggctatttatttttgtagtcctggatttgacagccagccctcacattcttttggaattctactcagagatgcctctctgcaggagtcttcttgtgatgtgttccctcagcaacatgagatgctactgttcttagtaactagattccagtatttttttttctactcagcgctcgtttctgggtgattaacagcttttggaaaatagattactgtctttattctcctgagatttgagtaggcacacttcaaaaaatatcttcatgaaggaatgaaagggaaatatgaagtcaaaagagtgaacagaacttctggagattgcctgtggcataagcctcctacgctgggctgttctctacaaagccatcctagcggaagctaggtgaaatgagaagaatgtggggagtggtggaagaacactaggctggaaagaaaagacaggtttaaattcctgttctgccatttcctagctgcgtgaacttgtgcaggtcactgaagttttctgaaccttggtcagtttcttcttctgtagaacggagagaatgtgacctacttctccagcatggcagattccagctaattagacatttttgtgcaaaatataaaaacgcagttgcactgtgtggacataaccccaagtgcacacagctgagtgagaggtacacaagctagatttcagcctacacttgcccttcacctgggcaccctggtacagtgcataacctaaaatcctgtacagcagccaagttccttaagtccaccgtcttctttcattaggagcaagcccgggggagggttcaggcaggggtacagaaatggggcccacttggttgctttgcagggacttagcaccttaagaagatgcctccctttttaccttctatttaatcttgcttttacctgattcctctgtgtttttttgttttgttttgttttttgtgtgtggtttttttttgtttgttttttttttgtttgagacggagtctcgctctgtcgcccaggctggagtgcagtggcgcgatctcggctcactgcaagctccgcctcccgggttcacgccattttcctgcctcagcctccccagtagttgggactacaggcgcccgccaccgcgcccggctaattttttgtgttttcagtagagacggggtttcaccgtgttagccaaggtggtctggatctcctgacctcgtgatccacccgccttggcctcccaaaatgctgggattacaggcgtgagtcacggcacccagcctgtattttcatcatatcattaacaatatcgtattaacaatagttcaagggtgtcagtcttgaagaatgtgctcaaggaagcacattccttttccctgctgatctctggccccctccctctgcctacttcccaagttgttggagcattaaatgagatcatgaacaggaaagcatctggaacatgatcccaagcccttctcttttatgtttcttcaagatagtctagtatttgtctaggctttctcctggtggttttgtttgttttttattccaggggtttccaaagtatggtgcttgaatcccgagaaatatgatgaatgctccattgagatgtgaaataaaagtgaaaacttttctttatatttattttaaacatgaaataagttagctttttgattatttgacatattgattgatgtggatatgcagtacctataggaagacataaatatagtgggtaggcatactcaacattttcctctaagtaatagaaatgtttgaagaccactgctgaattctcacatctcagtagccaccaaatcctctaaatgcaatcacatttgttgttcttacctgcattcttttgatttaaatagtaaatagttgcccccctgctttatcttcccaagacagaactttataaatcttgtggtgaaacctcctctcttcccgcagcctcatttcttgttatctgcataggacatggcaagcagtaagccttttctgtaaaatgtgacttgtttgtgcaaaataatttgttaagataaagtcaaattgggatccctctggcttaatcctagacattcaggaaaatacgaatatgggctctgggtctaggtgattctgagctagatgacattaggcaagttatttacctgagtccatgcctcagtctcctcatctataaaatggaaataataagagtacttgatgtcatccgattaagatggcaattaatagaattaatgcccatgactttatgaaaagacggtcagctgcatattaagcataacagaagtttgctgctgtcatcactataatcaccatcttcatcttcatccctagaagaggtgtcctgcacacccagactgatacaaccctgaactagaaggactattggcaattttggattcactccctttctcagaaaatatacactggtggtcttccatgtaccagacactttactaagtgttgggtgcttcactctgtcatcagcctcatgcttctatatggtaggctgctagttcccttgtcaccagctcgtggtcaccttactatgctatagcagaccactttacaacgggttcagtttctatgcctataatcaaggtaaaggactttgatgttgcaaaatcactaaccactgctggtgcagaatcactaaacattgtggattttcagccatttgacattttaaggtacaaatagactttccctgcgttattaattccacatgtcattgctgaatactctttggttattcttcctttgaaatgtcaactttttgttcatagagttctactaacataggcctcctcctgaagatgattcgtctaggtttattcaatgtgtattctgtcacatcctaccttatcttcaaaaagggtgcattcattctctctttcccaaattgcaattttttgagtctgctggacattcttacagcttaatattcttttcaacaaactgaaacgttggtggaaggatggtggttctcaatgtaatattttgtgtgtaacgctagcaaatctaaacaattattttactgttgaacacttctgtatttttttatctctctctgaaggaagagtttttataaaagcttagcaaaatttccattaaattcattcatattgatctatacatacagtctagaatatgtgtagaaaaagaaggggaaaacaagacaggaattcctttcatagttcctccttcttatctttctgcttctcatacacagactgatacaatctttgaggacatctaactcaagattgagtctgagaagatgatatacttcccagactgatgactgattctcccagaatccagtgagagaattgataattttttttttgagacaaaattttgttcttgtcgcccatgttggagtgcaatggcacgatctcggctcactgcaacctccacctcctgggttcaagcaattctcctgcctcaacctcccaagtacctggaattacaggtgcctaccaccatgcccagctaattttcgtatttttagtagagacagggtttcgccatgttggccaggctggtttcaaactcctgacctcaggtgatccacccggcttggcctcccaaagtgcttggattacagttgtgagccactgcgcctggctgctaattttttaaaaatacatgaatgttccctaaatatttattttcttgtgagttgttctatgacctgaaaaaacactgttttatgtcataacttagacatgatgaacaaagtcagatatttcatttttccaatatatgagctaagaataaaatcttgatgctggaattgaacgttgtattctccaggcagaggaaatggacatttattggatgttactgctgctttctttgcagtaatgacattatgaaagctctttgagtaatataatttgaaaatattatttccatgtttcaggtcatgcaagacaagataatctgccttccgaaaagagtgcagcctgctcagaaccactcttccctttcgaatgtctctcaagcagttgccagtaccactccactgcctccacctaaaccatctcctgctaaccccatcactgtggaaatgaaaggcctgaagacagatttggaccttcagcagtacagctttataaatcagatgtgttatgagcgagccctccactggtatgccaagtatttcccttaccttgtcctcatccataccctggtctttatgctctgcagtaacttttggttcaaattccctggttccagctccaaaatagaacatttcatctccattctggggaagtgttttgactctccttggaccacacgggctttatctgaagtgtctggggaggactcagaagaaaaggacaacaggaagaacaacatgaacaggtccaacaccatccaatctggtccagaagacagcctggtcaactctcagtctttaaagtccattcctgagaagtttgtagttgataaatccactgcaggggctctggataaaaaggaaggtgagcaggctaaggccttatttgagaaggtgaagaagttcaggctgcatgtggaagaaggtgatattctatatgccatgtatgttcgccagactgtacttaaagttatcaaattcctaatcatcattgcatataatagtgctctggtttccaaggtccagtttacagtggactgtaatgtggacattcaggacatgactggatataaaaacttttcttgcaatcataccatggcacacttgttctcaaaactgtccttttgctatctgtgctttgttagtatctatggattgacgtgcctttataccttatactggctgttctaccgttctctacgggaatattcctttgagtatgtccgtcaggagactggaattgatgatattccagatgtgaaaaatgactttgcttttatgcttcatatgatagatcagtatgaccctctctattccaagagatttgcagtgttcctgtctgaagtcagtgaaaacaaattaaagcagctgaacttaaataacgaatggactcctgataaactgaggcagaagctacagacaaatgcccataatcgactggaattgcctcttatcatgctctctggccttccagacactgtttttgaaatcacagagttgcaatctctaaaacttgaaatcattaagaacgtaatgataccagccaccattgcacagctagacaatcttcaagagctctctctgcaccagtgttctgtcaaaatccacagtgcggcgctctctttcctgaaggaaaacctcaaggtcttgagcgtcaagtttgatgacatgagggaactccccccctggatgtatgggctccgaaatctggaagagctgtacctagttggctctctaagtcatgatatttccagaaatgtcacccttgagtctctgcgggatctcaaaagccttaaaattctctctatcaaaagcaacgtttccaaaatccctcaggcagtggttgatgtttccagccatctccagaagatgtgcatacataatgatggcaccaagctggtgatgctcaacaacttaaagaagatgaccaatctgacagagctggagctggtccactgtgacctggagcgtattcctcatgctgtgttcagcctactcagcctccaggaattggacctgaaggaaaacaatctgaaatctatagaagaaatcgttagctttcagcacttaagaaagttgacagtgctaaaactgtggcataacagcatcacctacatcccagagcatataaagaaactcaccagcctggaacgcctgtcctttagtcacaataaaatagaggtgctgccttcccacctcttcctatgcaacaagatccgatacttggacttatcgtacaatgacattcgatttatcccccctgaaattggagttctacaaagtttacagtatttttccatcacatgtaacaaagtggaaagccttccagatgaactctacttctgcaagaaacttaaaactctgaagattggaaaaaacagcctatctgtactttcaccgaaaattggaaatttgctatttctttcctacttagatgtaaaaggtaatcactttgaaatcctccctcctgaactgggtgactgtcgggctctgaagcgagctggtttagttgtagaagatgctctgtttgaaactctgccttctgacgtccgggagcaaatgaaaacagaataacttatttttcgttaaagtttgactgaaacacgcttctaccaaatacagtataaataattaggtagtcttaatgcctttcctatttttttttccttttcacacaaaatgtacacaaagatcgcgtaaggagtatgtatttttaataaaaatttaattgtattttttcaatattaatattttgaagttttatttgtcttgaaacacaatgtatctattatctactgacagaaagagatagttccatttggttttttgtttttgttttttagttcattcttgtggaaaggagggaattataagttgcatgctttttggcattttttaaatagaggaagatattttgccaaggccatttttaacttgtttacacctgtacagggttcttatttttgttttcattgtgtgtgtatcaaggaatctgtgctagttattttaataccagctgtcttctccattggccaagaccttcattcagtggaacttcacataagtgcattgtcatagagtattttaaatgttcccctgcctccaacaaaaccctaaactgtgttatttcatatacctgacagttattgttatccttaggcaaggtttttgtgagtgaagaaaataatctttagtggctggtatgtttgaattaggcctttatcatttaacttatttgcattttaccaacattttaaaaatatttaaaacctaaatacagaggctcctcccaccttaatctcaatactgctatgtagtactggtgaacaactttggagaacctagatttattttaatattctaatagataattaaattttaaaatgatagaagcctggtttttgagtaatttaatcatctttcatatcttgatatgcagctgcatcagatggaaaaaaatttactaaagcaccctgttaaagagctggtgtgctccagtgaggccctctccttttctcagcacagtgtggcagtggtaatctattcaaattttgcctgttacacaaaataatccacttatatatacagtcagccttccgtacttgtgggttctgcatctgtggattccaccaactgtagatggaaaatactcaaaaagaaagttgtgtctgtactgaatatgtacagactttttccttgtcattattccctaaacaatacagcgtaacaactatttaaataggatttacattgttttaggtattataaattacctagagatgatttaaagtatatgggaggatgtgcataggttatatgcaatttctgcaccattttatgtcaaggattttgaacatcctcagattttggtgttcgagggcatcctggaatcaattccccatgataccgagggatgactggatattaatcgatttccctcaagcctgcccttgctgtgaactagtgctacaagcacgggaaatccatctgctctctggcttaaaatatgtatctcatctaaaattgttcgttaaagtgtgaataaattgctaagtggttgttgcttagcaataatttttgcctgaccatcagcagagcttttatttacctttgtaattaaaatccaacatagcatgggctcttgggcaaaatatttcactgactattaagcatgtagaacaggaaaatgaaatacactatttgtgcccttgacctagaaccagaaccactaattatgagaactttgttttatgtaccaaattcacattgaccattttctgagctctagactgatcgtagtaagttttgtgacttaacaatgaaagaaagtagaaagatgctttgggttttcaaaatgttgttttttaaaattgttctttgggtgaatttactcagtgctgctttgagttgtatacagaaacaaatgttgggaattgctcctcagaaatgtgttcctaagttgtgttttcaactttacatcatgaggtgaagcattagggaaagagattctttcgatttgtttaataatctaattatacagaccaaagtgctttacgtttctgctctatgttaatgttttagaatggtgattttgctgattaatttagacctggaattgaaataatgtgctcagaataataacatggttatagttcttgtatgataaagtattcaatttcagaatagtgttggaatcctgagtttgaataatgtgttgtatttagaaacatagccctatctgttttaaacaaataatttgttggccgattgtccatggttgagcatgacaaaaatacctcgtcgaaaggcaagcttaggtaactgctggcaaaacactgggtgcactatttttctggataaaatttatagttattttctatattacccttcaaaagggatctcttcaggttaaaaatcacgcttatgctgaagtctttatctggtgttaactaaaaatctcatatgggttcataaccgaggtcactaataattcatttttatcacttgtaaaaatttgctcaaaattccaaaaaaatattgatttgttttttagtgattttgcaggctgacccccaacctaagttttgataacatctggtaagtcagtatagttctgtgacttcatgttttaactaagaaggaaaattcatagatattcctattagattttataaaccttcaaaagtctgaaacttaattttgagtctaaattttctgacactggccccttttaatattgtaagtttttgttcactttcttaagtaaaaaaaacatttaattactagttagcccttaactgggaaactcaggtaatgaactgctgacttttctaaagttctttaactgatcaattctgtatagagggatatttatctaaccactttccgtattttacaagtgctctttctaaaaaggaataactattatagctctatttccccaatctctataggactcatgagagattgcttgtgtaaatataaaagcaccatatgtgttcttaactcctatggctgcttgaagctcatgatgaaaaagtctttttgtcagttttaattgttaagtacagaacaaacaattgtttggtgatggcctggttgaaagagagcatataaatatatcccagtggaactcaccaaagaagaccacacctcagaaattattgcattttctcattatgtgttgggtttgatttgcttttgtttttaatgcagctcttttaatataaagattcttgatacagtgaaatctcttatttcaagtgtaagttattcttcacccaccccttcccctgccattgtatttcccatctgtttcaaggagtttcaacaatttacattgcatcgtatgcagtaggtactgctttttcagaaagacctggaaaacatacctgctatgaatattttgttcagatgtagccatttacctggctctcaagttgtctttgtggagagggtcttagacaaaaatcttccttgtatttacttgggttaagtgaagtccaaattcttacagtatgctatttcaggatttctgatattaaaaaagaaaaaacaaaatctttatatctcttattaacacttcccccaagaagggttgtgctgttatttattttctattataagaaaagttcattctttaagtagtttcttttacctctaatctaatttcataccaaatacctgatcaatagaaatgatatatttaagcagcaaagattcctaatccatcattatgaaaagtgtcagcatacttagtagtgaacagataaagtcaatttgaatataattccactttgtttttagagactaaattaagattcaattaacattatcctatgaattctgaatgtgataatgtgattcaaacagtcaaattttattaagctcttagtaactcaggatagcattccatactaacctcaagttagcaaaacaaattagttaaacagcttggttcttagcagactgcttaaaagatcaagaaaattttctcatcttttctttctacttagaaacattgcaagaaaccttggacagtcttcaccagacctgccatgattttataagatttaggcctcagtgacatgctcctgaaagtttcctgccagccatccaaactaagcatccactcattccatcttcccaaagtcactcaccgataaaggtagcatccttaagttcatttttgaaaggtggaggaggatctccccctgcccaaaggaatttttttatcagaataccttgaaaggggggtatataaatttggaaaacttaatttcttggctgtgtttgataacagttcctatgcatggtttttaatgtgaggtaaatttgtttctttcttcagaatacctctctccaccccccaccttattcttcctctttaatgaatatttttattggagctcaaactccatgacttacgtgctcactaagttttcttttttccccttgtttactctgtctgtatgtatgtcaaaagctggcaaaacctctaaaactgtcaagaaaatgcttgaaagttgatttgtcatagtgcaaattcatgataaaatgtcttaatgttatttggatatgtagtacatagaaacagaaaaataaagtcatttttataacttaaaata

SEQ ID NO: 4 - DNA, 115419 bp, Homo sapiens
  LRRC8D
ccatgttacgctttgtctaattcccctcgttacagagtcatgtgctgctgctcccgtcgccgctgctgccgctgccgctgccgccgcccgtggtgccccggctcctccgccgcgcttcgaggtggcagccgcgggcggggccgcgggagcagggtccagggtgcagcgcgccttcgccgccccggcgcgtcccggtgcagccgccgcccgccgcgggtgatgccgccacctccggcctcagcataagccgtggcttggcggccgagctgcaccccaaggttggtgcgcacctctcccggcaggggccgggggctgcgagggaaggcgacgcgcaggtggagccgcgccgagccggagcgcgcaaccctggcgcaggcgccgcggcccgagctgcggttgcggccgggaactcattcggggacgtccgggggtcgggaggaggtggcggcgagcggagtgggctcccgtttgctgccgccagggcctgggagattgttccgctttctgcagtctgagcgtcggccgcttcggccccgggcggccggccctgcgggaggggagtgcggcttcctggctcctgaacccctcgcgcgcgctcccttctctcacctccctccgccctatcggaaaggcggggagagccggtttcggctctgctggttcctcccggaggtgggtgtttggtgtctggccttgctggcccggcgtccaaaggtgagccctttaacttctccagaatcgcctcccctccgcttccttcctgtgcagggtgggctgcgctgggagcccggcgggcgggggcggggaggtgaaggcccgcgccccgccccccgcgcctgtgggagggagctgggggcccgcaggtgcacccagccgtccgcgggccgtctccgggttgggtaccggtataatccgcttctagcttgtttggaggcgtgggctcttgggagcgggacttctgctcaaatcctgtccaggggcttgaaaaggaggagaattgggggtggttgccgggaggaccagcacctccagttactggagggttgataggggccttctcctttccgtcaccctttactagggggtatcccttgcagataaatgagattttttgatttcttaaaaagccagctgcagtgaagaagaactgctgcaggtcgaggggaagagaggtaggtccttgctggagagggagaggtgggctgtgaggttccaggagcacgcacttggggtgtgtgtggccgaaagtatggaggtagggttgtgggaaggaaacagccgagatgagagggagggcgcactcaggatgtaggcggagagatggggaataggtactggggagtcctagcggaatgtggggcccagctggagccgcttggcttatatttggctcctcgttgcctcgcagatttcctgctcttggatcttcaaaaatatttttttgttggacaaagaaaactctgaggacatattttggaagagctgatttctggaaactttccttggcagctttgatagtaaaattccgtacaggcagcgtttattgagcacctattacgtggtttgtaccggactttggaagagtctggacaggcagggaatccccgtttgcagtaatgatgagggctgtctaccttataaacaaataaggggcccctcccagtgccaagaatgagagtgatcagaccttcctcgccatagcatctgggacctctgcttcttacagacctgtgggaagaaccctgcaccatatcctgtaaatggggtcctcaggcagcatttatttttgcagtccaaaaatagtgaccaaacacctactgtcagctaggcgagagttggggttcttgcttcatctgtcttgacaccaactaactaaatccctgttaaaatccaagtgcaagatggggaggaatctgagatgaaaattaggtggttaaaacaggggtcacgaaaagttgatttggggtgtttggtctctgtaggttacttatggagccccccataaaatgattaggtaatctatggagccagggaaaaattccaaaatcttgaattggcacagtttatttatatatttgaacactatgttagatttgtctcgtttttattgagagtgtatgtgtgcgtgttgattaaaacctaacagattagaaggttggagaggaaactgtccttagatgacccagccttccattaccatgtggagtagcatatgatgcaacacttaaagtattgagccatattcatatcattttctccctgctgtgttttccagcatttaaaaaacatggtcaaaacttggccctttttttctcaaaatcgctaaagtttaatggaaagaaaaaagaaaaaaactaggccccttttcccttaatttttatattatgcagcatttttaaaaattaattttagaaaattctgatgtgtttgtcatgtccaaatagttaaattgttttggtagctatagtttggaaattagactcgattctgttctcagtactgtagacaaaatagctattaaagtcaaaagtttgtcaaaactttgtaatcatgattattatgttgttgaagtggtagcttgtaagagatttcagttatttttaattggagctggtgtttgacattctaagaaacaccagttaatatttgttgagtgccagcaggaagcagagaactggagtgggtgctgtgggatcttaacaaggcaggagaggattctcgtagtaatcacagagagcagataagacaggcacacatgaggatgccagcagaagcagacagttacagttgtctaagaatccagggtgtgtggaaccagataaaagtttatgaagtatgctgagggaggagtggaaatctgaattgacttttgacagtggtcagggatgtggaacagaatatacgactaataaaggagggtagagggaacctagttcgtgaaaaacttaaccacaagtcagtggtgttaatcatttgcctcaagaccagtggctctcagaagtaggcaattttgcttctcagcagacattgaggaatgtcggagacatttatggttatcatagcgaggtggcaggtgctgctgtcatctagtggctagaggacagggatgatacataagcatcctactgtgcacaggacatcaacccccttcctcctaaacaaagaatgatcctgtccaaaatggcgatagtgcctgcccttagatctcagaataggtcttgaagggaggtggttcttgacatgggaggagtaataacattgagcttaggaagctgatgtaggagacttgtgctcaggtctgatgtgattagggtctgggcaaatccttattgaaagaaaggggcagtttggagggagatcatagcagatgagcacagtatgatagtggagtaagttgggaatcattgaaatcagaattggcattttagaggggccctatccaatttatttattttagtaaattcttaccctttctaggtaacttttgtttaaccccaggaaccgtgaacttctcctgaagacaggttattacacctttggactgtttgttgtatttttacttgagctgaagactgtctccttgaatttttttttccaatgattttcttcttttggagctgcctactctctttgaatgcaccaagacaatatgatgtccccttacccccagttgactttgctaatgcaaactagtcccaattcttttcacagtcttttattgggatgtggtttctcgatgtcttctcagtttatcggcatctcacatggagaatctggaattgaatatgtaattgagatgtgatctggccaacagagagtacagtaagacagctgtttgctgagacctaagcataaatgtctacttactgcaccttaagcttacaactgcctatgattttcttaggcagtgccgagcacaaacttctttattagataatcatgttttggatagtcaaaacccccagatatttgctctgtgagctgctgcatcctgtatttcttgggcagatttttttggagaagttttttttgttgttgttgttccttctcaatgttatcctatttattttgatccagtcttctagcctttgacatttttgaatttatgtcttctaattatatcagagaggaatggaggatgactttgttgaaaactgtagtcagtactatgtaaaactcagtagtttgggggctctattggtcctatatgagagctgagatcactgagtgatggaggtgagttctagggctaattagcccttattttgggggcttctgagaaatttaaatgccaggccacatggcatttagctcttgaaatgtttgcacctggggtaccattttgaaattgttcaagtctgttgcaagcttctgaaatctgaattccttcctctgggtctttgtagttctgtttatgtatctggaagagagtttctaatgttttgtgcaaatctgaagcttgcagggttagagttgcttttgaaatttgtgagaagataggataactggcaaaaatcaactatggacttgtttcccactcaggctaagctttgttttcttttttgctaatatgcctcaaatatttagtgacagccttatatttagagaaggagagaggattgaggtttagaagcaaacctttttctcagagcttggctttaacttgccgttgatgtaagttcatacttaacttgaattttacagtcttcactacaaagacattaggaaattaccattcccagatgtcttctgaagctggcaggtaagaaatcccaggtaccttagtgccaagttctccaaatactctagatctatccagtgaactagatttaataaattacttgatttgggtggtagctgtctcacagtgttgaaggagagctagaggaaaatttcaaatctcatggccaaatgtgtgtcacctctttgtgatgagtgtccattgtgccccaatcctttgttagtgtgttggctgtccagatggtgcagtctacttggtttcccttctttaccagtccacgtgggcaacctctttatatcctctggagatgaaggatattccaatgaaaagaagggatgccttttgaatatttgaaatttcttgaggggaaaagtgttgctgtccttttatattgtcaaaggctgtgtcgaagttggagtcttagaattaaggatgcagttgtgtcactttgaatagaaagaatattatagctggaagggatcaacccttaaattttattttattttattttattttattttatttttttagacggagtctcgctctgtcgcccaggctggagtgcagtggcgcgatctcagctcactgcaagctccgcctcccgggttcacgccattctcctgcctcagcctcccgagtagctgggactacaggcacccgccactgcacacagctaattttttgtatttttagtagagatggggtttcaccatggtctcgatctcctgacctcatgatccgcccgcctcggcctcccaaagtgctgggattacaggcatgagccaccgcgcctggctacccttaaattttatagttgagcgtatccaggcacagggaggtgatgtgcttagcgtaagtttacacatgtagtgacagagtaggcattagaatctatctgccttaaagaccacaataaaggctaaggatcaggatttcttctctgcttagaggtgtgtaaacagtggggtccctcgggtcttatttacataattgatctaaaagaaggaacacacagtggaatttctaggtttgcagatgacagcttttctggattatgaaatgtctaaccactgggtatttgctccagggaaaccacactaggatgtattagtcatcagacgatttgcaggtgagtttcttgtgggcaaataggtggttgggtattaaggggaaattacagaattttcataggatggtgttttgttggataaactcagggaagggctctgaaaataccttcatgttgttgtcctgacttgctgttgcagaaaaacaaaaaccatgaaatgctaggcttcatcaaaaagattttggggctgggcatggtggctcaggcctgtaatcccagcagtttgggaggctgaggtgggcagatcacctaaggtcaggaattcgagaccagcctggccaacctggtgaaaccccgtctctactaaaaataaaaaaattagccagatgtggtggtggtgcctgtaatcccagctactcgggaagttgaggcaggagaatcacttgaactcaagaggcggagtttgcagtgagctgagatcgcgccattgcactccagcctgggcaacagagactgtctcaaaaaaaaaaaaaaaaaagattttggggaaaaaaatacaaaatatagtcttaattgagttttcactgtgttttttcccccaaattcgtgggcacctaaccttcagaaagacactagagctggaatgaccattgtaatgatacctacctagaatgtttggacattgctgctagaatgtaagcttcatgaggacagggtccttgctcatctgtttctttgataaaaatattaagatttctagcacctggaaagagtcatctgagaggagatggcatggaggcctatgaaaccacaaggatagggccataggcagccttgatagttcactttattctaataagcccaagcgtgctattttaattgcctccttgtcttcctgctccggtactctgtcagtgtggttaggatagggatttatctctcttgttctctactgattttctagtgccttgcacgtggcaggccctctattagtatttgtcacccagaggaatgaaagaagaggtataccttaaaactttacaagatataaattcaggatgaatgccaagaaatatggtttttctcaaccctggctggcatcagaatcagtctggggaatattttttaaatacatatattcagacccttccccatgcctattgaatcagaatctctggatatggagtttagttttgaaaacagccctccctgcctccaggtaattctaatgtgcagctcagctcagaaccaccatcttagtagttgtgcacgaaggctgggtactgactgaagacaccttcaaatgggtttgaaacatttgtggccagcaagcccatcattggttattaagacaaaagagaagctttaaggatatgtcagtcttcttggaaggtatagtgacccaaacagcacatggtccaccatggcccttggtgcctccatgagatagtgcatcctgggctggatggatgatgggaccaactcccagtccatccttgagcagtcatttccagagtgggtccctgatgacagtttcaccagcccatgaaaaaatgagaagagtgaagacaatgcaacaagcttttctgtttcaactttagtgctaagtttattcaattataagagtctggttttttgttttgtttttgttttgtttaatcttgggttgtccttcctactggaaggttggcttgttttttcattttatgaaaggatgatgatggttttttggtgtcatttctaggtccagttttaagctggcaattttgtgatagtctttcttttgacattttactggcctgtgaaatcaaaagtctgggaactaccactcctctgaggttttcaagatgtctgtgtctaccccttgacacctccttttggggaacgctcataaatatggtgttaggttccattacaagcattttttttttcttaaaacatttttttctttcctcttatgcttgctggtaatagttaggaacattgccagagaaagcttcacttccccaacaatgcataaatcccattgctacatcttagcatcgacagatagccagcactgcaacgagttctgcagaacaaggagaagaaaggatgtacaagaagacacctttttctctgatggtcattggccgccagaaagctgtgtctcaaaatctctcatgctatagagataggacagaactcttcagattgggtattagatgccgtcagtcaaaattctcttcactgattgcttacaacccaattttaagaaatactagaaaattaaaatgcttggaaaaatggtccccatggtgcttgcaatggcactctactgagtaggcttttaaaatcagagtattctcagatatggctgcttttttgttttcgtatttaacttagctggttaagaagtacttggacaatgtatggaacacctaaagttattaataataacagctattaggcacttaatgattgtccaaacctatgccctggaatttacatgcattctcatataatcctcaaaacaattctgagtgaaagatagacttattcccactctacggatgaggtcataaggctggaaaggttatgtcatttgcaacaacgtcacatagaagtggcggaggtggaatttgaaaccagactcattttataccagacttttttattttaaaccagactttttaatattttataatatatacctattctcttatctactgtaccctttctcatttctgcacatttctttgtatttattttataatacctagtagtatttctgttacatgaaaaagagaatcaggttttgaggtgtaaaggtacctgataactcctctggttcgtgctgcgtgtttgaagagtagagccatagcggcatcccggcatttaggcttccaggccaatgctcttgtcaccccaagttggtcttagaagtatgagcctgttggtaaaactacttgaatgaaaatttatcctggaattgagagggtaactttaaatcacttaattattctggtgttttctctagccctgttctcttttgtcacccatattgatgatagaatgccacttgtaaagtacatcttaagcaccctcctcctagttagatctcagccagaataactactgtacataaacaaatcatgtaaagatatacctaatatttgctaaggtgttatgtcttagctgaaaaaactgctgtaacaaatgtttgctgaataaatgccataaatatattcaatataaaaaaactatatcttcaaagtaaatgaatgaataaataaatatccatgtatctacatcatattcataggctgtttcattcgtgcctttattttttaaatacaagaaaattatacttcattatactcaagtataatctctttgctggagtgaatttattctgttcaagttttactttaactattgaattatttatcttaacatttagccagtaataaagtacaaattgggaactgggctgcagttttatacatcttgcacaaaacacacccttcctacaaacacacacactcagagacagagagagatgcacttgttctttaacttagctctcttaagatatttttcaattccaaaatagtttctctttcacctgttctaaattcttttccttataacttatttatacctgctaagttctgtaccaagcagaaattagatctgtaatttctgtgctaatcagagttggatactagcagtagcctgggggtcttgagcaaatagtgtgcctcagagatgatttgtggcagcaggcactcgccctcagggtggggtcatcaggagtgggaggcccatcagaagtgggcctgttgagcagtgcattgttgccgcgtgggaatgtgggccggtgttgccaggtcttctgatttttcactgtaagccagaaatctggattattgtgtaaaatctgattttaaaggttggctaattcaacattttagaacttcctgtaaaccaaacaaaatagctttgtgggctggaaaccgcctctttaggcccttagagatagccctttagtggccagataaatggaagcatagggtttgaataaaaagcacaatacacttttttttttttttttttttgagatggagtctcactctgtggcccaggctggagtgcagtgctgtgatcttggctcactgcaacctccgcctcccgggttcaagcgattctcctacctcagcctcctgagtagctgggattacaggtacgcaccaccacacccagctaatctttgtatttttaggagagagggggtttcgccatgttggccaggctggtctcgaactcctaacctcatgtaatctgcctgcctcagcctcccaaagtgctgggattacaggcatgagccaccacgcctggcccacaatacacttttaaacattaatttaaattgacatgaattgaaacttgatttcatccttttctttcccaaccccagacacaaagcagagcaaagcaacccgcaagccccgtactttaatgataacaatttctgacctttaaatgtggtgtattacatctggagtggttgcatttgaacaggttgtctggcctgaggaggaataggtttagggagaaataggtagagtatgaatataaaaggggagaggaccggtgggacacacacagagagagaaggaagggtaagagccacttggagttgagtaaagagatcatctaccccaggagatacgggaagatttgttcatatcttaaaattgatttggggtaaatggggaagaaagagaaagaaagaaaaaatgtcaatgtttagaaatagtgcagaatctatgttggcagggggcaaagagtgctattattttgcaattcctagtgtatcatgttaatgtgtacggtggcttctacaaaaggggaattggaatgaaaaggaaggaaaagagtttatgtcagaaagaagactagggagtgagataaagaagaggggtccagagaacagctgatagcaaaaagtaaaccaagaatgctggaaaatgtttgcataggggaaagctggctatgttagagtacaaagcatcatttttttcccccatccaaactatgacattgagcgggtatacacagcagtccctaggaaaaagacggtttggaacaccagcttgttttgcttttgaagggccagcctccagggtccctttgtgtgaagcagtgtcagcagggctgcgtctgcgctgtcagggcacactgccgggaggatattgtctactgggttttagcccctctccaccatctgggtatcttggtgcaggacaggacctcagacctttactgtgaccccaatagtgctcctgtctttagttttatttgcttttctggggaaaatctaacatcgtttgtgtaatttgttgtacatacaccctctttgaaagagaagagagtaataatttgaacagatttctgctactagcagaatcctagtctgagaggatttttaaaagcattttgctcttaatcatcccaagtatatagtaccttgttcctggaatttcagcattttctgtggggaggggaacaacgtcttttcatcatctgcaggttttattccagtcctggggctatcatccctgtgtgatctgaactatgtctttctagactggaataactttaaatttctaatactttggttgctttttagaataaattcattttaaaatgtccccttctatttgttactttagtgcagtaaggatctttatggatttcttttcccccttatggaaaccatttgtagtattgagagaatgggttcaggttttcttgcactgttgggaagaagaaaaattcttcaaattccactgctcggccttctagggactctactctacagatggaagtcctggaagatcctatcaggaagtgaaacttgtaaataatgtttgtgctcgtttggggcattatgcgttaacaagttgtacaagaagtcttctttaaccttcttataaaatacacttaaaaaagaatccagtcctagtgacgaatctggaaataagactaggaaacacttcaccatgtgccaggcactgtttgcagcactttacatatattagctcatttacttctgacaacagccccattttgtagcagaggaaaccaaggctggcttctctagtctggctgctcctcccaatcaccatgagcagccaaggtcattacaggaagtggcagaactgtgatttgaacccaagctgccatggcttcaggatctgtgctttttaggactccttcattgccccttgaactctctcacaacaacgacgaacataaatttgctttagaaacttcttttgccttccacattacctcagtcttaaagtcagcatcagagtttggttgaaaaaagaaataaaattatattccttccctgatactgcctccccaaaccaaatgaacaaatgaaatcatggagacagtttccatctaggaacagaaacctgactaaaggatcttcctggaacaccgtactaaaacgctggactgatcattacaaaggctgtagttggagccttgttgctgtgacaacccgcatccttggcaccagacttgtttttaacagcacaaaacccatcttgttttacctttacctttacaactggtagatttggtgagcttctgagagcactttttaaattgctttttgcaacctatttggaggcagtgggaaggacctaggatttggagcttaaaagacttgagttctaatcccagcactctcttttgtggactcgtgtgaccttgggtttcatctcctgtaaatgggggaggggacagtaacggcactaggattggtttggtgaggataagaagctattatatatgacagtattttattcacaatagccatgtgtgtcaggcagtgagctgaccagcaagtcctttgtgagtcttggaatgcattttccatgggtgggtggtgaggtaggtggttggggaaggtttgtgttgatctgaaccccttttctttcctgttctctccttacctgtattccagtgtgaatgatagtgtgggtgtcaactcagtttttggcccaaattccttttccttggttttcccccctcctactttgggctctcttccagtttgcctacaaagtgctatttgttgcaagggtgaagcaggcaattctctgggcagcgacactgtggctgcaggcagttgctgtagttcaaagcaaacagccaagctagagtaggagggacactgacggggtttgttcattagaattttgaacattaatttttgaaggaaagaaaggggagtacaaatagaataggaatagaaatattgtgaaattttctttataacagaacaaaattgggtcaggagaaaggactttaagaaatcacttaacatttatcattactttgaaagcaaatgccttttttgagtaaaagcaaatgctttttgagcatttactttgtctgcagtgcactctagtggggtgggaaacgaaagttagcactaaatattaacaacggctatcatgttagtgcatttcaggtggtccacagtgggccacttttctgttctattcttatatcagccatgaaggttggtgtcattggtgttccatttcaaagattacaaaactgaggctcagtgtgtttaaataacccactcaaggatacagtgctaataactagcagggagagctaaacttcgacctaggtctctttctcatccacaatatgaaagatgagtaagatatgcccctgctttgaagataagacctgcatgtaaacagtttagtataattcagcacagtttgcagtatatgttgtaattcttgtgagagctacattttttggacatttgctcagaaggagagactgattttgactggaaagatcaggggaggtctcacaaggaaaaataccttgaatagggccttgaaggatgacaaagatggtgacaggtagagaattgatgggagagctgagggaacgttggagcagcaaagcatggagttgtagaagtacagaatgtgtttgggaaatgctgctgggatggagtgagggatggaggaggaaaggaagccagagaagtagtttgggacagattacagagggcctagagtgtcctgctgaggaatttagacttcacttggttgcctttgaataaaggttattccattgaaggttcttgagaagggaaataattttgccttgatctctgccttcagcaattctggtggtagcatgggctttcgccagcactaggctgcatctaaggatcacttgggagtttcctaaatgtgcagattccaggagcacccctccacccccatgatttcagtggttcacatcttgggtagggcctgggaaagtgtgttttgaaggctctgcaggtggttctgagggcagcctggtgagggaagggttctagagggcattggggcattctagagaaaggtctgagctgaagctaattttgagtttgtccctgtatggatgattttttaaaccatgggagggggccagtcatcaaactttgtggaagttctgtctgtgtaggttctatggtttgctttttgtcctctgtaagtattaaacagtaatctctcagagcattgtcatttggctgaagtttttcacatgtatttcctgttcaaatctagaatgtgggatggctctaatccttatgcccctgtgctagatttctccttttttgttgctgtttcttttagaaggcagaggaatatatgggattttattgtgggctgtacctgtcttcaatgacaatctcttggagagcatgagctctgtagtttactgcctgggtttttactctgcctttctagccatgggaacatgggaaagttacttaacttactataaaatgaagatacaataatatttacctcctagggttagttgtgaggatggtttatataaaatgcttagaacagttcctggcacatagttaacactcacgtgttagctgctgttgctgttattattgtcattaattctgcagatacctaaaggtattccttgcctgcattgctcgcgtattgagggcttcaccttaaaaaataactgtacaaattatgattgagagtagtaagggtgacatattagaaagttttaattattcagtgaccctctttcctttcagagccttttattttctagaaacatatgctttcctcccatgaacaagcctgctaggacatacttaaaacccatatctggtcaagaatctcttcctgcatattggctacagagctaatagtgcagttcttcagtctcttaggagctacatgaaatgatgctcacttttacatttcctaggacccagcctgaatataggtcatttttttttcaaatgtttcagagctctcttactctcatttgttagttctaaatcaactgctaacatttaaaatacaatttttgctgggaggacagatagttctaccaagggataagctatttctcaacattaaagaaattagaaattataatttatgcttaaaaaaataatgttagttcaagttttagcttgcttctctctgccttctctactgttctttgtcctgccatgtgtttgcaaaccattctcatgaccttctgcctgtgcggcatatttgtagtggggccagattatacatacgccactgacattttcatctgacctcctaaactcagcctttgagccccttcttttctttgggttttcctaacgaagtcttgttgaaaggaatattatttcccatggaactttactgatagtgtatggaataatacgatcatgctctgtgaagttcagacctgaagtcttaacattttaaacatgtatctatttgctattgcttacttagttcagtcttggcattttaggtttagaggtggttactacccattggacatccatagccactccctgcaaattaaaggggacacaaaaatgaaactgaacttctgtttttcttcacctctctgctaatttaactaagtcaattggctcgccctcatttgccctcttcacacctcccaaattaatgttatgtgcaaatttgattaaaacagcgtgagtaatttccaagttacttttgaatacggattgaattccgttccttgtggctttttcttcatcatactagtagatgtagccagtgtttttgtttctagcctgcttaacttgtgtggttcttggaacaacataagttttggttgatttaggatcctgatttcacaggatactgtatcaactgctttaccaagatcaaagtacatgggttcactgcattcctttcagttgctaattcagttgtgctgtagccaggcaatcctggagccttgaaatccaggattctggtcaaggtgaggaagcagcccttcctcctggccttttcattctgcaaataccttttctttccttcttaatatttatttattcagcaatgtggcccaggctctgcatccctgacttcctaaagctttcagcctagtgcccctagacaagtggttacattcttgctacaaactggaaagtctctgagcccctcaccccactcctccccacttcttttcttctgctgtatttatggcactgtacctcatgaactttcacacaagggaaagcaaactcaatccaattgggtcttatgggtaagaaaatgtgttggttaaagtagattctggtcagccaggctatcgaagttttagtatttactagcagtgtgactttgggcaaattacttaacatctccatgtccatgtctcttttctcatctgtaatatgaagataacaataactcctacctcatagggttgttgtcaggaatcattgaagtgttaatacatgtaaagtgcttggcatttattaaactagggttagttaaaaaattgatgaataagctattaaaagttcataataaagtttctggagttttcaggtactccgtttctctttcgtgctatttgccttggctattccactacaggcaaaggaaaggagatgaaatttaaagtatctaattttgtaaccttaaattattcatgaattgtttttcttcgtgtagactccattataatgtataatggagaattgtattcaagatatgatgtgtgtgacttattccttatgaactctgaaagtcatttttaagaacacttcttaataatgaacattatatttatttgactttttaggatccaacccttttacattgttgggctttttaatatgttatttaaaaaacaagattgcccaggccaggcgtggtggctcacgcctgtaatcccagcactttgggagaccgaggtgggtggatcatttgagtccaggagtttgagaccagcctgggcaacatggcgaaacccctgtcccttaaaaaaaaaaaaaaggggggaaaaaagaaaaattagctggatgtggcgagctcctgtagtcccagctactggggaggctaaggtgggaggattggttgagcctgagagaacaaggctacagtgagctgagactgtgccactgcactccagcctgggaaacagagtgagactctgtctcaaaaataaaaataagataaataagattgcccaaagagaattaaaacaatgtttcaaataaggaagcagatactaccacagaagattaacagacaaaacatgctttccaaagaagaatgaattataggacagtatttcatctatgtaatacacatcaaaatcaacagtcattaaataatccaaggctattcattcctagcagtctgtgctgggagcattttgaacagtcttttatacaggtatttgcatttagtcagaattttgggggatcttacccttctttcacagtattatgcctagtaaatcccagtacatcaaccatcatgtcctactttacatgcatttttttgcatactaattgaagaatgagagaggaagcttggaaaaaatgtattaagattttgtttatcacagtttgatcagtggatcagtcttattattttataaaataatatccagctgcagctcaagtgcatattttacctaatttatatctacttatattcctatggcttaaagtcagttgacctattacaaaatacatgtttatcctttacactttaccaaaatctggcgaggtgatgagggtgtatgatgcacttgagagagagcattacaagttatctttttaaagtgggattttttttctgagcaaaagatatgtagtgcagaataatatatccacagccaaaatgcacccactcatttctattcaattaaaagaaggaagaacagacccagggtgagttttcgttccattacctaaggctttgagaatatcacttaacatctgtgagccctgctgctgcttctgtaacacattaataatgacacctccctgcgaaggtagttctgagaattcattgaggcagggcctgtgagggtttgtcctatgtaatgttagtaattttgtgcaaggtgtgtgggttcattgtgtgtgtttggaagctgaggaaattggagcactgagcttagagagataccagctcaggcctgtagccaccctggccctacctgcctggtggttggatcttctaagtcatgctgtggggtgctttaagtagtgctttccttcttgctttctcaaaatcaggaaactatcttacaggcatatttttttttaaacatgatttgcgaccccttgctgcattttgtcatgtgaaatgacacttactctggttctttaaagatggctaataatcctctatgggtaaatgccaatcaggccagccagttggctctgcgaaaggcttccttgaaagccatggttcaggttttgcagagaccaggaagtgaatgtgagtgccagagacctccttcccagaaaatcctgttgcatgtgctaccccggaggtgaagacaaggtagggacactgctgtgaaggctgatagtgcagagggtcatctctttggcaaccagacagcttgtcattttttattttcttgtttgtaaaatccatgtgtaataatcaccaggactcctgatattcacacagagaattgcttgtggcaaacatttttaaccgccaaactaagatctttcttaagatgaagaaagataagcagcattgttttttagagatgagtttatcatctcctcctccacacccattagtcctcagaaaagctatttcttgaaactgaatttggagcttctaaaatggctttcacagtgttggtctcttcatctggggattatggtacttagggagtggagactttcaaagtgaaccttccacatttttccaaaatcttttgtggcataatttgtgcctacctgagagcgcattttttctttttatgaggttgtgattttaatatatatacatacacgtgcacacacacacgtgcgcatgcacacacacacacaaaagcataacagcggctatgagaaagaaacacttctgcatgaaacacagaaggagcgggcgaatagctgaccaggggaaagtatgagatggtgttcaggcctcatggtagttaggattaggaaatgtcaggggctgtgttgtattctccaaactgtaaactttgggagtcgtacagagtgaatgttaataaatatttttgataaggaggagggtttgggtttatagggagcctaagaaagagaaatgtggctgcccttcccccacagctggccactgctctggaggggagagtattagacatttgggtgtggacatagcagcataggcgaataccattgttagtctcccaccaaatgctcttctgtattttgttacctattaaaccatacaaactctcctatatatcattaaattatcactcagtacagtacttggacaaaataggaatagtctactgttaactgtttttagattaatgactttgaatttttcctaagagagattttaagaagttagatagtctaatcgcatgagtcccaaatataaaaaacaaaggctctgcaggtttaagttttcctttgccttttcttaagaatttaaatatttttcttcagaggttttccttgggctcttagataatgagtaacaaaaatgaaggagggataccaaaagatatttcaaaacataattgtgtccttaaaaacatggtaataattgacagggtttttaaaatgagagaagtttctagctaaatgccctgcttcacatccccactccccacactgagtactttgtggaaaatatgtggtagctgtttgggaagaagtattaagtttgtgaaaagacatgaagagttgccaggaccacggtaagcaaaggattgagagaaggaaaaaggtgaacagagctagaccctaaaaggatgctaagatcccttgagaagtcttttagtaagaagccctccttagactgcgagtgcctagtggacaggaatgcatctcactggtccttgcagtcttcttccccgcccctgcctttgtacttgaaggccatggtcatctgggtgcactacaattcaagtctgtgaggtgaggagaaaagggtgggtgaggtgtagttcatcgtctggaggcctcagtattgtcaagaccacccccccccttttttttgtgagtgcacaagactcttaatcaccttttcatttggttgcgtcaggagcctcatagcagcatgctactgacaggtgattgtgaactaagtgttatagcagaaggaaacagaaaatgagagaatgtcaacttgaagccatgtgtgtttttgtgaacatcatttgccactttcttaaagagccccagttagagaaaaaataatgcaggaatcgaattttagtcagcttgttaggttctgaactggacagactttgtaccttggcatatgagtagccattctagaaccttttgaacttttgccaccaagtaattctggaaaggctgacagccagcagtaggtaatggaaaaagccagtatcctctgcccgagaaattaaggtgtgtattattcaggtggttaaagaaactgcctaccacaacagcaatagctggtaagtagcaggatcattcagagatgaagggatggatcacaggatggttgctgtgctggactttctccctagaattcatgatcattggccaggagtggtggtttacgcctgtaatcccagcactttgggaggctgaggcgggtggatcatgaggtcaggagatcgagatcatcctggccaacattgtgaaacctcatctgtactaaaaatacaaaaaaattagctgggcatggtggcgtgtgcctgtagtttaagcttctcgggaggctgaggcaggagaatcgtttgaacccgagaggcggaggttgcagtgagccgagatcacgccactgcactccagcctgggcgacagagcgagactctgtctcagaaaaaaaaaaaaaaaaaagaatttgtgatcattgtaggaattcgcagtgtaggggaaatggtacagattttataatcaggaaagacctgtattcagttctccatttgaccactttctgccagagccatgggaacagttttttaatttctctgaccctgtttcctcacttgggaatatgacttttgcagatctgttggaaaatattaaggccaagagggatttaaaatgagatgttgtctagcactgagtaggagcttaatatctatccgttcctttctgcttcagtcaaaactgagcttcatatgtagaatgattattgtttgtattacattctccattttgagtatggaaccatgaatgccaacagcttgataataaaaggagccgtttgtcaagagacagtcctgtaagcgctttttaacctggtagactgtgcatgtaagggcaggcaggttacaaatgcgaatccccatagccagcacactctacatacagcagattcttaagccatctgtgtactctttttaacactgtactgaagtccacttaagtcagagttcaacaaaatgaatgaatgtaatcccaggactatggaacttctctgtgattacatagcacagagaactacggaatcattttctggaagaatgcaccatgcgattatgctccatgtggaattggtatatataaatgttggtagactgtgaggatgaggttgacaggttgatctgagtagagaccctaggaggaaccgaggatactatgtagttttgttgttgttactgttttgcagttcctggctgctctgtgttggcagctgtgtgcacgctttctcttttgagccagcgaggcacaagacaagtaatttcccatcttgaatctctcacagctgctgagctgtagtggatatactgggatgtggctatgaactttaaaaatttgcataaatccagcccctactcccctgcaaaggctcagtcttgtctttacaccttccccgaggtaattattttctggggggtaggggtgggggttgggagggtgaagaaaggaagaaaaagaaggccgatcacactgggcaccggcggaggaagcgtggagtccattgatctaggtacttgtggggaggggagaacccgagcagcagctgcaaacggaagggctgtgagcgagcgggcgggcgggtggctggcagcgaggccaccagcagggggggcccgggccgaggccgcgccacctcggcaccacgcgggcagccggtgcggcggggtcgccacggccaggggagcgctgggtgcccaccatggcagttatgcaagcggtgaccccctggtcttgcctccccgccgccctgcactccttcctccccgctgccgacacttggatctctctagctctttctctcccctgtgttttcaaacaggaagtgcacggctgtctataacgtgctgccgggtctcaggatggaggagtgaagtctcctgtcgccgtggttccagcctccggagctcgcccaagccgcgtccccagagagcgccctgagagaacagggtggccgcttggtccaggtgcgcggggtcgggtctgggtccagggagcgggtcgggaagtctgcggcacggagcactgctagtgtcggatctgcatctccagctctgtgctgcagcttcacttgcccgccccccaccactggcttctcacccggggtctctgccaaactctggctgctgccgccctgggttcgggccggcggaaggccctgggcgtgcgctgcggagccgcctgcgaggactccactagggcgctttccaggctggactgccccgggctgcgctggagctgccagtgctcggggagtcttcctggagtccccagctgccctctccaccctctcctccagctgctgctgcctgaagcagcagctctttgttcctcccttactggtctggggtcatacacataaatgtcctgggaggcagggtggtgggagggtaaggggttgggacgtagggaagaggaagaagatgagttgtgggtgaagtgtaaggtggacaggagaaaagaatcttagtgtccatgctcagaaatcagctcttcagctgctgaaacagaggaccccggttccttggtttctgttttgggaaagccaggccatgccaaacagagggaattaatgatgcaggcacacaccggaaaggtagggaccaggaatgaggcacagcagcgagttctgagatgtttagtctggtgtctggcttgttaactccactgtggtttctgagccaggcctcctcgctgagacagaaggtactttaacatatgagccaggcatctttattaattccatttgtcatcaaagtaaaattgagagtgacttttctcaaattcaccaggtagattatactttcacttccacatcgtgttccatctcaaatgttcataggagaataaaacactagggttggttttatgctacagacgttttgcatctttatttccctggcaggtccaatctttctccagaataactcagattgacttctccccccagtgaaagcctagccttgttttaccgcctgtttatctacctggtgaaactctagtcttggggaggacactaaggggagtgggatggctgagaataatcagtttgatgggatcattatatggcctcaaattaaatgtttgagccctgttaaagccagtagcatcttcttaatttttatttccgaggaagggatgtgagcatatgactagaggggaggaaagtgagactctgccaaagaagatctctccctgtagcacagaggaaaagtgaatctgtgtagcacagtatcacatacactgcgcgctttcactttatcgggtagccaagccatctaaggagtcagggttaggtgtagaatttacattcatgacgtgggatgctgactatagcattctgatgctgaaaattttatcataaaacatatttactacataattaagtttgctttgaaaggaaaatttaccagggtggtacaaatttaccagcttgagtacaaaacatgtaatcaataagtacatggcattaagtagccaaaatgcactatgaaaactgaacaattaaacttatccttcctagatagacatgaacttcgtaagtttgaatttaattgtgagatggctgtgtttatgcctcttcttgccaaatacgtcatgaaaagtgagaaagtaatcgagagagattctttttattttgagacggtttttcattctgtcacccaggctggagtgcagtgacgtgatctcagctcactgcaatctctgccgcctgggttcaagcgattctcctgcctcagcctcctgagtagctgagattacaggtgcacgccaccacacccagcgaatttttgtatttttagtagagacagggtttcaccatgttggccaggctggtctcgaactcctaacctcaagtgatctgcccacctcagccttccaaaatactgggaggccaccacactcagccagtcaagagagattcttgaaagacctgtacaaccatagtgttttgcagctacttttttttttttatttttgagacggaggagttttgctcttgttgccctggctggagtgcaatggtgcgatcttggcttaccacaacctccgtctcccgggttcaagcgactcttctgccacagcctcccgagtagctgggattacaggcacgcaccacctcgctcagctaattttgtatttttagtattgatggggtttatccatgttggtcagactggtctagaactcctcaccccatgatccacccgcctcggcctcccaaagtgctgggattacaggagtgagccactgcacccagccactgcctctacttttatacctatgtgtgttccttgccccccagtttattaatttatttggggactttctttagcatctgtcactctgctctaagatacagtagaacagcttttgtgagcctccaaatctcccatttaagaacagagtctccttctattctagtgatatttaaactcagaaatcataactggcctttagattgtcatgttctgtttacacagtgtatatgatgtatttttttttttttttttactcctttcaacagagctaaactcttcttgctcctttcaatgatgtagtgaagtaggattcattttctccagtttatggagagaggaaacaggctgatgagatgtaacttgtcagtggtcgtattagtaaatggagttgttgggacctgaacaaggtcttctgatgtaatccgtggttcttttgtagtctggaaagccgctgcctaaggaagaggtggcagtgctgattcaggtggtcctttgccattccaaaagaaagctggattgccttaccaaatctccaggtttttcgttatacctcttcactcaggctctgcccagaactcaaggtttgttttcatttttaaagcagttttttttttcatatttgaaatttacacttcctgtgtgtctgtgactagatttagctgtgtttgcaccagtgtgatttgtattctgccaaaattctagtagtatattttaatttacagataccttttgtcaggttttagtgagcaagtatctttccaagggatcagattaccatattttaattttttagaattaaaaaacctcttctggcttactcctagggcagtttatatcccagaaaattaattaattagtaaatgatatatatgactctccactccccactactgctgccactgtgcgcatcccctactctttcattccagctcacagtaatccactgagaggctcacattacagaacaacgtgagcaaacccatgtaactttaaattatagattaaatatttttgtaacattccttagatgcaagtaagtttatttccttatctttgtagcgcttaaagttgaggctctctttagttacagttttaatgttgcaccattaactaaactgtaaaattatggtttaatttgttatactgcaaattaaaatttggagtacaattttcataagtgttggataaaatatttgtcttctcttcaataattggaatatttgtagtggcctataacttcccctctgtaaaactgtggtcaaaaccctcagggagaggagaaggtaaaggaggagcaaaagagaggaaatgagagctcattgttttgtaagggaggaatgaaatgatacatgtatggttccttaatacgcagtgcactgccttttctgtctgtcggtgagttgcagtggcttgtgaaccagccgtgcatctcttccaacagaaaggctaaaaatgaaagggtaactagctttgatgttaaatgccaaggaaaagttaatggccaagaggggactaggagtggagataaatgcaagttaagatttcattttttcaaaactttttttcccttcacgtatattataaagtaaaaaatgccttcttagtcaaattgtaaactgtaatgcagtggctcctgacaccaaaatcaggaagcttttatttaatattaaaagatttcaacgatcttaactggttcactgttttacttcgtatctcttataattgcagatgtttcatgagagttatttaatatcagccaagtatatattgcaaataatgacttcagcatcttcctgtatttgtaaaatagcactgcatgtatatatgaggtagggattcagcctacagacctcttgcagtaccctgaagtctacaggttgggaattgaggccaaagcaaaacaactgctccagactgaaaatgctgaaatggtgatgaaaaatgcataatatgttagccatcagattataaagattcccaggtggaatttttcctctttgttgtacattaaaaggatgatgtgttaagccctgattttggatctggtcatgtttccgggctaatcgtttttcatggtcagctttgagatctttgcagttagtcatggataactggatttctcttaagagcattttccctacaatacttctgcgaatatgccttccctgtttttgtagagtcaggactgtgaggccagtttcagaatcatgatggtcatagccactaccaccaccaccaccagccctatattcatttcccagaagctaaaagaggcttcccaatcttaacctttacaatttgtggtaagtttaaggaaagggagataggatttatccctgttctgcagtgggaaaaactgatgtgtgggttcatattgaattagtctctctgatttctgtgtgtgttgtattgacatgaatgtaagtcattctgcttgagataggttgagcagggcatggaaatctaatagggttatttcatgaagttcctttatttgttaaaagaaattacttcctatctattgaacaccatatttttcaggcattgacaaatagacactgattatctctaatcctcctggtgttatgcagagtgggtaataaccctgtttgtttttttggttttttggggtttttttttgagactgagtctcgccttgtcacccaggctggagtgcagtggcgcgatctcggctcactgcaagctctgcctcccaggttcatgccattctcctgcctcagcctcccgagtagctgggattacaggcgcccaccaccacgctggctaattttttgtatttttagtagagacggggtttcaccgtgttagccaggatgatctcgatctcctgacttcttgatctgcccgcctctgcctcccaaagtgctgggattacacgcatgagccaccgtacctggccaataaccctgttttatgtgaagaaactgaggcaggcagagactccaagtcacacagacaagctaaggcctggacttggatccaggtttgtgcagtctccaacactaaaacatttccctcactgtatatcctgctttccttgttcttgaactagactatgtaaagaaatgatcatggtttgtttagtcttacctaccctttcagcaaaagttaagttaaaactgtaaacaatatggagacagtacaaatagggacatgaaagggtcaacatttttatgaatatttttggcttccatgtagtatgtattaatttttttctaagcaacctaggcatatggaatttattggccttcatgagaatttcttgaggcgatattgaccacaaacaatattgctggatcataggatatatgcgtactaggaactgcataattttactgggaactgcacagatagttgccaactttatatctccaccaatactgcataatgccttttttttttttttaaacaaatttcctgattgctgccaacctgataaatgttgattatacctaatcaattataattatacctaaccaaaattaatggtttaattttgcctttttctgattgctagtgaggctgaacatgtcttcatttacttaatttaatttcccttttccatgaattgtctttttatgtctttagcctgccctttattttctgttgggtctcttgcctttttcttgcaagaacttcttgagagaatttttgtaatgtttcttgtgtattctagaaattaattcctctttgtctttagacattgtaactttcttctagtgtgtcactttcctaataactttgtctatggtgcctttcagtaatagcaacaataaaatctttagttttaatgtacttgtctcatcactttatattattattatggtctgtgcattttgagtcatttaagaaatcctcccctattctgggctcacaatgttgtctctcctcattttcttttattaacttcaaaggttactcttattatttgggtttttaaactctttgtaatttgagatacgaagtattaacccaatttttatttttaaaaatctggtcatattgtgagccagttttcccagcactatttgccaactagtacattctttctccacagagttttggtgctacccctaggatgtcagtgcatatatgtagatgtatctttctgagatctctatttggttccagttgacttaattgtatgttcttgggcctttgatacactgttttatttatttatttttcaacttttattttaagtttaggggtacaagtgtatactacaaaggtaaactcgtgtcatgggggtttgttatacagattattttatcacccaggtattaagcctagtacccattagttatttttcctgatcctcttccttctccacttttcaaaaggccccactgtgtgttattttcctctatgtgtccatgtgttctcatcatttagctcccacttataagtgagaacatgtggtattcggttttctgttcctgtgttagtttgcaaaggataatggcctcccactccatgcaagtccctgcaaagcacatgatcttgttcttttctatgaatttttatgttcttttttatcaaggaatactactccttggtgtatatgtaccccatttcctttatccagcctgtcattgatggacatttaggttgactccatttctttactattggatacactgttttaattgttgcatgtacctttatataacatgattttattagcatgacttcttgttacatgtacatttatataacttgaccttattgttatgattgatacattaatataaattttaaagttcaagactttaagattattttgaggcagattccttgcccttatttgtatgtgttaactttttgcagctacacattttctaaaaacacatttctaaaagacacatttttctagaaatgtgtcgacctatctaggaatattgtttaatttccttacttggtaacataattttaatattgctgacgttacattttcttctgtttttttgtgtttttttttttgtcttttcttttcttcttttgttagcagttatggctccctttcatacttggtcatctgtgtagggcagtggtgtgtcactgcaaaggcttgtcctcctctgttgctctgtatgtgcttgtagagctatgaggtacattcctggtacggcttttgaatgaatgttttatgaggtgtgatgcttactgactgcatgggtgaatactcttgaaagtctcttaattacagatgtctctttattattgggacaactttgtcagtttcagcttgattttctccaagtttactttgctctagtcttctcagtttagtgggtcttattcttccccgtctgcaatttatgtcacactgaatgctaggtcactttatttggtaagtgggtgtttgtgtgcctctgcagagtacaatgtattttccccttgtctttctttttctttttttttttttgagacgagagtcttgctgtgttgcccaggctggagtgcaatggcacaatctcggctcactgcagcctctgcctcctgggttcaagtgattctcctgtctcagcctcctgagtggctgggattacaggtgcacattgccacatctggctaattttttgtattttagtagagatggggtttcaccatgttgcgcaggctggtctcaaactcctgagctcaggcaatccacctgcctcggcctcccaaagtgctaggattacaggcatgagccaccgcgaccggcaccaccttggtttttaaaatgtgatgaaaccatgttgtgtacatagcatggcttattattataagtaaatttgaaatcatgatggggacaaatttaggtctcaaaatagatattgtgtgacaaggttaggctaagacaaagatgttaaccttggccaatagttataggattacgtagataatagctgtatttttttttttctagtgtctgtagttcttcaggtgctttatggacacaggagagagttgcagtctttttagttaactgctgagatttaccaagcttgcacaaagtctgataaaatcataatatagaatgttctagggtccgaaacctgctgagacggataagactgatatctgtgggttgttggaattgttctcgtattgtgctttaaatgggatttattagctattctcttgagtttgattataacagccctagctagttacctgactaaacttacatgctcattagaggactttatcagtatggaagttctccatttctcattcagattcctctgtgctccccttagtagctgtcctaccagccttccctcttattcaattattgtaatataattggcagccaaactacttcatccattcaacaggagacatttgctgagctccaaattattaaatcttagtccccgcccctccgtcatccccacctcctgtgtactggtcattttgttaagttctgaggatgcaatgtgagtatgaaatggtgcctaacctctgaagatagactcataaaccactaactggaacttaacttttttataacttaaggcactttatagcaatatattgtgacgataatgatagagagtagggcacatagaagggagtgttttgttctttcttgtttgtgaggaaggaattcctgaaggaggggattttggtgctgaattttgagaggctgagatcatttaccaggccgagatggagaaaggacagactgaggaaacagcccaggttcagagccccaaagggctcagtgaatgtggaacatggactaggagtgaatggagccaagcgatggtggtacagaggggagaagaggctggaggtgcctactggcttgggttcttggtcaaacccatgtctagggccatgcatttcatttctgaaagtgtgaactttatcctgacagcattgtggagtcattaaaggtaaaataagtgttgcaatcactgttttcagatatgtgaaaagaaagcagggagcactttagtggctaccatcataatccaggcagacatcatgaggacccaaactaaggtgatgaaggtgagaaaggaaaggcgagactggattgaagaaatgattaggagaaataattagttattgattagatggaaagtaggaagggagtaagaggaaggaaaaagatgattcttgattcttctggttttgatgacttacatctgaaggtgtcattagtaaggatggaaaacataagaagtttagggacaattgtgagttcaagttgaagtggagttggggtgtctgtggggcattcttttgatcttaagaggcctgagccccaggagagaagtgagggctggagacaattattgaagagtgactaggattgatcgtggtagccatgaatgtcgttgaaactccacagggatggccaaaggatcaaagactgagcagacaaaagaagaggaaaatctgaaaacaaaagtgacaacattgagtacccggtgctttttgtttgtttgtttttatatattatacaattcttacaacaatctcctgtgaaataattatgttattgtcttttacagacagggtgtctaaacctcaggtgagcaacttgccacgctgcccagagagtaaatagggaaaagctgtcaggtcctatcagggcacagaagggtccggtagagtgaagattgaatataggagattggatctgacagttaggtattcagtgggtagtcagaagacctctaaagcctaacactggattgtcaccagtgaggaatgaggaggagttaagaagtgggggcatcaaatggagagacagcgagagagcacaggcaggagagcgagcagatacacgcgggtgtgtacacgtttgcgaggagttgtgtgtatgtgtgtgatagtgattagagcttatagtttggttgagaatagtggtgaggaagtgactacaagtagaagaggaaatcattaatggagtaaagttccagaaaagataggggggccaagttctagagcacaagcatactcaaaggggttgttataaaagggaattgggacgtttattccattgggacaggagaagacgaggaagagatatgtgtggctgtaggtgaatatgcaagtggatgggaaagaagttgagaaagtttgttcctgatggcttggatattttcagtaaaggaggagatgaaatctgctgagagtgtggggccttgaggagacagctggaagttagtaacagttacctttgtaaaaggagcccagtgctttaggatgagtgatagctgtgtgtcattgaaggcccagttgtagttttgtcagtggtggttaggtgctgacggaggatttttttttttctgtttgatagagtactgtaatatgaatagcatgaatgaccttttacaatagaacgcacaggccctgcctgaaacattaagtttggatggtgatttaaagattggacaggaggagagaaggagcagcagacttaacattagggaacttgagttctagtcctagttcttccatttaatggaagacggggagttcttcctcctcatctttcaatggaggaagttgcatttgatgtcctctcaagtcttctgtagctctcagaacttctgtgagatttgcacatcattagacaaaaatgagtggagaagagagtaagagcagctagactcccatcacttatcatttatgggttcttctgatggcagcgctaatcccagagcctttgggaagttgacagcatgaacagcaggacagctgttagataaccgtacccaggtgtctgcagttgtgtctgtacctggcaatgggagaggggtagcaagtcagaaaaggggaaggacaggagcatgaatctgtgatgggatttgcgtgtggggtagacagtttggatttaccctttgcccctcctgctgagtgccacggtttcctgaggaatcactggtagcagataatttagtcgtttcataagagaaggtgatgctctttgcattcatctctatgttgtttctctctctctctctctggttgcattttcttttttctaggctcatttctcactctttgttaaaagtgtagtattgtagaagagaatcgaacagttggagaaggataagtattcagaggatcttaccttaagagtcctttctcctttccgtttccatcactgcaccttgaatgtctttgcctatagattttgaaggatttcatcactcttctgagttggctgtattcttcctctggaaggtgtacttattcttcaaagccatttaactgtctgagcccccttttaagaacagggataacatcttaaagccctcaaggtgtggttagcattaaaaggtgaatccagtcagatgagtggctccagagattattgtaataatttaacaagcctaagcaggtgataggacactcctttgtaaattaagcctctaatctttctttatgctcatgccgatttgagagaaacctgggaatttccttttcccccattaagctattacacaacttaatgcttggaaaattcttcacaggaagaaacagggtgtaacagtttttgtgaggcatcttttaaaaatgcacccttttagctgaagtctgactatacaattgcctctccacctctttgctctagtttctttttgagagtcttaatttctccacataattatatactcttcaaagtgaaaatagttaaaaaattaaaaccacttaaactgaaagttaaaacattgttcataaatatgttcttccatttcctgagaaacaacaggaaagattgctttctcaaaataaactaagtagcctgagtatgaagtcagaccaatagagatcaaagattcacgcatcatcacacaatgggaaatttttagtcatttttctaaccacaaaaataagtctaatgctagttctgggggtttgttttgttgcctgtggcatcaagatatgtatgtataattcagaatgtacctttggcatatttgaaattgtcttagcctaatgtgactatctagtgtgtctgaaagagcctattttcaagatatatttgatgagtgtgaacatactacatatacagcaatggaaaatagccttcttgggcattaaatttaggaagaatgtagaaaaccagctgactttttttatctgtttagcatctagaattaaaattaaacttgctagatcagtaaataattgcatagctatgctagtagcaaaagtttagcagctcatgttgaacactgaaccatattgaaaaaggcatctgctacagtaatctctctctctcacacacacacacactctcaggtaaggataacagcatttattaatatttctctatttccagaaataagatatgagaggttattttaaagaaagaaagtaaatgctcttatgattggcagatttttatttgtgggatacaagcaagatgagggagcagtatatgcctgcagtattcatctgtcattaatttgtgtcaaataatgtgattctgtattcctcaaatgttgtaaaataggtaaacattttgtctttcacagaaagatagaaaatcagatctccaagcagtttactgctatgatcttagcttccttttcaaatcccttaggtattttaaacctaagtctttatatttaaaatgggtctcttgtagacggtatatatttgggtcttgttttctttatctactctgacaatctgtgtcttttaattggtgtgtttaggtaatttacatgtaaagtggttattgatacagttggattaatatccaccacgcttgtaactgttttctattcattgcatatgctctgtttctccccccacatttactgcctactgtggttttaagttaacatttcatataattccattttatctcctctttgagaatgtcaattatactttctttttaaaaaggttttagtgattaccctagagtttataattgattttagtttcttaaaacaaaaaaataccccaaaacatatgtatgtgtatgcgtagaaaaaaatttgaaggcttttcaacaagacattaagagtaattatcggggtcaggcacggtggctcatgcctgtaatctcagcattttgggaggtgaggcaggtggatcacctgaggttgggagttcaagaccagcctgaccaacatggagaaaccccgtctctactaaaaaatagaaaattagccgggtgtggtggcgcatgcctgtaatcccagttacttggaggctgaggcaggagaattgcttgaacctgggagatggaggttgcggtgagctgagatcacaccattgcattccagcctgggcaacaagagcgaaactccatctcaaaaaaaaaaaaaaaaaaaaaagtaattatctctgagtggagattataggtgattttgactttttataattttctgtattatctgatttttaaaaaaaataacaagcaggtattttataatcagaaacaaagctttttccattttggtgagaaagaaagaggtaaatcacagcgaaataagctgaaaatctatgattctaaaatgtataccctctctctgcttccaccttgagataagcacattatcccataattacttgcactccactagtgggaaaaaggaaaggggtttactgcttttaagcttgcattggaaaggttgtcagcccctgtcctataccttctgtgcctacacaatgaaaattgaatcctcagaatgacttggaatcagtctaccctgtggtttgacataaagttttctacatatgtaaagttaaccaggccccactatttctgcattattgaggttctcctctaagtagaatgtctttttgctcttcactatctttcaaaaaataatgagtaggattagagcacttgaaaaccactttaaggtatagcctttgccaggcaaggtggcacacgcctgtaatcatagcacttttggaggctgaggtgggtggatcacctgaggtcaggagttcgagaccagcctggccaacatggtgaaaccccgtctctactaaaaatacaaaaattagccgggcgtggtggcaagcacctgtagtcccagctagtcaggaggctgaggcaggagaatcacttggacctgggaggcagagattgccgtgagcagagatgtcacgctactgcactccagcctgagcgacaagagtgaaactccatctcaaaaaaagtatagcttttcatgacaactttgtaatactttttttgtttgctgttatgaaatatctccttttgggtggggtggagaggggatgtcaggtagtatctgtggtaataggtgaagagatgatagctaatgtgaggctgttggtccatctggatttcagggaactcacctccccaccttgtgaaatattagagaaaattaccattactgttctcatgatgttaattatatcttctgaagttttagaggtttacatataaaccaaaccccaattaaggtttaagaaagtggcatgacttagtgcaaacagatgaaatgttgatttctttaaaagttgtcttaatactaatggatcttacaggtgttttcaagaaagttataagttttgtaagtataattttagtaaataaaattttgtaaatataattttaattttggactttttttttttccttccttacttgctgcatcaaagtgaacaaatgacaagtgaaatttttttgagtcttctgagagaaggaagtttataaaagcagatgcctttttctgtacttccaggtcaaaaattgtaggcctccattttaatatagcttattgttagaacaaggaaatttcatttaagacaagaaatgaagtcatagttacattctgtttaaaactaatagaattaaacattttgacataaataaaacaattagaaaactttacaaaataaatgtaaaaagaattcattcacaatacactgattattacagctatgttaaagtctacaattgaacacaaaatataaacaattgttatttaagggattataagtaaatttaataaattttctttacaaaatttttggtgagatatcttataagaagtagaggggtcagaaatcaaatttctgggatgcttattaagataagcttgaaatgtaattgaatttttaaattcgcattacaagtcataatagtagactattcggtaaatgtagggagtaatggtaggtaatagcaaagctaggtaatttgactctttttttttttttaagctcattttcaaatgagagactccatacacggagatagtagtgtcgaacaatgataaaaattctaggagttgttactatcttaacacatttatctaagcatttacctatgttgccttggtgcctttgactgctgacattgcaccctttcgtctgtttttaacttgtcgaaattgttgttataacatttatctgcctctgttcctcaggtatagctgtgaaaatagaaatggttcggggttaaggattgtgaatgggaggaagaaataatcctctcattattttcaatacagggaaagaggattatttgtaaccctgagcttttaccccaaaaggacataaagtataataggtgatctttgcttttataaatgtgaaccataataactactcttgggaaaggattgataggccctattttctacttgccttttcatccatttattttgtgataattcctctatgtttcctaatcatttgctctttgaaaaaagtgagagcttgtgatcagtcctgcacggaagccagtacacaatctaggtattagagcagtgcattgacagagaggctaccttccattttgtgcttccaagaggtggttgtaaataggaaagtgaaaattgttaaatgacaaaaattactttgaagtaaatttctctgcctgttttagaacttaactctagatgaagaatcagtatattttggggggcagtaatagtaagaccaaaaagcaacgtttttacagcagtgtcagaatatctatttgccaagtattatttccactttatggatggggcctaaaagccagatagtttgtttgacttgaaggtcactggttaatgagccttttattactacgtaggttttcaccggtgctgagcaagaaagcttacaaatctcctacctaaggcatgcttatatccgaaccattccaagataaagaaaatgtacaatttaagcttttctggaagaaaggaaaacctcaacactttctgatctctctcagatcaatagcattttaaactaaatcttttatgcgaaaagttaattaaggcaactccaattcctttcttaacagaaatggttttacatcatcctccatgaagtaactaaccaaagacaggcccaccttcatctttctccttagtaagtttattgatcccagggctctttactcttacacgtccaattttaaaatgtacacgtggccgggcacagtggctcacgcctgtaatcctagcactttgtgaggccagggcgggcagatcatgaggtcaagagatcgagaccatcctggccaacatggtgaaaccctgtctctactaaaaataaaaaaattagctgagcgtggtggtgcgcggctgtaaccaaagctacttgggagtctgaggcaggagaatcacttgaacctgggaggtggaggttgcagtgagccgagatcacgccactgcactccagcctggcaacagagcaagactctatctcaaaaaaaaaaaaaaaaaaaaaaaaaaaaaatatatatatatatatatatatatacacacacagacgcattttgtacgtatttttaagattttccttgaaattctttttaacgttctccattcccccttttaaagttacttagaagggctgttataggaaacctcatttgctgcagatctgattatcttctgggataaagataggtagaagttgcagggagacatactttaggttaatgcaaggaacaaatcaacttcaaagccatgcagagctgggctgaattgctgggtgctgtccagggccttagcatcaagggcacttttgagagtctgtccatccttggccaagggagtagagagatttgaaacttcatgggagggtggggcagagatgctctctaaggttacacagtgtgtacataccattcagtcacaagctttgtgctagaaaagcccatgcttaaataccacctctgtcccctgctaactgtgtgaccctaggaaagtaagctaaattcactaagcctcagtttccccctctttaaaatgaggtcaaaatgataagaggattatcatgtgactttaacatacataaggcacttggtttagttattccttcccactgactttgtgattctatagtttttcattctgtgccttttttcctttctttcttcttttcttactctccagttcattcattgtagagccattaatggttttttcaccttgtattgtcctcttctttattaatacctgtgccttagggcctactagtggtgccagggattccctgagctcctttggtagcatcactgtccatgtcacatatctggacaagtcagcgaagaggtattggtcctctgtatcctttggacttggcatatgggcatccctgaagacttttacaaagccaagattcattgctactttagctttcctgtgttgtgtactataacaagttctgctccatgaaagcatcagatagcagttggatatagatggagaaaaaggttttgtacagggacttatggatgtagaaatctatgctggtaacatttattttttcttcatctaagtttctgtttttggagccaggcttctaattacaaataattgtatttgacagagaagaagaatatcagatcctgaagggccagaatgattcatcctctttaaggggaacatgtaacataactttggggaatagctatagatactaaaggggcaacataaaacagttattgattacaaagtgtatgaagacccagttgcttggcagagtgatatcatttagaaaagtggtagaatctcattcgtacacacttcatgactaaaagagctggttataccgttagattgttaaagctagaaggaggctttcaggtcactctatgcagcgattttcttctattaataataaaatggagatcagaagagtgaagtgactttttccttgaaacataactcttaatactaaaaaaggcaagtcagggagatgtgggccctaattttgttgttattttcttacatctagtgtaccaccctgactccagcctctggacctgggagtcatttcattgtagctcaaagaaggctatatataccctgttagcccatactgcttactaagttgttaatgaaaaagttattgttcttcataagagatccgtcttttgtccatggtaaatgatgttatgaaatttttggtttatttggttgttctaggttaggaataattgaagtaggccagtctttggacagtttagttctcctgtgtttagcactttttgtatttcttctctgtgctggatgtatttatgtttttcagtctttctgtgagttaactctaggagatgggtataactgtatccatttttgtcagatgataaaaagaggcttgaaatgcttaaacaacttgttccaggttatacagcttataaactggaactggatcccaggtgagttttccagagcacatgttcataattgctgtcttttttttacatttcctgcttcccttactacctgagtattgggggaacaggctgtggaactgaagattttactgtaacaaatgatccaaggaagatcatctggcctgaccccttggcttccagcaggaagggtgttttctcccgtgatttcatggatgagatattcaaggccttgtttttatttattttttggtttgctgtttcaaaataaaaaggcttttagttctttgttattataaaagtaataaattctcatataaagaaaatataggaaataaggagaattaaaaatgacctttgtagttccctagtcagttgatgaagagtggattcagatagctgtatcccctcactagccatttgactttgggcaagttttatagccttttcatgcctcagttccctgtatgtaaaacaagaataataatagtgcctgccttatatagttgttatgaggattacatgggctcttatttgtgaagcatttagaacagtgactggcacacagcaagtgcagcacaagcattggttggataaaataaagggctgcccatgtagtcttctggtctgtggcccctggaggaagaggcagaggcagacccaaaatgtagatcacgcagtacacaagtgggtaatgggctgagctgtgtgttgaagcagagacaggaagaggcggcctgggaacctcagtttctttttgcctccttagtgatcctaataacctggaatattaataaacttatgtccctcactttcaagatgctgaaaatgattatgcttcttttttttttcctcctagctttcagagagctcatggaaaccaagcctgtttatttgagatttttctgttgcagggtaggccagacatgaatttcctttcatgtgatattttctgtggtctgtgttatttagttcttcctttgttttcttgtgctttggtgtgaagaaagaagatgccctgtttataaagccttggtctttcatgtatcatgcattcttaaactaattccagttgctctgaccctttgagattttaaaattgctttgttccggtttttaagtacttttgaaatattaaaaatgttcatgagggagtgggtcatgggaaaaatttcccagggttgtttgttcttagaaaattgtgaatttgcatggagaggttttatgctgggttaaattctgccaggtgcattggtcacttgaacttgagtctggttcttttgatatttaaatccgtgttgttatggcataaagtgcagagctggaacctcttaaaactaaatgcctgctatgttgagctcttgccccattgtcccatcaaagaaaggtcttaaccagtacttgggtttgcattgaggcaccatgtcaatagtgaggatcaaaagattggtctgttctgttttttgtctttgaaccaagactttccaaaaagaacatagacttgttttgaatattgccgcctctgggtgtgagtgattatgcaagtcagtagcatttttgtttttttgttttgaaaaaactgttcaacatagatgcagaggtaagaaattgcaagaaaaacaaaaacactttttcaaacttgtacaaagccatctttgaatgcaggtggtcctcagtttacaaggagttgtaattcttccttcatctgatgaaagacaccactatccaccagttaccatttgaacactttccatgatgtatccttctcatttaactcccagatgcaatgaagtgccaagccctctcaattctatcttaaattttatctggtatctcttaactgtgctgtgggctgctgcagtccacagctccagttgcggcctccatcatgttttgcctggactgctgcagctgcctctgaactccagtgctctctatacattggaaccttatgtcagtgacctccttaacaccctccagtgaattctcattatccttggaactccttaataaggagttaaaagccagagtccttaagacatcaagaccttgccgcaggaaacctcagcctccatctctagtctagagtactagctttacctacccccgacccctcaccatcttccagacacaaaaaacttcttcctgtacttcttattggctaagttttcatgacaataagctttgactacagggtatgtctgatgtctagcacactttttccccttctgctccttcacttttgtttgggtaactcctgtgtcttcttttggttttaatgtccttccagaagctgtctaacccctaaactggattaggtccccttgctgtttccatagaacattttacatctccaacatttcacttattacattgatttgccttttctaatagatttcaagccccataagggcataaatatgtctatcttgtttcccactgtatcccaatacccagcacacagtaggatctcccgtgtttttgaatgaatgtggtccagatcattcccttattattacttgtagtagtgcttagcacacaacctgtgaatgaatttcattcctttaaagggagttttcaaaaaccattcttgttgattactttgcttaattgtactaaatatgtataaacatgaaattatatatatatatatatatatatatatatactttctgtgcttatagtttttataaaactattaaacattttaaaatcgcaaatacaaataatatattaaaacctttgaaatttaaattaataagatgaatgcttttttgccaacactaaatcacttatgttgattttttaaaatatgaagttgttccattgggtccaaatatgtacttaaggcatatgagtcttttgactgatagtagtaatgtagagaatacttatttgcataaataagttattcagaagcacattatgaactcgaaagccttgcttgctagctcaggactatcagaacttatcagatcgtaaatctgccagtgaatagttctttagttacaattttaatgattttaatgacttacaacttttttgttagagctgtaaatgtcctgttcattcaaagatgatgttaccatttcagaattactgacatcaactccaaatatatatctaattcaactattggcagaatcaggaacaggaatatgtttgtaaattttagttcttagagatatggaaaatttttaaggttttcagcatgcagccaagtgtcagactgttgaaggaagtcaatagattatagaaatgactcattcagattattttgaaaacaacttgaacagaactttttgttatcattgcctcctctttataatttttgaattgctgaaacaaaacctttgctctacaatacctgcatgtgtagcacataccttcatgtcatttgaccttcccttggtgtgtttctgcagattatgtatcaagtctgcctctgtcctttcctcatcagcctgaagcagttactatagtacattccttcactgtttgtcattttatggtgccatgaaattaaagtcttgctcaatgacagtcttaaattacattttatcattgccgtgtgtgcttatcagcaccccaggagtactgatcactgatgaggaatgcttggtgatgtggcaattataaatacacacttgggcatggtattgctaagggggtcattcggatggtccagtgtgcatttatatcagtttcctttagattaacattaagatgaaaacacaaattgaaagattattctggagaagtcatagactcttgaatgtgtcggtagttttttagagatccttagacccctgtttgagaacacttgcttacagcattcatagactgtgtacaaaaatacagccagattattgtgtttctggaagtgttctggcgtttctaggccattcctttagttctctacttttctttagcctttgagcctatccctacatcttagaaaaccccttcttggtgagaggaatgtcaagctttgttttctggtctcacgaaggcccgataggctctctgttggggtctctctctggctatttctggttacctaggtaaccctgcagcccatcccctcaggagcatggagaatacacacctgttgaggagggaatagagagagcagggcagcaaggagagttaagggcggcatgaggatttcaagtgtatcaatcagatagatacttacacttaacattaaaaggaaaaatcggtgtgatgtcgaccccaaagtcgaaggtattttattaagccaaacatcatcagggtagcaagtcacttagtatttgtgtatttttttaactgttttattgagatataattcatataccatacaattggcccacttagagtgtataatccaacagtgtttgtagtacatttagaatcatgtgaccattaccacatcaattttagagcattttatcacccgaaaagaaactctgtccccagttaacatcattctccatccttttcagtctcaccagcccctgaccaacattaatctgtagatttgcctattaaggacatttagaataaatgaaataatacaatatatactttttcataaattatgtagtcactttttgactggcttctctcatatttttaaggttcagctacactgcagcatgtgtcagtatttcatttctttttattgccagatcatatcccattgtataaatatgccagattttaaagtgtaccattctgtaatgttaagtatattcacattgttttgaaacagatctctgggactttttcatcttacaaaactgaaactatatactccacattttatttacccattctcatagattgggttgttttccacatttgggcagttgtgtataatactgctgtgaacatttgtatgcacgtttttaggtggagatatattttcatttttcttgcgtgtattctgcctaggagtggaattgaagggctagtcatctggttattctttaacattgtgaggaactgccaagctgttttccaaagtggctgcaccattttgcattcccaccagcaatgtcgtttttgatttgcatttttctaataactaaggatgtagaacatcttttcatgtgaagacatttaattaaaatgttttccttaaaataatgttttactagattctcagggttataacttagaaaggaaacatccttttagaattcttcaaggatactaattgtaacccaaattatgaggattgaaagacaaagagtaaatggctgccctcattgtaaggactgactgttggcaggttaggttttaagtagaaatggtttagcagtctcattttcaggaacatactaaatggagtcctgaactgggttatctgtgataagcaaaggaggaaagagaaaaagaggcagatgggccaatgtaagccattccctttcaagatgctcaattactagcacaatcaagccaaagaatggccttcaaatgatgagaaaatacataagccatctatacattttctagtaccttgtttggtgtctggcacaaaatatttattctgggttttgaagaagatagaaccttgaatgcagctgacacaccaagattttgaatcataaaatgtaaggattagaagctaatgattcccacaggaaacattataataagtaaaaacaggctgcgcaaaccgtccctgggttaaattcttaaagctactgggctccttcagattatgcaaatttcttcaaaactacatctgatctctcccatgattccctcaacacatgcatgaaaggtatatgtgaattccatgacacgaatgaggtttatttatgtaaatatacctcagcatattcccccaccccccaccaaataaggcacatcaattttttacaataataaagagctaattgttgagttctaactatatgccaggcacgtgctaagcagtgtacattgtgtcatttaattgacatagcaactcgatgaagttgatcctactcttattcctgtattacagaaaaggaaatttaggttcagcaagaccaagtaatttgcccagtatcataccagctctaaagaggctaccggaggattctaggcttctggtcagtgctctcaaccactgaacacaaaaatgtctttcttattccttttagtctgctgcagtaattgggaggatatagaataactttttaagtattgacactgtattttttgttgttgtttttttgttttgttttgttttgtttttcagacagaatctcactctttcgcccaggctggagtgcagtggtatgatctttgctcattgcaacctctgccttctaggttcaagtgattctgctgcatcagcctcccaagtagctgggagtacacacgcatgccaccatgcccggctaatttttgtacttttagcagagatggggtttcgctatgttggccaggctggtctcgagctcctgacctcaggtgatccgcctacctcgacctcccaaagtgctgagattacaggtgtgagccactgcacccagccaacactgcgttattttgactgagattttgtgattgctaccttggcctgtgctgactagaataaccagtttagagaagaacgtaaatgacctgatggagctgaaaaacacagcatgagaacttcgtgaagcatacgcaagtatcaatagctgaatcgatcaggatcaacttaatgaaataaagcgagaagacaagattagagaagaaagaataaaaaggagcaaacaaagcctccaagaaatatgggactatgtgaaaagaccaaatctatgtttgattggtgtacatgaaagtgatggggagaatggaaccaagttggaagatacacttcaggatattatccaggagaacttccccaacctagcaagacatgccgacattcaaattcaggaaatacagagaacaccacaaagatactcctcaagaagagcaaccacaagacacataatcatcagattcaccaaggcagaaatgaaggaaaaaatgttaagggcaaccagagagaaaggtcgggttacccacaaagggaagcccatcagactcagcagatctctcggcagaaaccctacaagccagaagagagtggggaccaatattcaacattcttaaataaaagaattttcacacggcgtggtggctcaatcctgtaatcccagcactttgggaggctgaggcaggcggatcacaaggtcaggagtttgagaccagcctgaccagtgtggtgaaaccctgtctctactaaaaatacaaaaattagctggccatgatggtgcacacctgcagtcccagctactcaggaggctgaggcaggagaattgcttgaacccaggaggcagaggttacagtgagcaaagattgcaccactgcactccagcctgggtgacagagcaagactccgtctcaaaaaaaagaaaaagaattttcaaccctgaatttcatatccagccaaagtaagctgccttttagtttttctaaaaaactgatagagatgccaggtgtggtggctcacacctgtaatcccagcactttgggaggctgaggcgggtggatcacgaggtcaggagatagagaccatcctggctaatgtggtgaaaccctgtctctactaaaaatacaaaaaattagctagacgtggtggcacgtgcctgtagtcccagctacacgggaggctgaggcaggagaatcgcttgaacccgggaggcagaggttgcagtgagccaagatcgtgtcactgcactctagcctgggcgacaaagcgagaccctgtatcaaaaaaaaaaagaagaagatagaaactaaaggtgtctcctggcctcagttttctaagtcaagaagagagtctagttaaattttgtgtacttttactacagatttcctatatttttgcaaaattttctcttaagttggtgcatttagaagggaaactcaataaatattttgtcactgaagagatgagagacatttgacaccccctccccaccttcccctcagaattcaacctgataatgcagaggctttcacagccatctagtcccaacattgtctataagatgcttcaagatagaactttaaggaaaacacatgtaggaggcagtgaccaggggtacttggaaataggtaccctttccaatttagctttgtgaacatttgttcaatttggtagtttttaagctaactaatagcattgtgggcattttattcttggatttaaatttgggtaatttctgtaggacgctgaaataccttctacctagacatagaattcaactttaaataatcctactttaagttggttcctgggatgaggtacttagtaatacagtattgaagtggtagatggtgaagttacttcctgtggtggtgtttcattcatgtctcaggttgcaaagttatgtaagttgtttacagcaaaatactagtgaggaagagttcatttgccactgctagaaggtacagactccccgcatttagtatgatgactatctttggtcttttcttgcagctgtcttgtttatagagtcaaattatcttatgtctatctagggttaaatgtcacagtctcatgggcaatgtaacaaagtttgtattttgtgctttgttaaagaaagctagtaggtgattacttgcggttgaatcggtatttggagactcattaatgattaattgaaaacacacacaagcacttttttggtaaccgcctgcgcatagcgttcttttggggttcagatatttagcagatacccccttccctctcctaaaaatatacattcattttatattgtgttagacttaaagtatatttgaacagaatatgaagtttatgattcttaaactatttgcatttacatatggtataattactcttaggtctctgacggtaatagatgtccattttttttcattaatactcttgtttctttctactgtttacaggaatactaacaaaataccttaactgtcatctcaaaagttaatgataaatgttaataaacttcattataaaactgttcggtgatttacaatttgaaaatattggcaacccatagtacctggtacctacggtttggaacatatggtgaatggatttatttcaataccttcagtggctcttttctgaaaaagtccattgaaaatcttatgaggccttttttgtacttttgcctgtttgtttgtttgtttgttttgttttgcttgcttactcctaaatactacctcgtatttgtccacttttacttttgggcacttacagcttttaccttcatttggcattggggccattatgtttctattgacctagtattgttcagttccttggatgatctcatcctcattacctggcaatctctggcaaagtttcacacttaaattttataaattgggatatcactttcctgagagtgtattccgtctgcaacgtaaatgcaagttcttttgggacaagtgccatagtttaaacgtaaattattcattgacagtagcaagcgttgtgcaaactatgcagtgctttctcagtaagtgctcattgagttgccctgtgtgcatgatgaccagctgtgttatctggatgtggaacatagcgttctgctatctgttttcagaaatgtcgcatatcccttccagggcagaccactcattgccaaggcataaggtcgaaaactgcccagtggggtggaaagcaaaggcagttggttagctgttggtgctttctttactctctacgaggatctttcaatggcagtgaccaggacttcattctgttatttgttatagaacaggcaagacatgaaccaacccagtcatattctgtaccaaacacatctgtggtccattctgctttccccagctttggtttcttcatttcacacgtccaagagaattttcttaaggataaccatcttattagaaatatgtctaattgataatgaaatgtctgtatttagctatttaactttgttctgggttctctctgcttacttaatgtgactgcatttccctgagatgaatgctgtgggtttttgcagtctctccccaactttgcccctcccctcttagatgaaggtgcatcactagagaacctgcaccactgtgtctcctacactgtgtcaggtagacagtacatgattagtaatgtgtgtgcaaagctttcatgaatgcttttaagaagttgaactcttaatattttgcttaatttccacatgtttaggataggattaatgtagagttgaaattaagatagcatcttgggaattttagataggaatcgtttgtgacccaaagtgaataaaaattgatattacaagcaggcttttaatccagaaaatttacttattctgtccagactgtgaacagttgttttgttctcaccaaaggcaattttggtgaagtgttcttagaaacagtgtgtacaggcttctgtaattgtacaggggagagatcatagagatccagggaacatcaagatactaattcaacctgtcctctttgaaaaatttgaccccctcccctgtaactcaacaacccctcaaagctgtcgatgcaaagttgcaaaataacaaaggcttctgtattaatttctgtgttacagccttctgggaagagaatgctgggagaaaaaatatttctaaatgtataattgcatatatacactttttaggttctccttgtatttttaatgacctaatactagaacattttgataagtttagttcttaaacagaactaccaaacacaaaagccagctggaggcagcaaatgactaggtggatgttgaagctcatgacttcctttcagtggcctcttctttgtatacagctccatgtttatgtccagatggactgactatattgataggttccagataaacaatggacacattagaagaggggtgttatcatcaaaaactaacaactgaagagttaaaatatgaaacatatctttgtcatgtaggattgaaaactatcatattttattgaatcaaatttatggcatgtttctcgtaaacctaaattttataatcctgtctgttaagttggttggcaaagcccaaatgttttcccttacattcctgaaacaaatttataaagtcaaagaatcagccatatcatagcaaatttagagtgccgtttgataatgaacgacctagtaggtcattaatcttttctatgggcagtgttttttttcctcatcagctgcctaaagtatttgggaaattgtatcttgtgggagttgccattcatcaggaaatgcttgttcatagatctaagccagaccacatgtagcagtaaatgttatttatcactatggaaaccaaaaatcacaaatgggatagtctaagtgttaagcattctgcttcctatggcagtaagtcatacccaaatttggttcacttcactcaaatatttgtgaggcacttaacgattaaagggtttgtaggtactttgtttaatgaataaatttctcctacacaaagctgaaaacatcataaagggaaaaactaggaggaagagttgccagtttatgtggcttatcaagctcataattgtagacccctaaattcggaggcagaggactagagttttagctgtggtgctgccagaagctagctgtgtagctttgaactagttctctaacttctttggaactcagttttctcatctataatgagatggagttggattctatgacctcgaagtgtgaatctgttttcctttattattccttgagaagtaagaaactatttgtgtgtgtgtgtgtgtgtgtgtgtgtgtgtgtgtgtggtaagtggggcataatatttggattcattatgtcaggagtagtgaagtttactattacccttcagtgactttatcacctaaaaaaagaacaaaaacccatgcttagtaaggatagggtccacatgttgccatttatctatggattctgccttcctctccactcagtatgtattgacaaaggatgtcattattatctagcaatgatgaagtattgcaataatctgaattgtagatgttgaattgactcgtagccaaaaggtggcatttaaggagtataattgctgttcatagataaaagtaagaataatgacttaagcagtaatccaaaaatgttaagaagttggtggaagaagaggctggctgaattgatgagtgagaatgtttattcataaattcatttaattcttagttctactgagactttaccatgattgtcattgcagtagttcctatacagtgggaatatgttcctataatgggaatttgaatcattcagtgttgctgatctggttgtatctggacgaggcaacttctcttacctgttgtctcattatttagtggggggaatatctatcccatccttacttctcaagaataatatgaggataaaaaaaacctgtataaagctacaaaccaatattattttgtacaggttaagtagcccggtagaatcttttggaattgattgaaaatacaaatgcctttttgacaggcattgctgttggattttttttaacatgggagaagtttactagatttcaaattttaaaatatgcatgggattgaaagttagaagctctgaaacatttaactatctggtctttacaaaatatcataataggtgtccattaaatgttcttcattcactcattcatttattttatagtttttgatcatttgctgaatgccaagcagtgttttagaagctagattatacagttgtgaatataacaaaatccttatccttatgaagaccatatagtagatgagtctgataagcatgtgtttcatggatatctattagatatcctttaataagtgctaataacaaaattaagcaagataaagagatagggcaatgagagaagggggtgttacatggtcagagaaagcctttcatatacaacagtgtaacttccacctcttttaaggtcttctttatttctatatgttgtcatctgacactgtacccaacacatagaaggtgcttattaaatgtttaaatgaaatttttatttatttaacaaacacttatagaggacttactctatgccaagcagtgttctaagtgccttgcaaatattattttatttgattttcacaagaacgctttgtatactgtttcattttatgaggaaactgaggcaccaggatgttgaggaacttccgcaaggtcacagctaataatgaatgaatcagtttgtaaaggaatttaaaaaataaagattcttaaaggttcttagtgccacctcttgctttcaggaacaccagatagggtgggactccatattttgggatcatgttgggtagggagcatgtcagcccctgaggtccttttgaaaagctcctaatacctctacctcccaggccaggcagataccactcttctccaggcttccatccactcagagctctttcctgctggacccgttggaagcttcgtctgctctgctgacacctggactttcctggggctgaggttaagtgatagctttttctctgtggttgaggaattacagctactagaaaagaatgtatcctcttcatccattctcctacctactatttaaatggcaagtagtgtttttatactgtggtccacggacccccaggattaaaaccaccgtggggctcatttaccatacagattcctgggctccacctcagacccactaaatcaggctttgcagatggggcccaaaaatctctattttaagcttcttaaaaactactgggttagaggatgtgcacttcccgtgtgaggggaaagagatgaagaaaagagttatataagacaggtttgtctccacagagagtgcagagaggttctgtaattctgatggaattctttccctgcctggttagagcagagaggtggcagggtagtgggagatcagatattaagggaaaatccatgtgaaaactccagagtcatttagaataagggttagttaaccgtggtctacaggacagctgcctgtttctgcaataaagtttctttggaacatagccacattcattggtttatgcattgtctgtggatgctttgcactacactgatagagtcgagtagttgttacaaagactatactgcctccaaagcctaaaatatttagcattcggtcctttatagacaaagtttgttggatagcttaccaaataactacatgaagattgaaaagtttctatctgctctccaatttggtagccactggcccttgaagtttggatagtgagcacatgaagtgtggaaaatgtgaccaggtaactgaaagtttcatattatttggttttaatcaattttaaaaattaatttaaacctcaatagccacatttggctgatggctaccatgttggataatgcagaactgcagaactaaagtgaagctttcagattgggattggctgtaaaatgtcaaatatgacaagggtctgctttaggcctctggaaagagggagtctaaggaaaggaacatctagataggatatattttagaagaaacttcaaacagttctttctctctctctctctttttttttttttaaatttgtgtttattaagagttacttgtagggatccagttctgccacttacagctttttgaccttggtcaagttacttagcttctctctgcctcaatttcctcattggcaaaacaaggataataacagtaccttttaaaatttttatgagctttaagtgtgtatgtgctttaaaaataccttgcacatagtaaaagctttgtgagtgtttgctgttcttaattattgttattattatttcatgattttttgtggcctaatctaagaaaataatccttacatatttgccccactcccccttttctgttttaaggaaagtggattctgaatttcagagtaatttgtatattaacctggctaatgcagcctgttcctaaaacgtggctaatgcagcctgttcctaatttagggaccagcggtggttcacctccttccacgcaggtttgttacagttaacacggttgggtccagcacgtgacatacatggtatggtattgtctaggttttattgatggtcactgtaaacttttatagataactgaaactcttcaaaaataaactaggcatttttatttagaagcagactgtttcttagtgttagttcttgttcctaagcaggaatcatagtaatatttgaaaggaagtatgtttctattttatcttgagacgtgtcagaatctcctaacatgagtatgcttggtgccttcccttttatagaagctgagcaggactggcaggaactgtttgagaggtgcgagttccctttgtttgtgtaaatgatagcagtgatagaaattgaaaaatgaaagaaaattcagaatcatgtcactatgggtttttaaaatccagtggctatttcttaactctttcatcttcagtattttttactaattagttccatagacttgcagtggataaacaatatcacttaaaaacctcccagaagccagtgagtgcagcttcttgaaaatctgcttctaagttttctgagacagcagtgatgtgaaaatactttatagtgcctagatctgttatccaaggctcgaacagcagagtgttcagcaggtattatgtgctcaacagttgctcatcaagctgcctttttgacctgtaagatggcattgccaacacctgttgatcagtacataatatgagaagaaagcccttggctagatggcaagaggtattttcttatgcaagtagtcccattctctaggccatattgttctgttggttgtcttttctaattctggattgtgtttgaatgtatctgagatgcattgaacttgcttagagtttccatggactcattgtacttagagcagaaggaatgagaacatggtcaactattaatatcctctagcttgaaactatctacagtctgaaactttagtcatcttagggaaggaggatatggaagtatagaagtctctgatgagggccatgtgcagtgtctcacgcctgtaatcccagcactttgggaggctgaggcgggtggatcacctgaggttaggagttcgagaccagcctggacaacatggtgaaaccctgtctctactaaaaatacaaaaatttagccaggcatggtagtgggcacctgtaatcccagctacttgggaggctgaggcaggagaatcacttgaacccaggaggcagagattgcagtgagctgaggtcgcaccactgcactctagcctgggcaacaagagcgaaaatccgtctcaaaaaaaaaaaaaaaaaagtctctaatgagatgtggcccatgaaggactggattgcgcagtaagcacagctgtttggctgcagcccctatttttctgtgcacatccgtgcacccccctgctctgtatctccctgctgcatttgtccgctagactttatctatgcttttctagcactcttatctacagccgttgacacccatttgtatctcttcagctgaaaaagagtaagcaatgaaagagtttatcctgctataaattcaatgtctgttcttctccttttgtttggggatgcagcttgcaatttagaaggtggttccaattatcaatttcctgggggtataaaccttattaatataaattgcagagctagaattaatgaggagagcttttgagcagaccgacatcagctcactgtaaggaaacaattgttgagaactagagccgtccagtagtttgctactgaaagaccttggaaaagctcagatgattctggtagagatgttgaaatggataggaagctggtgagatggttttttatgttgggatattgtatacttcaaagatattataacaaaaatactttcctggtaaatctgttgtgttaccttatgtatgatattgtgtataagaaacaatttcttttttcttaaatcataggtgaatgagactatcaaatgtggcacactagtagacaccgttgttctaatgatgcatgttgtataatcatttcttccctacaaatttattttaaaattaattaacaatttactaattatagtatacttggctgtattcagtagttatttgtttgggacctgctgtacccatcacagtgtgctagcttcatgagggataaaaacataaatatggtgctgcttttgcattctaattggaaaaagagacatgtaggttagctagaatataaatgaaactatgaaatgtattaaaattgaattacaaaaaggtaagtagaagccatttaattcttcctggagcaatctagagagatagtgtggactgaacttcaaaagaagtaaggttgaaagaggcaaggataataattattaacatttatttggtatttagttatgtgtgagacatttatctatgtgctttgcatgtctctacgacgtttggtactgttaccctcatattactgattggaaattggttggggcaaggaggaaatttgatatagatgtgtaagcagggaatgttgagtagtctcgtgtagctgactatagaatttgtggggacagggaactgcatacgagatgagaaatgtagactggagtcaaattatctaatccaaaagaaaaatttgactttccagattcatttccaggatcatctgaggcaaagtgtttaatgcaaaatacattggcattggagtgatgagctttagatttcagtgctagactgtaataatgtgaaatctcattaggttggacatttgtcataaatcagaaagaagagacctaggtttgatttttttttttttttggcctaccatttacttgctgagtgttctacattctgcagagatttaggttcattatccataacatgagtaataaaatttccccatgagtatgtgaaagttccatgcttggtgccttatgtgtatataggaaggatgtaataaatattagtggatctaaatcagaaaatagaatttaaagcagcaactcttgcccaaccagctagatacccttatatatatttcctttgtatcactcttcaaccttttgaattcaacaccttatttcttcttgcatggactgttgtgctagtctttgaactgatctattcattctattccaaatacagaatgttagattaattttttcaagcacaacatgttattgctaacatacgtatgtcactgtattacataatacagtgtacatattacataatatatgtaatacataatatatttattacataatacatgtcactagcactggaatctaaagctcatcactctaatgccaatgtattttgcattaaacactttgcctcagatgatcctagaaatgactctggaaagtgaaatttttgatggcactccatttcctacagaatgaaaagcaagttcttacctatgggctcttaactattccattattaatctcttctttttactggcctatctcatttccagctccatttctatccctaatcctcccagttcaatgatttctcctttatggttctttacatctttggacatttttcatccagtcatctattcagccaacaggaaggctggtaaaagtgggagtatgtggaaattctccttcgattgcatattctttggaaaatacaaagtgcaaggttttccagctgagggtggggaggagatttctaaagaaaggagaatgtatgaaatagtcatctaaaaaacgaaagaatgatggacttgggaaatatgctaggattgctaggctaaactaaggacccccctgagattagtggtcataagtttaaaacagtacttgtcagtcagcatggtgactgattttctccaaacatgttcagcagcccaggtacagttgcagagtaggtggagatttggacttaacaaaactgtggtttgtcagatgagtaagatggagaaaagaagaagcaaggggagctatggtgtaaggaaggaaggaagtgtttatagtaataaggcaagtggaagggagaacctaagggggctcaagacatgaatggcctgaaaaggcatgatcatgccttttcttaggcatgaattgtgatttccatccattaacctggaacagcctcactgaacttcatttttagcttctgtttttcatttccaaattcatagttgagtggtcattggcctgatgacccttggcccagaacagcaaggccatgctgagggtgcccctggctatgaagtaagagatggagttggccctgttaactttaactctgcgagctgttcatgagtcttcatctggaagcagtttatccatatccactgccttggtgatggagtagtgcactgggaggggctgggactcctgaggccctgctcagcagagcagctgccagagagaggaagataaggcctcactgccatcagagttaggaccaggacctatgtttctggttgtatgctgcctacggagcttttggatgcactctgggacaggagactagaaactttggcagttgtttgagaaacaagttcctgtagaacagtagctatggctatttagcatatgtttgttaggtccccttatgtgctaagccctgtgctgtgccccaggaacacaaaggtgaataaggtagagatatccctgctgtcatggtaatttatttcttgcttcctaccattcatttgtttcattagctttgctgtttaagtactacgtgctgtgcactggactggctattggaattttaaaaattaatatgagtctactcaagggtaagcagataaattataatacctttatatttactctgtgagatacactgcaggcgctgtgagtccacagatggtaaatgctgtgggagataatgaatgcagaaaagggctcatcccctgccctcagggaggttatagtccatctaggagaagaaaaccacagtacctccagaagcatatcccgctatttaaatttttcactgtttcattatttgtatgtcaaactccaagtgtttttgtattcccagcagagttctggcgccaaagtgctaataaatgttgattgaattaatggataaataactgtgtattgcaagacagaaattgacatgttgctataatagagagtagttcttttagaaagtgtgacatttgagccaggcctaggaaactagatggccgttccatatgagaaaagaagaggcatgccaaacaccagaaatagccacttgttaggcccaggaaaccacctggggttctggctggtaatgagcctagggcatatggagaaatgtggagagattaggagggaggacgagggcagatagtggatggggcttcaactgccagtcaagggagtttagactttatttagcaggtagtagagtttttaaagaggaattccagagctgggcatggtgatgcctgcttgtagtcctggctactgaggaggctgagacgggaggatcccttgaggccaggagtttgagggtatagtgcatgatcacgcctgtgaatagccactgcactttcaccggggcagcttagtgagacccccatccctcaaaatacgtgtgtaggcgtgtgtgtgtgtgtgtgtgttaagggacagtattttcagaaacttatttcagagggtactttgagaaactacttttgatagcaatgtagtgggcagattggagcagttagatccagttggatcaactaagatccaatctgatcttcgttggattaaaataggattttatttttgagaatgggggatatccaagcatgaccagagggtaaagggaaggaacccctgggttttctgagtgcaggatcacgaccagatggtaagggaggaggagatgaacagcacatctcattgggggcctggtcagcactggtattggaacctctcagtaaagtacctgctgaagagagatgccctggaggagccggcctaggctgtggattctgggcctttaaggctgctgttaaaatcactaactgtggggtttcagctaggtggggaaaccagaaagaaaaaacaggaggggctaatgatagaccccttgcatgctccagaacatctaacatgttgttcaagtggtataattgacatattgatgatcagacagaaaatgcagagcactgaacgttcgtctcttaggaccctaattacctcctttgtaaaacaacccttcagattctgttcagcactagcagcctgtgaatctgtcagtggagaaacatagggactttagaaaagctgggtgctttctgaaaacccaaacaaacacccactgccagcatccaactcaggaaggtcatccgatcctgtcagtgtgggtactttgatccatttagttcttattcattattactgattctttgtaggtaaagtttataagaaagcaaaccagctaggttagagcgtgtttcatgttaacctttttttcctttctcattgctgaagaaagtagggcacgttgtttgaacggcatctagtcatgctacagcctgcacctaaaattgcagatgggcctttctgcaagaatccttaatgcgtgaacagagattcttcatttgttctttatatctggttataaaagaaaacaatctcacagtaagccttagggtgggctgtagatgaggtagactttgtgtgtaatggagccctttctatttcctgacattagtcactgaaggaaaacgcccacataataataaaaataatacctaacatctgttcagcactttggagctcccacttgccatatatattcacatccagatatcagaagtcatatatgcgatgttacactctaactaatgaaagcaaagataagtattgaatatttaggggcatgcatttatttaggtaaggatttatggcgaagagcaggttccttctgttttttcaggcgttgacattggggaagcgatagagtggataagaaccgtggagttagacaaacatgggttcaaaccccaactctagaagcagggtgacctttagcaagctgtttaatgtctcaaagatcccagctttctcattgtaaactagaattataattcctctcagacttgatgtgacaatggaaagaaattaggtttataaaactcagatacgcttggaaccagagaacggcacagtaactggtaataacaattacttaaatggtgtactgttggatcttggcatcaggaacctttccttgcctttgtgcccttagacccgaactattacagttcccacctacatttctccttttaagatgctgtgtttgcactttctacctcagtcccactgatatcatgcttgatgttgcctccagctaagttatgacagcaacctcccagacaattgctagtatctctcatcctcccatctccccacaaccacaccacagatctcatataccatggtatgactatacattctaaacctattttatgttattgtgaatctgtaccatctgtctgctgcctattaaataaaactacactccctttaaattttgatatcctctgaaatctggcaaaatcttaccctttgactttatttcttatgactactcagtacgaatttttggcttcatccagggatcttaaccttagacatagaatgaactcagttctcctcagtgcctttgttcatggtgatcttcctgcctaaaatgctgcatatagcattgtttataaagtatggatgcatggatgcatgtattatgagtcataaaagaaacatgagggcccagaaacacattttccttttaaagaatgatgaggaaatccatatctagacacattcctctgctttccatttcatttccgccttgacaattaatatcctgacagtgttagaatgcattaacttaataatggtgtgaatacatcctagtaaggatagtaccagcgcatttaacagcctgaagaaagagattttaagttagggtgtttggaaagggcaaaaaggcaatagtaagtttcctatatatgtgcactaagaaatatctgtctggacccatcagagaacccttattctactgttgctggtgtttactgtcttccttaacgttgttttctttgagagaataaacacattgtgcttgggcatcagtgaagacctggtgtgcaaagagtgccctgctgtctttctcttacaagatcctcaattcctcaaaccatggcttcaggctgcaggctgtggaggcagaatgggcaaggagtcattttaagttcactccctggtttcctttacatcagttttcataagatgtaacctgatgtgtcttggacctatagacaaatgctattttgcttggtcttcacaatgtaaaattattctggtttgttgagatgctttacctcacttacttgcatttttccttctattcctttactccttaaagaaaagttaaggggtgagagtggggagaggaatagtaatgggagaatctgttccatattatacctcatccataatgaatctcattgccttcgaaatttttcctcccatttttaagagtgtagtgcctcatccatggcagttagtggaaacttaaatatcctaagatttatcctttgccaattcatttagtctctgaagagtgagagactcttctcaagtctcaagacagggaggtgctcttctcaagagccccggacgggggaggtcatttgtaacttcttaagggatggaagttacatttaggattttcttttacctccaagtttcttcttagaagtttaagtagtggaagaaggatctagtagctcttatactgtagcttactcagtaaaatttttccttatccaataaggtaagtacttgtaaaatcatagtagcaattaagaggctgcttctctctttctgttggcattcatttattgtaaagacaagaacatctttttagaaaccttaaagcacttaataataagcaatttaaataaggtgagaaggaagaaacattttcctcagtttttatctcaaaacatatggtgtattcgcagcccataagttggcagtaacagcagcctatttaataaaacggaaatatgcacgtgaggttaatcaggtcaaatagaagtatgcatatgtagaatgataacctcgaagaaaactgcaaatttattcatgcaaagagtggaggggaatgccataaatattaacagtggtccgaaacttacctgattttttattcttttacttgtatgtatttatattttccaaattttccttattgaatgtcattaactttataatcaaaaagtcactttttaaaaagacatttttaaaatggggcattttaatgtttttaaaaatgttttaaaaagacatttttaaaaagtgatgccaaatgatgggtgtgtgctgatttggtgatttgttatttaaattttatttcttagaagaaaaaaatgggagaaaagtgtaaaatgtgggagaataaatgattaaatatgacttgttgaatatactagtcgtttacatgggcataaatctggaaacctcagatatcttgaagggtcacaaagagccgatttatctgattaagttaattaatacttatgaagtcctttgagcagtgcttggatatgatatgcccttttttattgtttttattttcgtcattatcacgcttggacactagcacatattataataccttatacgaatggtagcattcactaaatgcttgttgagtgaatttttctagacttttcacaactctggttcatttagtagttgtgtcacatttggactttgatgcctggaactgaactgaacacagaatgacacacatgatctaacaagtgcagactgactgtctcctcccctgtcctggccattgtcctggtaatagttttttgatgagcaagtccctttgttgcctcataccaagctttcaacctatgaaaacactgaagtatctcatagatgcagtaactaaactagttatctttccctgatgtgtactcttgaattttttaatcaaattattaacttccctggtaatctaacctttttttttggtttggcccacctttcagcctgttaaggtgttttcattgctaactctttctatcctgtatagtatcctcttaagccttatgcaatcaaaatttctgcagcagtatcatgaatttaaaatatgggataggaggagagccctatggcatggcattcttcttcttattttctttataatgttaccacctcagttatttagtttgttggctaatgacagattagggagccacagggtgggaggtttcttagaagaggggtggactgatcataccctgagtttattatctgatgccacaggagaaaacagaaaagatgtaaaaaggcaccaagggagacgtgacactgtgcatttgtcaaaacccataaaactttacagcacatatattgaacattaatgtatccagatttttaaaagccatttaggaggttggaggatcccaggatgaaattcagactatgacaaaagaatccaactgtattacaaagtacagaataaactcactgaagaaagtgaatgaaaaagggttaccgacttaaataacttaggaactatatgcaagcactatactctattcgataaagttgtttcccatgaggatataagttaatgattctgaaactattatatatgtacactggaattgaataataagtaaatggatagtggatgatgagggccttggtttctcactttggaatggaaagttatagataagcaaggagggaaatgatccatgtggtaatggatttgaattggagataccgatatgaattcatgtttagcttgatatagatacaaaatacaaagaaatatttatagatatgtgtatatacacaagttagtacgcacacaatttcttcgctgtgtcacctgagagagcctaaaagcagcaatatcccagcagcaacaagcatatctggcatccagatcttggtttctaataccattatcctgtgaaagccaccaggtctcctggccgaatgacagattccagattctagagcgggggcagggaatgtgcaagatgaacttggagcatcttgtagtgctggaaagtaaggaagagcttaacaaacccacaatgacgaaggtttatcagagggacacaggagttactgaaatagctcacagtggccaaagatgaaataatttgagcaacaaaataaagtagtaatgaactgtaacccaaactatgaaatatgtacccatcctgatataaataaatgattgaatcaataaataaatgagggaaatagacaaatctgtgcagaaaaattccaaatgatttatgtaggtattccactctcaaagaagtgggttataatacttgtcatttccttaagtgtgggctgtgcatggtgacttccctccaaagagcaatgtgggaaggcagagaaacctgacaaatactgctctagtctggtgagcaaggttaacatcagtggtggtaagtcaggctagtgtttatgcttgatatgatgtgatgagaatggcactttacttctggggtcttactcccagaaacctataatttcaggctaatcatgagaaaagcatgagactaattccagttggtggtcattctataaaatacctgaccagtactcctcaaaactgtcaaggttatcaaaaacaaggaaaatctgaggaactctctcaaccaaaaggaacctaaggagatatgagtaataaatgtaatatggtatcctggatgggatcctggaacagaaaaagaacattaggtaaagaccaaggaaatctgcaaaaagtgtgggctttagtggataatatatttttattggtctgtgaattgtgactaatgtatgatattattaatgggtggagtatattgtaactcctggcagtaacttaaatttcctctaaatctaaaactatactgtttaaatgtttgtttaaaaaaaaaaaaaaaggcagcctggatgcactggctcattcctgtaatccccgcactttgggaggccgaggctggtggatcacttgaagtcaggggttcgagaccagcctggcgaacatggtgaaaccccgtctctactaaaaatacaaaaatttagccagctcttttggtgagcgcctgtggtcccagctacttgggaggctgaggtaggaggatcgcttgagcctgggtagtcgaggctgcagtgagccgagattgtgctcctgcacttcagcctgggtaatagagtgagaccctgtctgaaaaaaaaacataaaataaaataggccggatgcggtggctcatgcctgtaatcccagcactttgggaggccgaggtgggtggatcacgagatcaggagatcgagaccatcctggctaacacagtgaaactccatctctaataaaaatacaaaaaattagttgagcgtgttggcgggcgcctgtagtcccagctacttgggaggctgaggcaggagaatgccatgaaaccgggaggcggagcttggcagtgagccaagatcacgccactgctctctagcctgggcgacagagcaagactctgtctcaataaataaataaataaaataaaataaaagccttgaggattccaaaaacattgtaggagagggcagcataccacaccctaggagcctgggggcctccgtgttacaaaagtgttgattgatgctcttgataatggagctgagtcatcaagaacaggttcaactttgtcctgagaaccactaattcagggatgttctcactacattttcctggttacagccaactttttggtatttatagccccaggctttgtatgctatatcattcattcagtctttttcctgacatggtttaacctaggaggctcaatatgtagaattcatgtctgagtcagcctctccgtatcccctttatctcctggcagaatctctggttcctggctcaggcacatccatgtttttgatttttgatttttggttttggaattatcttttgacttaagaagtcacaaaaataatacagagttctccagtacccttcactcaccttcccttatgttaatgccttacataccgtagaacaattctagaaaccaggaaattggcatttatacaatactgttaaccaatctagaaaccttatgcaagttttgccagttttcctactaatgtcctttttctagtccaggatccaattcagaatcccacatagtacttagtctcctccaagccatgatgatagttcctcattctttttttgttttttattttttttagagacggagtcttgctgtgtcatcactctggagtgcagtggggcgatttcggcacactgcaagctccgcctcccgggttcacaccattctgctgcctcagcttcctgagtagctgggactgcaggcgcccaccaccatgcccagctaatttttgtatttttagtatagacggggtttcaccgtgttagccaggatggtctcgatctcgtgaccttgtgatccgcccgcctcagcctcccaaagggctgggattacaggcgtgagccaccgcgcctggcctcagtttttgtctttcataacatcaacactttggaagcatactggccagttactttgtaccttgtctatccatttgggttttctggtattttctcatgattagattggggttatgcatttttggcaagagtaccacataagttatgtgttcttccaagttcgtcatatcaagatgcacgtgatgctgatatgttttattactggtggtgataactgtgatctgttagtaaggatgatgtctgccactccattatagaattattgttttctcttttgtaataaatatcttgtggagagatactttgagactatgcagatatcctgtttctcatacttttgctcactacttttagtgtttatcagtgtcttgtgcctgcaacagttattgctgtaatgtttgcctaacagtgatttttgtctttccatcatttcctctacatatattatttggaattctgctttaagaaagagctgtcaaggtgcattcatttttagtaataatgataaccacaataatgttgattataattataatggtacccaacattgttattatgacttgctttgtgcctggcacagtgctgagttcttggtttcacttttataacaccttcttaattagctcatctgtctgcaagagttgtatttcctggagcaccctctccagcctttcctttccctttgttcctgtaactttcatttgcccctctgtcatcatccccctgccctctgtattaacaactctgggttctctcactccatactagactttggtctcttatctctgtatctccggttccatgtataatgtccatcacatcataagtgctcagaaactgttattgaatgaaataatttgttttgagcctgtgtaaccccagttcatcctacctgagcatgctacattgttccgtttcacattctgataattgttctttagtggtcaagccatatgtttgccccatatgaagctcacaatgataaaatgctggatatttcacagtgctgctgctgttaagccaggtgtcctgaacgccctcgctccatgtttgagtttttaaattatgtacaaggctttaggctgatccctgttaaatatcatcttgtttgttttagcctgccttttcaacctatggagatctttttgatacctgattctgttccccagcccatttgctatccttttcagcattgtgtgatttaaaatttttcatcttccaaatgattgataacagtgtgggataaggtaggaccaagttccccgcttggcattatttagcactctaggtgcagtcatttgacctgtaaggaatctacagaaccgtcactagattgatatttcctttttttttttcttttgagacagtctcgctctgtcgcccaggctggagtgcagtggcaccatttcagctcactgcaagctccgcctcctgggttcacgccattttcctgcctcagcctcctgagtagctggggctataggcgcccgccaacacgcccagctaatttttttgtattttttagtagagacggagtttcactgtgttagccaggatggtctcgatcttctgacctcatgatccacccacctcggccttccaaagtgctgggattacaggcgtgagccactgtgcctggccgtagattgatatttctgtcaaattcacaaggttattatatgtaactttgttaaaactttcttgaaatcaagtaaaattcaatcagacaaagtttatttgaaattacatgtcctcagctttcagttgacatgtggttaatttgttcattcattcatttatttatttattaaacagataatctataaacatctatatgacaggtactgtcctaggtgctgagaatagagtgtgaacaaaatagacaggatctcagcttgtggatccataagcaaccaaaaacataaacaagataatataaaatagctgtaaaagctatgaagaaatgaaatgagatggggactaatcaggaaaggcctcaatgaagaggtgccagctgagctagggcaggagtgttgagaagaagggaacaatgagaagatctggggaatgaacagtcctgcagagggatgagcacgtgcagaggtcctgagatacaggtgagcttggcatattcaaagaactgaagggcagccagtgggcgaaaacatggttatagctttcagttacttgaaaaatattcgttatcacttttttcctagaaagcatctaacttcatgcttaaaatttgactttatttgctcatttaactaggaaaaaaaaggtagaccagaattgcaagggaattagcatatttccccaggaacaaagtattctgaaaacaaaacaacagaaaatactggaaattatgcattttcaagattgataagaatgctcttgttttcccaagcatacattttttccctgaagtagatacagtggaacccatctatattgtaatgggacatacgttattgttctgaatgctttaatatctagtattttagaatgtcttttatactggatagtagtgtaaaatgccttgagacatgcagttggtttttagacttaagacctcttgatgattgcagacttactcttttttcagtagagcatagtggaaactaaagagttttttagatgaatatctggagtaatttaattaccattttaattgttctgtatttgcagataattggtgatgtgagtttttgactgacttgagattaaaatgtgctaaacccttacctgataactctcattagagcaatatatgctggaagcagagggctcttgttatggactaagtgtttatgtcccccacaaagtgttgaaatcctaaccccagtgtaatggtattaggaggtagggcctttggaaagtaattagggcatgagggctctgccctcatggatgggattcatgctgttataaaagggaccccagagagttctcctgcccttttctcaccatgtgaggatagacagggaagtcagctgtctgcaatctggaagaaggccctcaccaaactcggctgtgttggtaccctgatcttagatttccagattccagaactgtgaggaataaatgtttgtcatttaaggcacccagcctgtgtaatctgtaatagcagttcagactaagacagccccatattactcactctccatctgagagtgagccgagcacatagcattcttggcatcagtagcacaagggtatgaagggaaatgcttgaagataactggaatgtatagtagaaatgtgattcctgtttatctctgagtgttaactttcagaacataggaaattgagatgctcccttaaacattcttttagatagcaatttagggaggcaagctgaacttaaatcttttaggaaattggacagagtcatagcccattgggtttagaggtagtataagaaaggttgctgacattatttggctaaagtgaatggagaacagagtaaaccaagaagatagtgttcatgtacatcatcagcacaatactgagtaagctgaaatatagaaaaataatgtatgcttactattattggaatgtgagtgacataagtaaatttcataaagggaatataagtcaccactcaaatagttgtttctttcctttccttaagatacaagtgttccatatcttacgggtgagtacaatattggctgatgtgatacggaaaattctgtcaagttcggagacagccatagtgttgaaagttctgagtgctttcaacactatcaaatagtgctcatattctttcattgtcagattccatgacccaagtgtgtctgtaggtgaaggtctcccacccccaccccacccactgatcactcagtaagcaagcaggctgatgtctcagtgcctacagtgagcagtgttaactgactcctgtacttgcttgtctgaggacagacaggtgtgctagataattaacattttttggaaacaaaattattacattatcctaaaaataaatcaacatactgggaacacatgcaggacttttgtattactacgcaagaaaagataagcctactatattcatataatatattatgaagtctgttagatgaccttaaatttgttcatttttcttatttgacaagagttttaaatccctatgtatttacagatttggcagacttggtatgttttcttcaagctatcataaacaagcaaatggctcagaaaaattcccagctaggtaattttatctcaaatatctgcttaatttcaaaggcaacttgagttagccctgtgcaaactcaaaacactgtgtgtaaatatatgtgtaataattttttttctacgtaccacatgtatcttctaagtagcataatgttgcttttaaagatgtactgtttgtttttttacttttaattttctatgtgaacaaatttgacatgtacttttaaaaattaaaatatcttcaagtttctattcatatataatatttcattttaggcaacaattgaacttctgaaaagttgatgagacgttgaacttctgtaagctaaattatttttagatgtcttgaaagaactctaagaagatgaataatcataccaatttttttcatgattttagtaatctctttatcatgtttcttaaagcagtttacatctatgtataatttgtgaggcccaggatagaatgtctctgagtctgatactccagagtagcatactcagctctacagggaaaacttttccataggaaacatcagaaaaggaaaggtatgacagcacatgtttattaaacatattacataatatatgtctcctcctgacaggcaagcgttagaacctttattagacattgtcatcttaatataatgtaatgtccacaaatacattctctaatagacaagaaatgatacatgagttttaggtttccagactaacaacccaaaatctatggcttaagtggctttgtataaatacatggccaataacggcatattgccgggacttggagcagaggtaagagtgtggggagaggatcagccttgaagactcccttgtgggtgagtgggagggtagaggggtggatgaaggtggtaaaggatagatgggaagaggccaggaggtggggaagggaggggtcgtaaactttggttctggaggagcccttagagatcgtgtcgtctgacatcatcattgcacagaaggggaagcaagataaaaatgtttatgtgcctggctcgtggtcacgtagctagtggccaagcaggacctagaatgcagatttctgactcccagcacactttacacactgggagtttgctaagagtagctgagaaatacacacagtggcttggcagggagagagaggctgtgaagacttaacacaggtgaggggtgttcaggccgagggtagggcaagctcctgtgtgaagatcagggtttaccacccctggaggacaaaggtagtgagaacccagcacagaggtcaggactgtagtcaaatagattagcattcaggacagagtccagtctaagagcgggaaatcgccatatcacactagtgatgataataattactctttctttctccctttctgtctttctccctttctctctctctgtctcctctcttttcttgtgaatcatagttgttaataaacaagctaaatctatatagtgcgaaataaacatattcacatattctcaaaaaaaattactacttgttggatgcctattgtgcattagacattaggctagacactttattgcagcataatcatttaattgtcataataatccctttggaaaggtgtggtggttcagtttttgcagatgggggtgctgattgatattcagagagttaaataatttacttgaagtcagcagagtgtattagaaactattcattactttgggaccagaggggcagaaggaaatccaaaggaagagaagtatgcagatgtgttcagatacacactttgatgataactattgatgtatgtacatctctcttggctatgctataggaataccttaagtaattcagtggcaatataccttcttgctagtattttaatagtatagatcggttaatgaattatcttagaaacattatacttggtgtattctgttgctttatggtttcattttaggttgagtattaagggaacacagtattttaatcgggactctgccagtgcttttatctacaggcctgttgccatttttgtcttctgtgaaatttttgtcacaagaaaggcgagattatgttttttcctagcagattgagttggtgtagtatattcttggttatcaaaatacttacatagatttgagattttgaattggtaaatattcatgtggtgtttaaaagcatgatacatattgcacaatcttaatcccattaaattggatgctgtgtctatacacacgcaggacctaggaggacacgtcaaactgtaaactgcttgtgattgtggataactttgttctttgcttcttgtgtttttcagtttcctatcaggcatatattaactttttttaaaaaaggttatttttaataacctgaaaaaaaaactgatttgctgaaggtcaatagtttgctctgagagaggacaccatgatttgaactggtctgccttagccaaatctcatttgatttctgcagtgctatgttgccctcccagaatcacagataaaagtggtgggcagccgaggtgcagtggaggtagaggctgggacaaggtgcatcccggtgagtgcctggtacagggaaggagcagcatggtcatggtgtgataattggaggccacctctcctagcacagcagataacagaaggaattcccacctaaccgccatactcgctcctaagcagctgaataaagatgtctctgatatgtggctgaacctctgtatgtggtttagtgctcacaggcagccgatactgagaggaaaaaccttgggttcctaaaacttttaaaacattgaatacgagccagtaagcagtcattggtatactgcagttattgtaccagagttaactgatgctgtagaagtgaggccaccacatttatcaaatctaaaagcaattcatgggaggatggtgaaaaatggaaaaccgcaacctgatgaggaatcgtgaaatatctacgtgtggatgtttagccttgtactccacctctatggggaagggaaggaaagcgttattcattgagcatctcacaatgttccagatattagatgcttgccaatcctcgcaaattctacgaaggaaatactctcacttcctgtggatcaggacattgaagctcagaaagagctcatttgctgtggatcacatagctagtaaatggattcacacccagatatgcctgaattcagagtcaagtggaagaggaattagacttaaatctgagtggccccagcagcaaggctaggatcagtgagtgtaaaggagagatcggttaatttcatagactgatccaaaagaactttcttgtaataagtgctgttcccgaaatcaagcaaagtaccttaaacacagatacacatacacaaacagctttactaagatttaatttacatactgtaatattcacctgttctaagcatacaactacaaagtaccttttaaggtagtacatttcctgtcattagaaatgtttgtgcagaaagggctggaaaatctcccagatagagtagaggaatgttgtgtttccatctctatgaaagccagtgttgagtaactcaaaagcagtgtccatactgtgatgtgctgagtggctgtttacttaagtgttttaaatgtcgctggatgtaaaggaaaggaaagttccaaaaggtgttccagttgggcttttcatcttaaatttagatcaacgagatgagctcttcagcttcggaaacacccaggcctttcattctggctgcatactacagttgcagaggaacttttcaacaattcaggtgtagattcaagggcctaccctaggtccactaagttggtacctccgggggtgtggcctagcattacattagtttctgaggccaccccgcattattctagggtgcagctgtgctgaaaaccactttctttggctgaggagaggctcaagtgtgccagagggtcatggagggtcaaagttacaaaccaatcccattctgccggtgacctgtcttttctgcatggtcaaatcagccacatccaagttacttctagttcgccagccattgctgctgtgccatgtctggctctttgaagatgtctgacagtgttggcagttaaccagagacagttttgtgacttaataaccagatcaactttgtatgtaattcatgtccaccagacatttttgcagagctttaggatggagaggaccttgaggaaagaattaatgtaaactgcatcaccttagctgatcatcattcctgcttttagaacaatttgaacaattacactcacatttgaaaattttgtgcatctgttcttcagttgtgcaggtcacaatgtataggcccacatcaaaggatcaatgctttcagatttgtcttctatgtggaacttgtatggttaaatgattataatgtagtaaagtgaaaatctagtctgggagctagagggtgtcagaactgatggtcctggggatcctttgaaacctttctgagcttgtttctttgtctgtaaaatgtgaccaacaattcctgccttgcagtatcactctaaggattcagctaaagaatatgtgtgataaagtcttacaactatgtatttattgcaccccttccatgtgctgggtagtatactagtccctggggaaacaaacgtgaatgaattatgatccctgccctggagaatcttacagctttagtgaaggagatagactcaaaaccagataattgtacgagagtgtagaggggcagaaatgcacaggatactctgggagcatcaagggaggattaaaaggactgggttattggtgtagagatttccaggaaggcttaccataggcactgatggcaggcaaagacaggtaattaacaaggtaaattggagaaaactggatactttaggcagaggaaatgcaatgggcaaaagcctgtggatgagaacctggtagagggacgatgtgttacaaatgctgatggtgttttattcaattcttgctttcccctgggagaaatgacttttgggtacagtgcatcaaggaagattcagaaggtaaaggtggtgagagatgtaggttaaaaagtcatcgtctgttttcttagtttgcctgatttcttagtttgcctgatttcttagtttgcctgctacacaaagtgaagcagaatgcagttgagtgatttatgtccatcataaaaaaaaaaactgactaaaaataaatcttagttcaggccagatgtagtacatgtttaccacttgaggtgaacatcgaagcatagtggcacttactggaagtatctctcagagaaaggaggctgattttataaagaaagtctttggagctcaggaagtctgaaagctaagagttggtccacagagcactgaagataagcttgttgagcctcacagtaatttttgtctgtcccaatttttaggcagcaaaaccacagcagcatctcatcaagactattgttaatgcaggtgtctccattggtttatgtgtcttatccttaccctgtttccttcacaactgtaccatcttaggaatcaggaatcacaaagccaggtatccaatggtgatttcagttctttgccatggcgactggaggccagaggggtcagggaatgtcaggggtgcataattcttgtgctgtaggctttcctgccatacgttatcaagtgtactctacagcatcctgataacagcctctgccctgggaaacagactgtgaccatgcatttctagtccagcatatcctatcagaagaccaaatggcttcatcaaaaacagagtgacaacctcttctctttgcctcttctgctgactctgttaacaggcagcattggggcaggagagcctgcaggcctttcacggctgcttgagttctcacctgtttgtctgagctctgattcctctgccctgtaagcgtaaaggagatgtgctgagtggaaagacctctaaacaggcagccaggaagccagatttcaggtccatctctgcctctaactggcagctttgccttgggtaaatcatcagtgggcaatagtttctctcctgtaaaaggaaaagattgggtttagattgtttctgaagttctctctagatttaacctggaaggagttgaaattgctaaccttactcttaactgcaccattgcccagtccccagctgtcctcacctctgccccaccctttcattgccactgtctccttcatacttccctttccacctgacctgtccctgccaactccattctttcatgcctgcctctctgtatctggagggaatcctttggtgcccaagcagggttagattaccagatacgcagactaaacatgtgtttggggcaccagaaaagcagacaccgtgctcaaatattttttttaaaaaataatttgatatttgctgcttaaagaaataaagccgtgatcatgagaaaaagcagaccttagctggactttcttatacttattttatggtttattatttaaatttgatttctgtttggaggaacgctataatctttgcagtacgtcgtgcctctgaaggcttcgtcttcttcctcctcctaaacttgttgaactcagcctttccaccgaagcctcacagaggtagggagatgctgctttgctctacccttttctctgtactggcctcatgtgtgctgcttttagtatagtaacacatttctctatagcactttctaggtgagcatctccatgctgggggctaggccctggggcagaagtaggccagggagagaggcacaaggtgtgagacacactcttcctcctccagagtttataattgaaacatatgttttctccacaacaattaaatgccagaaatggccatacctatttgaaggcgtgcagaccagtctctggaagatgctcctgcagcagggaccttgaagagaagatggaggaaagtttaaccgtattaacaaagtgtgcatatctgaatagcctgctactttatttctaagccctaaatcccatgattgtggactgttggctgtttcccattttcagagctgccccaaaccagtttctcccagactcaggcattctctctgtgtcttcatatctcttctgaactgacttttgtcctttgagcctgccctctctgataagctgccgtagccactgagaagaatgaccaagttgagtccctgagcccacagaataataaaaggatgttttctttttctttttttctttttttttttttgagactgagtctcgctctgtcgccctggctggagtgcggtgacaccatcttggctcactgcaagctccgcctcctaggtccacgccattctcctgcctcagcctcccgagtagctgggactacagggcccggctaattttttgtatttttagtagagatgggttttcaccatgtttagccaggatggtcttgatctcctgacctcgtgatccacccgcctcagcctcccaaagtgctgggattacaggcgtgagccaccgtgcccagccaataaaaggatgttttctaatgggttcagtctggttgcctagggggaacatttggcttttgcatagactgtaataatccccatttctttattttcatagtctggttaatcccatgtcattgcctttgaagttaaataaattgattgcttaatgtgcacggggcactgtgctaagtgtggacatgtatgggctcactaaatccccaccacaacctctgaggtgagtgctgttgtttcccacttattgagaaagagggaaacagagatgttaggtaacacacctgacatcacacagctaataaatggcagagccaggattcttcgctctggcagtgtgactcctgtgtctcttctcttcatcattgcactgtatgttctctgagggtagagttgtgggtgtgctggagacagcatggggaaagtagtagcactctccatttgagttgttggactgattttgagtttctttctttcacagtgaaagttgtttgaattgttaatgagaggaaattttagaaaatgcttcacctttaatatattaggttatatctcttctctgtaacggcatcttttcaattctcttcatcactttttttgtttacctagtatgtgatcaaaattaaagttgtctgctgtggctgccttttcaatgaggtacacagtatcacattgccacatgtaatttccactcaggtgacactgttctcactgctgagagaaatggcaggattagcagaaagggtggtattagatattttatctcccttaatctacaagcaaacatggttacacatattggaatatgtcttttcaggattctggtggactcggttggtttattttaaggatgagagtttcctccagaaggttaagcattattacatctgctgctttaccacacctacttttggggtgtttgctgatgttcataactagcaataactgtgattcataaaaaggaatctcttatttttatgaacatttcaatctggtgtcttgagttgagcccagaaactttatagctggctctgagtttatttctcccaaaacataaatgtatttttgagatcctcttaatacctctggtatctgatattcacacatcattttatttaatgattctagaggcttggaaggctgctaaaagtcattgttttcgcctttgagaataattaccatcctggaatccccagtttagcctgagaccacctaacttccccctactcaggattcaagccagttctgtccaaggacaaaccagagagggcctaaagaattaaagtgggtggggcaggatgaaaacactaatagtagagagagctttctgagctatctggaaggatcgtcctcaccctctcccaatacttctgtcaaagttaaaaacatttcccccttagtttggctccttcgggaatccccaggcccagttgcagttttcattgcagctgtaagtgaccatctgaccactggtgaattcaagaaagttggaccttcaggctctttttggagttaatggagcatcctgtagtttaacagatgtgaaactgtttttagagatatcagctgcctgctggtctggttcctgggctgtgccttttcggttcagcccaggggaaagggaggaaggaaataaaatgaaatgagttcattcacttttcttccttaaaaggactgcaggaatacaggaggacaggggcatctgaactcagtgaattaacttgttcttctgtctgctccaggttatttcctttggttcttgctgacatgctttacctggagctctattggccactcctgcactgcacaggtattaggaccagactctgtaaggctgcagggagggatgtaaacaccttagcatccagagtcatgacttagtagaaaacacgtcagttttattatgagacctaagtttgagtcccagtgccactactattaattagctgggtaatattgggcaatcttggagttgaactttttgaacagttttttccctaacttgaagttggtaataacatttactttacaggattgttgaatggttgtgaggatgaattaaaatgttgaccgcgttaaagtcattttgtaagccatccagtatccttgctgttatagttctatgcacagctgattttttagttgcaggcaaacgaatttttacatggaagtacttgattttggaggttttaaataacccttttataaaggattgatagtttaaaatagttggttgtaaaaatgaaaaggaacttggaaagaaatgtcatgctgcccttcagatttatttcagcacacataaaagaagcctcttaagagttaagttttcacctgtgtcatttggcttagaaaatagtttgaagaaacttttcaaggggctaatgctctacaagggtcaaaaacctatttccatgtgctaagacatcaataatatttttaatagggctgtttctcttttgaatatatgttaattgtttccctctaagaattcacacagaaattggatatcatattttggactgcctcccaagacttttatgggatggaatatggtcaaatactcattcatgaacaaataaaaccaaacagatttgtttccctctcaccacatttttgcacatatatctttaataaatattcactgaaagctgtgagctatacaaagcaaggaaaactacccactattgaagtttgagggagccaagtgaataacccctctttccacaccagttagaaagataaaggagtcaggctggtagattgcaaaatatcatctttattactgatgatgcactttggagaatatggctttgggtctgcagccaagtaggcctccagaaattttatccctgaattagataaaactacccaccttttcttggcatgccagaataatggaagctggtaccctgcaggggaaagctgctttcccccagcaggagaggagtccagaattcgtgacctttgcagacaggcaaatctcaaagaggacgaagaggaggaattagtgagggggcagcaaggaaggcccggtgtattgtaaggaattctctcttggcgatgtaacatgaagagagtgagtaagtgtcctcttacctcaacacctgcagtagggtcttcaaaggtagctgagacctgacccctgcttaccagggacattgaatggggaaggaagagaagacctctctacaattgtatttgaacagttgtccttcttcgcagctttagctaaggctgtaaatgcaagtgggagcacatttttatggggcagccgcattgttccctggtgttttaactgccccccaactgctgtttacttctcacatctgaatcttctttcaagcatcacttttaggcttaaaaaaatccctatttgactaaagaaaatgagcggaacaattcctctttataaaatgctaaacagaacaagacatctggtactgtacacactgtagcccaaaggtgaattttaccctcaatataagcagagtatgctgtatgtcaacagtcagctcaaacagaagctcgagcatcgctgtagagccgctatcatcactgaagggaattcatcattatccctgttagtaatttcatagctttggaaactggtttatagtacttaactgaaacaaccccaaaatcccacccctgactctgccttttctattttccccaacttctgttccctttctaaagaacaagattttgtggaatttaaagtagactgtgactggccaggttgctgcttgctcacaatgcctgtcctgtgtgttggctttctcagccaaacttttgggagccagttgtcacccaggacccaaggctggcattctttggaaaaacctgcctgtgtgttgtgccaagttgtgttagccactccatgttattcttagatgggctctggagtatgaccagcctgtgtgcatgcatttgtagtaggcactggataagaactggagtaatgactacagaagtgaacggtcacagctctaacctgctgagtcatctcagctcagccttgctagcaaggcctcccaggctgcatatagtttgcttttcctgttcgttgcattccaacctgtcaacggctattttctgaaggtctgtgggggctcatgtagaaaaggaataggtctgggtggaggccaagaatctgaatttgtatcatgtaccctagctcatcttaattcaagtgttagtctgtaagaaacagaagaatgggtaactcaactcctccaaacaaatctgtcagcatttactcatgcaggaaggctattttcatgtatcacgttcagaacacaaagggtcccaaagggcttcccatctatctttcaggtgttgggcttcctgtcttccactgtgtcttttttttttttttttttttttttttgatactgagtctcactcagttgcccaggctggagtgcagtggtgtgatcttggctcactgcaacctccgcctcccgggttcaggcgattctcctgcctagcctcctgagtagctgggattacaggcacagaccaccacacctggctaatttttgtatatttagtagagacggggttttgccatgttggccaggctggtcttgaactcctgacctcagatgatccacccacctaacctcacaaagtgctgggattacaagtgcgagccaccatgtccggccagtcagttgttttaaatcgtgtattgcctatgagcagatgaggaagaggaatcacactgtctattgagattactgacaacctttctaagcctcattttctcagagttttagcctcaaaagggacattgatcaatttgtagtctgatttccttcttttctcagtttacaaagaacttagaggaattatttcatgagtcccccaaaattagtcatttcctactttgtgaccccacagcattttgttcatactccatattttaagtaaacactgttttgtaattaatgtgtttatatgtcacttctttccaccatcaccaccagcccccttgcgccaactgatttcttcaagagattgaaccaggtctaatttatgtttgtctcccaccaacccagtgacagatagatgaatcatgttgcctggagaggttaagggatttgcctagagtagcagccctgaaatgtgagtggctttcataactcccagccatgtacttaccactcgtatgttgccagaaatgtttttaaatgatgtgaattcatgcaaatgagttaggacacaactttcccattatgttcctctacactagctcatttataaaacattcataatagtgatgatgatgatgatgatgatgtcattaccacccacccaactgtatcattttaaggatcaacctaaataatatttgtgaaagctttgaaactggcaaagccaccttatgcaggtatatagattgctattactctagtctgttgttatttctgtgtaatttacaaagtatttcatattcatcatctcacttctcgttacacctctgtgaaataggtggtctcattgtcatttcatagacacaagaactgaggctgagcgagactaagtatttcaaccataatcacaaatggatgaaagaagactagaacccagccagccctcttgctttcttccagtacagccactagatcaaggctgcagagtccctcaggggtgttgttatcatcatcatctgttgaatctgttgcatcttctgttgtgtgtttaggtcctcaggtccctaaagccctcctgggtgagcccaggaaagagacaacttcatgagaaatgagagtgaggcccttctaagaccagttatgtatgtgtgttgaaatgctagtcaggtttttcattttatcaaaatgcagggcttagacacagaatgctttataagacaaagcaagtttgattttgaattggccgaccattgaccatgttggaccagcagcagcagcatgtggatatgtgggtccaggcaggaggggctaacctctccatggcttctgtctcaatggggagttccttactgtgactctcagaggaaatgaaggatgataaatccagctgctgcttcctgtttctataatttggggatttatttttttaaagggtagaatgctttatttttaaaccagtggaaaataccctcaacgtgttccttgttagtatttccttgaaatctagatgcactcatttgagctttaaaggagtgggtgtgtttgcgtgtgtgtgtgtgtctgtgtgtctgtgtgtgtgtctgaaattcccatgatgctgcctgagatggctcgctctctttcagtttttcaacaacaataacaacagaatgaaaaccaattaaggcagatttcactgggcaggaagctctatgtgagattgatgacagtatctgatttagaggaggcctgtaatttatatctgtggtattactatctctctgggtatgcttgtgcaattcaggtctggtttttcttctgcttgtgattgtgtcattagtaataaagtcccgaggcatttaggaaacacactgccttctcacatatcattgtttacagacagtagctgtttggtatagatgattaggatgccatttttggagtttgattatgtagcccctgactccataactaaaaccaataggtagaaatatattttaccagtgccattttttttttcatttcctaatttaagagacagaggatgtatatggggttccaatggtacatcgttcaggagcagcataggaaatttatctggcaaaatagaattataagaatttagtagggccaggcacggtggctcacgcctgtaatcccagcactttgggaggccgaggcgggcggatcatgaggtcaggagatcgagaccatcctggctaacacggtgaaaccccgtctctactaaaaatacaaaaacttagccaagcgtggtggcgggcgcctgtagtcccagctacttaggaggctgaggcaagagaatggcatgaacccaggaggcggagcttgcagtgagccgagatcatgccactgcacttcagtctgggcaacagagcaagactccgtctcaaaaaaaaaaaaaaaaaagaattcagtaaatataccaaatcccctcactgagtaagaatcttggctctaatttaagtcccttagcccctcctttcatgttcttaaaatcatagctaaggtactattcttcttatagtgggtgatcagtacatattttgtacttacgaaccagtaggcctaagtgagttgtttattcttttaagaattatggttacactttaatgaattcagaataaaaaataaatcttcacagcctaaatttagtgaagagattataccgaatatttggtctgactttgttaacaactttttgaaggtaagatttaaaaactttgagactatgacacttactggaggactgttccagcacactggatgttgtagaagtctggaattctcatctccattctgccatttccttgtaacaagtttcttggctataaaataaagttgatattatttgtttgtctcatagtagtttgtgaggatcagattagttggaatccatggaattgtcttttaaaaagtgagattatgataaagaccttaggtaatattgttattaaatgacagttgggttgttgtttcattttttaagactttactccttgtgcccttaggcagcctttgactaccagcattttgtcaccttcactttaagggttccttctagtgattttttttaatttgtagaagcaaatttaatttaatttgagttaatctctagtttaaaataagctgagggggcaggtatgggcttcatagtgtcagtcagatctgggtttgaatcctggtccaccttgtattagctgtgtgatctcggtcgaattactcaaactttcaaaacctgttttctctcagtaaaactgaagcagtaataccactgctcaggatggataggacaatttaataggctcaagtatgtaagtgcttggttattcaggaggggtaattttcttcctgtcttcccctgcctattagcagtagcctcagtgttgaatggcaagatacagcccaagattttcctgtaaatatgccagcagtgcacagctcctcccctctctacctcatctttgtccctgtataccatggtcaaggaaatgagaagtaaactagccgctgtaggtcactgacactctccttagaaatgctaaatatttcttttcccaccttcttttcagtgtaatctgacctgagttctctgaatgcttgaccttcccagggagtttcagcgggtgagaaagcctgacactgcttggattacttgaatatatacagagctactcactcatatgtccatgcatttttggggtttttttttttttagttatatagtttgtggaattattttgcaagttacctttttccctttagacttttcaaaaaagaaccttttaaaattttatgacgggataagcaatatggtggttagcaagattaatgtttaccagtttgctgaagggaaaatcaaacaacatatgtccaaactctgtttaagaatttccatgggattctgaattccttaatctctgtagttttatgaagtcatagcaacatgccgcatacttgagttatagctccttgtcgaaatgtgtttgtataatttgactttatgttcaaaataagaaacaagtagccgtaagaaaaacatcttggtactctctcaccctgctacttccaacttttacccctctactcctttcctatcctgaaaaatacaccctcccatcatacctgcctcccccgcttgctgttgtaccagccttttgtttcatccccagctgagatttgagaggtgaatattcactttcccacatacccttgcacacgtgagccagactttttcaccgaaagtgctcaggccaaagtgtcaggtgactctcagctgccagatccagcggtcacttttgccccttaacgtgcgtgacttgcttttgacacctgcaatgctgcttaccactctcttcctgaaggggttgcctcttttggcttctaggccaacattctcttcatagttcttgtcctctgacccacccttcttcctctcctctgctgaatgcttttcttctcccagttccctaaatgctagtgatcccctggattctgccattggccttttttctctgtgtatgctctccttggattatctcatcctgcatatgtggattagtcacaaatctgtaaatcccagatctttctcttaggattcaggacaaccaactactagataccttgacttaaatttattatgccactaccagctattccaactgaaaaattgaattgagtggaaatcatgttgactatagagtagttttctccttcatctccctgcctccagtctttcctcccttcaatcttgcttctacattcctggtagaattctttttaaaacggaatggtgacctcattgcctcacttgttaaaacccttcaggggttccttactgtccctttgtaattaggatcttacaatattgaactttccctgagttgtaccctggaaaatctccccatccttcaattttccaaccctcaccccccacccccaccctttggtgttctgggaaaccacaaaggactatcttgttctccaatattctgagattagacccatctctgtccttcctgactcccagatgacttccttgtttacctgcctctatagaactccagaccctttccttttctttgagacagccctcattagtgttttattccagtagcttgaagaaaatcacctccttaattgtctggtgcattttggctctcacaagtcttaccctgttaatttgttacagttatcattctatcatgttatgtttgtttataatataatctctacattgtattatttataataaattacaatatattgttcataaattgttagtataaaatactattttatgtttatacttccactttaagtccttagtgtttataaaatacttgtatttataaacaaggatgaattataaaataagtctgtccaaatctatttctagatgcttttaagaggtgttttttagtagttggaggttttattaattatgttaccaactaaactgctgagacacccaaatagaatgacttaaataagatagatgtttatttcctccttacagaacagtccagatatgagcagccagcaactggcaggatagcactcctgtgctacacacgtgatttctaaggttgtcctgctgtccccatttctagcccgcaggaaaaaaggaaaaggaaggccaagtcaagtagccttgtcttgtagccttgtctttatgaaggttaccaggaaattagttgaactgcttcttacatcctgttggtccctcctcattaggtggccacatctggctgcaaatgaggctgggaaatgttatctctagatgggcacctttgcacctagctaaactgggtggtgatggtggcaggtgtatgttactaaataatgattttgggggcacacttagtagttatcatagaaatggaagactattttgattaaaaagtcaacttgggaggaaatgagtgattaaaaaacatctgggcctaccctataagcttcagtacttgccatatgtaagtaacaggactcaatacttctttcttttttccttctagaaaagtatgccatgcagagttaagagtatgtaaatttaaaagacagtttgtagctcttactctcaaccagtgcatttgtggtgaaaatgggtggcataggatacatgtgcatattacaaacaaatgcttgcaacatggaattctctctacacttttttgtctcattacttatattttcagattcagtacaacttgtggtatcagtttatcaccatcttcgaaatattttctgaaagatgatatattttctaagtggaagaattattcctttcctgctctcttgtattcctcactctcaaatgtcttagataatttgggtcaatttcctggctgatgtcccctggcctactagtaatggaatgaatacatgcttgccgacatgcagatcctctcaggccatggtacagggcagcctctaaccatgagcagcttcagctgtttcctgctttatgcagtacggatactgtcatgttgtacatatattctgatacggaaaagtgtgggaaatgctgttttaatggtttatgccaatttcatctctttgtactccaaattttttactagaaaataattgtaatatgtaacatcctaccatggtaaaaggtcagtccccttggtaaagaagtaaaaattatcttgctatgttatccactttggatggactgcaactgtaaaatatctaagatatgatgttgtgtccggaattggtgggttcttggtcttactgacttcaagaatgaagggctggccaactgctccgagtgcggggcccaccaagcccacgcccacccggaactccagctggcccgcaagcacagcacgcagccttggttcccgctcacgcctctgcctccacacctccctgcaagctgagggagtgggctccagccttggccagcccagaaaggggctcccacagtgcattggtgggctgaagtgctcctcaagtgccaccaaagtgggagcccaggcagaggaggtgccgagagcaagcaagggctttgaggactgccagcacacaatgtgactacagattatttgcctaaaaatatctatacttacgtttatatgtcagccatacataatggcatcagacctttctagcccatgtggcctataattagggtgacagtaacacttggttttcctggaaacgtccctggtttttgtttactattactgcttgttgctggtctcagttatcacagtttttttttttttttgcatttatacactttattgtctttttaaaaattttttacatatagtgaagatggggtcttactatgttcccaggctagtctcgaactcctgggctcaagcaatcctcccaaagtgctaggattacaggcgtgagtcactgcacggggccgtgtcattttagatattagcactgcttaccctaaataattctgggcagaatggtggtagtggtccatgctccgtcttttccaaactacttaagataagtatgggtggatctttaagctcttggagggcaaggactctatctatcttgctctactgtgtgtgtgtgtgtgtgtgtgtgtgtgtggtgtgtgtgtatgtgtgtggtgtgttcagcacctagcacattacctaagctaaacatttgaaaatgtaaatgattatgagcttgatggcttcatgacaacacagatttggtaaagtctatgatgggcaagatactttgttgccaactacaaatcctaaatacctaacagagtgtagcgtttaggaatgaatagattaatgattggtcacctcagtcttatatatttcttagcatttgcagtctctatctaaagatattgtggtccatcaacacattagacaatcctacaattacagttgtaacatgttgtatataaagtcacacaaacaagcaactgtttcttgggtataatgatccgtgtttactgttttttgattttatgaggaaaaaagatggcatttaattactccaataatgatcacatcgtagatagataaattctatcacttgtaagatttagaccttaatttgtaaccaatgtatgatattttaggctggaattcccagtggatgtgccaggaatggctactggatggctgaaatattcactcctttgggcagggctggagaccctttgcctgactgtattcgccctcttcagtttaccccagggcatatataaatgtcctttcctctgtgggccataaagtgaaacagattgggaaacactgttaatgacctctacaaccaaaatagcaggtgcttaaataaatgcagtggcacccttgacatttgagtatgcaaaataatttttcaaaaatgttatcttgggccattttttgagagttaaatagatgtgctttatcagaaagtaaatatccaagattatcaaagtcaaagaccataatcttcaaagaagcaattcatctaaaataagtaacaacatactgaattagaaagcatgttctcacatgaagttcacaaaaaagtctcacaaaaaaagagagaaagatagtatgtttgtttatattttggaccttcccaccacaggaaggtttaagcgtgtttatgaactgcaggctgctcaattgcgattcagtcctacatctctactaatgctttattgcagggggaaatgttagaaattatagcatcatgacagtggctatctactaaaattctgccagttaagataatttggggaaagggaagaaaccattaattccaagcacaccttagtttgtgaagggcttccagttaaatgtcctatcagatctgcttttctaaagatcatttgttattttcataagagctgcacaaatagatgctaagtacttagaaacagaattaaataaatcaaatgtgaataaaacatttgtttgttttatcaaatcctttgaaatagaacaaaagttatatttagccaaatttaccaatttatcactaaatcgaaaataaggacttgtttgtattacttcgaaaggccaaccctttaggggaatttttatttagctctaaaatgttttttaatgtcaataatgaattccactgcaatagcgattatattgccaaaggtcattaatgatttttaagtcttcattacgctttaatgagattgagattaagatctctgtaatatttgactgatgtatagtttaactctgtgtctcctcatcttttgattttcttgcttttctgttctctcttttcttcgttgattttttttttcttctcaaagccttcatgatctttatttttcccctgtcaccactacatttcaaatcctgggttttcctcaggggccgccaccttccccaaatttttctggacatttaaatccaattttgtggaattttcagggtaggaacattcaatgagcgggcctaccatgtgttcaacattagactgggtggtgtatgtaccttgtcacagtggggttttatttccatttcattgataaggaaaatgaaggcccaagtttacacagtgatgtgaaacattatctgtgttcctattgtacaggtaaaaggtggaaataggggtgaaaactcaagcaagctaactacttaagccacattcttatccagtatactaccaacttaagaactgaatttctagtttaaattagctctataatagagactctaggctatttgtgtttaaattattccatctgttagatatctttatacaggtgcaaactatttgccctaattagcccattaatctatccattttattcaataccttttctaccactcacaacctagttttaactacctacatatatcctttgcctggagtcatgcaatagctgctttcctgatcttatatcttccctcactaatctgtatgctaagcccagagtaatctttaaaagggacaaatttaattttcatcaacctccaatctgcagctgaaaacacaccagtgacttaaaactgcccttagtttgatgacatcaaactatgcttctaagcatcatctcacactgcatgttcctgtgactcttcaagctttgggcactagccttctttcagttctctggtcattctttgttcccctctctccacagggcctttgcacattctgggccaccatactcctatactctttcttcacacctcccttccatcacgatttcctcagaaaattcctccctgatcttgccagctggaggaaacaccccattatgcactttcatagtaccatgcaagcaggggaaggtccaagcttcctgggatctgaaatttttgtaatttgaggggctgtctttaagaaaaataaaattagagatacaaatatgagtatagggccttggaagcggtcagtgcgaggatagggtcctgacccttaacctttcactgcatggcaaatctacctcagcctgtacctcttgttggtagctcttcacattgacaattcaatttatatacacaactcggtgaatatttctttcccctacactatgcactgtgtgagagaggaaatatgtctgaatttactccccaagctttctccaggaatttgcatagtgttaacacttgagtatacagtatatatttcttgggtaaaggaataaatgagcaaatgaataattagaagaagacttagagttaatgcttataatgcccgtataaaattgtccgaattttttggaacccaaagccaagtggagaacatctgggttccatggctcataccatttcatcatgaaagacaaacttctcagtaccaggtggtaaagatgaatgttgataggtttatttatataaagcaccttattccacacaatgaataataaccagcagctgttactcagtgcttaccatgtgctgggcacttgctcaacacttgcacatggtattctgccttaacacatatggcattcaccactaactgcagagttactctcagtcagaatttctgcctccatggtccatgcaatgcagaagggcagaaaaccccatgcaggacaaggaaaaggaggcctgaggtgctggcaccttcactgtctcaagggtcctccctctctggagctccttcctctctgccattttatatcaccttttatgtgctaaaatggtgtttgtttgccactgccccagagatgatactggcttatcacctgccctctgcttagttcttggtattatctcctagtgagggtatgcccaagcttacctttgtctttgttttcctgtagagtgcttaattatgagtcaaactacatgcaggtggtctatccgaatgagttagcatcagaatgacctgttaaaaagagttctaatatgttgcccagctggacattgaaaaatgcctgttagagttgttgatcattttgactttggtataattaatagtgaccttttctcactgccagtatttggttcaggttgggtgccttctgagcacccttctggaatgctgtacagttgtgagtgattcattctgttaacaaatctttattaagtgcctaatctgtgctgtgccctgaggatacgcagataaaagaaactgtcctcccattaggtggaggcattgaaacccaagtggaggaagacagggagacagaactgacagttctgcaagtatcatgagcagccattgtctgactttgcattcatttctggtcttactggctagaatctcctaataaactaccaagtaatccttttaactgaataagctacctatccaaggattagaatttgtatctgagtgtttttctctcatactaactttgagaactttttgagggttagtgaggtaagcaccagagcaagatactcaggttcttttccagcaatctgtaccaaaccaggaataaggactgacaggctcccagctgtctgtcaccagagcagatatttatgggtttatgatcagaggttcttttggtcccaaaaagtaactggaaaagaatgtggcaacttcctgggaccatggaaagctgcttgggtgtcagggatatgctatgccaggtatatgctagaagaaatactattaaatgtcagtaaacttaaaaggaaaatggtcaagtttaatctaagatgtccattttgcagatggaaagaagcagggaatttaaaaatgcaagcaaagctcccattacaaatcctactaactgtctcataaactggatgaagttaggcgataaagttgaaccaaattcttcactcagtttgggatgactttctctggagcctttattaaggtctcttagctgccagactgttttagggactaatgaaactattgtcattatcccttgctctttttatgtttcctctaagttgctttctcagtaaggacagcttttaattttttaaaaaattatttccctttttaatccccagaaccctatagcaaaaactccataattatttgttaaagtttctagtagagggtacactaagtggggtctatagatactgaagctcttgtgaaattctgctggcctgagttcatgatttaagggaatgctggtgatagcttgtagaagaaactgagattgtatggaataaaatgtggaagccattgattttagagcctcttttttattgttctgtgaataaaatctcatttgtaagccctcaaatgtgggccttgacttttagaattgagagctagaagtaaaaacaaattcacagcagtgtactaacacatttttgttaattcctaagatattttgctttagacctattaacatatttagagttatgttccttagttctgaggcaaatttgctctgtgaatagcactacactaggaagaaatcctaaaagagacattaacatgtaatttgtgtaaagttctttttatatttcacaagttatgaattttctatagtccttttaagtgttaaatggatttataaaaagcttttggtggttagcaaaatgggtggtttttgcctaatgtgaaatctgagattgataagttttatataaggagtgtttttttaaatcttgagattttttatgtaaaaaaattgtttcagttatttttgatatctaaaccaaaatagcattagaaaggagctattttaaaagtttaatttcttatcataatcttttaagagggagtgacaccagtgacttacgtttcgtttctatatagaattatatcagctatagagtatattgtatacttgtaatacccataactattaacacttaaataatacagtaatacagaggcctattgggaaaaaaaaaaaaaaaaaaactaaatcattcctttttcccctattgcttctgaagcattttttattttctttaactgtataaatttttgataactgatttgggtgtgctgtgtgagtatctgtgtgtacatacacatatgtgcttacgtgtctgttgagtgttagggaaggcttgttgtagagatggtgtagctcattataaactttagtttgttctcatttgatttatttcaatgaaagatttgagaatttgatcctcgtaagtctcttgcatgttctccatttgcccaggttgaccaagagggctaacttttttccctgcagaaagaaagtgaaagttgatatttgattgggtagttaattcatgcctacatgaaaaattacaggccagatgtggtggctcatgcctgtaatcccaacactttgggaggccaaggcggatgaatcacttgagtccaggagttcaagaccagcctaggcaacatggtgaaaccctgtccctacaaaaaatacaaaaaattagctgggctactcgggaggctgaggtaggagaatcacctgagcccaggaggtcaaggctgcagtgagccaagatcacgccactgcactctagcctggacaactggagtgagaccctatttcaaaaaaaacaacaaaaaagaaaaactaagtataattgagtgttatattgattacattaatcaagccatttgcttctttatacattaaattcttatttaaataatatttcaaagccatttggcttccttcttttaataggggtactaagacctcctagtgcagataaacttttaaaggttttttttgtttgtttgttttgttttgttttgttttgttttttgagagagagagagtcttgctctgttgcccaggttggagtgcaatggcacaatctcagctcactacaacctccgcctcccaacttcaagcaattcttcttcctcagcctcctgagcagctgggattccaggcatctgccaccaagcccggctaatttttaaatttttagtagagtcatggtttcacatgttggtcaggctggtcttgaactcctgacctcaggtgatctgtcctcctcagcctcccaaagtgctgggattacaagcgtgagccactgcacccggccaacttttaaagttttaatttaggtgattatatttttctgttttaatgctagggtttcttttattgcttataacaatctcatgaatatatgttttctagcagtcaccaagctacccacatttaccctggctaacaaatgggaacaaagagcaagatttgaatccaggtctcttgggcatcggagttgtgctctttctagtatcatttgccacctcttcatataattttttttttgggggagatggagttttgctcttgttacccaggccagggtgcagtggggcgatcttggctcgctgaaacctccgcctctcaggttcaagctatgctcgtgcttcagcctcccaagtagctgggattacaggtgctcgccaccacacctggctaattttttgtatttttactagagaccgggtttcatcatgttggccaggctggtctcgagctcctgacctcaggtgatccacctgccttggcctcccaaagtgctgggattacagacatgagccactgcgtctggccaataattttgacttaaatacatctaaaagaataaataaaattaaatgccacatatttatcattagtcatttattccaggattactctatgtgcgtggtgctacatgctagttcaaaattcaacgaattggtctagtcatcttcagtaaagactaagacagacttttctcctcaatgattctttttttcttttttaaattcagattcagcaaacatctttgggggcttactttgtgccagaccctaaaccaggtgttcctaatacgttacactgtgaggtggtttttattattaagattttaccaatgaggcatgaatgttagagaggtaataatataagcaatcatttaaaaacatacttgaacttagaatgtagtatagcatgaatagttacaataggctacaatcatatctaccttcactatcctatttctgttgaaaggcagtttaggcagtaaaataaacaaaggttgagatctggtttctttattttaacagctttatggagataaaatttacataccatacaatttactcatttgaagtatataattcagtgttttttagtgtaatcacagaattgtccaacaaccatgtcttctgatttttgaactgttactgattttttttcattactactaaccatacatttgaataagaaaacaggatcataattctaaaagggttatattcaaccctgttgttttcagatttagttttgttcaaccaaagtccaataactttcccttttcagttctactacaaaaagcttccttgacttccatgagaaatgtttgcttacaggaatgttaagctgattttcatgtgtgccattgacaaggagaagctttggaatgtgaaatccacttccgcataatgtaattatttaagtcttttgcaatagatgtactataaaagagaaatcaaagaatatatgcgattaatacttactaaatatatgtttttcaagttaaaaaacaaaaatcccttgataaccatttatagccattctgagtcctgattcactaaggagatcaaacctcaagatatggataggatgcagagaaaccaggtgctgatcccttctctcattggcttaagttcacattagaaagaatcgtattgttatgatggccatacttcccaaagcaatatgtagatacagtgctattcctatcaaactaccagtgtcatttttcacagaattagaaaaaaacgattctaaaattcatatggaacccaaaaaaagccaaatcaccaaagcaatcctaaacacaaagaataaagctggagatatcacattatgtgacttcaaactatactacaagactacagtaaccaaaacagcatggtactgatacaaaagcagacacacagaccactggaacaggacagagtccacaaaattacacacatacaaccacctgatctttgtgacaaagtcgacagtaacaaacaatggggaatggactccctattcaataaatggtgctggaataactggctagccttatgcagaagattgaaactggaccccgtcctttcaccatatacaaaaactaactcacgatggattaaaggacttaaaggtaagacctaaaacaataaaaactctgaagaaaacctaaaaaatactattctggatgttggccttggcaaagaatttatgactaagtccccaaaggcaattgcaacaaaaacaaaaattgacaagtgggacctaattaaactaaagagcttctgcacagcaaattaaactgtcaacagtaaacagacagcatacagaatgggagaaaatatttgcaaactatgcatctgacaaaggtctaatatctagaatctataaggaacttaaatcagcaagcaaaaaacaatcccattaagaaatgggcaaaggacatgaacaaacacttctcaaaagaagacatacatgcagccaacaaatatatgaaaaaatgctcagcatcactaatcattagagaaatgcaaatcaaaaagtcagtgagataccctctcacaccagaatggctattattaaaaagtcaaaaaacaccagatgctggcaaggttgcagagaaaagaaaacacttacgcactgctggtggggatgtaaattagttcagccactgtggaaagcagtgtgtagacttctcaaaaaagttaaaacagaactataattcaacccagcaatctctattgtgtatatacccaaaggaaaacaaatcattctacaaaaaagatgtgtacttgtatgttcattgcagcactattcacaatagcaaagacatgtaatcaaccaagatgcccgttgacagtggactggattttttttttaatatggtacatatacaccatggaatactatgcaaccatagaaaagaatatatcatgttttttgcagcaacatggatgcagctgcaggccattatcctaggcatagtaatacagaaacataaaaccaaataccacatgttctcacttataagtgggagctaaacactgaatacacatggatacaaagaagggaacaatagacacaggggcctgcttaataggggagagaaaggaggggagttgggttggaaggctccctattaggtactatactcattaccttgattgggggtccccaacccctgggccacggactgctgctgattcatggcctgttaggaaccgggtcgcacagcaccaggtgagcagtgggagagtgagcaaagcttcatctgtatttacagccactccccattgttgacattactgccttagctctgcctcttgtcagatcagcagcagcattagattctcgtaggagtgtgaaccctattgtgaactgtgaattcaagggatctaggttctgctatccttgtgagaatctaatgtccgatgatttgtcactgtcttccatcactcccagatgggaccgtctagttgcaggaaaagctcagggatcgcgctgattccacattatggtgagttgtataattatttcattatatattacagtgtaataataacagaaataaagtgcataacaaatgtgatgcacttgagttagcccgaaaccatccccctcccccgtccatggaaaaattgtcttccatgaaaccagtccttggtgccaaaatggttggggactgctgaccttggtgatgggatcattcatacatcaagccttagcaactagtttacccatgttacaaacctgcacatgtacaccctgaacctaaaataagtcactagaggatcataatgtacttaatatactatattatataaatatgtgttaaatatgctttatacacatacacactcagtatatatgaaaagctacttttgttttcaaagtgcttgtgcatctttaattcagtcgatggttgaaatacccccctggtaggaagggcatatgtcattatccccatttttggtgggtaaaatggtataacagatgcattaattcaagtcctgtggctgacattgctggaaagtactggctttcttccttatggcctgggcactttcttaagcctatacttaacatccatgctttggtttccatactttggcttacccgtgagatgtgatatctgtacaggggtataccagaccactaactgggatagcattaaagttatttgtagtttctctaacgcatcatttatctcatgctatgcctttgcccttgctatttcttcagtcaaaattgttttccctctccccaccacctcatgtcttacccttccttcatctctcagctcaaaaccacacctctgtgaagttctcccagttactacatgcagacttgggcttgccccgttctgtgcatctgctatactttgtgttaacttcatattacttcgtattaaggtgatagcaatgcccttactattaggttggtgcaacttttgccattaaaagtagtggcaaaaactgcaatcactttcacaccaacctaatatgttgtaattgtacacttactgcttatcatcctgcattagactgggatctccttgagggcaaggatctatattagatcatctctgcattcccctccatggtgcttggcacggggctggtagctttcttgaattcctagtgaatgtcaggaagggatggtggtgattacataatgtcattgctttgtcacctctaagccaaggacaacagtgaggagttgcctccttcaatagccaacccttagccaataacacaggcattggatgatgcgtggtagagaaaaagatttagagatgaaagattctgttagcactctcacttaattagctcagagaggtcatttaagctttttgagcctcaggtttcctcatactgttgggcagtagtaatgcctagaccactgggttattatgaaaataaatagagatcttgtataggaaagtagcttgcaaaccgaaaagcactgcaaacatgagaaataaagaagaaaaggtaatctggtaggaagaacatgtgctttggccttaggtgggattttaaatccagtctctgctactaattatttaatgtccacaacttccgtgagtctatttcttcccctctcaagggtaaataacaacactttggctgcttagagtaatgttgtgaagtttaaataggattcaggcaatgttccactgtgttccacctgtgttcagctttttccacccagagctttcagatattgtatgttcctacctctgcaccttcttttctatggcagaatcagtcctttgtactccagagaaagcaggcacctactctcaggccagggaaataaatagtgaggggagaccaccagaggaagagacaggcttccctggcatgtaatcccagccagccgggatgtgccttgggacggggcggaggcgcagcctcaatgtcatgtcacagcacattcctaggacagctgtttctgcggcagcttcttttaccatttcctttgaaactttttattgaagtggaagcctgagtgtacagatcacattttataaattcaacacacccatgtaaccagatcattcattcttgttgctgcctagtattctattttgtaaatataccacatcttttattctacagttgataggcatttggatagcttacaatttagagctaatacaaatggtgctgctatgagcattctcatacatgtttttggatgaatatatacccgtgtttcttacaggtatatacctagcagtggaattgcagggtcataaagtgtgcttatgctcaattttagtagataatactaaaacatttcccagaatttcccaacattgacacactcaccagaagtatttgcattccagttgctccagcatccctaccagcacttagtattatctgtcattttaattttaggaattctggtggattttataatttcatacatttggaaatagagccattctcttagcccccaaaagcttaaactaaacttttgtacattcattttcaggaatgtgctctctataaaactgggaagttgtatcagatgtaaagcccctagtacagtgcttgccacgtggaaggaaaatgacaagtgatagccactattactgtcaccattatcatcaccactaaatgccatctgtgttcttcaagggtgagagtagggcatggtggattacaaaggtgaataagacatattctctgcctacaaggagctcaagatctagtggaagagaccaagattagaaacagattttgtttttgtttttgagacgggagtcttgctctgtttcccaggctggagtgccatggcatgatctcgccgcactgcaacctccgccacccggtttcgagcaattcttcctgcctcagcctcctgagtagctgggattacaggcgcctggcaccaagccccgctaatttttgtatttttggtggagacggggtttcaccatgttggccaggctggtctcaaactcctgacctcaggtgatctgcccacctcggcctcccaaagtgctgggattatagacgtgagccaccatgcccggcctacaaacacattattattagacaacatgatagcaatttgaaggaaaaggtagtgcttgagctgggacttaataaggaaaagttcaatagaaagaaaaaagaaaaatgggctggagaaaacagcaagggcacaaaacttctggtggggggtggcagagtagacaatgtgctggaagagaggagctcatctgaggttggagtggtcatgtgctgtctgttattcagtgttaccattcttgcggggagtctggggccggagctggtgactgtgtgtgaagttacagtactctgtactctgttgctatgcccactgccactacctttgaacttttcttttcccactgggaaggtggacagcctcttctatttcctgggatcaaagcctgtcctgagtacaaggtgcttgaaaactaccattgacacttcaacagctgcaaaataacactggttgcaagaatccaacaatgtctctgctgtgtggtcctttaactgtttatttttatgctgggacctggcttagagtgttcattcttgcatagatccaggttgctttgtggtagcaattcaggttaataaaatgctcctctgcccaataccatctgtagatgttaataaatgttctcaaggaatttttaggatgctgaagatgatgatcaagatctcaaaatcatttcattgattaagtacctactatgtgtcaggcattggatacataggacaaagaaggaagtgggcatagtgcatatcttcattgaattggtaatccaatgggagatcagacaagtaaatacagtgtgataagggctgtgatgggcggaggtacagagtattgtgggaacacatggaagaaacacctcacccatactaggagatcagggaaggctttctggaaaaggctgcagaaacctgatagcctagtgaagaggagtagggaggaagtaatcaaggcagaggaaacaaagtgttggagaggctgaacacaagggagagaatggcttgttagagaaactggcagaagttactggcctggaacagagagagcctgaagagtttttaccaagggacccatggcaagatggaaaagggattaaaggaggtaaagactggcagcagaaagaccagttgtaataatcatggtgtaaaagacaaataaccaatgtcctcagtacactgactttcaaaattcacttatctctcaatctgccaagccagacatcctggcctccctagaaccagatacaattacagtcacatgtctcttcatggggataccttctgagaaatgtattgttaggcaatttcattgttgtgtaaatatcagagtgtacttacacaaagctagatggtgtgtgtgtgtatatgcatgtatgtatgtattcatatggaaaaccaaattcccaacatcattactgaatatcagtcctttcccctacttcaggagtccccgggccccaggctgcagactggtacctgtcgagagctgttaggaactgggcttcacggcagtaggtgagcggcgggagagtgagcattactgcctgagctccgttttgtgtcagatcagcagcagcattagattctcatagtagcatgaatcctatcatgaactgctcatgtgagggatctaggttgtgcacaccttgtgagaatctaatgcctgatgatccaaccaagatgaaacagtttcatcccaaaaccatctctccccccaacccccatttctggaaaaattgtcttctacaaaacttgtctgtggtgccaaaaaggttgaggaccgctgccctacttgatctgtaatgccagtgattagtgctctgtatcaggtttctatgtacttcattataaaatggagtctcactctgtcacccaggctggagtgcagtggtgtgatcttggctcactgcaacctcctcctcctgggttcaagcaatttttctgcctcagcctcccaagtagctgggattaaagcgtgaaccactatgcctggctaatttttgtatttttagtagagatggggtttcaccatattcgccaggctggtctcaaactccgcctcaaatgatccacccacctcggcctcccaaagtgctgggattataggcatgagccaccatctcgtatcttatgggaccaccatcatatatgcagtccattgttgaccaaaacaccattatgcagttcatgactctttttaaaaagtaaagaaaaatcctatttgaaagaactattacatgcaaaaaaatctattgttcctattactcatgcagatggccctttaaagccatggtccccaggtatgactgttacgttccttgccagaaataaacctgtgcaaggtgtactctctagtctctgtatgtgaagtaagagtcattctgtgcttgatgctgtgttttgtgcattctagataggaactgaagagctcaaaatgacatgaggttgagctgtttctcatttaggttagcttgctgtgattccagtagcttttcttcctgcttatccaagccattcatgaaagcaaaacccacacaagatacaggagctcagaataaaaggagttcaagtgggcgtgttatctcccacttcccctactaaggtacaaagttatttttctttttttcacttccttggtaattttttttttttttttttttttttacaaagactggttaccaggcatttcatacttaagccctgaagtctgggaatgtgatttctaacaattttatgtttggagttcttagacttgtgtttacctgcttacataagagataattttgcttacaaaactaactacagatgtgatcattgctgtagtattcattgtattttattaaaaaatattatgaacctactctgtgcagtactaggtatgtggaagggtttcggaaaaaaaaaaaaaaaaaaaaagacctggtccctgccttcagaaacttaatttaaaacagagcagaatcaaagtactaatgccacctttcttcattgtgtcatccaaggaagacacaagtaatcagtcacagcactgtttcctactgaaaccagaatcagcctcagaatcctcctcaacacagcattccagacaacccatgaatagataattcatcttcctggttgggtgtggtggctcacacctgtaatcccagcactttgggaggccaaggtgggcagatcacaaggtcaggagttcgagaccagcctggccaatatggtgaaaccctgtctctactaaaaatacaaaaattaggtgggtgtggtggtggatgcctgtagtcccagctactcgggaggctgaggcaggagaatcgcttgaaccaaggaggcagaggttgcagtgagctgagatcgcgccactgcactctagcctgggtgacagagtgaggctctgtctcaaaaataaataaataaataaaaatattaaaaattcatcttccatccttaatttaaaaaaatacatgcattcccaaaactcttgggtacttgccttgatttttattgcttggtcttttgctagtttttaaagaaataacatgatttctttctttaaaaaagacaaagcaatataaatatggaaacccaagaacctctcaagaaatgaaaatatggtcatgtggtttttcaagaagttgaaacattcagttcatgcacataattagagaagcccattcccattacttgccactagaaacactgtttttcttggttacttccaggatgattgtccttgtcctttattatttgtataaactagttccgtgctataaagctgtcagctgatagtgaataataaccacattttcaagtttcctggcaccggggcttcattcatggctgctcgggtcacctgctttggtagagatcgtatgtcttcctggtgctgacttattcagcatcaccatcattgattatttcatggccacctgagtcttgagctgtcttctcaatccacatgctttccataggactgcatcaaaggccagagtttcagctatatatattgataaatcccaagtgtgtatagtaagactcctcctaaacgctagacccttagtttcaactgttcatggacacttccaccagagtgattggagctaacgtaaacagctaacaaacacttgtggccagaggatgtgctaagagcgttacaatcattacttaatttaacctttccaacaacccttagaagttttcatccctattatacagatgggaaatacacaaggcttaaggaatcaaccattagtaagtgttagaactaagtctggaacataagactatccaaagtttaacggtatttaatggtatacacagacctttcaattttggcttatacaaaataagagtcatcccactccttgacctccaaatttcagtcttaacctttaatgagtccacaacaaatgccatcctgctgtcataccctccttcaaggtccactaagtaaccactatggacttggtgcagtctgtcataatttcttacctagaatgcaattcttactcattctttccctcttcccttgcactaggagagaaaagctgtatattccccttttagtttattttctactacatttatattggataaaaagataggacctgaatgagaaaactttagttaggagcaggcatagggtttttctcatttactcttttcatttgcatctctagaataatgtcttttgtcttcttgttttctaggaatgtttacccttgcggaagttgcatcacttaatgacattcagccaacttaccgaatcctgaaaccatggtgggatgtgtttatggattacctagctgttgttatgttaatggtagccatctttgcaggaaccatgcaacttaccaaagatcaggtggtctgtttgccagtattgccatctcctgtaaattcaaaggcacatacaccaccaggaaatgccgaggtcaccaccaacatcccaaagatggaagcagccaccaaccaagaccaagatgggcggacaacaaacgacatttcctttgggacatctgctgtgacacctgacatacctctcagagccacatatcctcgcacagatttcgcacttccaaatcaggaggcaaagaaagagaagaaagatccaacaggtcgaaaaacaaacttggattttcagcaatatgtatttattaatcaaatgtgttaccatctggcccttccgtggtattctaagtactttccatacctagctcttatacatactattattctcatggtcagtagcaacttttggttcaaatatcccaaaacatgctcaaaagtagaacattttgtttcaatattaggaaagtgctttgaatccccttggacgacaaaagcgttgtctgagacagcatgcgaagactcagaggaaaacaagcagagaataacaggtgcccagactctaccaaagcatgtttctaccagcagtgatgaagggagccccagtgccagtacaccaatgatcaataaaactggctttaaattttcagctgagaagcctgtgattgaagttcccagcatgacaatcctggataaaaaggatggagagcaggccaaagccctgtttgagaaagtgaggaagttccgtgcccatgtggaagatagtgacttgatctataaactctatgtggtccaaacagttatcaaaacagccaagttcatttttattctctgctatacagcgaactttgtcaacgcaatcagctttgaacacgtctgcaagcccaaagttgagcatctgattggttatgaggtatttgagtgcacccacaatatggcttacatgttgaaaaagcttctcatcagttacatatccattatttgtgtttatggctttatctgcctctacactctcttctggttattcaggatacctttgaaggaatattctttcgaaaaagtcagagaagagagcagttttagtgacattccagatgtcaaaaacgattttgcgttccttcttcacatggtagaccagtatgaccagctatattccaagcgttttggtgtgttcttgtcagaagttagtgaaaataaacttagggaaattagtttgaaccatgagtggacatttgaaaaactcaggcagcacatttcacgcaacgcccaggacaagcaggagttgcatctgttcatgctgtcgggggtgcccgatgctgtctttgacctcacagacctggatgtgctaaagcttgaactaattccagaagctaaaattcctgctaagatttctcaaatgactaacctccaagagctccacctctgccactgccctgcaaaagttgaacagactgcttttagctttcttcgcgatcacttgagatgccttcacgtgaagttcactgatgtggctgaaattcctgcctgggtgtatttgctcaaaaaccttcgagagttgtacttaataggcaatttgaactctgaaaacaataagatgataggacttgaatctctccgagagttgcggcaccttaagattctccacgtgaagagcaatttgaccaaagttccctccaacattacagatgtggctccacatcttacaaagttagtcattcataatgacggcactaaactcttggtactgaacagccttaagaaaatgatgaatgtcgctgagctggaactccagaactgtgagctagagagaatcccacatgctattttcagcctctctaatttacaggaactggatttaaagtccaataacattcgcacaattgaggaaatcatcagtttccagcatttaaaacgactgacttgtttaaaattatggcataacaaaattgttactattcctccctctattacccatgtcaaaaacttggagtcactttatttctctaacaacaagctcgaatccttaccagtggcagtatttagtttacagaaactcagatgcttagatgtgagctacaacaacatttcaatgattccaatagaaataggattgcttcagaacctgcagcatttgcatatcactgggaacaaagtggacattctgccaaaacaattgtttaaatgcataaagttgaggactttgaatctgggacagaactgcatcacctcactcccagagaaagttggtcagctctcccagctcactcagctggagctgaaggggaactgcttggaccgcctgccagcccagctgggccagtgtcggatgctcaagaaaagcgggcttgttgtggaagatcacctttttgataccctgccactcgaagtcaaagaggcattgaatcaagacataaatattccctttgcaaatgggatttaaactaagataatatatgcacagtgatgtgcaggaacaacttcctagattgcaagtgctcacgtacaagttattacaagataatgcattttaggagtagatacatcttttaaaataaaacagagaggatgcatagaaggctgatagaagacataactgaatgttcaatgtttgtagggttttaagtcattcatttccaaatcatttttttttttcttttggggaaagggaaggaaaaattataatcactaatcttggttctttttaaattgtttgtaacttggatgctgccgctactgaatgtttacaaattgcttgcctgctaaagtaaatgattaaattgacattttcttactatatcaccttttttgaggggtcttaatttactttgcttaattggtgcaatattgctttaactccgttcactaaagacaaacacatcaagtccaaacttgtaaatttagatttgctgaaatactgatttatttcctgttttaatttaaagtgcctcaagtaagcacctctcacaacagttgactggtactgctgctcctgagcacctcgtgtcatagattttatactcttacagacttggaatgcagtagaggtatgtggatttttaggggttttgtttttttaagaataagtaacaagaaataacacatttcttaataatagcttttttgacatagtttggagttctgattatatggtacatttttctaccagtaatatagggttgccaataaatagaaaatgttttctaaaaataaattttattacaacaaaa

SEQ ID NO: 5 - DNA, 13519 bp, Homo sapiens
  LRRC8E
gactcaccctcgcggcccgcccgggaactctgcgggagtcgcggcagctccgctcccagagacccaggagttgggcccagcgtccctcccggaacggtgagtaggtggctctttgcctctggacaccccacgaggtggcagggtgggggtcgggctgggagtccccgggtctggacaaggggcagagccgctccaggtgcgccggccctgtggccggggcaggccgtcccgacacaccaggagctggggtgggggtgccggccggtgcaaagggaaacctggcccacgaggtgcccgtcctgggagccaggcagacgcggccaagtctcagagtgaagataaacaggctgaagataaacttgactttgggggtctcaaccactttcccctccccttggcatctgggaagacattgacaactcatccccgggagactctcagagctccagaacctaccacttggggtgggttcaaccaagcctgacctctggtgctcttgaacttacacatgtcccagatcttcaagtctgctggcagaggcggcttggaaaaccactcggctgggaatccgcaggcctggggcctgacccaggacgggctacaaagatgccaccctcccccaccctagaccctcagtttctcccttgcataaatggttgaggctcagccggacaccgtgccttacgtctataatcccagcactttgggaggccgaggtgggcaggtcacctgaggtcaggtgtttgagaccagcctggctaacacggtgaaaccccatctctactaaaaatacaaaaattagtggcgtgtccctgtagtcccagctactggggaggctgaggcttgagtactgctcggacccaagaggtggaggttgcagtgagttgagattgggccactgcactccagcctgggcaacagagcgacactccatctcaaaaaaaaaaaaagattgaggccaggcacagtggctcacacctgtaatcccagcactttgggaggctgaggtgggaggattgcttggactcagaagttcaagaccagcctgggcaacatagcgagactccatctctacaaaaaatctaaaagttggctgggcatggtagcacgcacctgtggtcccagctactggggaggctgaggcaggaggatcacttgagcccaggagttcgagactactatgagctatgatcgtgccactgaactccagcctgggtgacagagcaagactctatctctctctctctctctttttttttttttcaagatagagtcttgctctgttgccaggccggagcgcagtggcacgatcttggctcacggcaacctccacctcccaggttcaagcgattctcctgcctcagcctcctgagtagctgggactacaagcatgtggcaccatacccagctaatctttgtatttttagagatggggtttcatcatgttggccaggatggtcttgatctcttgacctcgtgatccacctgcctcaacctctcaaagtgctgggattacaggcgtgatccactgtgtccgtcaggactctatgtctaaaaaaaaaaaaaggttgggccttgttttctacaaaacccctcttagggctgaaaactgctggatgtttcttgcttcttccctgcctcttagcatgggctgtgggtgtggagggtctcactctcccacccttcccccagcaggaactcatcaccacattttttggaggcaagtggcttgaggaggacctgggagggaggaggggtgaggcatgggtggatggagccagggctggtgggagccacatggtctcaggctggaaagccttccagcctggcctgccacaatgcaacaccgctcttaggtgcttttggcaaagatgctgtggtttttttgttgttgtttgttttttgagatagagtcttgctctgttgcccaggctggattgcagtggcgcaatcacggctcactgcaacttccacctcctgggttcaagagattctcctgcttcagccgggcacggtggcgcacgcctgtaatcccagctctttgggaggccgaggcaggcagatcatgagatcaggagattgagaccatcctggctaatacagtgaaaccccgtctctactaaaattacaaaaaattagccgggcgaggtggcgggcgcctgtagtcccagctactcgggaggctgaagcaggagaatggtgtgaacccgggaggcggagcttgcagtgagccgagatcacaccactgcactccagcctgggcgacagagtgagactctgtctcaaaaaaaaaagagattctcctgcttcagcttccccattagctgggattataggtgcccaccaccactccagctaattttgtatttttagtagacatggggttttaccatgttgggctagcctggtctcaaactcctgacctcaaatgatctgcctaccttggcctcccaaagtgttgggattacaggcgtgagccaccgtgcctggcaagatgctgcattttaattagaggctgtctcccaagagggaacagagcagttgcaatcctacaggcaggacctggaggttggatgggtcaggcttcgaatgccgcttggaactgtagaagcttgagcaagttatttctctcccctgagtctggaaagcaaagtctccgacatccacgctgcctgggttgggataaaatgaggcattcagtgtctggtggagaaagctgagcgtatacgtcccatcgttttattttccctggctctgccccaattcttcgagcccctgttcctggaccttcccaccagccctgctcagacaaagtgggggcctattttatgtctgtggaaatttaatggcaggtttgtgaggatcaagtttgagtcatggagacagataatgatagcccctgtggctgccccccggtcccctgagtgttgctgccttcttgtctgcgattgtgcacctgggatggctattggtggagcttacaccagggtggcaaggacaggaagttggtccctgctcgggtgtgtgtgtgtgtgtgtgtgtttgtgtgtgtgtgtgtgtgttgtgtgatttcttctgcagcctccagagtcaggagtttttgttttgttttgtttttttgagacggagtctcgctctgtcgcccaggctggagtgcagtggcgagatcttggctcactgcagcttctgtctcccgagtttaagcaattctcctgcctcactctcctgagtagctgggattacaggcatccaccaccatgcctggctaattgtttttgtatttttagtagagacgaggtttcaccatgttggccaggctggtctggaactcctgacctcaaattatccgcctaccttggcctcccaaagtgctgggattacaggcgagagccaccgcgcccggccccagtcagtagtgttttgcttgctgtgtttgagatagggtcttgcccaggctggttcatggctcactgcagcctctaactcctgggttcaagcaatcctcccacctcagtcttccaagtagctgggactacaggcatgcaccaccacacctggcaagattttagattttttttttttttttgagacggagtctcgctctgtcgcccaggctggagtacagttgtgcaatcttggctcactgcaggctctgcctcccaggttcacgccattctcctgcctcagcctcccaagtagctgggactacaggcgtctgccactatgcccggctaattttttgtatttttagtagagacggggtttcgccgcattagccaggatggtctcaatctcctgacctcgtgatccacccacctcggcctcccaaagtgctgggattacaggcgtgagccaccgcacccggcccaaatttttagatttttttgtagaaatggggtctgactatgttgtcctggctgctcaaactcctgggctcaagcaatcctcctgcctcagcctcccaaagtgctggaattacaggcctgagccactgcacccagcctggggtcagcattcttaggtaaagcaactgagcagtgtccctgcaggatccttggaggactcaatggatggccggaaccaaaacaggacgtgagggtccctggtgctgggtccctccgaagctcagtgtgtgtacttggactttataccctcgtgcacaaatatgcatgacccaaaatgaccccaagcgtccattttggtgggtgcaatgtacatattcttcccctggggacagttgagctatcctggtatgtttctaccagattacatactggtgacccaggcagactgggatatttgtagtcgggtttcttttctcttctcttctcttttttagagacagggtcttgctctgtcgcccaggctggaatgcagtagtgcaatcttggctgactgcaacctccgcctcccgggttcaaggggttttcgtgcctcagcctcccgagtaaccgggactacaggcaccctccaccatgactggctgttttgtatttttatttttattttgttttagttttgagacagagtcttgcactattgtctgggctagagtgcagtggcgcaatctcagctcactgcaacctctgcctcccaggttcaagcgatcctcctgcctcagcctccctagtagctgggattacaggcacctgccaccacgcccagctaattttttgtatttttagtagagacggggtttcatcacgttggccaggctggtccctaactcctgaccttgtgatttgcctgcctcggcctcccaaagtgctgggattacaggcgtgagccaccgcacctggctaatttttgtatttttaatagagacggagtttcaccatgttggccaggctggtctcgaactcctaacctcaagtgatctgcccacctcggcctcccaaagtatacttggctttttgcttagggcagggagtgtgaatgagcatgagggccaggccatggtgcgaaggaccctgtgctctgggcacccccttgcatggatctgggtctgggtgagtcactgcctgagtcagggaacccatgacttttttttttttttttcccgagacagagtctcgctccgtctcccaggctagattgcagtggcacgatctcagctcaccacaacctccgcttcccgggttcaagcgattctcctgcctagctgggactacaggttcatgccaccatgcccggctaatttttgtatttttagaagagacggggtttcaccatgctggccaggctggtttcgaactcctgaccttgtgatccgcctgccttggcctcccaaagtgctgggattacagacgtgagccactgcgcctggcccaacctatgacattttacacttgcatggtgagtccccgtgtgggcaggctgtacccacacccttgcctgcctatctgattccccagactgaagtgggggcctctgagggggtaaaaggatgtggtctttcgggactgcttggcctttagcttggtgggtggagcctgtatcctaccttgttttaacccaggagtcaccccaaaatatgggggggacacaaactgaaaagggaggctcagacatgtcagaaccattggctgagtcgtgggtcaggggaccagggtttccctagattcttcctggggagatggatggggagccagagataactccctgatgggcagatgccctccttcttggcccaactcctgtcccgttacagattatctttgtcttttgttttgtttgtttgagatggagtctcactctgtcacccaggctggagtgcagtggcacggtctcacttcactgccacctctgcctccccggatcaagaccctcccacctcagcctccagagtagctaggattacaggtgtgtgtcaccacacctggttaattttttgtgtatttttagtagactctgggtttcaccatgttggcctcactggtctcgaactcctaacctcaagtgatccacctgcctcagcttcccatagtgctgggattacaggcatgagccaccgcgcccagccccagttacagattatctatctggacaaggacaggaggctcccaggagtgggggaacttggatgttggcagaagtgggctggtctaaggttccccaccttccttgttccacctccagcctctggggaagtaggtacctgcctgggagagtgacatctcagctcatgccccacgccccagtgtgatttgtggctggcctgtctgtatctgtaatagtgatagcttggtctaaaaggagaccctgggaacagacagagtgggccggccaggagcctgacagcacagccctagcctggtcgcagatgtcacctgacaactttccatttgcaaatcctccttcctcctcggacatccggacaccccattgccccctctcagagcccaggtctctggtttctgcctgtgattctgggctgtagcccctttagaaactcaggtgtcctggtcacctcctacccccagggctgactagccttcagcttggcagtccctgcccctggggccctagtaaccagagccaggctccagccggcttttccggttagaggttggaggtggtgcccagagtgcccctggagggggtggaggggcctacgccccggaagtcctgagccttggaacgctttcaggcctggtggcggtgggagccggggctggcccagacgctgagtgtctgcagtcagggagcatcgaggcagatgttttcaggccttggtgtcaggggtgggagagggatgcctcgttttggggagggggccacccgaggaggctggagggcggcggccctgtggactatccctcatgtgctcttcgaggtcagacaagtcagatcaggtgcaggggtgcccagaggggaacagtggccgcttggttctgggcaggtggtgacgtctgcatggagggtgactaaggccaggctaagagggaagtgggggcacacactttggtggtttggacaagtttgggcagggagggtcacaggcctgcctgtgtccggctcctcggaggaccccctgcagagcctcccatcctgagggtctctctctacaccccccgtctcgtcccgtcccacaggcagcatgatcccagtggccgagttcaagcagttcacggaacagcagcctgcgttcaaggtgctcaaaccctggtgggacgtgctggccgagtacctcaccgtggccatgctcatgattggggtctttggctgcaccctccaggtgaggccctcccctggcaagggggtgtgaccagaggggcggggcaggtgtctggggaagtcgggaagccttctcatcacccaagaaagagaggaaactgaagacagagccccactcaaaggccaatccagaccccttatcttccttacctccatagccaggagcaccggagtccccacattgctatgccattccatatctttttttcttttttgagacggagtctcactctgtcacccaggctagagtacagtggcatgatctcagctccctgcaacatctgcctcccgggtacaagcggttctcctgcctcagcctcctgagtagctgggattacaggcgcccaccaccacgcctggctaattttggtagttttttagtagagatggggtttcaccatgttagccaggctggtctcgaactcctgacctcaagtgacctgcctgcctgggcctcccaaagtgttgggattacaggcctgaggcactgcacctgaccctttctttttttttcttttttttttttttctttttggctaaaaagagacaggggccgggcatggtggctcacacctgtaatcccagcactttgggaggccaaggtgggagtatcacttaaggccagaagtttgagaccagcctggccaacacggtgaaaacccgtccttactaaagacacaaatattagccaggcatggtggtggacacctgtaatcccagctacttggaaggctgaggcaggaaaattgcttgaacctgggaggtggaggttgcagtgagccgagatcatgccactgcactccagcctgggtgacagaacgagactccatcttaaaaaaaataaaataaaataattgaaaattaaaataaagaaacacggtctcattctgtcatccaggttggagtgcagtggcatgatcacagctcactgcagccttgaactcctggactcaagtgatcttcccgcctcagcctccagagtagctgggattacaggcacacaccaccacgtccagctaatttttaaatttcttgttgacacaggggtcttgctatgttgcccaggcttgtctcggactccgggcctcaagccatcctcccacctcggcctcccaaagtgctgggattacaggtgtgagccactgtgtccagccccatattttcacaatgctcagttttatccctagaatctggaaaggactgcagttgctatatgagtttgttacttctgttgtaacaaagtagcagggattgagtggcttaaataaccagaaatgtttcctcttctgggtctggttctggaggccagaagtctgagatcaaggcgtcagttgtcagtgagggagaatcttccaagcttccctcctagatttttgcagctccaagagttccttggcttgtagatgacattctcccagtatttttctttttctttttcttttttttttagacagagtctccctctgtcgcccaggctggcatgcagtggtgcgatctcggctcactgcaacctccgcctcccgggttccaacaattctcctggcttagcctcccaagtagctgggattacaggcgtgtgccaccacgcccggctaatatttttttgtatttttagtagagacgtggtttcagtatgttggccaggctggtcttgaactcctgacctcgtgatcaggatcagcctgcctctgtctcccaaagtgctgggattacaggcatgagccactgcgcctggtcgagacagttttttttttttttttttaagcaagctctgtcacctaggctggagtgcagtggcgcaataatagctcactgtaaccttgtactcctggcctcaagcaatcctcctctaccacccccgcttggccccccctcccactgcctcccaaatcactgggattacaagcctgagccaccgcacccagcctctccctgtatcctcacgtcatcttctgtctttgtgtctgtctctgtgtctatgtttcccctttctctaaggcgcagtcatactgggcccaccctactggctttgtcttgactacctctgcaaagaccctattaccaaacaggagccgcctgacccagctcaaataataccacattctaaggattcggagttgggacttcaacatcttttgtggggacacgattcaacccagctccttttctcagacaaagaccagtgctttgtcagttgtggtggctcattcctgtaatcccagcactttgggaagctgaggcaggggatcacttgaggccaggagttcgagagcagcctggccaacatggtgaaaccccatctctactaaaaatacaaaaattagctaggcgtggtagtgcgtgcttgtagccccggctactcagcaggctgaggcaggagaattgcttgaaccaggaggggaaggttgcaatgagccaagattgcaccactgcacacagcctgacagagcgagactcttgtctcaaaaaaaaaaaccaaaaaaaaaaaaaacagtgcttagaggtttagcaaaactcttccaaaatcacagctggctggcaacagtcagagatggggggtgcggggggtctcccaccttcagcctgctttttccccatccccctcattttctctcttttttaaattaatttatttttgagacagaggctcactctgtcacccaggctggagtacaatggcgcgatctcggctcactgcaacctccatcttccgggttcaagcgattctcctgcctcagcctcccgagtagctgggattacaggtgcccaccaccatgcccggctaattttgtatttttagtagagacagggtttcaccacgttggccaggctggtctcgaactcctgacctaaagtgatccactcgccttggcctcccaaagtgctaggattacagccctcattctcttttgctcctcaggtgacacaggacaagatcatctgtctacccaatcatgagctccaggagaacttatcagaggccccgtgccagcaattgctgcctcgggggatccctgagcagattggggccctgcaggaggttaaaggccttaagaacaatttggacctgcagcaatacagctttattaaccagctgtgttatgagacggccctgcactggtatgccaagtacttcccttacctcgtggtcattcacacactcatcttcatggtctgcaccagtttctggttcaagttccctggcaccagctccaagattgaacacttcatctccatcctgggcaagtgtttcgactctccatggaccaccagggccctatccgaggtctccggggagaaccagaagggcccagcagccaccgaacgggctgcggccaccatagtggccatggcagggaccgggccggggaaggcaggggagggtgagaaggagaaagtgctggcggaaccggagaaggtggtgaccgagcctccagttgtcaccctgttggacaagaaggagggtgagcaagccaaagccctgtttgagaaggtgaagaagttccgcatgcacgtggaagagggcgacatcctgtacaccatgtacatccgacagacggtgctgaaagtgtgtaagttcctggccatcctggtctacaacctggtctatgtggagaagatcagtttcctggtggcctgtagggtggagacgtcagaggtcacgggctacgccagcttctgctgcaaccacaccaaggcccacctcttctccaagctggccttctgttacatctcctttgtgtgcatctacggacttacctgcatctacacgctctactggctcttccaccggcccctcaaggagtactccttccgttccgtgcgggaggagactggcatgggggacattcctgacgtcaagaatgacttcgccttcatgctgcacctcatcgatcagtacgactccctctactccaagcgcttcgccgtcttcctgtccgaggtcagcgaaagccgtctaaagcagctcaatctcaaccacgagtggacgcccgagaagcttcgacagaagctgcagcgcaatgccgcgggccggctggagctggccctctgcatgctgccgggtctgcccgacaccgtctttgagctcagtgaggtggagtcactcaggctggaggccatctgcgatatcaccttccccccggggctgtcacagctggtgcacttgcaggagctcagcttgctccactcgcccgccaggctacccttctccttgcaggtcttcctgcgggaccacctgaaggtgatgcgcgtcaaatgcgaggagctccgcgaggtgccgctttgggtgtttgggctgcggggcttggaggagctgcacctggaggggcttttcccccaggagctagctcgggcagccaccctggagagcctccgggagctgaagcagctcaaggtgttgtccctccggagcaacgccgggaaggtgccagccagtgtgaccgacgttgctggccacctgcagaggctcagcctgcacaacgatggggcccgtctggttgccctgaacagcctcaagaagctggcggcattgcgggagctggagctggtggcctgcgggctggagcgcatcccccatgcagtgttcagcctgggtgcgctgcaggaacttgacctcaaggacaaccacctgcgctccatcgaggaaatcctcagcttccagcactgccggaagctggtcacgctcaggctgtggcacaaccagatcgcctacgtccctgagcacgtgcggaagctcaggagcctggagcagctctacctcagctacaacaagctggagaccctgccctcccagctcggcctgtgctcaggcctccgtctgctggatgtgtcccacaatgggctacactccctgccacccgaggtgggcctcctgcagaacctacagcacctggccctctcctacaatgccctggaggccctgcccgaagagctcttcttctgccgcaagctgcggacgttgcttctgggcgacaaccagctgagccagctctcgccccacgtgggtgccctcagagccctcagccgcctggagctcaaaggcaaccgcttagaggcgctgccagaagaacttggcaactgtggggggctcaagaaggcggggctcctggtggaagacacgctttaccagggtctgccggcagaagtgcgggacaagatggaggaggaatgaagctggggtggggccgttttaggtagagccttaaaaatgcttctgccctggaatctcaaccattgtcttccaagataggaagccaagtgggtccaggccaggagatggggggggcgggggcagctgtgtcatctttctggggcccaggaggatctgggctggtttgtctggggagacagacaggatgttgtggagctggggtggaacctggtatggagggattaactcagtcatggcattctccgaccaaaaccacacctgtgtctctggcaggctggctggccttgctcccatccctagaactgctgcctctccctggatattccagctcaattagtgccacatatgggggaaacgacacatcccagtgggatttccaacactccccctccccatgcaacaaagcaacttacttctggagttctctcccaaggagaggacacagacacagttgtttgctgtgttatatgttagctccgaacaatggttctcatttggctaagcatcaaaatcacctagggagtcggtgcaaaacaaaatatcccagtcccctcccctgaaacactgactcaggaggtttggttgggggccaggagtctgttcctaaatattccaggtagttctggtgcaggtaagtggccctgagacagtatgttgggaaatgctgacgtaaaggtatcagggccgggcgctgtggctcatgactataatcccagctgtttgagaggccaatgcaggaggatggttgagctcaggagttcgagatcagcctgggtaacatagcgagaccccacctctgccaaaaaaaaaaaaaatagggtatccaaatatctagattctgatcccttttgaggtcctagaccctttgagaaactgatgaagccaggcacctccttcctcaggaaaatgctggtgtacaaatacacacaaagctcttcaggcagctgatagatttcccccagagagctattcaaggacttcctaaggtgggtggactgcagggttaggacacctgctatagaggtgacatttttccaaggacaagcagggactttggtcttgactgttctctgggtgcttaataaataatactaattatgcaagttctag

SEQ ID NO: 6 - DNA, 25036 bp, Homo sapiens
  LRRC8A_inton
tgagtggtgcagtgagggacaaacaaaaggaggcgccggagcagcgctgcggccggcggcgggacggagcggccggggcctggggctgcctgccgggcggccgggcgcggcgagcccaggtgagtggacggggtggggaaaggggcgcgagctgtcacctctcgaaacccacttacacacgccctcggccagctgcccggcccggggcgcccggggcatctggggctctgtctccgggccggccccctggggacctcccgcggtgttcggcctacctctgggctccccagtgcccggagtctccggggcaccaccttctcgccggctccgcggcgcgcgagccccctcccaggcacccctgtgcctccttgtgcttcagaaaccaggagtttccgcctcggctcccccatgtccccttgtcatcccctgggtccccccagatcctcacccctccacacacactctcccgttcctagaggttccacctctgggctctctccttgccatttctttttcaaagatttccttagctgtcttctcctgagttcctggtccccttctcttcccttgcacccctccttcctatcgttccgatgggggcagtgccctgacttgggggcaggatccccggctaggctcttggggcctttctgggatgggatatttgggaagaccggtccggaatctaagaacccagaccctgtcccagtcctgtgcattcaggtgggcccgagggcgaggcgagatccagtgaggtccaggcctggtgcagccctaggtgagctggatacccagcaaactgagctcccaggctgaaggggcatagctggggtcatggagttcatgatgattctgctgctttcgctgactgcttcttccccttgatctggaccggggtgaaccgaggtctgccggttctcagctgttgcgattgttgccttttttgggaaggagggatgggtcgggggttgtctggtagccgcctcctgatggagacttggctggaagggtgggcggttcctgggtgagccacacttccactgactcctcttcccctgccctacctcaggaagccttttgagaatgaggtgtggagggagagcacctggtaagaggaagggaactgggggagcccgaagttcccaaggacctcttccacctcttctgttagtagtactcttaacccctgccaaactgtattctgccctttggagcagacaacctggcagacagacaggctgtaagaagcgggaggagatcggctgttgctgatacttcctgtggaggggatgtgggagtgggagtttgttcagagagactgggcagtgctgattgctcagtctcactggtgttgcttccatgaaggtcaagtcttcctctggctcatgaagtgaggcaaggacagatgacctgtgctggaggaggctgttgtggacatgccagggatgagtgactgggtggctggtctctgcttccttgttttcctggtctcagggctttggcatggggtctgatgccctctgcgttctagggaccatagggtacagagcagggggtgcagggccgggcgtggtggctcacgcctgtaatcccagcacgttgggaggctgaagcgggcagatcacgaggtcaggagttcgagaccagcctggccaacatagtgaaaccttgtctctactaaaaatacaaaaaattagccaggcatggggctgcgcacccgtaatcccagctattcgggaggctgaggcaggagaattgcttgaacccagcaggcagaggttgcagtgagccaagatcgcgccattgcactccagcttgggcaagaagagcaaaattccatctcaaaaaaacaaaaaaaaaaacagagcgggggcacaggaaatgttcatgacttccccccagaacttgactagatgtggtggttctccatcccttaggcacacccaccaggaagccgtttcctttgcaaactcttgaatgacctttctggactgtagggaatcctggggatcactgccgtgggtcagaactcagggaagtggccggaccctgggttgctgcctggcttggcctggaccttgcctgtaggagttggagggaaatcagcctggttcctgactgcctcgtcttagagtagagctgattccaggttttcacatggagaaactgaggcacacactgatgggccaagacctcttggagattcaggtgggatggatgtggggctcttagagcccagcacagtgtctctccatgccttgactcattcattcattcattcattcattcagccattctttagaaagtgcctgtcttgggcctgggatggctgggaaaacatccctggcctcctgagagctgcagtctgggaggacaggcaaaccagaagacacataagttctgatatactgtggccaccatattaatagaaatattacagaggcagcccagaggaagaaggaattagctctgggtggttatgtagggggtggtcagggtatgctttctggaagaggagggactagaataattagtgacagggagtccaggggcccaagttctggacctggctctgccatgggcaggtgattggaccacccagcctcagtgggtgccccatccttgacaaggagggtagagtgaggtagaggttggcttggatggcctccgtgcctatatagctgtaggtactttactgcctagtcaccaccaccccgtatggaattgcagggttattgacctacctcccatgacggcggagaccatcttccatgtctgccctcccagcacagggcctggaacttagtcaatatagtaaatacccagtggtagaaatgatggaaaaacagccatccctcctagatcagtccaggtgtctttgtctacccccgccctcctccttcctgggtaactcctgctgatcctttggctctctctggtggctgggccactctcccagctcctggctccccgaaacccgtgtcgtagtgatcacagctttccctaccctgccactcccatgcacagttctgcacagtgtctggcgcatatggagtagatgctcaccagaggctggttgactgtcggagatgatgggtggatttggtgtttaagttggagtctgggcactggctggtctctgcctgacccagccaccccactgtggctcatctcttgccacttcccctctctggctctggatctcgccatgccgagcccgcaagccttctccagatgccatgtactctctcctccaggtaaacatgtgctgttcctcctgcctggaatgctctttttccagctcctactcttaggagtcaactcagacaactcctcctccagaaagccttccctgaccccactagtctgggtctagtgcttctcctggctcccccgcagcagagctctgagctctcagaccgtcgctactgacttggcacctcctcagtagaccaggttctggagggaggtgctctgtcagaacctcttttgtctgccaggcgcggtggctcatgcctgtaatcccaatactttgggaggccgaggcgggcagatccctggaggttaggagtttgagagcagcctggctaacatggggaaacgctgtctctactaaaaatacaaaaaattagccaggcttggtggtgcgcacctgaaatcccatctactcgagaggctgaggcaggagaatcacttgaatgcgggaggcggaggttgcagtgaaccgagattgcaccactatacaaaaaaaacctcttttgtcaccccagagcccagccaagggcctgctaccaagcaggctctcaggaagtgctggcggtgtggcaggtaactgcgacagtcacacatgtgcctgctgacggcagctctcgcttccttgtagggaggcagcgtccatggagcaaaaggaatgccaggatcctgcacaggcagacgcgggccagcctcagcaccgacagccgacgcgcagatagcagagccatccttggggtaaggactgggcccctctccttctcagccagctccagctccagtcaagtgcccttcccagcctggggagcaaagggtagcatctccatccccacagtcctttgcttttcttgctgtaggtccagggtagagtggcagacagcagaattactctcttcctccttctcccgctgagtaatgtgtgcttgccgcactcattgaatgctgagttgtttcattgcaaacctctgttttttctccttcaccccagatcttatcagtctgggtcttgctaatgcagcctcatgaccacatttcccaaaaagcgtggaccacgtttcccaagtgtgttccatgagacaccagtttcttcggtcacagtggtttgataattccaggaaacaaagacttcttgttatccttgctgcaggacttatcagagtcttgaatcagagccgtcatcaggattgagctgatcacgataagggagctagcgactcgtgtttccccacacttatttgatcgtgtagacatctttggggactgggattccttggcctgtgctttggaaaacagttcccagaggcaggagagtccctttagaacatttggaggattaaatatgttgacatagagcaggcagttggcagagtgcatgtcctagaaggtggtcccgagtcatagctggtgtgtgtatagacggctgtgtaatttgccagacatactcaggtcggtgatctcatttgaccatttgaggatgcttaggcagtggccattatctccagtttacagatgtggaaactgagaatcagagtgaggaagcagcttgcccagactcatctgtaaagaaggcaaggacatgcggcttgaatccagaccctggaatcctgggccagggtcttctgttgtctgccctgaggccttcctgcctttatctctcacctcagttttttgtgtgaaggaaggcctgagtctcccagcctggattttaaaagaaatagtaattttttttttttgtaattaccaacatagttgtatattcattatatggttttatactgtttggctatttttgggggacagggtctcactctgttgcccaggctggagtgcagtggcaccatcatgactcactgcagcctcgatctccaaggctccagcgatcctcctgcctcagcctcccaactagctgggactgcaggtgtgcactactgtgcccagctaattttatttatttatttattatggagatagagcctctctacattgcccaggctggtctcgaactcctgggttcaagagatgctcctgccttggcctcctaaagtgctgggattatgggtgtgagccaccatgccagacctgtttggatctttaatgtcatctaggaaatgagtctctgctgattctcatcttcagaggtgtgaatgggaaggggtatattcttccacatctattgttaccatttgcttatttttataaaaatctggtcatgtttgagttgctgattcacagtttgcttttgtttcatataatgatatcccccggacctcttttttttttgagatggagtctttgtcacccaggctggagtgcagtggcacaatctcagctcactgcaagctctgcctcccgggttcactccattctcctgcctcagcctcccgagtagctgggactacaggcgcccgccaccacgcccggctaattttttttgtatttttagtagagacggggtttcaccatgttagccaggatggtcttgatctcctgaccttgtgatccgccgccttggcctcccaaagtgctgggattataggcatgagccaccgcgcccggctcccctggacctctttcccatgcaagaggtagatagagctgtgtgtggtcctggcagaatgcacacccttgagcccccctggcccctccccctcacatgctgggagttttgagctgtttgggcatttacagcagcagagggaggtcggaagtgcctggtgtgtgattgtgtcgactctcctgtttatgggataaggtcagccaactcctggctctgccccataggagtttcctgttggttttgaggaactctgtcccctggagagaggggaggcctgagctgtagtctttcatgctgggcagcgctgagcaggaagcagcaggtcctgcttgtcagatcataattcctcctttttgccaaggatggcgtggaatgaaccgcatgggccggcctgaggggttttggcctccacccaccacctgggttcaggggacacacatgaatcaagctgagacaaattgagtctgagccattgttctcctgagcccagcccttcctggggataggaaggaacaggcagaccctgactcagtttacccttgctgtatctggaggaagtgttggttgtggtccttttggcatgctcctttcaagttttagttctgagagtgggcaggtgaggagcaggaggagggcccaccctgccacctgggtccaaagatgctctcttttggggtatagctgacctcaggaagggtttgggcaaggagagcatacaggcgtggcagcctcgttttgcggggctgcgagagcgtctggcaggatcgcgctgggagtgaggtctgtgccagagctcctgacatctcacctgtgtttttcaggctcactgtcctgtcgctttccctcccccacgtcacttcctccattcttccccaggagtctcttttctctcccatacactgcttgggagagagctgcagagctggcccctccacagatggtgctcagcgtctctggcttctccttcctgcagtagcaccttcatagtccgtggtcattctcagcaccatgtctcagtgctgagccacaggctgggatgctgcacgcatttgcccatcttaccttcgccacagccttgcgtgtgaggttgctttaccattttacagaccaagccccagagggatgaggtctctggcccaaagttatacagtgtacatgtggcaaagctaggacttgaacccaggttggtatggcttggcagctggggctctttctacttcaccctccattaccctcttttctcaggttcccaggcttttgggggccttcaaagtcacctggaagcttgtctaaaatgctgttcccctgggcccaccaccacagacctgcagagtctgccctggaaggttggccccaggaatctgaactttgaagatgcttccccagcagttgtgaggctggtgggctacaaacaatgctgagaaacactgaccggacgccattctttcagccttagtcccagtcgtctaccgcagaactcatgcccttggtgtggaagcagggctgagctccccacaggactgggactggacaggtttttttttttttgtttaattaattaattaattattttgagatggagtgttgctctgtcacccaggctggagtgcagtggcacaatcttgctcactgcaacctccacctcccaggttcaagctattctcctgcctcagcctcccgagtagctgggactacaggtgcccgccaccatgcccagctaatttttgtatttttagtagagacggggtgtcaccatattggccatggctggtctcgatctcctgaccttgtgatccacccaccttgttctccaaagttccaggactacagccgtgagccactgcgcctggccaatttttttaatttaattttacttttttgagatggaatcctgctctgttgcccagactggagtgcaatggtgccatcttggctcattgcagcctctgcctcctgggttcaagtgattctcctgtctcagcctcccaagtagctgggattacagatgtgcaccaccacgcctggctaatttttgtatttttagtagagacggggtttcaccttgttggtcaggctggtctcgaactcctgacctcaggtgatccacccacctcggcctcccaaagtgctgggattacaggtgtgagccaccgcacctggccagacagttgttttttagtggcttcattttgtgtgtgtgtgtgtgtgtgtgtgtgtgtgtgtgtgtgtgctgggaaggcgaggggcttaaccttccctcctcaaggacgtgtggggctgaggtttctagaagggcttttactcccagccacaaatgttccctgctgtcttccagatgcgtttctgttgcgtcttgcgtcttgcagcaagtttgtctacatctaaacgtctcttgagggcttaaaacctcaaaattaaattgcttgctgcaatctaagcctcacgcctagagcaatgttccagtggagtaaaagtggctgtaaaattccatagctttcacgtgccacctggacgccgtcagagtgccactgccaaacctgcgccctctgacgggcagcatgacaaacgcactgaccagcagcgggggcggtgggcgggaatcctggccctgaagggacacctcgcatctcccagctgactccatgagtcaggatccctgggggtagcccccttccatcccagagagtcgggtaccaggcagctcaccctctgtcagaccccccagtcctccccctggctcacctagggcttggccagtgcccactcccctgggatctcagcagaagatgtgtctgctgcctctctagggtactggtggccacatttcataacctgtctaagaaggcagcgctggtattcataacaggttaagacctgtaaaccgtgtgtggccgtgactccatgtgcaaatatgagccaacaaacgttggctggacgtggcggttcccaccagtaatcccagcactttgggaggctgaggtggaggattgcttgagcccaggagttccagaccagcctgggcaacatagtgacctgtctctacaaaaataaaaaaaaaattagtccaggcacagtggctcagcactttgggaggctgaggcaggtggatcacctgaggtcaggagttcaagaccagcctggccaacatggtgaaaccccgtttctactaaaaaatacaaaaatcagccgggtatggtgacatgcacctgtaatcccagctacttgggaggctgaggcaggagaatcacttgaacccgggaggcgaaggttgcagtgagccgagattgtgccactgcactttagcctggtgacagagtgagatcccgtctaaaaaaaaaaattagccaggtgtggtggcatgtgcatgtagttccagctacttagaggttgagactggaggactgcttgaacccaggagttccagaccagcctgggcaacataacaacctatctctacaaaaaatttttaaaaatagccaggtgtggtggcacatgcctgtagtttcagctactcggaggctgaggctggaggatcgcttgagccaaggagttcaaagttgcagtgagtcatgatcgcaccattgtgctccagcctcggcgacagcgcaagatcctgtctcaaaaaaaataaaacggcctgccttctaggcccttctgcccccagggcccagacccaacactgccttctggccctaaacccagggctaggctcccttccagagcttccttatagcagtacccgtgagaccagaggcatctgagtatggcctgatggggttccctgtagagccctggtttccatgtaccccctaaggaaaagagtatcttgtctcaagaagtatcttcaagggcctgggtcccaggtgggtccgggaggcctgcttgtctttgaggcaggttggcacaatggaaagtcgggcttggggtgcatcttcccagctcttcctagctgtgtggccttgggcaggttaccaaacttctctgggcctccgacatcacacccataaaaggactgatttaaactctaccataataataacagcagtgaacgttccagagccttactgtatggcaggcactagtccaaaagctttatatggactgtctccttctgtcctcatcacagccctccaagcaatagacttgtcaacccattttacagatgaggaaaccagagcccagcgaggtgaagggactttctcacggtctcgtggtgaggtgggtgccaccttgctgctatgcatgaaggttgggagatggggcaaggacatactgggcacttagttgacggtcagtaactgggcgctcctgccccagcctggcagggcgagggcaggtcctgaggctgcacctccagccccacctgctgcctctctctgcttggctccggagctgctgggggaagcccaggtgggtgtccctcacctgttgcctttcaccctagaaggcgggccacgcccttgcaagttggtgtgaggatgaacctgtttgggagcccagcccaggtgagagcagtttggaactgacccgtgctcccctccccctccagccctggcgtctcccctcttcctctccccaggaagtgagggtgtacccttctacacagggcgagtcccaagccaggcaacacccccagccagcagcctctgactcaccccagctgggtgtgggggcctccagggcagtgtccgccctggggagggtggaggctgtgtgtgccagacgaggctgagacctctcctcccactctggctctgggacaggggaagcacatgacccatggcccgtggtgggagcagatccacaggcttggaaccctcccgggacagggcctgttgagagcggccccttcgcgctccctgggagagcactggagggccagccttcccatctctggtacaggaggggtgccaacctcccgcctcccctcctgcttgggaagttttcctggctcagccttgtttgtgctttcacaaaaggctctctgtgccctgtttacccgcagcccgactgcctgccgcccaggtgtgggagccacgccaaatccctgtgggcccacatggggtctggtgcgtgctgggggggtctctctctgggacagagtgggcagcttgtcatttcttgactccctgtcttcgagaagcatccaggggatgtggactggggctgaacctaatgactggctctgagagcagatggccaactctgccacacggggtcctgagcctgggagggtctctgagtggtctgcgagttttcgaggttctgcgagtcctggggggtcctgtgggtctctgagtggtctggccctgctcagagacatggtgcttgaggctccgccctcacccaggtcagtccccactgaattctgggagggtcatctgcgatggcaggtgggaccgtggaacgacagcctgagtgtgcccggtgctgtattgacacatgatgccttacttttaattctcacgataacccatttcacagctataacccattttaggatgtaatatttccatcttgcagttgaggacacagaggctctgaggccccagtgaggcagtagtggggttggcgtttgagttgcccaactctggggtctgaactcctccctgctctgctcccaggaggtgaggaccgcaagctgctgagcgtgagtgttacgaggcagggcagggaggatgaggctgacctgtttctaatgtacggatggcagctacgcataaattcttttatcatcctgagaaaccacacagtgaggaaggtcttttgatttatttccaagcagtttttctactcataaacataccagatttaaataatggaagaatgtataatatagacagccctctacagtctcaccacgagtagacttaactatatatagttctgtgcatagctttctaaattcttttctgtacacagtaatgagtttgttattgatataattttattttttgagacagagtctcactctgtcacccaggcaggagtacagcagcacagattcggcccactgcaacctgcgcctcccgggttcaagtgattctcctgcctcagcctgctgagtagctgggactacagttgtgtgctaccacgcccagctaatttttttgtatttttagtagagacggggtttcgccatgttggccaggctggtctcaaactcctgacctcaagtaatccacccacctcggcctcccaaagtgctaggattacaggcgtgagccaccgtgcccagccatcattcttgttttaaaaaattgccaggcatggtggctcacacctgtaatcccagcactttgggaggctgaggcaggcagatcacaaggtcagtagatcgagatcatcctggttaacacgatgaaaccccgtctgtactaaaaatacaaaaaaatttgccgggcattgtggtgggtgcctatagtcccagctacttgggaggctgaggcaggagaatggcgtgaacctgggaggcagagcttgcagtgagcagagatcatgccactgcactccagcctgggcaacagagtgagactccatctcaaaaaaaaaaaaattataccagtaacagaccaggtacagtggctgacacctgtaatcccagcactttgggacaccaaggcaggtgaatcacttgaggtcaggagtttgagaccaccctggccaacatggcaaaaccccatctctagtaaaaatacaaaaattagccaggagtggtggcacacacctgtagtcccagctacttgggaggctgaggcaggagaatcgcttgaacccaggaggtagagtttgcagtgagccaagaccacaccactgtactccagccttggcaacagagtgagactctacctcaaaaaaaaaaaaaaaaagatatcatatttcctaatttttttcattgtggtaaaatacacatgacataaaatttaccaccttaaccatttgtaagtgtacacttcaatggtattaagtacattcatattgtgtatccatcactaccacccatctccagaactgttttcatcttgcaaaactgaaactctctgcacccattaaacgctcactccccatttcctgctcccccaggccctggtatcccttctgttttctgtctattcacttatgtgaataagtgaaatcaaagtatttgccttttatgactggcttctttcgcagataacacattttatatactgctcagcaaaattcttttatgcttaatatctcttagatgtctctggggtcactgcatacacatctacaaggaaggtggcgttattcctgatttaaagttgtagaagctcgggaggctgaggtaggagaatcacttgaacccgggaggtggaggttgcagtgagccaagaccgtgccactgcactccagcctgggccacggagcaaagcaagactgtctccaagataaaataaaataagttgtaaaagcagaggcttaggctaaacaatttgcctggggtcacagaattatttcttgctggagctggatttgaacctacatgtcccaaactccaagtccttgctctctctgctccctgagatccacctgtgcctgagggaaggctggggctctctgggttactcatttaacccccaggcacagagatgccctggccctggccaggaacctggtcccatttgtcctaggcacctgcttgagctgccttgtgggaagggcctgttctgcatgatgcccacttctacatctccctgggggcagggcaggaccaggtagggagtgcttacgagttctgcagggtcctcaggggctgaggagcagcagctggtaggaggagctgcctctgctgctccggtaacctggtaagatgcagtagctgctgctccccgctgagccttcgcctgtgggaggttggggagagcactggctatgcagggtaggaatgcatgagtgtttcagtgagttttgtgtgatgaaatggtttaagaaaagtataaaaactagaacagatacatccatatcaagaactcagctttgtcagacttcagtgttttgcagtaattgtttcagatacacacatgcacgcacgcacacacttgaatgtgcgcaggcacttaagtaaaatattccaagcacagttgaagtcccttccccattttcttccctccctagatacaatcattatcttggtttggagactttccatgcatgcttttgtatcttcggtatacttgggtatacaccttgtagaaaaccttatataacatatgcatcatctacaacttgttttttggatcaacattgcttttgaggtttatccacattgatacatgttgctacagttcattaactttaattgatttaaagcatttccattgtatgattagaacaacgtatcctttctcctgttgatgaacatgtgggtcatttctgattttttgctattacaaacaattcttcagtgagcatccttgtgtgtggtttctcacttatgtgtggagcagctttctaagatgcataaataatgaggagaggagttgctgggttgtagagtacgtgcatcctaccttctctaagagttcacagtttttgccaattgggggagtgaagtgatatgtgatcgttttaatttacattccttgattactagcaaggctaagaattatttcatacattggttccatttgcatttttcccctgtgaattgcctatgcgtatattttgccatttcccctcattggattgtttttgtcttatttatgcaggatttctgtatttattctggatactaatcttttcttttttctttctttctttatttttttctttgagccagagtcttgctctgtcacccatgctggagtgcagtggtacgatgtctgctcaccgcaacctccgccccctgagttcaagggattctcctgcctcagcctcctgagtagctgggactacaggcatgtgccaccaggcccagctaatttttttgtatttttagtagagatggggtttcagcatgttggccaggctggtctcaaactcctgacctcaaatgatccacctgcctcagcctccctaagttctgggattacaggcgtgagcagttgtgcccggctgtattctggatattaatcttttggtagttttaaactttggaaataactcttcctagtctgtggcttcttttcacttgtttattattatgtctttcgatgtatgctttaactttaatgtgatcaatacgttacgtttcctataaggatttatacattttgtggtaagatattcctatcttaatgacattaagacagtctctgatttttcttttctttttgagatggagtttcactcttgttgctcagactggagatcttggctcactgtagcctccacctccctggctcaagcaattctcctgcctcagcctcccgagtagctgggattacaggtgcctccaccacacctggctaattttttatatttttagtagagacaggattccgccatgttggccaggctggtctcaaactcctgaggtgatcagcccacctcagcctcctcaagtgctgggattacagacgtgagccacagcacacggcctgtcttagctattcttgatcctttgagatcgtgatttaaaaaaaaattttttttttgagacagttttattcttgttgcccaggctggggtgcaatggcgcaatctcggctcactgcagtctccgcctcctgggttcaagtgattgtcctgcctcagccttccaagtagctgggattacaggtgccctgcagcacgcctggctaattctttgtatttagtagaaatggggtttcaccatgttagtcaggctggtctcaaactcctgacctcaggtgatcctcccacctcggccttccaaagtgctgggattacaggcgtacacctccgtgcctggcaaaataatcttttttcttaattatgagtaacaaatggttataataaaaaaatagagggcaggggcagtggctcgcgacattgcactccagcttggatgacagagtgagactctgtctcaaaaaaaaaaaaaaaaagataagtttggggtgaaaaagggtaataaacagcagtctaacctcacttctaagattgttcagtgtgtgtgtgtgtgtgtgtgtgtgtgtgtgtgtgtgtgtgtgtgtgtaatttatggcagaggggttatgtactttttagatttaacatactttgaacatttcctcttgttatcaagtgttttgttctaatgcacttttgggattgcatagtattcccttatctgatcattgagtatgttttttgtttgtttgttttgtgagacggagtcttgctctgtcgcccaggctggagggcagtggtgcgatcttagctcactgcaacctccaccacctgggttcaagcaattctcctgcctcagcctcccaagtagttgatccagtagcttctttaaccagggtgtgcttattgtccacctactgcgtgccaggccccgcctgtctctttaccaggtgtcctggagccctctgatggttttccttcttccatttcccaacttaactctcgaatgcacttttcattcactcaccaggtgtctcctgaagtcagggttgctctggcagggctgggccaggcctattttgctctccacttcctgttcacatccacccgcaggctctggctggccttggcacactggatgtgttgaatgtttcaggccctggccttttagcagccctctaaggcagggcctatctttagccccattcaaagatggggtaactaaggctcagagaggtgaagtcacctgcccaacatcacaagtagcagagccagtctggccctggcccggcccttgcctccaagctcatgtaggcttgctctccactgacctgggctctcacacaggtagtttgggaagcctcatttctgtgatcttcatgatagtcctgagagcccacagggcatttgtcaccttccgccatgttaccatggggaaccaaggtgctgagaggggaaggcgtcctggctcctggatagcccctccctctctgccagctggaggccagaacagtcgggacaaggggtgtctgtgatggtggcggcagctctgcagtcacagcccagcagtctgacgcgggggtgggcagctgagggggaaggaggagtccaggctgggcagaggcctgggcgcacccaccccttggcccattttttacacaacatggacacatgtttaacaggtttgctgctcctttctatacagggaaactgaggccctgggagagaagctgttccacagctccgccaggacgcagtgccagctgagagggccgagcacagggaggataagcaagggcactttccgtaaggcagagatagcccagccacccccaccccacgcctcaaaggctcccttcccttccggggcatttctgaccacacccaggtgcaccccaagaagggctctcggtggggtctgctccatctgaatggcccaccctggaggtgctgcctgtctgggcccactgtaaatccaggtttgaacgccacctctgtcacttataggcggtatgaagttgggcagaagacttctgtctctgagccggtttccgtggttataaaatggacaccttaaaaaaaaaagccttaacaaaaggacattctcatatgtgttagaacgtggtgagccttgaggacattacgttaagtgatacagcccatcacaaaaggacaaataccttatgattccacttacatgaggtccctagagaagtcagattgacagagagaggaagtgggatggagtgcttggaggcggtgggaggaaggggtgtcgtgtttaaggggtgtgggtttcagtttaacgagatgaagagagttctgtgtatggtggtgatggttgcacaacagtgaatgtgcttcatgccactgaactacacccctgaacatgggtaagatggtacattttcagttacatgtgttttatgacagtctaaaaaaattgaggggaaaaaatggacctggaaacaatggggacctcctggctcagtgcaggggagaatctggccttggccttggttgtctgtttctgaactggaactgctctcttgtgggtatctagtcatctgcagtcctgagttcctgggcaggtccttgcactgggcccctccctgtggggattcctcagggctgggcagggaaaggaagggcccaggcttcctggctttagctgccttccctaccccagccccaacccacatccacctgaaactgctgctggcctctccaccacaccgggggcccacgccctgcagcaccccaccccaccttgtcccctttcaggtgctctctgggagagactctgagtgtatcgagtcctaacaattgactcttgtcctcaaagagaatctggcttcactaggtgctgaggaggtggaagaaagattataccagcctgggctgactgcgccccactctgcacaaagcactttaaaagcatttgggggctggacgcagtggctcatgcctgtaatcccagcactttgggaagctgaggtgggcagatcgcttgaggtcaggagtttaagaccagcctaaccaacatggtgaaaccctgtctctactaaaatacaaatattagccgggcatggtggtgggcgcctataatcctagctactcaggagactgagggaggagaatcgtttgaacccaggaggtacaggttgcagtgagctgagatcatgccactgctccagcctgagtgacagattgagactccgtctcaaaataaaaataaaaataaaagcatttagttcctaagacttgctgggtgcagtagctcatgcccgtagtcccagctacttaggaggctgaggtgggaggatcactcaagttcaggcgtttgaggccaaatgggcaacatagcaagaccccatctctcaaatttttattttaaattagtccagtgtgaggcccagcgcagtggctcacacctgtaatccccgcactgtgggaagctgaggtgggcagatcatgaggtcaggagctcgagaccagcctgaccaacatggtaaaaccctgtctctactaaaaatataaaaattatccgggcatggtggtgcacgcctgtaatcccagctactccagaggcagaggcaggagagttgattgaactcgggaggtggtggttgcattgagccgagatcgtgccactgcactccggcctgggcgatagagcaagactccatctcaaaaaaaacagaccgggcacagtggctcatgcctgtaatcccagcactttgggaggccaaggcgggtggatcatgaggtcaggagatggaaaccatcctggccaacatagtgaaaccccgtcttgactaaaatatgaaaaattagccggccgtggtggtggcgcgcctgtagtgccagctgcttgggaggctgagtcaggggaattattcgaacctgggaggcggagattgcagtgagcccagatcgtgccactgcaccccagcctggcaatagtgtgagactgcctcaaaaaaaaaatttttttttaccctagtgtggtgctacatgcctacagtccagctactctggaggccaaagcaggaggattgcttgaggccaggagtttgagaccagcctggacaacatagcaagaccccgtcccatttaattaaaataattccatggcctcctcccagcagccctgcgtggtatcatgcccaccatgtatgtgaagaaactgaggctgcaagcagtgaagagcttgtccaaggtcccacagagaggctaccagagtccaggcccacaccttggtactggcttcccctcttcatcctcggcaagagccgtgtgggagggagcctgcccctgtagggggcaggcaggtggctctgaccacccggctggctcctgttggggtggggctcctttccccacacccttgggaactcaaccctggatttggtgcctgcaccctctttcctgatctcccccgaagccccatcagagaggggcagtgacttttccagaatcacacagcagaggacaaggaaacgggtgcagctgggggtgacccctccagacaggttacccttaccaggggtaggcccaggcaccagccgggccaggcctgtgagagactccagtccaggcccttctcagctctgcagccagccccaggctctgccattggaaccccagggccctcctccactccccagacacactcaaactggagagaggaacctgaagcagaagtcccaccttgatcatttttttttacccccgctggacctagccaagggctgaacagagtccggatgttcccagaccaagcacggagttggggcgggtggtcagaatcagacctgattgggacggtgtttcctgccttgggcaggtggttttgcctctgtgagcctcagtttccctcacctgtgtgactgagctgggaacacttcctgcctcagaaggaggagtctgggtggtacagacaagccccaggtcggcggggctggggcggctcctcgcagaggaagcaggtgtttctgtgagcgaacacaccccactccctgaacacgagggaggtgttgcagtatcattgtcaccacatcctgcccgctcaaacagccgcgcccgccctggctgtctcattccagacttgggggcaggcaggctgctgcatgcaccgggctcctctcctccctcttctgttccccagtctttaggtcctggccctggatggggactccaggtgtccacatgtctcagtggaggcagctgtgtgattcagaagctctcccagtgccactttctttcccagcagtgtaggtggtgcgtggacccacctggtcattagagggcccgggccttcctgtggggcgggtgcttgccctggcccgtgagtcctggcatcactaggaggactgacttctggcctggagctgccagcccagggcgggcggtggtgtctgctggcccctttgtgacctgagcgctggtaatgctgaggggcggggaagctgcactcccagtcctgtttctcttggcaccgcctgggtgagaccttgggcaggcggagggcaagtcccacatggtggcgaggacctgagcccggtggtagagggacaggcctgctgggtttgttcttactctactctgccccttaatagcagggggatgttggcaggtgatgtcaccttcctgagccttagcacccccatctctgggatggggctatagaggcctggcctcttgggcatcaaggatgaccctaggagggcctgcaggggaggcctgcctggtgcctggcacacttccaggccctcagcactggctgtggccatcatcacccttgctgagtgccccattccacctgcctcacatggggtcctgctctgccttcctcctgtcctccccatccagcatagcctagaaagattggggctggggagtgtttggcatttgcagagaggtgctgttcttttttttttttttttttgagtcggcacctcgctctgtcacccaggctggagtgcagtggcgtgatctcggctcactgcaagctccacttcccgggttcacgccattctcctgcctcagccttccaagcagctgggactacaggcgcctgccactatgcccggctaatttttttgtatttttagtagagacggagtttcaccttgttagccaggatggtctcgatctcctgacctcgtgatccgcccgccttggcctcccaaagtgctgggattacaggcgtgagcctctgtgcctggcccgagaagtgctgttcttaaagagttcctacctaggccgggcacggtggctcacgcctgtaatcccagaactttgggaggccaaggcaggtggattggttgaggccaggaattcaagaccagcccggccaacatggcaaaacaccgtctctactaaaaatacaaaaattagctggatgtggtggcgggcgtctgtaatcccagccacttgggagattgaaacatgagaatcacttaaacccaggaggcagaggttgcagtgagtcaggatcatgccactgcaccccagcctgggtgacagagtgagactgtctcaaaaagggaaaaaaaaaaagtttgttactattctggttattaacattatcagtaatcaaagaaaggtggcccatcatctgggcacccctgttcctctcctaagagagacagacacatttgacacaggcatttaaaacttggtttagataaaaatctaggttctgggttacagtgtaaagtaaaaattgaatgtaaagtaaaagcacaatttggccaggcatggtgactcacacctataatcccagcactgtgggaggccgtggcaggtggatcacctcaggtcaggagttcgagaccagcctggccaacgtggcgaaattccgtttctactaaaaatacaaaaattagctgggcgtggtagcgtgtgcctgtaatccagctgcttgggaggcagaggcaggagaatcatgtgaacccaggaggcggaggctgtggtgagctgagattgtgccactgcactccagcctgggtgacagagcaatactctgtctctaaataaataaataaataaaagcacaatttattctaatacacagaaagggctgggtgcagtgcctcactcctgtaatgccaacactttgggaggctaaggccagaggattgcttgaggccaggagtttgaaaccaacttggccaacatagtgagatcctgtctctacaaaaaattttaaaaattagccgggcatgggccaggtatggtggttcacacctgtaatcccagtactttgggaggccaaggcgggcggatcacaaggtcaggagaacgagaccatcctggctaacacggtgaaaccccgtctctactaaaaatacaaaaaaaaattagccgggtgtggtgggcgggtgcctgtagtcccagctacttggaaggctgaggcaggagaatggcgtgaatctgggaggcggagcttgcagtgagccaagatcgcaccactgcactccagcctaggcaacagagcgagactccgtctcaaaaaaaaaaaaaaaaaaaaaaaaaattagccaggcatggtggcacacacctgtagccccagctacctgggggaggctaaggtaggaggattgcttgaccaggaggttgaggctgcagtgagccatgatcacgccaccgcactctagtttgggcaacagagcaagatccaatctcttacaagaaaaattataaaattataaaatacacagaaaggactggaaggttattattcagtgaaaagttaacagtgtgtaaccctggtggggattgtaaagcggattcttctttttttgctgctacgcttttttacaattttccgtatgttctccagggaacatctagtaccttatccctccgtgacagccatacgtctttcattgtaagagcaaccagccttggccgtgtgaccacgaatggccacagagaggcattctcacagagatcttgctgcgtgcctcctgtgtagcacagtctgggtttcctaatgtggcttctcagccagtgtttcccaagcttggccaatcattagagtcaccaaagttatgtgttaaaaatcaggttcccagccaggcacggtggctcactcctgtaatcccagcactttgggaggctgagacgggcagatcaggaggtaaagggatcgagaccatcctggtcaacatcgtgaaaccttgtctctactaaaaaacacaaaaattacctgggcatggtggtgcaggcctgtagtcccagctactcgggaggctgaggcaggagaatcacttgaaccctggaggcagaggttgcagtgagccgaaatgatgccactgcactccagcctggcgacagagcaagactccgtctcaaaaaaaaaagaaaaatcagatttccagcttctcgccatagtagtcaaactagactctctctgggagagcctgctgttgctgctgtttttaattcctttatacacacgcaaccagagatgattttacattaaggcctctctgtgatcacccagtttgtgagggaggagagccttattcttaatcccctctcttgaggccgctcccctaggagcctgtcattaagacccactatgtgacctttggtcttccgagtgttcccagaatcttgtacacacataactatacacacatgtggaattttgttgttgaagcagggtctctgtcacccaggctggagtacagcgacggggtcatagctcactgcagcttcaacctcctggactcaagcgatcctcccacttcatcctcccaaaatgctgggattacaggctaggattacaggcgtgagccaccatgccagcccccatgtggaatttttttttttttttttttttttgagttggagtttcactcttgtcgcccaggctggagtgcaatggcacaatctcagctcactgcaacctccacctcccgggttcaagtgattctcctgtctcagcctcccgaatagctgggattacaggcacccgccactacgccaggctaatttttgtatttttagtagagaccgagtttcaccatgttggccaggctggtcttgaacttctgacttcaggtgatccacccacctcggcctcccaaagtgctgggattacagacgtgaaccacgtggaatttttaaaccttgctctttgttttatggaagtgagttcatgttgtgccccctctgcatgttgctttctttcctcatattttgcgggaatccctccagccctttgctgcagctttacagcagctggttcccagtgaactgggagttttaaatgtgtccctctggtcaatctgttcattaaataggtggactttagaaaggagttcccgattgctcttactggaatgcaagggatggagcagagaaccctgaggctgcactggggatctgtctaatctcactgtgctcagtcttcctgtgtattaaatggggagcctgcctgcctcccccagggtggtttcccagccaagtggccacatgctttctgcagaagggctgataagtgggctgccccgtggagtaggcaggctttggctcccagctagatttgaggtggaattttggacaatggaagaattttcattgtcttcttcccctgacccctggtcctaggaaagccaggccaccctgtgctaacccccctcctatggctcccttttaggttgaaccatg

SEQ ID NO: 7 - DNA, 11442 bp, Homo sapiens
  LRRC8B_inton
tgtgagttttaaaatagttttttctagttctgtgaagaatttcaatggtagtttaataggaatagcattgaatctatacatatattgctttgagcaatatggacatttttaatgatacagattcttcttatccatgaccatggaatgtttttccatttcttttgtgtcatctccaatttctttgagcaatggtttgtagttctccctagttatctgtattcctaggtattttattctttttgtgccagttgtaaatgggagtttgtccctgatttggctctcaacgtgactgttgttggtgtataggaatgctagtgattttcatacattgatttcgtatctggagactttgctgaagttaccagcttaagaagcttttgggctgagactgtggggttttctagatataaaaccacgacatcacaaacagggatactttgatttcctcttttcctatttgggtgcactttctttctcttgcctgattgcccggcaaagacttcaatactatgttgaacaggagtggtgagagaggggattattgttgtgtgctggttttctttctttcttttttttttcttttttgctaaggaatgttttacttccaattatgtgatcaattttagagtttgtaccatgtggcaatggcattgagaagaatgtatattctgttgttttggttggatagttctgtagatatctatcaggtccatttgattcagtgctgagtttaggtcctgaatatctttgtcaattttctgtctcaatgatctgtctaatattgtcagtggggtgttaaagtctcctactatttttgattgggagtctaagtctctttaaggtctctaagaacttgctttatgaatctgggtgttcctatgcttatgaagcttcatttggccagatatgaaattctgggttaaaatttcttttctttaagaatgttgaatattgacccctaatgtcttctggctggtagggtttcagctgagaggttcgctattagtctgaagggcttttcattgtaggcgacctgacctttctgtctagctgcctatttaacattttgtctttcatttcaaccttagataatctgttgattatgtgtcttggggatgatctccttgtgaagtatcttactgaggttctctgcacttcctgaatttgaatgttggcctctctagctagattggggaagttttcatggatgataccctgaaatatgttttccaggttggttccattctccccatctctttcaggtaaaccaatcagctatagattcagtctttttatataatcccatatttcttggaggctttgttcattcagaaaggccatcttccaactttgagattctttcgtccacttggtctctgctgtctgcctgtcatttgaaaggtgagagtgtgattcagaagggactacatagggagtgggttttgttctacgtattatggcttgcttactgtggtgttggggggtggtcaggaaaatagtcttataaacagcaaaaggtgatggagaaggatttgaggttgccttctatttacaccttctctgtaagaattatccagcaactagtggcttatgcttgtgttaatattacaagaatatggctgcaacttcttttattttaaatagattttagaaaatatggtagcttactaaaaacttcaaagaattgagagatcttttcctaaagagaaaaatattatctacattaaatatatgatgtattctataactgccagaaataagtaaaaatgaagcaatattccttaaagtttaggataactcaaatgtcggccaggcacggtggttcacgcctgtaatcccagcactttgggaggctgaggtgggcggatcacgaggtcaggagatcgagaccatcctagctaacatggtgaaaacccatctctactaaaaatacaaaaaattagccgggcgtggtggcaggcacctgtagtcccagctactcaggaggctgaggcaggagaagggtgtgaacctgggaggcggagcttgcagtgaaccaagatcgcgccactgcactccagcctgagcacagagcaagactccgtctcaaaaaaagaaaaaaaaaaaaagaacaactcagatgtcattaggatttctagaatttttccatggttttaattcactcagtaacttttttatttaatttattacttactatttataattatttattttatttcatttattcaataactttctccaagggcttctgatgactagggtgattttcctaatgtttagagtattcatcaaattgtttcccagacccattatcttactacagttttgggtcagaagctctggaatggcagttgtatgtctctcttctcctttggtttctgtgtattattttatgatgatgtcagcttctttccttgaattctgatttttgtgttccagaaatactgtgcttctcctttacctcactgatcctgcttctctatcctttttcaatcttccttatctaataaaccttccttatctattttatagaggggacagcaattctgtccttcactcactaaattctttctctcagataattcatttctgtggcactgaccaccacttaagtgttcatgacttcctacggtgggctaaacatggctgctaaggcctagcaactctgcctatcaagaggtggagttgatatccccacccttggaatctggctggctgtggtaggttttttgatcaatagaatgtggcagaaatggtgtggagtaatttccaaggctgggccttaagagacttgtagcatctgcttttgtgtgcttgaaacattcctcttagatcttagtctcttgctatgaggaagcccaattagccatatagaggactggcaaggcccctggtcgacagccgaagctgggctcccagccagcaatcagtagaagcaccagtgttgtgacttgaggcactttgggccttctgagtattctagcattccagccctcaccatgtgaactgccagatcatcccatagaatcatgagaaattatgaattgttactttagccactaagttttgatgtgacttgttacacagaataaagaatcaaaacaaaaattggtacctagaagtgagatgctggcataacaaaaatctggcattttggctttgagaccaggtgtcagtgaaggcctgaagggcagttagaatactattgctattggaggatgtcaaatggtacccttgttttatattgagaaaaaagtcagcaagaccatgacctacagtaacatggaacatagaaaatgaacctaatgaacttgtggatctagctcaggagattttccagcaaaatatcagaagctctgagtgacccctttaactccttttttaactcccctttttttttgatactgtgatcaaagtatttcaagaaagagactaattaaagaagaaactattttaattttaagcagactttagaggaagtatttccaatcctgaattttctcagattgtaaatccgactgtttctcatccccagctcctctacccagcaaaagaccagttcctctacccagcaaaagatactcaaagaaagaagtggcctcaggtccaaagatcagatccagaacattactgccagtaaaacttgggctcaaagtgaaaataaaagcaaaggcatagctgtaagacccttcgttaaaacgtcaaaaagattgaaggcagtgcctcatagaacctctcagctagacggaagggcttctaataatttcaagggtcttgtcccacagcattctggaactgatttagatgacaagatcctggacttcaagcctgaatttgatgccataaggggatgaagtttttggggggcttgggaaagggtttagtgtgctttccatgtaagaggaatgtaaatcatttctggccagagagtagactgtggtggactaagcatggctgcagactctttgccactcctcctattgaaagatagagtccacttcccttccccttgaatctggactggccttgtgactcctttggccagtagattttggtggatgtgtcactgtgtagcttcaagcctatgccttaaaagagctgtagctttcacttttatgcttggaatgttgtcttagaacccacccaccatgctatgtgaggacactcgaagagccacatggagtctcacatgaaggagttaccagccagcagttagcaccaactaccagcctaaggaatgaggacattttgtaccttccaaccatctggaaggtgtgccagccaacactatgaagtaaaaaaacctcccacagaatctcaagaaataaaaccttttcttaagccattaaggcttggaatatgcagaacagttagcaaaaactcttccatctctattgttgctgagttcttgcctcttcctaggaaaggctctatcaccctgaatacaccctaaaagtttgaatggttaggggatctttttcatttttgtacatttaattattacataaatatttattgtgccccatgtgctggaaactatgtaagatactatcatatcataggatcaaaatctgaatccacttgactcctgcctctccgtggacctctttatctgatgttgctaggtggtactgaattttaatttcccttgactattttattctttgctttctttttaaacatattgtaaactttagccttattattctattataaagtattgagtttaagagaaaatgtattcccattcccgtgcctgacaaagacatgttttaaagtacacattaaacaaaagaaaactgcttttcaattactaaatgcagtgctcagtaggacagaaaacgtactgcttctcttaaatgctagacctttaaaacatttgctggatgacagcactattgaaatagcctttcagctgctccctttgtttctaatccctctcccataatccatcctgcatctctccatagctcagtgattcactgttctactgcttcacgttcctgagcctctaaaggattcatgtggagatggtagctgttactttgtagtctgtagaggggagagagcatcctagaagtctagtgcaacttccaagaagacttaaaagacaggcacttttctcagacagtttattgtctatcttgtacgctttctcctcctggggagatttgggggaaaaaaaaaatctggcctctcctagaccaattgacttgctctttgggagtaaggcctgggtttacgtttcttttaaaagtgccccaagaggttctaaggtgaagctgagattgagaatcactgtcttaggtttgtgattcttgaaaagtggtccttggaccagcagcatcaacatgaactaggaatttgttagcaatgcaattcttgagacctacttcctaccttacaaattggcaactctgagggatggactaatcacttcatgttttaataagctcttggtgatttttatgtacattaatgttagagaaccactattagaaaaatctttctttctaggtctcaaaactacgactgagtttgggaagcatgtttgttatttgaaaatcactgtactaacaagatttgaaagaactacaaagggacataaggaaactagtatatcaaatgttattgccggtgatcatttaagcacttgaaatatgtttaatgcttgtcattaaatctctcaagaatggtgaaatgagttacttagcataaagaaaaggggcatattaatcagagatactgatgcattgtttcctagagagagaactcaacaaaaggcgaatttaaatttcagttagatatatgttagtcccaagaaacatcttaatgggaagtagcactaaacattttgaagggatataaggtgatttttaaatcaattttcaatgagagtagtgtagagaaccgctatttggaattgttcggctataatccctgcctggggtcagagacagatctttgaagtagcgtcctcttctcagtatataatgacactaaataccatctgatcatcatttgtttattttttcacttgttattttagaaatgctttcaaagtaaaataaggactactgggaagtaaagatgcctaacttgaatgttgatgttacagtcttagagactttggagtaagattgtcaagtgaattcatgtgttttgaaaaagcttttatgacttcttaatgaggtttaaaagctgaaataatgggattagaaccgtaaaatccattactcttaacatttctttggattgatttagtgtgtttgtatattagaaatagatattttatttggattgaatcatcattgtttactaaaatatggtctggaaacaatttcctggacagacagtattaaatggcataatgacaaaagatagctatgcttaaactctctagttaagcacttcaaataggaaaattatcatcattttatacataggaatgctgagatgttttatatatacttaaatccagatgctatatatttaaatattaagttttagttcagtagaaaatatttagtaggaaaatgtttagctgagaaagtagattatatgtaacggaagacattacagaaaactacatttgcatccttattcaaacccagttataatgcagttataacccagttataatgcagatattggatgagttgggaaagagcgctaattagctttttcttcagcttttgagagacctaagactgtggcgtagttaattggattgactgaggccatgtggtctggtgtgcatgaggttgggatttgagtatgaggacctaagttccagttgcaactttatttcttagaggttttgtcaccttgggcaaggttaattctttgggactgccttaatttctatgcatataaacataaagagtaatactatttatttcaatggcttgttttaagtataaaacaaattaatgtaaatgagacatgctttgaaggtagaaaaacagatgttaaattattaataaagtagaaagtatatttctaatatttaaggagaactgtataagaagaatcatggctgagtaacatttttgaattatgcaataaattcttttctggcatgttacaaatgcagccgctatggaagaatatgtaaaatgtttcacacatcagatgtctttatcatgctcagtgcacataaatgcagtcatgtgatatatgttgtaagacttttataaaggcttgtgaattgtgaatgagaaatcttactccagttgtgccctgtagttatgaattagtgtacagctaggttcttatacatactaactgtgctttctaacctgcaaaacaatttgatgctggatggtgactaattgtaaaataggctttgcacatgacttggtaaacccaaagtaagttttggacatgcccaaatttttgatatttgaaatgacaagcttaaaagattattgctaaaagtatcttaaatacaatttggggtcagtactcttaggaagatttgaagtactttttaaaaaggattgtataatacaaaatctccctagaaactttttttttgaggtttcataggcactaaaagatataatttttactttcaattaaaaacaaattctagcattttacaattttctcacagttcaaattatcaatatatatgtaatttaactatcacaaattattgaactataaatgttacaaatggcttttattaaactctaaattaaacaaatctaatataaagcctaagtatgtttaataataacataatttcaagtaatttcagccatatttctgagctgtatctgaaaaagtatattaacttatcactttttctaatgagattggtaggctaaattttaggagcactgagacttatttggatagatgtttggttttaaatatgggtacagaaatcacttggtgtgacaagctgtctgaaaaatgcaaataagaacgactgtgtttgaggaatgagtgagagaaattaacatatgtcgactacttagaaaagctgtgttcatctaagtgttgtagacacagaattttaaaatatgttgtgtttaaattgctgtgggaactaacctgtaattgaaaggaagttcagggagtgagtatggtttaatgaatgtgagtggggccctgtagatacacctttgccagaatgtgccccagtgtgaaaagtaaactgacctatgaaaatctaagccaggcacagtggctcacacctgtaatcccagcatttgagaggccaaggtggaaggattgcttgaggcccagagtgtgaaaatctatttgccatgtcatgtcttatgactgagagggcttctggagacagtatctccagtttgtttgtttgaatgactgtagctatttcaagatatctatgttttctcatagaatgaagacaaacacagagatggtgtgtctaagaaacttcaaaaggtgtagacctcctgactgaagcatattggatttatttaatttttttcactgtagtaagtataaatatttaaaacactttttttggtcaatgttgacttgtatttttattgaataaatattcactctgtcaaatacccttatcattgttagagctattgttgttttcattcttctacaatttcagttgcatatcttaaatgaatttaattctcaggacctttgagaggacaaatgaaacatgaatttagagctgaaagaaccctttgaaattatctgcttcactctttttatccattacaagaaattcctttgcaactctcctgatggatggtcattttatttatcaagattctgctcaggacatagagctgaactgtttcacagagcagctagttcgatgactttccaccacagattgaatcaaaatgtgattttctaaagcttccatctacctggttctaccttgggaacaaatccactattctttcacctgtgaggttgttgcgcaaaaatgtaaagataaattctcgggctcacctagtcatctattatttcagctgaatatcatcgattgcccgtgttcctctgtaggaaacggtttagaaacaacagctcctggttggttttttgcccattttatctttaacctgtaactgagcattgggtttcagtattgtgatctcacgaggcaggtacagaaggactgcagctcctttatctgggtttacctattaatatggccagatgttataatcagtatttcagcgtaacagctgtctcatgactagctgaaagtatgcatacatgtcaactaaaatcctaaactgtttttcacatgttctttctttcatccagcagtgttttctaccatccctcacacccctggccacatcatatttatagtattttgaggtctaagcccaggactttctctttgcttcatgtattcctctcattttagtttctgtcttacctcgacacacaattatgtaaggcagagaggtgaggcagaatgagaagagttgagagttagaaatagatccactaaactggagtgaaggagagaagccctgaagagctatataatccacctgtaggtcctaggaggaaggtgaatccatataatattatgtgatacccagagaggacacaagcagaaagtagtgctccagtccagcagttaagctcagaaattgtaaaatgccttaacaggtacaagagcatgaagctgtagggttcagggtcagatggtcaggaatatgctgaggatgagacttattaacagggagcagggactctgtaatgggactctaaggttcaaggcaggataccagaccccagaaagagggcaaatgagagtaaaaaagtcttcctggccaggcacagtggctcaggcctataatctcagctctttgagaggccaaggtgggcagatcatgaggtcaggagttcgagaccagcctggccaacatggtgaaaccccatctcctgtctctactaaaaagacacaaaaattagctgggcgtgatgccatgtgcctgtagtcccagctgctcgggaggctgaggcgggaggattgtttggacccaggaggtggaggtttcagtgagctgagatcacgccactagactccgtctcaaaaaaaaaaaaaaaaaaaaaaaaggcttccttggggatgctactaatactagagtgtctagagagtaactctggcccagaataaatgataagacttcagactcaagaaaatccaagagaaacccttgaaaatagattagagctggactaccagcaagggggcaccatgctaccattattatttttcttctttttcttatcagatattttctaagacccacatatttccttttactttactctgtgccatcttatagatcatctggattttactgattagcaatttttgaaacaattgatgtatgtaagactcaacatagaaattctcaggaatgttgtctgaatacttttcagtatgaaaatacaagataatcttgttttcctgaaggcaaaagtaatttaataatggaatatgcctccatcgtttatccttttagtactttgccatcacaacattaaacatgagagggagtccccatgatagaatattctagcagagcttttctttcttattcttttttaagccataacttttaaatacttcccaacttttaaatactctaagttttttcatactcatgtacttaaattacaaagcgaaagcttgtgtggaaaggagataatgcattcagtgacaggttgttctgggcatagaattgtagaccccactaagcctaaggggtaatttcacaaagcagagcttgcttaaaagaggctccttgtggtgactaaaacaactcttgggctgtaagtgatgcttcagacatgggacaaggtgtggaaaggagcttactcagacattttggaaggtaaaatgtgaccactggaaattaggtggctggctgtggtgactcatagctgtaattccagcactttcagaggccaagcagggaggatcacttgaggccaagagtctgaggcctgtgagaccctgtctcaaaaaaaaaaggaagaaaaaaagaaagaaagaaagaaaaaaatattagtagcataggaacaagtttttcaaatgtcagttgaagaaaaattggctcctggtttttagtagcctagcctggagttgagatacaagaggtaacgagataattacctcaagggctacccctaaatttctttctagtcagtatcactaaatggtcccccatctaagagactatggttcagagttggatggcctatagtttcaaatgaaaccagaagacttatgtgccccttaatcagctcttccttttaggttttaccccagagctaaatcctacttgggaaggagggcatgtgtgtatgaatattagagtaaaatgcttatttgagtacttgcttcagtagtgtttcttttatttccctcttccagtttctgtcctcctacaagggaaagtcatg

SEQ ID NO: 8 - DNA, 11491 bp, Homo sapiens
  LRRC8C_inton
ttaacaaaaacaaaaacaagtgaaaaagtgtttgacaaaaagttgctttagcttccttgtcacactttttggttgacagtttgtataagtaccagtctaccatctatttttatggaaaggtatttgaaaaacaagctacatttcttgtcagaacagaaaagacaactttctccctatgaaatcattaagagttcaactcaaaaaaacaaaaaaacaaaaacaaaaacaaaaaacctttttttgagtattctaggccccagagcaaggaatataaaataaaaggtgaccccttgccttttcagaaaactgtaaattccagaagttacaaaataaacaagaaactttagttttaatgctcttgagggcaggagtcttcttgccttccgcaccctcctctccttgctgcccttcatcagaaagaaagcagcattccccgcacatggctttgccagctcctgtcatgcctgggttttctgggctgcgctgtgtgttcatggtgtgcctgctgcttagaaccctgtgccctctttgttcaaggggtaaaactggattggaggtaaacctcagtctctttctgtacattgaaggaagagatcaatgatcttcaaaccaatcattacagatttccccagttgaatttgacttagcttttggatgttgagactgataaggtgattgaaaacctggccttaaccacagccaatgatattgagagttcaaggagaaacacacagccttttcaagggtttagaatccgattgggattgggaggaggtgtgtatccacgctacctcatggaagagtgtttctagtttaaaggagacagaaaagaaaaaaaagaaactggagtgaagaggtgactaagaaggccagtaagatgcagagagctgagccatttctgagagagactataggtatactcagaataatagatatatggaaagaaatgactgacatttgaggataatcgtttatgaacagtttttgaaggactattagatctcagcttagcctcatttcagtaaggatttctgaatttaaatgtacttgaattgtgcggaataggcattaaataattattagtatcatatacatatagctgaaactatttgacaaaaatcccaaagaaattaaagctttcttttaagatctcagtgagatttagaaaagcaatattccaaaccattattttgttaataatactttaatgtttagatttgttttctatcacctgtttcttaataggttcatgttctgacaaggtagtcacaacatttggccattttcttgggtacaagctaagacctcagaagacattactgatcaacacatcctgtttagcagtagtgacagaacattgtgcattaaaaattcaatgatatttaactttgaaattataaattattcctgacttctttcctcatctcaaacattctcttcagtagtggatgttctttgaataatcattatttacaacaaggagctatttggtaatagtctctctcttatttattgcaatgtcagggtctatctaaggggataataacgtaatgaaagagttggtccttactacaaaatgctcgattgggtgtgtctttatctgtgtgggtgataagcatggataagtcgattttagtgtaaccaatttgtattgagggctgcttctaagacactgtgctactttcttgaagatacagagatgagatataaattctatccttggtaaaataaaaatactgtaacaacgtctttaaatgcaccattggcacaggaaacaataatcaaccccatttacaaaggagtttcaattcatatttttataagaatttcttcaatgaatattctcccgtgagtgtatatgtgggtgagggattctctcagcacctagcacagagcctgacatacaattccctctcataatttatagtaagcacatgagtgaatgaaagtgaattatgcttgtgagagggaaaaagcagctactgatcatagatttttagtgaattcaaaggcctgtggctacaataatataggtaactgtggaaactgtcatgtaaagcaaaagccttgaaatgtacagcctgaagcacacacaattaaaaggatttttctttaaatatgtgaagagcataatcagaaacgaataatatcgtcattgtttgagagaactaaaattgctaggtagaagtacttttgccacacccaccaacacccttagaaaaataggttaagctcaacttgaagactttccaaataatgtaattggtccattaattgaacatacaatatcatggagatatccaaggaaaagatggaagtattcattgggttgcaagttccgcttacctcttaagattcctttgagtcaaaagttctatcattttatgatgctatgaggacagtatagcttaagaatcatttaaatgtgttaatgttcgtttattttttcttttatattgtcaaataagaaacatgaaaagagaaatttgagtacaggaaaagttctatttaaactctttgtttcttagcacagtttaacttctttcagttacagtcagcatcatgtggtattagcaaagagctctagatggaaagaaaaagatttggttaaaagttatagttcaccattagctgtgttccttgggcaagtcacctaatctctcttggctcaggattttcacccataaagtaaggggtctggactggatgatctctttgacctttttcaggtcctaaaattttataacccatttatgcttttctcatttaattaaatacatttctacagaaccatataattatgttctgcttcagtggatttttccagaaatattttgtcatgaatgccaaaactgtacaataaaacacaatctacagtcatgctctcccaagggagaaaggtataatttttatgtaattgggaaattttgcctttttgctcctgctttttatttccatcttttcactgccaaaagaaaagcaatttgttttacaactgatgttttcagcatattctgatgactttttgatttgtggagactttgggtacaactgaaaagaactcataaatttaaaatcatggcatttctgttcgttacctcttttgcccttgttcacactggctttagctatgctaatgtcctggccctgtgtgtgggatcttcctaccatgacagtgtcagacatatactttgcttttagcttcagtaataaaaattgcaaaagcaattatatcccactttcttaactaaaattactatgtattcacccatttattcatcaaacattagttgagtatctaatacatgccaggcattattttctaggtacttgtaacacatcgatgaacacaatagaactgaaaaattaagttctgccatcatgaaacttgcattcttgtggaagtagagatgcaatacatcaagaaacacaaggccgggcatggtggcccctgcctctaatcccagtactttgggaggccaaggccggtggatcacctgaggtcaggagttcaagaccagcctgaccatcatggagaaaccccgtctctactaaaaatacaaaattagctgggcatggtggtgcatacctgtaatcccagctactcgggagactgaggcaggagaatcgcatgaacctgggaggcagatattgcagtgagccaagatcgcgccattgcactccagcctgggcaacaagggagaaattctgtctaaaaaaaaaaaaaaacgaaaggaagaaacacaataaatgagggttttttgacagactgcacgtgattttatgagacaggagtcaaggatgactccaagatttgtggcctaagcaactggaaagatagacttgctatttactgaataggaaagctgtaagtggagaagggtttgctgtggggatggggaataggggactgggacaggagtaggttttggacaggccaagtttgatgtgtctagtggatatccaagaggagatgtcgagtacgtagtgggaactctgagtctggatggagatatgaatttgtgaagcgtcaagtatagttggtattgaaacattgaggccagatgcaatcacaaggggagtgaatgcattagggaagacaagaggacctaagagtaagcattgggatgttccaaacaagggggtcggggagaagaggaggaaacaacaaaggaatagccagggaagtagaaggaaaaccaaaaggctgtggtgtcctttaaaagccaagggagaaatggtcaaattacatgaagtgctgctcataaataagatgaggaatgaaaggctaggtgcagtggctcatgcctgtaatcccagcagtttgggaggccaaggtgggtggatcacttgagactaggagtttgagaccagcctggccaacatggtgaaaccccttctctacttaaaatacaaaaattagccgggcgtggtggtacatacctgtagtcccagctacccaggaggctgagacatgagaatcacttgaactcgggaggtggagattacagtgagccaagatcacgccactgcactccagccagggtgacagagccagaccctgtctccaaaaaaacaacaaaagaaaagataaggaattagaactgaccattggatttaacaacatagatgagcaatacacatgggtttcttttctgttgaattcctatttcatgtgctcaggaactattaaattgttttgtattcgttttgatcagggccattcactgggttactgacaggctacctaccatcatttgaaattggttcttgttttcttgaatgatatatgattgtgctaaaggtgatactatttacccactgtcagaaaaatgtggaataaaacaaatgttaactctacccacctgatagccacaaaacagtgttagcaatcgtgttggaatcacactagaataagtttcatattctttaggtcatggattgataaatagagcagaatacattctgggatattaattctaggcaagaaacacaaaaattatcctggggaggaggatgggaacatattattttaggaagagccatataaataaaataaactctatgtttgtacctaactttaccaatattcttttttttttttttgagatggagtctcgctctgtcacccaggctggatggaatgcagtggcgcaatctcagctcactgcaagctccacctcctgggttcacaccattctcctgtctcagcctcccgagtagctgggactacaggtactcaccactacgcccggctaattttttgtatttttagtagagacggggtttcaccgtgttagccaggatggtctcgatctcctgaccttgtgatccgcccacctcggcctcccaaagtgctgggattacaggcgtgagccaccgtgcccggccaactttaccaatattctttaccaaaatatatatctttgagaagtaaagttatgtggaatatggatctactaaaatagttttttgtaatagttttttttttttctggtctggagatggccacaggatatttgaattggtagttgataagtgccaggtgtttcatgctttcatgcttttttttttttttttttttttctgttttgagatggagcctcactctgtctcccaggctggagtgcagtagtatgatcccagctcgctgcaacctctgactcccgggttcaagcaattcttctgcctcagccacatgagtagctgtgattataggtacacaccaccacatctggctaagttttgtatttttagtagagatgggattttaccatgttggccaggctggtcttaaacctctgatctcaagtgatccacccacctcggcctcccaaaatgctgggattacaggcatgagccactgcacccagcctacaataagtattttaaataaggaaatgaatctgaatgtctgcaaaaggttaccgaagcagagatgtaataattttcatttttaaaattaggataccagaatctcttgatttaattaaaaatattacacttccaaagacattactaataagataaattaaaatcctttgtagcaagttgaaaatcaaggttggaggaaatataaaatagaaaaatgtttaactagaaataaaattatgctgattgatctttatctccctatttaaacccagtagtttattcagaagtggctcccttagcttgaaatgataactcttgaaattgtcgcaaatcacttagaaatgacagcagcaatgaaagagcaattgatcaagtgtggctgtcactttttcagaggaactgaaagttgtgaaagagaagtacaaaaaaagttttcattgagcaagcaaaaggtctatctaccttctctttagatttgtagttttttattcaagtcttcccttactcacctttttgcatcaaaaacattaaggattaatgctgtgttagatattggtttggggccttgtaatatgttaattcaataaaatctggtacatctgtgtattagtctgtcctcacgctgctaataaagacatgcctgagactgggtcatttataaagaaaaaggtttaatggactcacagttccacatggcctcacaatcatggtggaaggtgaaggaggggcaaaggcacatcttacatggtggcaggcaagagagtgtgtgcaggagaactgccctttataaaaccaccagatctcatgagacattcactttcatgagaacagcatgggaaaaacctgcccccatgatttaattacctcccaccaggtccctccgatgacacagggggattatgggagctacagttcacatgagatttgtgtggggacacagccaaaccatatcaatctgtatatatatttatatgtaggatacatgtacatatgtagacatgtagacatacatgcacatacgcatttactcaaaaaaatttaaatgcggctgggcacgcctgtaatcccagcactttgggaggccgaggtgagcagatcacgaggtcaggagatcgagaccatcctggctaacacggtgaaacctcatttctactaaaaatacaaaaaattagccaggcgtggtggcaggcgcctgcagtcccagctgctcaggaggctgaggcaggaaaatggcgtgaacccgggaggcggagcttgcagtgagccgagatcgcgccattgcactccagcctgggcgacagagcgagactctgtctcaaaaaaaagaatttaaatgcaataatagttgtgttgtagcactaaactttgccatcttgaccagctgattccctatcaagtcttatattctatcttatctagactgtgccaatattaagaagcagtagataatacttatatttcttagatgccttagtttgactttatttcccatattaaaagcatgaggtctgtgaagcaactcagacaaggacccagagaacctcacctgttgtgagtattcaggtctttgttttttcaaaggaagacttagataaaggaacagtgaccctctacctcggttctcaaagtatggttcatggaagcctggcaacctagacctttttaagggaccccatgaagtcaaaactgttttgtaatgctaagatgttttttgcctatttcactgatggtgcaaaaaccatggttagtaaggcaccttcacattactcaagatagaggcactagactgtgctgaagggattgtgttcttcactggtacacacttgaaattaaaaaaccaggttcacttgctgttcttattgaggcagtaaaaattactgttttaataactttcaacctttgactgcatgttttattaatacactatgtgatgaaatgggaagaatacacaaagcacttctgctgcattccaaagtacaatggttgtctgaagagaaagctcttgtgtaatggagttgaaagtagaatgagctactttatcacatcatactacttttacttgaataaaacaactgattattgagacttgagtatgtggcatatatatcctcaaaatgaatgaagtgatcctgacacttcagggagaacaactaaagtatttgttgacagcaataaaattcaaactttcaagtgataattagaattttggaaaacttgtgatcaccaccatgagcttgacagcttcccaagacttaaacatttttctgatgatagcagtggtgatattaatgaatgtgatttttaaatattattactaaaatgtgtcttaatattttattaggatgaaatgtgtcaacatttggaaggtctggataactcagtgatccaatgttttccaaattaccaatgcatgatgttacaaaatcatgcgttacaaaatcatgcatatgttacaaaatcatgcatgtgtaaaaagatccattcaaagtacaagatagactaatttttttttttttttaagatggagtctcgctctccaccaggctggagtgcagtggtgcgatctcggctcactgcaaccttcgcctcccaagttgaagcgattctcctgcctcagcctcccaagtagctgggattacaagtgcatgccaccacgcccagctaatttttgtatttttagtagagttggggtttcaccatgttggccaggatggtcttgatctcctgacctcatgatccaccaaccttggcctctcaaagtgctgggattacaggtgagccactgagcctggccactaattaattttaatataatgaagtttaaaaagttgtttgatagagtttccagtttccatatcacaactaagttagtcaaggtttagtatagtatcaaaaataaatatctacaatgatataaaaaggatactcgtcccttttccaaccacatgtctttgtgaggccatgtttggtccatacatttcagacaaaacaacatatcatatcacaatagattgaaatttcaggcaaaacaacatattatatcataatagattgcagaagcagatatatcttctattcagctggcttttatattaagccaggcattaaagggatttgcaaaaatttagaacaatttcattgttctactaattttgttttggaaaatatggttatttttcataatgatgtatcttgatggatgagtttactttttaataaattatatatatattacatttattggtttttcacttctagtatggtaaatatgtatagatgtaacccatgtaaatacttcagggttctcctcattgttttagagcatgaggggttcctgagaccaaaaagttagagagccactgctctatattggtgcattttatttctgaaattgttgctcacttttataaataagtccaatttcctctacgatgtaaagaaacctatggaggagacaacaaccaaatctatcaaggaacaatagactataacacaggttgtttgctaattatccctgccgctgcagctgattaggatcaccccaaatctgagttggatgagcattgcttcacgaataagcatcagaatttattcaacagacagttgttacgagcttcttgtttttcaggcagtatggcaagtaatggggatgcaaaatagagtaagacagtaccttctgaagtggaacttggaagtcaagatgaatttgtgccatctgttattgccactgagacaactgtgactatcaaacatttggggcagtgggactgtaataatttactgagaaaaaaagaggccataaagtgtcaggaaaatcaaagtttggactagtctgaaattttaatcacactagaaatatacaaagtaatgtattgagcttttgtgcagtttgcctgcaatacatgttttaaggagacacataggcagaacatgtagattctaccctgaagaaacttgtagcctagtgggtggtaacagaaaaaagttaagaaaagttgtgtttcagtaaaatgaggaaattgtattctgcatagatgtcaagtaggtaggtcagagaggagaacatcgccaggccgttgaaattttcaacactgtgttaacagattcaaaaagaagacaaattattgtataagaaagtcacaatgcagcaagacggaatagataacattgtaattgttgccaaagctgtaatcaaagaagaggctttgcttttatgtgggatgtttattgtctatctagttctttttttttttttttttttttttttgagacggagtctcactctgtcgcccagcaggccggactgcggactgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcacgttagccaggatggtcttgatctcctgacctcatgatccacccgcctcggcctcccaaagtgctgggattacaggcgtgagccaccgcgcccggccattgtctatctagttcttaattcagtattagggaaggaaggtgaaatgtttttggccctccttggccacccttattccttgccataagataaagaaaggatatttcttctcacagtaggaggatgattgccataagatacagaaagaatatttctcctcacagtagcaggatgattaacctcaagtcctgggtcacactaaaggcagtaagaatgagtaagaaaggaggaggcacatcaagaaaagaatgtacctctttttcccttttcccagagccgtgggtgcaccctattcatttatcttctgtcatttggttaaagataggagtttccagactgaactgttgtctcagcggagaaaattaggataagtttggggttcacagtgaaatgtgagaagtacaggaaacagagaaaaaaatcaagatacaagcatagagacaaaaaggaaacaaggaagaaaaattgaggcctcctcaatccaccaccatcaataagacctttcttcttaaaagacaaggaccacgtggaaacactgttttcctttcccatggaacaagcaaaatgctttataaatagggctcagtaaagaattagcacaaaagaatgctaaaattacacttaaaaatggtttaaagcaaaaaaaaaaaaaaagcttaaagcaataacccaagatggtaagataaacagcaaaaacatttcaaaggatgagctctagtctttctggcatcttcatgttctgtttaagagaaatgagggtccaggaaataactgggacttggaaagtgtctggccatgttctttctgtcccacctggagcacaaagggaaccttgtcattggacaagaaaaggcaagtacttgccttttccatgtgggtctccttatttcctcaactggtgattttattcagtggctcatcaaaacattgatgtgtcttgacagctcttgaactaattcagacctctgctgtgaggagttggagccacatttggtaacaatgcagtatgttgtactggaagataaattgatttacaagtaatctctcctttctcagaaacatg

SEQ ID NO: 9 - DNA, 11668 bp, Homo sapiens
  LRRC8D_inton
catttgcttctttatacattaaattcttatttaaataatatttcaaagccatttggcttccttcttttaataggggtactaagacctcctagtgcagataaacttttaaaggttttttttgtttgtttgttttgttttgttttgttttgttttttgagagagagagagtcttgctctgttgcccaggttggagtgcaatggcacaatctcagctcactacaacctccgcctcccaacttcaagcaattcttcttcctcagcctcctgagcagctgggattccaggcatctgccaccaagcccggctaatttttaaatttttagtagagtcatggtttcacatgttggtcaggctggtcttgaactcctgacctcaggtgatctgtcctcctcagcctcccaaagtgctgggattacaagcgtgagccactgcacccggccaacttttaaagttttaatttaggtgattatatttttctgttttaatgctagggtttcttttattgcttataacaatctcatgaatatatgttttctagcagtcaccaagctacccacatttaccctggctaacaaatgggaacaaagagcaagatttgaatccaggtctcttgggcatcggagttgtgctctttctagtatcatttgccacctcttcatataattttttttttgggggagatggagttttgctcttgttacccaggccagggtgcagtggggcgatcttggctcgctgaaacctccgcctctcaggttcaagctatgctcgtgcttcagcctcccaagtagctgggattacaggtgctcgccaccacacctggctaattttttgtatttttactagagaccgggtttcatcatgttggccaggctggtctcgagctcctgacctcaggtgatccacctgccttggcctcccaaagtgctgggattacagacatgagccactgcgtctggccaataattttgacttaaatacatctaaaagaataaataaaattaaatgccacatatttatcattagtcatttattccaggattactctatgtgcgtggtgctacatgctagttcaaaattcaacgaattggtctagtcatcttcagtaaagactaagacagacttttctcctcaatgattctttttttcttttttaaattcagattcagcaaacatctttgggggcttactttgtgccagaccctaaaccaggtgttcctaatacgttacactgtgaggtggtttttattattaagattttaccaatgaggcatgaatgttagagaggtaataatataagcaatcatttaaaaacatacttgaacttagaatgtagtatagcatgaatagttacaataggctacaatcatatctaccttcactatcctatttctgttgaaaggcagtttaggcagtaaaataaacaaaggttgagatctggtttctttattttaacagctttatggagataaaatttacataccatacaatttactcatttgaagtatataattcagtgttttttagtgtaatcacagaattgtccaacaaccatgtcttctgatttttgaactgttactgattttttttcattactactaaccatacatttgaataagaaaacaggatcataattctaaaagggttatattcaaccctgttgttttcagatttagttttgttcaaccaaagtccaataactttcccttttcagttctactacaaaaagcttccttgacttccatgagaaatgtttgcttacaggaatgttaagctgattttcatgtgtgccattgacaaggagaagctttggaatgtgaaatccacttccgcataatgtaattatttaagtcttttgcaatagatgtactataaaagagaaatcaaagaatatatgcgattaatacttactaaatatatgtttttcaagttaaaaaacaaaaatcccttgataaccatttatagccattctgagtcctgattcactaaggagatcaaacctcaagatatggataggatgcagagaaaccaggtgctgatcccttctctcattggcttaagttcacattagaaagaatcgtattgttatgatggccatacttcccaaagcaatatgtagatacagtgctattcctatcaaactaccagtgtcatttttcacagaattagaaaaaaacgattctaaaattcatatggaacccaaaaaaagccaaatcaccaaagcaatcctaaacacaaagaataaagctggagatatcacattatgtgacttcaaactatactacaagactacagtaaccaaaacagcatggtactgatacaaaagcagacacacagaccactggaacaggacagagtccacaaaattacacacatacaaccacctgatctttgtgacaaagtcgacagtaacaaacaatggggaatggactccctattcaataaatggtgctggaataactggctagccttatgcagaagattgaaactggaccccgtcctttcaccatatacaaaaactaactcacgatggattaaaggacttaaaggtaagacctaaaacaataaaaactctgaagaaaacctaaaaaatactattctggatgttggccttggcaaagaatttatgactaagtccccaaaggcaattgcaacaaaaacaaaaattgacaagtgggacctaattaaactaaagagcttctgcacagcaaattaaactgtcaacagtaaacagacagcatacagaatgggagaaaatatttgcaaactatgcatctgacaaaggtctaatatctagaatctataaggaacttaaatcagcaagcaaaaaacaatcccattaagaaatgggcaaaggacatgaacaaacacttctcaaaagaagacatacatgcagccaacaaatatatgaaaaaatgctcagcatcactaatcattagagaaatgcaaatcaaaaagtcagtgagataccctctcacaccagaatggctattattaaaaagtcaaaaaacaccagatgctggcaaggttgcagagaaaagaaaacacttacgcactgctggtggggatgtaaattagttcagccactgtggaaagcagtgtgtagacttctcaaaaaagttaaaacagaactataattcaacccagcaatctctattgtgtatatacccaaaggaaaacaaatcattctacaaaaaagatgtgtacttgtatgttcattgcagcactattcacaatagcaaagacatgtaatcaaccaagatgcccgttgacagtggactggattttttttttaatatggtacatatacaccatggaatactatgcaaccatagaaaagaatatatcatgttttttgcagcaacatggatgcagctgcaggccattatcctaggcatagtaatacagaaacataaaaccaaataccacatgttctcacttataagtgggagctaaacactgaatacacatggatacaaagaagggaacaatagacacaggggcctgcttaataggggagagaaaggaggggagttgggttggaaggctccctattaggtactatactcattaccttgattgggggtccccaacccctgggccacggactgctgctgattcatggcctgttaggaaccgggtcgcacagcaccaggtgagcagtgggagagtgagcaaagcttcatctgtatttacagccactccccattgttgacattactgccttagctctgcctcttgtcagatcagcagcagcattagattctcgtaggagtgtgaaccctattgtgaactgtgaattcaagggatctaggttctgctatccttgtgagaatctaatgtccgatgatttgtcactgtcttccatcactcccagatgggaccgtctagttgcaggaaaagctcagggatcgcgctgattccacattatggtgagttgtataattatttcattatatattacagtgtaataataacagaaataaagtgcataacaaatgtgatgcacttgagttagcccgaaaccatccccctcccccgtccatggaaaaattgtcttccatgaaaccagtccttggtgccaaaatggttggggactgctgaccttggtgatgggatcattcatacatcaagccttagcaactagtttacccatgttacaaacctgcacatgtacaccctgaacctaaaataagtcactagaggatcataatgtacttaatatactatattatataaatatgtgttaaatatgctttatacacatacacactcagtatatatgaaaagctacttttgttttcaaagtgcttgtgcatctttaattcagtcgatggttgaaatacccccctggtaggaagggcatatgtcattatccccatttttggtgggtaaaatggtataacagatgcattaattcaagtcctgtggctgacattgctggaaagtactggctttcttccttatggcctgggcactttcttaagcctatacttaacatccatgctttggtttccatactttggcttacccgtgagatgtgatatctgtacaggggtataccagaccactaactgggatagcattaaagttatttgtagtttctctaacgcatcatttatctcatgctatgcctttgcccttgctatttcttcagtcaaaattgttttccctctccccaccacctcatgtcttacccttccttcatctctcagctcaaaaccacacctctgtgaagttctcccagttactacatgcagacttgggcttgccccgttctgtgcatctgctatactttgtgttaacttcatattacttcgtattaaggtgatagcaatgcccttactattaggttggtgcaacttttgccattaaaagtagtggcaaaaactgcaatcactttcacaccaacctaatatgttgtaattgtacacttactgcttatcatcctgcattagactgggatctccttgagggcaaggatctatattagatcatctctgcattcccctccatggtgcttggcacggggctggtagctttcttgaattcctagtgaatgtcaggaagggatggtggtgattacataatgtcattgctttgtcacctctaagccaaggacaacagtgaggagttgcctccttcaatagccaacccttagccaataacacaggcattggatgatgcgtggtagagaaaaagatttagagatgaaagattctgttagcactctcacttaattagctcagagaggtcatttaagctttttgagcctcaggtttcctcatactgttgggcagtagtaatgcctagaccactgggttattatgaaaataaatagagatcttgtataggaaagtagcttgcaaaccgaaaagcactgcaaacatgagaaataaagaagaaaaggtaatctggtaggaagaacatgtgctttggccttaggtgggattttaaatccagtctctgctactaattatttaatgtccacaacttccgtgagtctatttcttcccctctcaagggtaaataacaacactttggctgcttagagtaatgttgtgaagtttaaataggattcaggcaatgttccactgtgttccacctgtgttcagctttttccacccagagctttcagatattgtatgttcctacctctgcaccttcttttctatggcagaatcagtcctttgtactccagagaaagcaggcacctactctcaggccagggaaataaatagtgaggggagaccaccagaggaagagacaggcttccctggcatgtaatcccagccagccgggatgtgccttgggacggggcggaggcgcagcctcaatgtcatgtcacagcacattcctaggacagctgtttctgcggcagcttcttttaccatttcctttgaaactttttattgaagtggaagcctgagtgtacagatcacattttataaattcaacacacccatgtaaccagatcattcattcttgttgctgcctagtattctattttgtaaatataccacatcttttattctacagttgataggcatttggatagcttacaatttagagctaatacaaatggtgctgctatgagcattctcatacatgtttttggatgaatatatacccgtgtttcttacaggtatatacctagcagtggaattgcagggtcataaagtgtgcttatgctcaattttagtagataatactaaaacatttcccagaatttcccaacattgacacactcaccagaagtatttgcattccagttgctccagcatccctaccagcacttagtattatctgtcattttaattttaggaattctggtggattttataatttcatacatttggaaatagagccattctcttagcccccaaaagcttaaactaaacttttgtacattcattttcaggaatgtgctctctataaaactgggaagttgtatcagatgtaaagcccctagtacagtgcttgccacgtggaaggaaaatgacaagtgatagccactattactgtcaccattatcatcaccactaaatgccatctgtgttcttcaagggtgagagtagggcatggtggattacaaaggtgaataagacatattctctgcctacaaggagctcaagatctagtggaagagaccaagattagaaacagattttgtttttgtttttgagacgggagtcttgctctgtttcccaggctggagtgccatggcatgatctcgccgcactgcaacctccgccacccggtttcgagcaattcttcctgcctcagcctcctgagtagctgggattacaggcgcctggcaccaagccccgctaatttttgtatttttggtggagacggggtttcaccatgttggccaggctggtctcaaactcctgacctcaggtgatctgcccacctcggcctcccaaagtgctgggattatagacgtgagccaccatgcccggcctacaaacacattattattagacaacatgatagcaatttgaaggaaaaggtagtgcttgagctgggacttaataaggaaaagttcaatagaaagaaaaaagaaaaatgggctggagaaaacagcaagggcacaaaacttctggtggggggtggcagagtagacaatgtgctggaagagaggagctcatctgaggttggagtggtcatgtgctgtctgttattcagtgttaccattcttgcggggagtctggggccggagctggtgactgtgtgtgaagttacagtactctgtactctgttgctatgcccactgccactacctttgaacttttcttttcccactgggaaggtggacagcctcttctatttcctgggatcaaagcctgtcctgagtacaaggtgcttgaaaactaccattgacacttcaacagctgcaaaataacactggttgcaagaatccaacaatgtctctgctgtgtggtcctttaactgtttatttttatgctgggacctggcttagagtgttcattcttgcatagatccaggttgctttgtggtagcaattcaggttaataaaatgctcctctgcccaataccatctgtagatgttaataaatgttctcaaggaatttttaggatgctgaagatgatgatcaagatctcaaaatcatttcattgattaagtacctactatgtgtcaggcattggatacataggacaaagaaggaagtgggcatagtgcatatcttcattgaattggtaatccaatgggagatcagacaagtaaatacagtgtgataagggctgtgatgggcggaggtacagagtattgtgggaacacatggaagaaacacctcacccatactaggagatcagggaaggctttctggaaaaggctgcagaaacctgatagcctagtgaagaggagtagggaggaagtaatcaaggcagaggaaacaaagtgttggagaggctgaacacaagggagagaatggcttgttagagaaactggcagaagttactggcctggaacagagagagcctgaagagtttttaccaagggacccatggcaagatggaaaagggattaaaggaggtaaagactggcagcagaaagaccagttgtaataatcatggtgtaaaagacaaataaccaatgtcctcagtacactgactttcaaaattcacttatctctcaatctgccaagccagacatcctggcctccctagaaccagatacaattacagtcacatgtctcttcatggggataccttctgagaaatgtattgttaggcaatttcattgttgtgtaaatatcagagtgtacttacacaaagctagatggtgtgtgtgtgtatatgcatgtatgtatgtattcatatggaaaaccaaattcccaacatcattactgaatatcagtcctttcccctacttcaggagtccccgggccccaggctgcagactggtacctgtcgagagctgttaggaactgggcttcacggcagtaggtgagcggcgggagagtgagcattactgcctgagctccgttttgtgtcagatcagcagcagcattagattctcatagtagcatgaatcctatcatgaactgctcatgtgagggatctaggttgtgcacaccttgtgagaatctaatgcctgatgatccaaccaagatgaaacagtttcatcccaaaaccatctctccccccaacccccatttctggaaaaattgtcttctacaaaacttgtctgtggtgccaaaaaggttgaggaccgctgccctacttgatctgtaatgccagtgattagtgctctgtatcaggtttctatgtacttcattataaaatggagtctcactctgtcacccaggctggagtgcagtggtgtgatcttggctcactgcaacctcctcctcctgggttcaagcaatttttctgcctcagcctcccaagtagctgggattaaagcgtgaaccactatgcctggctaatttttgtatttttagtagagatggggtttcaccatattcgccaggctggtctcaaactccgcctcaaatgatccacccacctcggcctcccaaagtgctgggattataggcatgagccaccatctcgtatcttatgggaccaccatcatatatgcagtccattgttgaccaaaacaccattatgcagttcatgactctttttaaaaagtaaagaaaaatcctatttgaaagaactattacatgcaaaaaaatctattgttcctattactcatgcagatggccctttaaagccatggtccccaggtatgactgttacgttccttgccagaaataaacctgtgcaaggtgtactctctagtctctgtatgtgaagtaagagtcattctgtgcttgatgctgtgttttgtgcattctagataggaactgaagagctcaaaatgacatgaggttgagctgtttctcatttaggttagcttgctgtgattccagtagcttttcttcctgcttatccaagccattcatgaaagcaaaacccacacaagatacaggagctcagaataaaaggagttcaagtgggcgtgttatctcccacttcccctactaaggtacaaagttatttttctttttttcacttccttggtaattttttttttttttttttttttttacaaagactggttaccaggcatttcatacttaagccctgaagtctgggaatgtgatttctaacaattttatgtttggagttcttagacttgtgtttacctgcttacataagagataattttgcttacaaaactaactacagatgtgatcattgctgtagtattcattgtattttattaaaaaatattatgaacctactctgtgcagtactaggtatgtggaagggtttcggaaaaaaaaaaaaaaaaaaaaagacctggtccctgccttcagaaacttaatttaaaacagagcagaatcaaagtactaatgccacctttcttcattgtgtcatccaaggaagacacaagtaatcagtcacagcactgtttcctactgaaaccagaatcagcctcagaatcctcctcaacacagcattccagacaacccatgaatagataattcatcttcctggttgggtgtggtggctcacacctgtaatcccagcactttgggaggccaaggtgggcagatcacaaggtcaggagttcgagaccagcctggccaatatggtgaaaccctgtctctactaaaaatacaaaaattaggtgggtgtggtggtggatgcctgtagtcccagctactcgggaggctgaggcaggagaatcgcttgaaccaaggaggcagaggttgcagtgagctgagatcgcgccactgcactctagcctgggtgacagagtgaggctctgtctcaaaaataaataaataaataaaaatattaaaaattcatcttccatccttaatttaaaaaaatacatgcattcccaaaactcttgggtacttgccttgatttttattgcttggtcttttgctagtttttaaagaaataacatgatttctttctttaaaaaagacaaagcaatataaatatggaaacccaagaacctctcaagaaatgaaaatatggtcatgtggtttttcaagaagttgaaacattcagttcatgcacataattagagaagcccattcccattacttgccactagaaacactgtttttcttggttacttccaggatgattgtccttgtcctttattatttgtataaactagttccgtgctataaagctgtcagctgatagtgaataataaccacattttcaagtttcctggcaccggggcttcattcatggctgctcgggtcacctgctttggtagagatcgtatgtcttcctggtgctgacttattcagcatcaccatcattgattatttcatggccacctgagtcttgagctgtcttctcaatccacatgctttccataggactgcatcaaaggccagagtttcagctatatatattgataaatcccaagtgtgtatagtaagactcctcctaaacgctagacccttagtttcaactgttcatggacacttccaccagagtgattggagctaacgtaaacagctaacaaacacttgtggccagaggatgtgctaagagcgttacaatcattacttaatttaacctttccaacaacccttagaagttttcatccctattatacagatgggaaatacacaaggcttaaggaatcaaccattagtaagtgttagaactaagtctggaacataagactatccaaagtttaacggtatttaatggtatacacagacctttcaattttggcttatacaaaataagagtcatcccactccttgacctccaaatttcagtcttaacctttaatgagtccacaacaaatgccatcctgctgtcataccctccttcaaggtccactaagtaaccactatggacttggtgcagtctgtcataatttcttacctagaatgcaattcttactcattctttccctcttcccttgcactaggagagaaaagctgtatattccccttttagtttattttctactacatttatattggataaaaagataggacctgaatgagaaaactttagttaggagcaggcatagggtttttctcatttactcttttcatttgcatctctagaataatgtcttttgtcttcttgttttctaggaatg

SEQ ID NO: 10 - DNA, 10070 bp, Homo sapiens
  LRRC8E_1_inton
ataaacaggctgaagataaacttgactttgggggtctcaaccactttcccctccccttggcatctgggaagacattgacaactcatccccgggagactctcagagctccagaacctaccacttggggtgggttcaaccaagcctgacctctggtgctcttgaacttacacatgtcccagatcttcaagtctgctggcagaggcggcttggaaaaccactcggctgggaatccgcaggcctggggcctgacccaggacgggctacaaagatgccaccctcccccaccctagaccctcagtttctcccttgcataaatggttgaggctcagccggacaccgtgccttacgtctataatcccagcactttgggaggccgaggtgggcaggtcacctgaggtcaggtgtttgagaccagcctggctaacacggtgaaaccccatctctactaaaaatacaaaaattagtggcgtgtccctgtagtcccagctactggggaggctgaggcttgagtactgctcggacccaagaggtggaggttgcagtgagttgagattgggccactgcactccagcctgggcaacagagcgacactccatctcaaaaaaaaaaaaagattgaggccaggcacagtggctcacacctgtaatcccagcactttgggaggctgaggtgggaggattgcttggactcagaagttcaagaccagcctgggcaacatagcgagactccatctctacaaaaaatctaaaagttggctgggcatggtagcacgcacctgtggtcccagctactggggaggctgaggcaggaggatcacttgagcccaggagttcgagactactatgagctatgatcgtgccactgaactccagcctgggtgacagagcaagactctatctctctctctctctctttttttttttttcaagatagagtcttgctctgttgccaggccggagcgcagtggcacgatcttggctcacggcaacctccacctcccaggttcaagcgattctcctgcctcagcctcctgagtagctgggactacaagcatgtggcaccatacccagctaatctttgtatttttagagatggggtttcatcatgttggccaggatggtcttgatctcttgacctcgtgatccacctgcctcaacctctcaaagtgctgggattacaggcgtgatccactgtgtccgtcaggactctatgtctaaaaaaaaaaaaaggttgggccttgttttctacaaaacccctcttagggctgaaaactgctggatgtttcttgcttcttccctgcctcttagcatgggctgtgggtgtggagggtctcactctcccacccttcccccagcaggaactcatcaccacattttttggaggcaagtggcttgaggaggacctgggagggaggaggggtgaggcatgggtggatggagccagggctggtgggagccacatggtctcaggctggaaagccttccagcctggcctgccacaatgcaacaccgctcttaggtgcttttggcaaagatgctgtggtttttttgttgttgtttgttttttgagatagagtcttgctctgttgcccaggctggattgcagtggcgcaatcacggctcactgcaacttccacctcctgggttcaagagattctcctgcttcagccgggcacggtggcgcacgcctgtaatcccagctctttgggaggccgaggcaggcagatcatgagatcaggagattgagaccatcctggctaatacagtgaaaccccgtctctactaaaattacaaaaaattagccgggcgaggtggcgggcgcctgtagtcccagctactcgggaggctgaagcaggagaatggtgtgaacccgggaggcggagcttgcagtgagccgagatcacaccactgcactccagcctgggcgacagagtgagactctgtctcaaaaaaaaaagagattctcctgcttcagcttccccattagctgggattataggtgcccaccaccactccagctaattttgtatttttagtagacatggggttttaccatgttgggctagcctggtctcaaactcctgacctcaaatgatctgcctaccttggcctcccaaagtgttgggattacaggcgtgagccaccgtgcctggcaagatgctgcattttaattagaggctgtctcccaagagggaacagagcagttgcaatcctacaggcaggacctggaggttggatgggtcaggcttcgaatgccgcttggaactgtagaagcttgagcaagttatttctctcccctgagtctggaaagcaaagtctccgacatccacgctgcctgggttgggataaaatgaggcattcagtgtctggtggagaaagctgagcgtatacgtcccatcgttttattttccctggctctgccccaattcttcgagcccctgttcctggaccttcccaccagccctgctcagacaaagtgggggcctattttatgtctgtggaaatttaatggcaggtttgtgaggatcaagtttgagtcatggagacagataatgatagcccctgtggctgccccccggtcccctgagtgttgctgccttcttgtctgcgattgtgcacctgggatggctattggtggagcttacaccagggtggcaaggacaggaagttggtccctgctcgggtgtgtgtgtgtgtgtgtgtgtttgtgtgtgtgtgtgtgtgttgtgtgatttcttctgcagcctccagagtcaggagtttttgttttgttttgtttttttgagacggagtctcgctctgtcgcccaggctggagtgcagtggcgagatcttggctcactgcagcttctgtctcccgagtttaagcaattctcctgcctcactctcctgagtagctgggattacaggcatccaccaccatgcctggctaattgtttttgtatttttagtagagacgaggtttcaccatgttggccaggctggtctggaactcctgacctcaaattatccgcctaccttggcctcccaaagtgctgggattacaggcgagagccaccgcgcccggccccagtcagtagtgttttgcttgctgtgtttgagatagggtcttgcccaggctggttcatggctcactgcagcctctaactcctgggttcaagcaatcctcccacctcagtcttccaagtagctgggactacaggcatgcaccaccacacctggcaagattttagattttttttttttttttgagacggagtctcgctctgtcgcccaggctggagtacagttgtgcaatcttggctcactgcaggctctgcctcccaggttcacgccattctcctgcctcagcctcccaagtagctgggactacaggcgtctgccactatgcccggctaattttttgtatttttagtagagacggggtttcgccgcattagccaggatggtctcaatctcctgacctcgtgatccacccacctcggcctcccaaagtgctgggattacaggcgtgagccaccgcacccggcccaaatttttagatttttttgtagaaatggggtctgactatgttgtcctggctgctcaaactcctgggctcaagcaatcctcctgcctcagcctcccaaagtgctggaattacaggcctgagccactgcacccagcctggggtcagcattcttaggtaaagcaactgagcagtgtccctgcaggatccttggaggactcaatggatggccggaaccaaaacaggacgtgagggtccctggtgctgggtccctccgaagctcagtgtgtgtacttggactttataccctcgtgcacaaatatgcatgacccaaaatgaccccaagcgtccattttggtgggtgcaatgtacatattcttcccctggggacagttgagctatcctggtatgtttctaccagattacatactggtgacccaggcagactgggatatttgtagtcgggtttcttttctcttctcttctcttttttagagacagggtcttgctctgtcgcccaggctggaatgcagtagtgcaatcttggctgactgcaacctccgcctcccgggttcaaggggttttcgtgcctcagcctcccgagtaaccgggactacaggcaccctccaccatgactggctgttttgtatttttatttttattttgttttagttttgagacagagtcttgcactattgtctgggctagagtgcagtggcgcaatctcagctcactgcaacctctgcctcccaggttcaagcgatcctcctgcctcagcctccctagtagctgggattacaggcacctgccaccacgcccagctaattttttgtatttttagtagagacggggtttcatcacgttggccaggctggtccctaactcctgaccttgtgatttgcctgcctcggcctcccaaagtgctgggattacaggcgtgagccaccgcacctggctaatttttgtatttttaatagagacggagtttcaccatgttggccaggctggtctcgaactcctaacctcaagtgatctgcccacctcggcctcccaaagtatacttggctttttgcttagggcagggagtgtgaatgagcatgagggccaggccatggtgcgaaggaccctgtgctctgggcacccccttgcatggatctgggtctgggtgagtcactgcctgagtcagggaacccatgacttttttttttttttttcccgagacagagtctcgctccgtctcccaggctagattgcagtggcacgatctcagctcaccacaacctccgcttcccgggttcaagcgattctcctgcctagctgggactacaggttcatgccaccatgcccggctaatttttgtatttttagaagagacggggtttcaccatgctggccaggctggtttcgaactcctgaccttgtgatccgcctgccttggcctcccaaagtgctgggattacagacgtgagccactgcgcctggcccaacctatgacattttacacttgcatggtgagtccccgtgtgggcaggctgtacccacacccttgcctgcctatctgattccccagactgaagtgggggcctctgagggggtaaaaggatgtggtctttcgggactgcttggcctttagcttggtgggtggagcctgtatcctaccttgttttaacccaggagtcaccccaaaatatgggggggacacaaactgaaaagggaggctcagacatgtcagaaccattggctgagtcgtgggtcaggggaccagggtttccctagattcttcctggggagatggatggggagccagagataactccctgatgggcagatgccctccttcttggcccaactcctgtcccgttacagattatctttgtcttttgttttgtttgtttgagatggagtctcactctgtcacccaggctggagtgcagtggcacggtctcacttcactgccacctctgcctccccggatcaagaccctcccacctcagcctccagagtagctaggattacaggtgtgtgtcaccacacctggttaattttttgtgtatttttagtagactctgggtttcaccatgttggcctcactggtctcgaactcctaacctcaagtgatccacctgcctcagcttcccatagtgctgggattacaggcatgagccaccgcgcccagccccagttacagattatctatctggacaaggacaggaggctcccaggagtgggggaacttggatgttggcagaagtgggctggtctaaggttccccaccttccttgttccacctccagcctctggggaagtaggtacctgcctgggagagtgacatctcagctcatgccccacgccccagtgtgatttgtggctggcctgtctgtatctgtaatagtgatagcttggtctaaaaggagaccctgggaacagacagagtgggccggccaggagcctgacagcacagccctagcctggtcgcagatgtcacctgacaactttccatttgcaaatcctccttcctcctcggacatccggacaccccattgccccctctcagagcccaggtctctggtttctgcctgtgattctgggctgtagcccctttagaaactcaggtgtcctggtcacctcctacccccagggctgactagccttcagcttggcagtccctgcccctggggccctagtaaccagagccaggctccagccggcttttccggttagaggttggaggtggtgcccagagtgcccctggagggggtggaggggcctacgccccggaagtcctgagccttggaacgctttcaggcctggtggcggtgggagccggggctggcccagacgctgagtgtctgcagtcagggagcatcgaggcagatgttttcaggccttggtgtcaggggtgggagagggatgcctcgttttggggagggggccacccgaggaggctggagggcggcggccctgtggactatccctcatgtgctcttcgaggtcagacaagtcagatcaggtgcaggggtgcccagaggggaacagtggccgcttggttctgggcaggtggtgacgtctgcatggagggtgactaaggccaggctaagagggaagtgggggcacacactttggtggtttggacaagtttgggcagggagggtcacaggcctgcctgtgtccggctcctcggaggaccccctgcagagcctcccatcctgagggtctctctctacaccccccgtctcgtcccgtcccacaggcagcatgatcccagtggccgagttcaagcagttcacggaacagcagcctgcgttcaaggtgctcaaaccctggtgggacgtgctggccgagtacctcaccgtggccatgctcatgattggggtctttggctgcaccctccaggtgaggccctcccctggcaagggggtgtgaccagaggggcggggcaggtgtctggggaagtcgggaagccttctcatcacccaagaaagagaggaaactgaagacagagccccactcaaaggccaatccagaccccttatcttccttacctccatagccaggagcaccggagtccccacattgctatgccattccatatctttttttcttttttgagacggagtctcactctgtcacccaggctagagtacagtggcatgatctcagctccctgcaacatctgcctcccgggtacaagcggttctcctgcctcagcctcctgagtagctgggattacaggcgcccaccaccacgcctggctaattttggtagttttttagtagagatggggtttcaccatgttagccaggctggtctcgaactcctgacctcaagtgacctgcctgcctgggcctcccaaagtgttgggattacaggcctgaggcactgcacctgaccctttctttttttttcttttttttttttttctttttggctaaaaagagacaggggccgggcatggtggctcacacctgtaatcccagcactttgggaggccaaggtgggagtatcacttaaggccagaagtttgagaccagcctggccaacacggtgaaaacccgtccttactaaagacacaaatattagccaggcatggtggtggacacctgtaatcccagctacttggaaggctgaggcaggaaaattgcttgaacctgggaggtggaggttgcagtgagccgagatcatgccactgcactccagcctgggtgacagaacgagactccatcttaaaaaaaataaaataaaataattgaaaattaaaataaagaaacacggtctcattctgtcatccaggttggagtgcagtggcatgatcacagctcactgcagccttgaactcctggactcaagtgatcttcccgcctcagcctccagagtagctgggattacaggcacacaccaccacgtccagctaatttttaaatttcttgttgacacaggggtcttgctatgttgcccaggcttgtctcggactccgggcctcaagccatcctcccacctcggcctcccaaagtgctgggattacaggtgtgagccactgtgtccagccccatattttcacaatgctcagttttatccctagaatctggaaaggactgcagttgctatatgagtttgttacttctgttgtaacaaagtagcagggattgagtggcttaaataaccagaaatgtttcctcttctgggtctggttctggaggccagaagtctgagatcaaggcgtcagttgtcagtgagggagaatcttccaagcttccctcctagatttttgcagctccaagagttccttggcttgtagatgacattctcccagtatttttctttttctttttcttttttttttagacagagtctccctctgtcgcccaggctggcatgcagtggtgcgatctcggctcactgcaacctccgcctcccgggttccaacaattctcctggcttagcctcccaagtagctgggattacaggcgtgtgccaccacgcccggctaatatttttttgtatttttagtagagacgtggtttcagtatgttggccaggctggtcttgaactcctgacctcgtgatcaggatcagcctgcctctgtctcccaaagtgctgggattacaggcatgagccactgcgcctggtcgagacagttttttttttttttttttaagcaagctctgtcacctaggctggagtgcagtggcgcaataatagctcactgtaaccttgtactcctggcctcaagcaatcctcctctaccacccccgcttggccccccctcccactgcctcccaaatcactgggattacaagcctgagccaccgcacccagcctctccctgtatcctcacgtcatcttctgtctttgtgtctgtctctgtgtctatgtttcccctttctctaaggcgcagtcatactgggcccaccctactggctttgtcttgactacctctgcaaagaccctattaccaaacaggagccgcctgacccagctcaaataataccacattctaaggattcggagttgggacttcaacatcttttgtggggacacgattcaacccagctccttttctcagacaaagaccagtgctttgtcagttgtggtggctcattcctgtaatcccagcactttgggaagctgaggcaggggatcacttgaggccaggagttcgagagcagcctggccaacatggtgaaaccccatctctactaaaaatacaaaaattagctaggcgtggtagtgcgtgcttgtagccccggctactcagcaggctgaggcaggagaattgcttgaaccaggaggggaaggttgcaatgagccaagattgcaccactgcacacagcctgacagagcgagactcttgtctcaaaaaaaaaaaccaaaaaaaaaaaaaacagtgcttagaggtttagcaaaactcttccaaaatcacagctggctggcaacagtcagagatggggggtgcggggggtctcccaccttcagcctgctttttccccatccccctcattttctctcttttttaaattaatttatttttgagacagaggctcactctgtcacccaggctggagtacaatggcgcgatctcggctcactgcaacctccatcttccgggttcaagcgattctcctgcctcagcctcccgagtagctgggattacaggtgcccaccaccatgcccggctaattttgtatttttagtagagacagggtttcaccacgttggccaggctggtctcgaactcctgacctaaagtgatccactcgccttggcctcccaaagtgctaggattacagccctcattctcttttgctcctcaggtgacacaggacaagatcatctgtctacccaatcatgagctccaggagaacttatcagaggccccgtgccagcaattgctgcctcgggggatccctgagcagattggggccctgcaggaggttaaaggccttaagaacaatttggacctgcagcaatacagctttattaaccagctgtgttatgagacggccctgcactggtatgccaagtacttcccttacctcgtggtcattcacacactcatcttcatg

SEQ ID NO: 11 - DNA, 7242 bp, Homo sapiens
  LRRC8E_2_inton
aagacggctgtcttcccccaaccctgctgatcgcgcggcgcctggagtagggtcgacctgggcgcgcctgcggcgaggcggggccgagtcgtccgggggcggggcggggcggctccgggtctcctgtccccgcctgccctgactcaccctcgcggcccgcccgggaactctgcgggagtcgcggcagctccgctcccagagacccaggagttgggcccagcgtccctcccggaacggtgagtaggtggctctttgcctctggacaccccacgaggtggcagggtgggggtcgggctgggagtccccgggtctggacaaggggcagagccgctccaggtgcgccggccctgtggccggggcaggccgtcccgacacaccaggagctggggtgggggtgccggccggtgcaaagggaaacctggcccacgaggtgcccgtcctgggagccaggcagacgcggccaagtctcagagtgaagataaacaggctgaagataaacttgactttgggggtctcaaccactttcccctccccttggcatctgggaagacattgacaactcatccccgggagactctcagagctccagaacctaccacttggggtgggttcaaccaagcctgacctctggtgctcttgaacttacacatgtcccagatcttcaagtctgctggcagaggcggcttggaaaaccactcggctgggaatccgcaggcctggggcctgacccaggacgggctacaaagatgccaccctcccccaccctagaccctcagtttctcccttgcataaatggttgaggctcagccggacaccgtgccttacgtctataatcccagcactttgggaggccgaggtgggcaggtcacctgaggtcaggtgtttgagaccagcctggctaacacggtgaaaccccatctctactaaaaatacaaaaattagtggcgtgtccctgtagtcccagctactggggaggctgaggcttgagtactgctcggacccaagaggtggaggttgcagtgagttgagattgggccactgcactccagcctgggcaacagagcgacactccatctcaaaaaaaaaaaaagattgaggccaggcacagtggctcacacctgtaatcccagcactttgggaggctgaggtgggaggattgcttggactcagaagttcaagaccagcctgggcaacatagcgagactccatctctacaaaaaatctaaaagttggctgggcatggtagcacgcacctgtggtcccagctactggggaggctgaggcaggaggatcacttgagcccaggagttcgagactactatgagctatgatcgtgccactgaactccagcctgggtgacagagcaagactctatctctctctctctctctttttttttttttcaagatagagtcttgctctgttgccaggccggagcgcagtggcacgatcttggctcacggcaacctccacctcccaggttcaagcgattctcctgcctcagcctcctgagtagctgggactacaagcatgtggcaccatacccagctaatctttgtatttttagagatggggtttcatcatgttggccaggatggtcttgatctcttgacctcgtgatccacctgcctcaacctctcaaagtgctgggattacaggcgtgatccactgtgtccgtcaggactctatgtctaaaaaaaaaaaaaggttgggccttgttttctacaaaacccctcttagggctgaaaactgctggatgtttcttgcttcttccctgcctcttagcatgggctgtgggtgtggagggtctcactctcccacccttcccccagcaggaactcatcaccacattttttggaggcaagtggcttgaggaggacctgggagggaggaggggtgaggcatgggtggatggagccagggctggtgggagccacatggtctcaggctggaaagccttccagcctggcctgccacaatgcaacaccgctcttaggtgcttttggcaaagatgctgtggtttttttgttgttgtttgttttttgagatagagtcttgctctgttgcccaggctggattgcagtggcgcaatcacggctcactgcaacttccacctcctgggttcaagagattctcctgcttcagccgggcacggtggcgcacgcctgtaatcccagctctttgggaggccgaggcaggcagatcatgagatcaggagattgagaccatcctggctaatacagtgaaaccccgtctctactaaaattacaaaaaattagccgggcgaggtggcgggcgcctgtagtcccagctactcgggaggctgaagcaggagaatggtgtgaacccgggaggcggagcttgcagtgagccgagatcacaccactgcactccagcctgggcgacagagtgagactctgtctcaaaaaaaaaagagattctcctgcttcagcttccccattagctgggattataggtgcccaccaccactccagctaattttgtatttttagtagacatggggttttaccatgttgggctagcctggtctcaaactcctgacctcaaatgatctgcctaccttggcctcccaaagtgttgggattacaggcgtgagccaccgtgcctggcaagatgctgcattttaattagaggctgtctcccaagagggaacagagcagttgcaatcctacaggcaggacctggaggttggatgggtcaggcttcgaatgccgcttggaactgtagaagcttgagcaagttatttctctcccctgagtctggaaagcaaagtctccgacatccacgctgcctgggttgggataaaatgaggcattcagtgtctggtggagaaagctgagcgtatacgtcccatcgttttattttccctggctctgccccaattcttcgagcccctgttcctggaccttcccaccagccctgctcagacaaagtgggggcctattttatgtctgtggaaatttaatggcaggtttgtgaggatcaagtttgagtcatggagacagataatgatagcccctgtggctgccccccggtcccctgagtgttgctgccttcttgtctgcgattgtgcacctgggatggctattggtggagcttacaccagggtggcaaggacaggaagttggtccctgctcgggtgtgtgtgtgtgtgtgtgtgtttgtgtgtgtgtgtgtgtgttgtgtgatttcttctgcagcctccagagtcaggagtttttgttttgttttgtttttttgagacggagtctcgctctgtcgcccaggctggagtgcagtggcgagatcttggctcactgcagcttctgtctcccgagtttaagcaattctcctgcctcactctcctgagtagctgggattacaggcatccaccaccatgcctggctaattgtttttgtatttttagtagagacgaggtttcaccatgttggccaggctggtctggaactcctgacctcaaattatccgcctaccttggcctcccaaagtgctgggattacaggcgagagccaccgcgcccggccccagtcagtagtgttttgcttgctgtgtttgagatagggtcttgcccaggctggttcatggctcactgcagcctctaactcctgggttcaagcaatcctcccacctcagtcttccaagtagctgggactacaggcatgcaccaccacacctggcaagattttagattttttttttttttttgagacggagtctcgctctgtcgcccaggctggagtacagttgtgcaatcttggctcactgcaggctctgcctcccaggttcacgccattctcctgcctcagcctcccaagtagctgggactacaggcgtctgccactatgcccggctaattttttgtatttttagtagagacggggtttcgccgcattagccaggatggtctcaatctcctgacctcgtgatccacccacctcggcctcccaaagtgctgggattacaggcgtgagccaccgcacccggcccaaatttttagatttttttgtagaaatggggtctgactatgttgtcctggctgctcaaactcctgggctcaagcaatcctcctgcctcagcctcccaaagtgctggaattacaggcctgagccactgcacccagcctggggtcagcattcttaggtaaagcaactgagcagtgtccctgcaggatccttggaggactcaatggatggccggaaccaaaacaggacgtgagggtccctggtgctgggtccctccgaagctcagtgtgtgtacttggactttataccctcgtgcacaaatatgcatgacccaaaatgaccccaagcgtccattttggtgggtgcaatgtacatattcttcccctggggacagttgagctatcctggtatgtttctaccagattacatactggtgacccaggcagactgggatatttgtagtcgggtttcttttctcttctcttctcttttttagagacagggtcttgctctgtcgcccaggctggaatgcagtagtgcaatcttggctgactgcaacctccgcctcccgggttcaaggggttttcgtgcctcagcctcccgagtaaccgggactacaggcaccctccaccatgactggctgttttgtatttttatttttattttgttttagttttgagacagagtcttgcactattgtctgggctagagtgcagtggcgcaatctcagctcactgcaacctctgcctcccaggttcaagcgatcctcctgcctcagcctccctagtagctgggattacaggcacctgccaccacgcccagctaattttttgtatttttagtagagacggggtttcatcacgttggccaggctggtccctaactcctgaccttgtgatttgcctgcctcggcctcccaaagtgctgggattacaggcgtgagccaccgcacctggctaatttttgtatttttaatagagacggagtttcaccatgttggccaggctggtctcgaactcctaacctcaagtgatctgcccacctcggcctcccaaagtatacttggctttttgcttagggcagggagtgtgaatgagcatgagggccaggccatggtgcgaaggaccctgtgctctgggcacccccttgcatggatctgggtctgggtgagtcactgcctgagtcagggaacccatgacttttttttttttttttcccgagacagagtctcgctccgtctcccaggctagattgcagtggcacgatctcagctcaccacaacctccgcttcccgggttcaagcgattctcctgcctagctgggactacaggttcatgccaccatgcccggctaatttttgtatttttagaagagacggggtttcaccatgctggccaggctggtttcgaactcctgaccttgtgatccgcctgccttggcctcccaaagtgctgggattacagacgtgagccactgcgcctggcccaacctatgacattttacacttgcatggtgagtccccgtgtgggcaggctgtacccacacccttgcctgcctatctgattccccagactgaagtgggggcctctgagggggtaaaaggatgtggtctttcgggactgcttggcctttagcttggtgggtggagcctgtatcctaccttgttttaacccaggagtcaccccaaaatatgggggggacacaaactgaaaagggaggctcagacatgtcagaaccattggctgagtcgtgggtcaggggaccagggtttccctagattcttcctggggagatggatggggagccagagataactccctgatgggcagatgccctccttcttggcccaactcctgtcccgttacagattatctttgtcttttgttttgtttgtttgagatggagtctcactctgtcacccaggctggagtgcagtggcacggtctcacttcactgccacctctgcctccccggatcaagaccctcccacctcagcctccagagtagctaggattacaggtgtgtgtcaccacacctggttaattttttgtgtatttttagtagactctgggtttcaccatgttggcctcactggtctcgaactcctaacctcaagtgatccacctgcctcagcttcccatagtgctgggattacaggcatgagccaccgcgcccagccccagttacagattatctatctggacaaggacaggaggctcccaggagtgggggaacttggatgttggcagaagtgggctggtctaaggttccccaccttccttgttccacctccagcctctggggaagtaggtacctgcctgggagagtgacatctcagctcatgccccacgccccagtgtgatttgtggctggcctgtctgtatctgtaatagtgatagcttggtctaaaaggagaccctgggaacagacagagtgggccggccaggagcctgacagcacagccctagcctggtcgcagatgtcacctgacaactttccatttgcaaatcctccttcctcctcggacatccggacaccccattgccccctctcagagcccaggtctctggtttctgcctgtgattctgggctgtagcccctttagaaactcaggtgtcctggtcacctcctacccccagggctgactagccttcagcttggcagtccctgcccctggggccctagtaaccagagccaggctccagccggcttttccggttagaggttggaggtggtgcccagagtgcccctggagggggtggaggggcctacgccccggaagtcctgagccttggaacgctttcaggcctggtggcggtgggagccggggctggcccagacgctgagtgtctgcagtcagggagcatcgaggcagatgttttcaggccttggtgtcaggggtgggagagggatgcctcgttttggggagggggccacccgaggaggctggagggcggcggccctgtggactatccctcatgtgctcttcgaggtcagacaagtcagatcaggtgcaggggtgcccagaggggaacagtggccgcttggttctgggcaggtggtgacgtctgcatggagggtgactaaggccaggctaagagggaagtgggggcacacactttggtggtttggacaagtttgggcagggagggtcacaggcctgcctgtgtccggctcctcggaggaccccctgcagagcctcccatcctgagggtctctctctacaccccccgtctcgtcccgtcccacaggcagcatg

(19) United States
(12) Patent Application Publication
(10) Pub. No.: US 2021/0163937 A1
(43) Pub. Date: Jun. 3, 2021

(54) ENRICHMENT OF GENOME-EDITED CELLS

(71) Applicant: B.R.A.I.N. Biotechnology Research and Information Network AG, Zwingenberg (DE)

(72) Inventors: Ümit PUL, Duisburg (DE); Torsten ERTONGUR-FAUTH, Darmstadt (DE); Sophia GRÜNER, Neu-lsenburg (DE); Janina TROTHE, Bovenden (DE)

(21) Appl. No.: 17/047,937

(22) PCT Filed: Apr. 18, 2019
(86) PCT No.: PCT/EP2019/060139

§ 371 Date: Oct. 15, 2020

(30) Foreign Application Priority Data
Apr. 18, 2018 (EP) 18168026.5

(51) Int. Cl. C12N 15/113; C12N 9/22; C12N 15/10

(52) U.S. Cl. CPC C12N 15/113; C12N 2310/20; C12N 15/102; C12N 9/22

(57) ABSTRACT

The present invention relates to a method for enriching cells within a cell population, wherein a gene of interest is genome edited, said method comprising (a) introducing into the cells within the cell population one or more nucleic acid molecules, said one or more nucleic acid molecules encoding in expressible form (i) a CRISPR nuclease, (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a′) introducing into the cells within the cell population (i) a CRISPR nuclease, and one or more nucleic acid molecules, said one or more nucleic acid molecules encoding in expressible form (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a″) introducting into the cells within the cell population (i) a ribonucleoprotein complex (RNP) comprising or consisting of a CRISPR nuclease protein in complex with a guide RNA for knocking-out leucine-rich repeat-containing protein 8 A (LRRC8A), LRRC8B, LRRC8C, LRRC8D or LRRC8E or a guide RNA for knocking-out an innexin gene, and (ii) a RNP comprising or consisting of the CRISPR nuclease in complex with a guide RNA for editing the gene of interest; and (b) treating the cells obtained after (a) or (a′) or (a″) with blasticidin or a derivative thereof, thereby (i) selecting cells wherein the LRRC8A, LRRC8B, LRRC8C, LRRC8D or LRRC8E gene or the innexin gene is knocked-out, and (ii) enriching cells, wherein the gene of interest is genome edited.

### RELATED PATENT APPLICATION

This patent application is a 35 U.S.C. 371 national phase patent application of PCT/EP2019/060139 filed on Apr. 18, 2019, entitled “ENRICHMENT OF GENOME-EDITED CELLS”, naming Ümit Pul et al. as inventors, and designated by attorney docket no. AA2586 PCT which claims priority to European Application No. 18168026.5 filed on Apr. 18, 2018, entitled “ENRICHMENT OF GENOME-EDITED CELLS,” naming Ümit Pul et al. as inventors, and designated by attorney docket no. AA2586 EP. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy is named Sequence Listing and is 47 kilobytes in size.

The present invention relates to a method for enriching cells within a cell population, wherein a gene of interest is genome edited, said method comprising (a) introducing into the cells within the cell population one or more nucleic acid molecules, said one or more nucleic acid molecules encoding in expressible form (i) a CRISPR nuclease, (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a′) introducing into the cells within the cell population (i) a CRISPR nuclease, and one or more nucleic acid molecules, said one or more nucleic acid molecules encoding in expressible form (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a″) introducting into the cells within the cell population (i) a ribonucleoprotein complex (RNP) comprising or consisting of a CRISPR nuclease protein in complex with a guide RNA for knocking-out leucine-rich repeat-containing protein 8 A (LRRC8A), LRRC8B, LRRC8C, LRRC8D or LRRC8E or a guide RNA for knocking-out an innexin gene, and (ii) a RNP comprising or consisting of the CRISPR nuclease in complex with a guide RNA for editing the gene of interest and (b) treating the cells obtained after (a) or (a′) with blasticidin or a derivative thereof, thereby (i) selecting cells wherein the LRRC8A, LRRC8B, LRRC8C, LRRC8D or LRRC8E gene or the innexin gene is knocked-out, and (ii) enriching cells, wherein the gene of interest is genome edited.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Novel genome editing technologies, such as Zn-finger, TALEN or CRISPR, enabled genome editing in a broad range of cells, model and non-model organisms^{1,2}. Especially the CRISPR-based techniques have substantially streamlined the targeted gene modification in different mammalian cells. However, depending on the cell type and the targeted DNA region within the genome, CRISPR can be inefficient, resulting in less than one percent of edited mammalian cells.

Successful genome editing with CRISPR (or other programmable nucleases) requires three sequential preconditions: (1) Efficient delivery of the CRISPR-encoding genes into the target cell (transfection/transduction efficiency); (2) efficient expression of the CRISPR-components (CRISPR nuclease and the CRISPR-RNAs); and (3) targeting of the gene of interest (GOI) by CRISPR ribonucleoprotein complexes and repair of the DNA by cell's own repair pathways.

The overall success of genome editing depends on the efficiency of each of these single steps. The frequency of successful editing events within a transfected cell population thus correlates with an efficient gene delivery (step 1), high nuclease expression and the formation of functional ribonucleoprotein complexes (step 2) and finally the introduction of double-strand DNA breaks (DSB) followed by the repair of the DSBs in the cell (step 3). Several approaches have been developed to isolate the subpopulations of cells that express the nuclease, e.g. CRISPR vectors that enable Fluorescence or Magnetic-Activated Cell Sorting (FACS and MACS). These and other available methods allow the enrichment of either transfected cells or cells that express the CRISPR nuclease³, which is necessary but not sufficient to obtain edited cells. This is due to the fact that the expression of the nuclease in a given cell does not necessarily imply the formation of functional CRISPR ribonucleoprotein complexes and the introduction of mutations at the targeted DNA site. Thus, since these FACS or MACS-assisted methods do not allow the immediate enrichment of edited cells, genome editing in particular in hard-to-transfect cells (e.g. human primary cell lines) remains still excessively difficult.

The present invention seeks to overcome this limitation by providing a method and means which enable the enrichment of CRISPR-edited cells (step 3).

Accordingly, the present invention relates in a first embodiment of a first aspect to a method for enriching cells within a cell population, wherein a gene of interest is genome edited, said method comprising (a) introducing into the cells within the cell population one or more nucleic acid molecules, said one or more nucleic acid molecules encoding in expressible form (i) a CRISPR nuclease, (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a′) introducing into the cells within the cell population (i) a CRISPR nuclease, and one or more nucleic acid molecules, said one or more nucleic acid molecules encoding in expressible form (ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and (iii) a guide RNA for editing the gene of interest; or (a″) introducting into the cells within the cell population (i) a ribonucleoprotein complex (RNP) comprising or consisting of a

CRISPR nuclease protein in complex with a guide RNA for knocking-out leucine-rich repeat-containing protein 8 A (LRRC8A), LRRC8B, LRRC8C, LRRC8D or LRRC8E or a guide RNA for knocking-out an innexin gene, and (ii) a RNP comprising or consisting of the CRISPR nuclease in complex with a guide RNA for editing the gene of interest and (b) treating the cells obtained after (a) or (a′) or (a″) with blasticidin or a derivative thereof, thereby (i) selecting cells wherein the LRRC8A, LRRC8B, LRRC8C, LRRC8D or LRRC8E gene or the innexin gene is knocked-out, and (ii) enriching cells, wherein the gene of interest is genome edited.

The method is preferably an ex vivo and/or in vitro method.

The cell is not particularly limited as long as it expresses the LRRC8A, LRRC8B, LRRC8C, LRRC8D or LRRC8E gene or, alternatively, an innexin. The sequences of the human LRRC8A, LRRC8B, LRRC8C, LRRC8D and LRRC8E gene are shown in SEQ ID NOs 1 to 5. LRRC8 genes are ubiquitously expressed in chordate cells and in particular in mammalian and human cells. In non-chordates (which include for example the model organisms Drosophila melanogaster and Caenorhabditis elegans), LRRC8 proteins are not found, but the evolutionary-related ancestors, the innexins⁴. Hence, in case a guide RNA for knocking-out the leucine-rich repeat-containing protein 8 A (LRRC8A), LRRC8B, LRRC8C, LRRC8D or LRRC8E gene is used the cells are a chordate cells, preferably mammalian cells and most preferably human cells. As will be further detailed herein below, the cell may also be derived from or based on a chordate cell, a mammalian cell or a human cell; e.g.

in the case of an established cell line and organoids. Although LRRC8 genes are preferred, due to the evolutionary relationship, also a guide RNA for knocking-out an innexin gene instead of a LRRC8 gene can be used. Innexins are transmembrane proteins that form gap junctions in invertebrates. Hence, in case a guide RNA for knocking-out an innexin gene is used the cells are invertebrate cells. The invertebrate cells may be cells from an insect, crab, lobster and their kin; snail, clam, octopus and their kin; starfish, sea-urchin and their kin; jellyfish, or worms. Preferred examples of invertebrate cells are cells from the model organism Caenorhabditis elegans or Drosophila melanogaster. In Caenorhabditis elegans the innexin genes unc-7, unc-9 and inx-3 can be found and in Drosophila melanogaster the innexin genes inx2, inx3, inx4 (zero population growth, zpg), Ogre and shaking-B can be found.

A cell population designates a group of cells. The cell population may be heterogeneous or homogenous and is preferably homogenous. A heterogeneous cell population comprises cells of different origin, e.g. from different species or sources and/or different cell-types of one species or source (e.g. body site). By contrast, a homogenous cell population only comprises cells from one species or source and preferably only cells of one cell-type or one body site.

Also the gene of interest (or target gene) is not particularly limited and designates the gene which is to be genome edited. The gene of interest is neither the LRRC8A, LRRC8B, LRRC8C, LRRC8D or LRRC8E gene nor an innexin gene.

Genome editing (also known as genome engineering) is a type of genetic engineering in which a gene of interest is inserted, deleted, modified or replaced in the genome of the cell. As will be further detailed herein below, genome editing uses the cell's own repair pathways, including the non-homologous end-joining (NHEJ) or homology directed recombination (HDR) pathway. It is preferred that genome editing uses NHEJ. Genome editing via NHEJ is illustrated in the examples. In a different embodiment, it is preferred that genome editing uses HDR. Genome editing may results in a loss-of-function mutation or a gain-of-function mutation in the genome of the cell. A loss-of-function mutation (also called inactivating mutation) results in the gene of interest having less or no function (being partially or wholly inactivated). When the allele has a complete loss of function (wholly inactivated) this is also called herein a (gene) knock-out. Genome editing of the gene of interest is preferably a knock-out. A gene knock-out may be achieved by inserting, deleting, modifying or replacing one or more nucleotides of a gene. A gain-of-function mutation (also called activating mutation) may change the gene of interest such that its effect gets stronger (enhanced activation) or even is superseded by a different (e.g. abnormal) function. A gain-of-function mutation may also introduce a new function or effect into a cell which the cell did not have before. In this context the new gene may be added to the genome of the cell (insertion) or may replace a gene within the genome. A gain-of-function mutation introducing such a new function or effect is also called gene knock-in.

Genome editing uses in accordance with the invention the nucleases of the clustered regularly interspaced short palindromic repeats (CRISPR/Cas) system (also called herein CRISPR nucleases). CRISPR nucleases (or CRISPR-Cas nucleases or Cas nucleases) are a specific type of programmable nucleases^{1,5,8}. In accordance with step (a) of the method of the invention a nucleic acid molecule encoding in expressible form the CRISPR nuclease is introduced into the cells within the cell population, whereas in accordance with step (a′) of the method of the invention the CRISPR nuclease itself (i.e. in proteinaceous form) is introduced into the cells within the cell population. Also in accordance with step (a″) the CRISPR nuclease itself (i.e. in proteinaceous form) is introduced into the cells within the cell population, however, in this case in the form of a ribonucleoprotein complex (RNP) together with a guide RNA. RNPs are assembled in vitro and can be delivered to the cell by methods known in the art, for example, electroporation or lipofection. RNPs are capable to cleave the target site with comparable efficacy as nucleic acid-based (e.g. vector-based) CRISPR nucleases (Kim et al. (2014), Genome Research 24(6):1012-1019).

Means for introducing proteins (or peptides) into living cells are known in the art and comprise but are not limited to microinjection, electroporation, lipofection (using liposomes), nanoparticle-based delivery, and protein transduction. Any one of these methods may be used in connection with step (a′).

In this regard, the CRISPR nuclease to be introduced may either be isolated from their natural environment or recombinantly produced.

A liposome used for lipofection is a small vesicle, composed of the same material as a cell membrane (i.e., normally a lipid bilayer e.g. made of phospholipids), which can be filled with one or more protein(s) (e.g. Torchilin VP. (2006), Adv Drug Deliv Rev., 58(14):1532-55). To deliver a protein into a cell, the lipid bilayer of the liposome can fuse with the lipid bilayer of the cell membrane, thereby delivering the contained protein into the cell. It is preferred that the liposomes used in accordance with invention are composed of cationic lipids. The cationic liposome strategy has been applied successfully to protein delivery (Zelphati et al. (2001). J. Biol. Chem. 276, 35103-35110). As known in the art, the exact composition and/or mixture of cationic lipids used can be altered, depending upon the protein(s) of interest and the cell type used (Feigner et al. (1994). J. Biol. Chem. 269, 2550-2561). Nanoparticle-based delivery of Cas9 ribonucleoprotein and donor DNA for the induction of homology-directed DNA repair is, for example, described in Lee et al. (2017), Nature Biomedical Engineering, 1:889-90.

Protein transduction specifies the internalisation of proteins into the cell from the external environment (Ford et al (2001), Gene Therapy, 8:1-4). This method relies on the inherent property of a small number of proteins and peptides (preferably 10 to 16 amino acids long) being able to penetrate the cell membrane. The transducing property of these molecules can be conferred upon proteins which are expressed as fusions with them and thus offer, for example, an alternative to gene therapy for the delivery of therapeutic proteins into target cells. Commonly used proteins or peptides being able to penetrate the cell membrane are, for example; the antennapedia peptide, the herpes simplex virus VP22 protein, HIV TAT protein transduction domain, peptides derived from neurotransmitters or hormones, or a 9xArg-tag.

Microinjection and electroporation are well known in the art and the skilled person knows how to perform these methods. Microinjection refers to the process of using a glass micropipette to introduce substances at a microscopic or borderline macroscopic level into a single living cell. Electroporation is a significant increase in the electrical conductivity and permeability of the cell plasma membrane caused by an externally applied electrical field. By increasing permeability, protein (or peptides or nucleic acid sequences) can be introduced into the living cell.

The CRISPR nuclease may be introduced into the cells as an active enzyme or as a proenzyme. In the latter case the CRISPR nuclease is biochemically changed within the cells (for example by a hydrolysis reaction revealing the active site, or changing the configuration to reveal the active site), so that the proenzyme becomes an active enzyme.

Means for introducing a nucleic acid molecule encoding in expressible form the CRISPR nuclease into cells will be discussed in more detail herein below.

Programmable nucleases are also known as engineered nucleases or molecular scissors. These nucleases create site-specific double-strand breaks (DSBs) at desired locations in the genome of the cell. The induced double-strand breaks are repaired through the natural DSBs repair mechanisms in the cell, in particular non-homologous end-joining (NHEJ) or homology directed recombination (HDR), resulting in targeted (i.e. site specific) mutations (“edits”) in the cell. NHEJ uses a variety of enzymes to directly join the DNA ends in a double-strand break. In contrast, in HDR, a homologous sequence is utilized as a template for the regeneration of missing DNA sequence at the break point. NHEJ is the canonical homology-independent pathway as it involves the alignment of only one to a few complementary bases at most for the re-ligation of two ends, whereas HDR uses longer stretches of sequence homology to repair DNA lesions.

The natural properties of these pathways form the very basis of nuclease-based genome editing. NHEJ is error-prone, and has been shown to cause mutations at the repair site. Thus, if one is able to create a DSB at a desired gene in multiple samples, it is very likely that mutations will be generated at that site in some of the treatments because of errors created by the NHEJ infidelity. On the other hand, the dependency of HDR on a homologous sequence to repair DSBs can be exploited by inserting a desired sequence within a sequence that is homologous to the flanking sequences of a DSB which, when used as a template by the HDR system, would lead to the creation of the desired change within the genomic region of interest. Despite the distinct mechanisms, the concept of the HDR based gene editing is in a way similar to that of homologous recombination based gene targeting. So based on these principles if one is able to create a DSB at a specific location within the genome, then the cell's own repair systems will help in creating the desired mutations.

The homologous sequence template for HDR is also referred to herein as “repair template”. Hence, it is to be understood that in case the method of the invention is to rely on HDR said one or more nucleic acid molecules further encode(s) in expressible form (iv) a repair template for the LRRC8A, LRRC8B, LRRC8C, LRRC8D or LRRC8E gene or a repair template for the innexin gene, and/or (v) a repair template for the gene of interest.

In the case of the presence of (iv) the LRRC8A, LRRC8B, LRRC8C, LRRC8D or LRRC8E gene or the innexin gene can be genome knocked-out by HDR. In the case of the presence of (v) the gene of interest can be genome edited by HDR. As discussed, in the absence of (iv) and (v) the LRRC8A, LRRC8B, LRRC8C, LRRC8D or LRRC8E gene or the innexin gene are knocked-out by NHEJ, and the gene of interest is genome edited by NHEJ.

The designs and structures of repair templates being suitable for HDR are known in the art. HDR is error-free if the repair template is identical to the original DNA sequence at the double-strand break (DSB), or it can introduce very specific mutations into DNA. The three central steps of the HDR pathways are: (1) The 5′-ended DNA strand is resected at the break to create a 3′ overhang. This will serve as both a substrate for proteins required for strand invasion and a primer for DNA repair synthesis. (2) The invasive strand can then displace one strand of the homologous DNA duplex and pair with the other. This results in the formation of the hybrid DNA, referred to as the displacement loop (D loop). (3) The recombination intermediates can then be resolved to complete the DNA repair process.

HDR templates used, for example, to introduce mutations or insert new nucleotides or nucleotide sequences into a gene require a certain amount of homology surrounding the target sequence that will be modified. Homology arms can be used that start at the CRISPR-induced DSB. In general, the insertion sites of the modification should be very close to the DSB, ideally less than 10 bp away, if possible. One important point to note is that the CRISPR enzymes may continue to cleave DNA once a DSB is introduced and repaired. As long as the gRNA target site/PAM site remain intact, the CRISPR nuclease will keep cutting and repairing the DNA. This repeated editing may be problematic if a very specific mutation or sequence is to be introduced into a gene of interest. To get around this, the repair template can be designed in such a way that it will ultimately block further CRISPR nuclease targeting after the initial DSB is repaired. Two common ways to block further editing are mutating the PAM sequence or the gRNA seed sequence. When designing a repair template, the size of the intended edit is to be taken into consideration. ssDNA templates (also referred to as ssODNs) are commonly used for smaller modifications. Small insertions/edits may require as little as 30-50 bases for each homology arm, and the best exact number may vary based on the gene of interest. 50-80 base homology arms are commonly used. For example, Richardson et al. (Nat Biotechnol. 2016 Mar; 34(3):339-44) found that asymmetric homology arms (36 bases distal to the PAM and 91 bases proximal to the PAM) supported HDR efficiencies up to 60%. Due to difficulties that might be associated with creating ssODNs longer than 200 bases, it is preferred to use dsDNA plasmid repair templates for larger insertions such as fluorescent proteins or selection cassettes into a gene of interest. These templates can have homology arms of at least 800 bp. To increase the frequency of HDR edits based on plasmid repair templates, self-cleaving plasmids can be used that contain gRNA target sites flanking the template. When the CRISPR nuclease and the appropriate gRNA(s) are present, the template is liberated from the vector. To avoid plasmid cloning, it is possible to use PCR-generated long dsDNA templates. Moreover, Quadros et al. (Genome Biol. 2017 May 17;18(1):92) developed Easi-CRISPR, a technique that allows making large mutations and to take advantage of the benefits of ssODNs. To create ssODNs longer than 200 bases, RNA encoding the repair template are in vitro transcribed and then reverse transcriptase is used to create the complementary ssDNA. Easi-CRISPR works well in mouse knock-in models, increasing editing efficiency from 1-10% with dsDNA to 25-50% with ssODNs. Although HDR efficiency varies across loci and experimental systems, ssODN templates generally provide the highest frequency of HDR edits.

Hence, a repair template for the LRRC8A, LRRC8B, LRRC8C, LRRC8D or LRRC8E gene comprises arms being homologous to the LRRC8A, LRRC8B, LRRC8C, LRRC8D or LRRC8E gene, respectively. Similarly, a repair template for an innexin gene comprises arms being homologous to the innexin gene, and/or a repair template for a gene of interest comprises arms being homologous to the gene of interest.

The CRISPR-Cas genome editing system was adapted from a naturally occurring defense system against foreign DNA (e.g. viruses, plasmid DNA) in prokaryotes. Prokaryotes with CRISPR-Cas system capture fragments of DNA from invading DNA and integrate them into DNA segments known as CRISPR arrays. The CRISPR arrays allow the bacteria/archaea to acquire immunity against the invading DNA (or homologous ones). The bacteria/archaea produce CRISPR-RNAs (crRNAs) from the CRISPR arrays to target the foreign DNA, which in complex with CRISPR nucleases (e.g. Cas9 or a similar enzyme) inactivate the invading DNA by nucleolytic cleavage'.

The CRISPR-Cas system has been harnessed for genome editing in prokaryotes and eukaryotes. A small piece of RNA with a short “guide” sequence that attaches (binds) to a specific target sequence of DNA in a genome is created (the so-called guide RNA (gRNA) or single guide (sgRNA))^{5,8}. The genomic target site of the gRNA can be any ˜20 nucleotide DNA sequence, provided it meets two conditions: (i) The sequence is unique compared to the rest of the genome, and (ii) the target is present immediately adjacent to a Protospacer Adjacent Motif (PAM). The PAM sequence is essential for target binding, but the exact sequence depends on which CRISPR endonuclease is used. CRISPR endonuclease and their respective PAM sequences are known in the art (see https://www.addgene.org/crispr/guide/#pam-table). Hence, the gRNA also binds to the CRISPR endonuclease (e.g. the Cas9 or Cpf1 enzyme). As in bacteria, the gRNA is used to recognize the DNA sequence, and the CRISPR endonuclease cuts the DNA at the targeted location. Once the DNA is cut, the cell's own DNA repair machinery (NHEJ or HDR) adds or deletes pieces of genetic material, or makes changes to the DNA by replacing an existing segment with a customized DNA sequence. Hence, in the CRISPR-Cas system, the CRISPR nuclease makes a double-stranded break in DNA at a site determined by the short (˜20 nucleotide) gRNA which break is then repaired within the cell by NHEJ or HDR. The CRISPR-Cas system can be multiplexed by adding multiple gRNAs. It was demonstrated that, for example, five different simultaneous mutations can be introduced into mouse embryonic stem cells by using five different gRNA molecules and one CRISPR endonuclease.

The method of the invention uses at least two guide RNAs. A guide RNA for knocking-out the leucine-rich repeat-containing protein 8 A (LRRC8A), LRRC8B, LRRC8C, LRRC8D or LRRC8E gene or the innexin gene, and a second guide RNA for editing the gene of interest. Further guide RNAs targeting additional genes of interest may be included. Within the context of the claimed method it is possible to adjust editing efficiencies of the at least two RNAs, for example, by (i) using a LRRC8-gRNA, which is less efficient than the sgRNA of the gene of interest (GOI) or (ii) by transfecting less LRRC8-gRNA expressing vectors compared to the GOI-gRNA expressing vectors or (iii) by transcriptional coupling. The aspect of transcriptional coupling will be discussed in greater detail herein below and is illustrated in Example 5.

The LRRC8 genes encode subunits of an ion channel being ubiquitously present in mammalian cells⁴. The LRRC8 gene family consists of five genes (LRRC8A-E). Orthologues of the LRRC8 proteins are found in the entire phylum Chordata⁴. The phylum Chordata includes the subphyla Vertebrata (which includes mammals, fish, amphibians, reptiles and birds), the Tunicata (which includes salps and sea squirts) and the Cephalochordata (which include the lancelets). The LRRC8 proteins form hetero-hexameric protein complexes, which act as volume-regulated anion channels (VRAC). The LRRC8A protein (also known as SWELL1) is an essential subunit of the LRRC8 complex, but it requires at least one additional subunit (LRRC8B-D) to form a functional ion channel^{8,9}. It seems that the LRRC8 subunit composition is not equal, but differs between cell types leading to slightly different, cell type-specific ion channel properties. While the LRRC8D subunit was implicated in blasticidin uptake in the chronic myelogenous leukemia cell line KBM-7¹⁰, an involvement of the major LRRC8A subunit in blasticidin resistance has not been addressed so far in any other cell type.

Blasticidin (4-amino-1-[4-({(3S)-3-amino-5-[[amino(imino)methyl]methyl)amino]pentanoyl}amino)-2,3,4-trideoxy-β-D-erythro-hex-2- enopyranuronosyl]pyrimidin-2(1H)-one or blasticidin S.) is an antibiotic that is produced by Streptomyces griseochromogenes. Blasticidin prevents the growth of both eukaryotic and prokaryotic cells. It works by inhibiting the termination step of translation and peptide bond formation (to lesser extent) by the ribosome. This means that cells can no longer produce new proteins through translation of mRNA. In prior art biological research, specifically in genetic engineering, it was used to select transformed cells which had been engineered to carry a resistance gene for blasticidin. Resistance to blasticidin is generally conferred in the art by the blasticidin resistance gene from Bacillus cereus (bsr), which codes for blasticidin-S deaminase. The presence of a blasticidin resistance gene in the cells used for the method of the invention is not required. The concentration of blasticidin used in the method of the invention is preferably 1 to 20 μg/ml, more preferably 2 to 10 μg/ml and most preferably about 4 μg/ml blasticidin. The term about is preferably ±20% and most preferably ±10%.

Derivatives of blasticidin retain the capability of blasticidin to inhibit protein synthesis. The derivative of blasticidin is preferably a salt or ester of blasticidin. For example, as the derivative blasticidin S carboxymethyl ester¹¹ can be used.

The term “in expressible form” means that the one or more nucleic acid molecules may encode (see step (a)) (i) a CRISPR nuclease, and encode (see steps (a) and (a′)) (ii) a guide RNA for knocking-out the leucine-rich repeat-containing protein 8 A (LRRC8A), LRRC8B, LRRC8C, LRRC8D or LRRC8E gene or a guide RNA for knocking-out the innexin gene, and (iii) a guide RNA for editing the gene of interest, in a form that ensures that the guide RNAs are transcribed and that the CRISPR nuclease (if being encoded) is transcribed and translated into the active enzyme in the cells.

The present invention is based on the surprising finding that genome edited cells can conveniently be enriched by using blasticidin as a selection marker. Experimentally it could be demonstrated that the LRRC8A-deficient HaCaT cells exhibit resistance to the antibiotic blasticidin (Example 1). Moreover, as Examples 2 evidences blasticidin treatment of mixed cell population enables the specific enrichment of LRRC8A-deficient clones (Example 2). This surprising effect was utilized in the method of the invention, in which the co-targeting of a LRRC8 gene and a gene-of-interest is used for the specific enrichment of the edited cells (Example 3). As a proof-of-concept, it is demonstrated in the examples that the method can be used to enrich modified cells even in hard-to-transfect HaCaT cells without the use of viral vectors; while without the blasticidin selection no CRISPR-edits were detectable, the blasticidin-mediated enrichment led to successful capture of co-edited cells (Example 4). Simply by the treatment of the cells with blasticidin, repair-capable cells with an active CRISPR ribonucleoprotein complex can now be specifically enriched. Thus, the method of the invention advantageously allows the capturing of rare editing events in the subpopulation of cells containing custom genetic modifications by the utilization of co-conversion or co-editing approach^{12,13}, which is based on the observation that the editing frequencies of two simultaneously targeted genes are statistically linked¹⁴.

Due to the ubiquitous expression of the LRRC8 genes in all chordate cells the method is advantageously applicable to a broad-range of cells. The system per se does not require any technical apparatus like FACS sorter or any special technical know-how in flow cytometry and is therefore is also easy, fast and cost-effective, further noting that a FACS sorter may be used subsequently to the system to further enrich or isolate genome edited cells. The method has the potential to further streamline Research and Development (R&D) on human cell-based therapies, gRNA validation, screening for essential genes or the optimization of producer or screening cells. Moreover, the use of LRRC8 genes (as phenotype causing gene) and blasticidin (as enrichment reagent) do not possess the disadvantages of the available co-editing methods, e.g. the adverse genetic consequences of the co-edited marker gene or the toxicity of the enrichment reagent for the user in the lab^{12,13}.

For instance, the selection marker ouabain (also known as g-strophanthin) is toxic. Ouabain is classified as an extremely hazardous substance in the United States as defined in Section 302 of the U.S. Emergency Planning and Community Right-to-Know Act (42 U.S.C. 11002), and is subject to strict reporting requirements by facilities which produce, store, or use it in significant quantities. Ouabain is very hazardous in case of ingestion and inhalation. It is also hazardous in case of skin contact (irritant) and eye contact (irritant). Severe over-exposure can even result in death. While also blasticidin is a toxic compound, it is a much less hazardous substance. It is only toxic to humans if swallowed and for safety reasons hands should be washed after handling. Hence, the method of the invention is superior to a co-conversion or co-editing approach using ouabain as the selection agent and the knock-out of the ATPA1 gene to render cells ouabain-resistent⁴.

The method of the invention is also superior to a co-conversion or co-editing approach using 6-thiogunaine (6TG) as the selection agent and the knock-out of the (hypoxanthine phosphoribosyltransferase) HPRT gene (encoding the hypoxanthine phosphoribosyltransferase) to render cells 6TG¹³-resistant. This is because the 6TG resistance phenotype of Hprt mutants is affected by the genotype of the neighboring cells. It has been established that HPRT mutants will be killed by toxic metabolic intermediates produced by the neighboring HPRT positive cells, which is known as cross killing or metabolic cooperation effect (Hooper and Slack, 1977, Developmental Biology, 55(2):271-284). Therefore, to select HPRT mutants in 6TG, cells have to be platted at very low density to avoid cross killing by wild type cells. Moreover, the housekeeping enzyme hypoxanthine phosphoribosyltransferase encoded by HPRT is known to have a key role in the purine salvage pathway and its mutations have been shown to cause aberrant expression of transcription factors, neurogenesis and Lesch-Nyhan syndrome ¹⁵⁻¹⁷.

As mentioned, CRISPR nucleases are a specific type of programmable nucleases. Currently there are at least three further families of engineered nucleases being used in the art: (i) meganucleases, (ii) zinc finger nucleases (ZFNs) (also called herein ZNF nucleases), and (iii) transcription activator-like effector-based nucleases (TALEN) (also called herein TAL nucleases).

Meganucleases are enzymes in the endonuclease family which are characterized by their capacity to recognize and cut large DNA sequences (from 12 to 40 base pairs). The most widespread and best known meganucleases are the proteins in the LAGLIDADG family, which owe their name to a conserved amino acid sequence. Meganucleases, found commonly in microbial species, have the unique property of having very long recognition sequences (>14bp) thus making them naturally very specific. In order to find the exact meganuclease required to act on a specific DNA sequence, mutagenesis and high throughput screening methods are available to create a meganuclease variant that recognizes a desired target sequences. It is also possible to fuse meganucleases to each other, thereby creating hybrid enzymes that recognize a new sequence. Moreover, a method named rationally designed meganuclease (U.S. Pat. No. 8,021,867) may be used to design sequence specific meganucelases.

The concept behind ZFNs and TALEN technology is based on a non-specific DNA cutting enzyme, which can then be linked to specific DNA sequence recognizing peptides such as zinc fingers and transcription activator-like effectors (TALEs). The key to this was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity.

Zinc finger motifs occur in several transcription factors. The zinc ion, found in 8% of all human proteins, plays an important role in the organization of their three-dimensional structure. In transcription factors, it is most often located at the protein-DNA interaction sites, where it stabilizes the motif. The C-terminal part of each finger is responsible for the specific recognition of the DNA sequence. The recognized sequences are short, made up of around 3 base pairs, but by combining 6 to 8 zinc fingers whose recognition sites have been characterized, it is possible to obtain specific proteins for sequences of around 20 base pairs. It is therefore possible to control the expression of a specific gene. The method generally adopted for this involves associating two proteins each containing 3 to 6 specifically chosen zinc fingers—with the catalytic domain of the Fokl endonuclease. The two proteins recognize two DNA sequences that are a few nucleotides apart. Linking the two zinc finger proteins to their respective sequences brings the two endonucleases associated with them closer together. Fokl requires dimerization to have nuclease activity and this means the specificity increases dramatically as each nuclease partner would recognize a unique DNA sequence. To enhance this effect, Fokl nucleases have been engineered that can only function as heterodimers and have increased catalytic activity.

Transcription activator-like effector nucleases (TALENs or TAL nucleases) are artificial restriction enzymes generated by fusing a specific DNA-binding domain to a non-specific DNA cleaving domain. The DNA binding domains, which can be designed to bind any desired DNA sequence, comes from TAL effectors, DNA-binding proteins excreted by plant pathogenic Xanthomanos sp. Tal effectors consists of repeated domains, each which contains a highly considered sequence of 34 amino acids, and recognize a single DNA nucleotide. The nuclease can create double strand breaks at the target site that can be repaired by error-prone non-homologous end-joining (NHEJ), resulting in gene disruptions through the introduction of small insertions or deletions. TALEN constructs are used in a similar way to designed zinc finger nucleases, and have at least three advantages in targeted mutagenesis: (1) DNA binding specificity is higher, (2) off-target effects are lower, and (3) construction of DNA-binding domains is easier.

As is evident from the above, in meganucleases, ZNF nucleases, and TAL nucleases the endonuclease activity and the site-specificity within the target genome are conferred by one compound. In an alternative embodiment of the method of the first aspect of the invention, said method is modified such that it makes use of these endonucleases.

Hence, the present invention also relates to a method for enriching cells within a cell population, wherein a gene of interest is genome edited, said method comprising (a) introducing into the cells within the cell population one or more nucleic acid molecules, said one or more nucleic acid molecules encoding in expressible form (i) a non-CRISPR programmable nuclease, preferably a meganuclease, a ZNF nuclease, or a TAL nuclease for knocking-out the leucine-rich repeat-containing protein 8 A (LRRC8A), LRRC8B, LRRC8C, LRRC8D or LRRC8E gene or such a non-CRISPR programmable nuclease for knocking-out an innexin gene, and (ii) a non-CRISPR programmable nuclease, preferably a meganuclease, a ZNF nuclease, or a TAL nuclease for editing the gene of interest; (a) introducing into the cells within the cell population (i) a non-CRISPR programmable nuclease, preferably a meganuclease, a ZNF nuclease, or a TAL nuclease for knocking-out the leucine-rich repeat-containing protein 8 A (LRRC8A), LRRC8B, LRRC8C, LRRC8D or LRRC8E gene or such a non-CRISPR programmable nuclease for knocking-out an innexin gene, and (ii) a non-CRISPR programmable nuclease, preferably a meganuclease, a ZNF nuclease, or a TAL nuclease for editing the gene of interest; and (b) treating the cells obtained after (a) or (a′) with blasticidin or a derivative thereof, thereby (i) selecting cells wherein the LRRC8A, LRRC8B, LRRC8C, LRRC8D or LRRC8E gene or the innexin gene is knocked-out, and (ii) enriching cells, wherein the gene of interest is genome edited. The definitions and preferred embodiments provided herein in connection with the first aspects of the invention are equally applicable to this method of the invention. For instance, also in connection with the non-CRISPR programmable nucleases the DSBs can be repaired in the cells by NHEJ or HDR. In case HDR is to be relied upon, the one or more nucleic acid molecules also further encode(s) in expressible form (iv) a repair template for the LRRC8A, LRRC8B, LRRC8C, LRRC8D or LRRC8E gene or a repair template for the innexin gene, and/or (v) a repair template for the gene of interest.

In accordance with a preferred embodiment of the first embodiment of the first aspect of the invention the one or more nucleic acid molecules are one or more vectors, and preferably one vector.

The method of the invention requires in accordance with step (a) expressing both the CRISPR endonuclease and the gRNAs within the same cell at the same time and in accordance with step (a′) expressing the gRNAs within the same cell at the same time, while introducing the CRISPR endonuclease in proteinaceous form into the same cell so that the CRISPR endonuclease is active at the same time. The method of the invention can be implemented by using one or more vectors. Preferably, the vector is a plasmid, cosmid, virus, bacteriophage or another vector used conventionally e.g. in genetic engineering.

The nucleic acid molecules used in accordance with of the present invention may be inserted into several commercially available vectors. Single vectors containing both the CRISPR endonuclease and the gRNAs are commercially available, thereby acting as an all-in-one vector. The method of the invention can alternatively be implemented by using two or three vectors containing the CRISPR endonuclease and the at least two gRNAs. It is also possible to use gRNA-only vectors and use cells in which the CRISPR endonuclease has been integrated into the genome. The use of an all-in-one vector that expresses the at least two gRNA and the CRISPR endonuclease is preferred since only one vector is to be introduced into the cells. A vector which can express the CRISPR endonuclease and up to seven gRNAs is, for example, described in Sakuma et al, Sci Rep. 2014; 4: 5400.

Many single gRNA empty vectors (with and without the CRISPR endonuclease) are available in the art. Likewise several empty multiplex gRNA vectors are available that can be used to express multiple gRNAs from a single plasmid (with or without the expression of the CRISPR endonuclease). Finally, also vectors are available that only express the CRISPR endonuclease (see https://www.addgene.org/crispr/empty-grna-vectors/).

Vector modification techniques are known in the art and, for example, described in Sambrook and Russel, 2001. Generally, vectors can contain one or more origins of replication (ori) and inheritance systems for cloning or expression, one or more markers for selection in the host, e.g., antibiotic resistance, and one or more expression cassettes. Suitable origins of replication include, for example, the Col E1, the SV40 viral and the M 13 origins of replication. The nucleic acid sequences inserted in the vector can e.g. be synthesized by standard methods, or isolated from natural sources. Ligation of the coding sequences to transcriptional regulatory elements and/or to other amino acid encoding sequences can be carried out using established methods. Such regulatory sequences are well known to those skilled in the art and include, without being limiting, regulatory sequences ensuring the initiation of transcription, initiation of translation, internal ribosomal entry sites (IRES) or 2A linker (Owens, Proc. Natl. Acad. Sci. USA 98 (2001), 1471-1476) and optionally regulatory elements ensuring termination of transcription and stabilization of the transcript. Non-limiting examples for regulatory elements ensuring the initiation of transcription comprise a translation initiation codon, enhancers such as e.g. the SV40-enhancer, insulators and/or promoters, such as for example the cytomegalovirus (CMV) promoter, elongation factor-1 alpha (EF1-alpha), promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gall 0 promoter, human elongation factor 1a-promoter, AOX1 promoter, GAL1 promoter, CaM-kinase promoter, the lac, trp or tac promoter, the lacUV5 promoter, the Autographa californica multiple nuclear polyhedrosis virus (AcMNPV) polyhedral promoter or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing, nucleotide sequences encoding secretion signals or, depending on the expression system used, signal sequences capable of directing the expressed polypeptide to a cellular compartment. Moreover, elements such as origin of replication, drug resistance gene, regulators (as part of an inducible promoter) may also be included.

In accordance with a further preferred embodiment of the first aspect of the invention step (a) comprises transducing or transfecting the cells of the cell population with the one or more nucleic acid molecules.

As mentioned, the nucleic acid molecule(s) expressing the CRISPR endonuclease and/or the gRNAs have to be introduced into the cells. Means and methods for the introduction for the nucleic acid molecule(s) expressing the CRISPR endonuclease and the gRNAs into cells are known in the art and these methods encompass transducing or transfecting cells.

Transduction is the process by which foreign DNA is introduced into a cell by a virus or viral vector. Transduction is a common tool used by molecular biologists to stably introduce a foreign gene into a host cell's genome. Generally, a plasmid is constructed in which the genes to be transferred are flanked by viral sequences that are used by viral proteins to recognize and package the viral genome into viral particles. This plasmid is inserted (usually by transfection) into a producer cell together with other plasmids (DNA constructs) that carry the viral genes required for formation of infectious virions. In these producer cells, the viral proteins expressed by these packaging constructs bind the sequences on the DNA/RNA (depending on the type of viral vector) to be transferred and insert it into viral particles. For safety, none of the plasmids used contains all the sequences required for virus formation, so that simultaneous transfection of multiple plasmids is required to get infectious virions. Moreover, only the plasmid carrying the sequences to be transferred contains signals that allow the genetic materials to be packaged in virions, so that none of the genes encoding viral proteins are packaged. Viruses collected from these cells are then applied to the cells to be altered. The initial stages of these infections mimic infection with natural viruses and lead to expression of the genes transferred and (in the case of lentivirus/retrovirus vectors) insertion of the DNA to be transferred into the cellular genome. However, since the transferred genetic material does not encode any of the viral genes, these infections do not generate new viruses (the viruses are “replication-deficient”). In the present case transduction may be used to generate cells that comprise the CRISPR nuclease in their genome in expressible form.

Transfection is the process of deliberately introducing naked or purified nucleic acids or purified proteins or assembled ribonucleoprotein complexes into cells. Transfection is generally a non-viral based method.

Transfection may be a chemical-based transfection. Chemical-based transfection can be divided into several kinds: transfection using cyclodextrin, polymers, liposomes, or nanoparticles. One of the cheapest methods uses calcium phosphate. HEPES-buffered saline solution (HeBS) containing phosphate ions are combined with a calcium chloride solution containing the DNA to be transfected. When the two are combined, a fine precipitate of the positively charged calcium and the negatively charged phosphate will form, binding the DNA to be transfected on its surface. The suspension of the precipitate is then added to the cells to be transfected (usually a cell culture grown in a monolayer). By a process not entirely understood, the cells take up some of the precipitate, and with it, the DNA. This process has been a preferred method of identifying many oncogenes. Other methods use highly branched organic compounds, so-called dendrimers, to bind the DNA and transfer it into the cell. Another method is the use of cationic polymers such as DEAE-dextran or polyethylenimine (PEI). The negatively charged DNA binds to the polycation and the complex is taken up by the cell via endocytosis. Lipofection (or liposome transfection) is a technique used to inject genetic material into a cell by means of liposomes, which are vesicles that can easily merge with the cell membrane since they are both made of a phospholipid bilayer. Lipofection generally uses a positively charged (cationic) lipid (cationic liposomes or mixtures) to form an aggregate with the negatively charged (anionic) genetic material. This transfection technology performs the same tasks in terms of transfer into cells as other biochemical procedures utilizing polymers, DEAE-dextran, calcium phosphate, and electroporation. The efficiency of lipofection can be improved by treating transfected cells with a mild heat shock. Fugene is a series of widely used proprietary non-liposomal transfection reagents capable of directly transfecting a wide variety of cells with high efficiency and low toxicity.

Transfection may also be a non-chemical method. Electroporation (gene electrotransfer) is a popular method, where transient increase in the permeability of cell membrane is achieved when the cells are exposed to short pulses of an intense electric field. Cell squeezing enables delivery of molecules into cells via cell membrane deformation. Sonoporation uses high-intensity ultrasound to induce pore formation in cell membranes. This pore formation is attributed mainly to the cavitation of gas bubbles interacting with nearby cell membranes since it is enhanced by the addition of ultrasound contrast agent, a source of cavitation nuclei. Optical transfection is a method where a tiny (˜1 μm diameter) hole is transiently generated in the plasma membrane of a cell using a highly focused laser. Protoplast fusion is a technique in which transformed bacterial cells are treated with lysozyme in order to remove the cell wall. Following this, fusogenic agents (e.g., Sendai virus, PEG, electroporation) are used in order to fuse the protoplast carrying the gene of interest with the recipient target cell.

Finally, transfection may be a particle-based method. A direct approach to transfection is the gene gun, where the DNA is coupled to a nanoparticle of an inert solid (commonly gold), which is then “shot” (or particle bombardment) directly into the target cell's nucleus. Hence, the nucleic acid is delivered through membrane penetration at a high velocity, usually connected to microprojectiles. Magnetofection, or magnet-assisted transfection, is a transfection method that uses magnetic force to deliver DNA into target cells. Impalefection is carried out by impaling cells by elongated nanostructures and arrays of such nanostructures such as carbon nanofibers or silicon nanowires which have been functionalized with plasmid DNA.

In accordance with a yet further preferred embodiment of the first aspect of the invention, the cells are chordate cells, preferably cells of a mammalian cell line, organoids, primary cells, cells from a primary cell line, or pluripotent stem cells.

A mammalian cell line is a population of cells from a mammal which would normally not proliferate indefinitely but, due to mutation (that naturally occurred, e.g. in a tumor or by artificial mutagenesis), have evaded normal cellular senescence and instead can keep undergoing division. The cells can therefore be grown for prolonged periods in vitro.

An organoid is a miniaturized and simplified version of an organ produced in vitro in three dimensions that shows realistic micro-anatomy. They are derived from one or a few cells from a tissue, embryonic stem cells or induced pluripotent stem cells, which can self-organize in three-dimensional culture owing to their self-renewal and differentiation capacities.

Primary cells are cells taken directly from living tissue (e.g. biopsy material) and established for growth in vitro. These cells have undergone very few population doublings and are therefore more representative of the main functional component of the tissue from which they are derived in comparison to continuous (tumor or artificially immortalized) cell lines thus generally representing a more representative model to the in vivo state. A primary cell line is a cell line that has been established from primary cells.

Pluripotent stem cells are cells that have the capacity to self-renew by dividing and to develop into the three primary germ cell layers of the early embryo and therefore into all cells of the adult body, but not extra-embryonic tissues such as the placenta. Embryonic stem cells and induced pluripotent stem cells are pluripotent stem cells. Embryonic stem cells are derived from the inner cell mass of a blastocyst, an early-stage pre-implantation embryo. Human embryos reach the blastocyst stage 4-5 days post fertilization, at which time they consist of 50-150 cells. They are preferably isolated from the embryo without the destruction of the embryo. Induced pluripotent stem cells (also known as IFS cells or iPSCs) are a type of pluripotent stem cell that can be generated directly from adult cells. The iPSC technology was pioneered by Shinya Yamanaka's lab in Kyoto, Japan, who showed in 2006 that the introduction of four specific genes encoding transcription factors could convert adult cells into pluripotent stem cells. The generation of iPSCs using Oct3/4 and/or a factor belonging to the Myc, Klf and Sox families of factors is described in WO 2009/144008.

In a more preferred embodiment the cells are keratinocytes and preferably HaCaT cells.

HaCaT cells (German Cancer Research Center DKFZ, Heidelberg, can be ordered, for example, via the CLS Cell Lines Service GmbH) are used in the examples. HaCaT cells are a spontaneously transformed aneuploid immortal keratinocyte cell line from adult human skin. HaCaT cells are utilized for their high capacity to differentiate and proliferate in vitro.

In accordance with another preferred embodiment of the first aspect of the invention which uses a CRISPR nuclease, the expression of the gRNA for editing the gene of interest is transcriptionally coupled to the expression of the gRNA for knocking-out the LRRC8A, LRRC8B, LRRC8C, LRRC8D or LRRC8E gene or the gRNA for knocking-out the innexin gene.

In order to reduce the amount of single-edited cells and thus to further increase the frequency of co-editing, the transcription of the LRRC8 or innexin-targeting gRNA may be coupled to the transcription of the GOI-targeting gRNA. Transcriptional coupling requires in its broadest sense that these two guide RNAs are expressed or can be expressed at the same time within the cells of the cell population. Transcriptional coupling can be achieved by using a single promoter driving the expression of at least these two gRNAs. Means and methods for ensuring that the two guide RNAs are expressed at the same time are known in the art. For example, the two guide RNAs may be expressed under the control of the same promotor within a vector in two separate expression cassettes or also in one expression cassette. The use of an inducible promoter may further allow controlling the time of expression of the guide RNAs within the cells. As will be further discussed herein below self-splicing RNAs and RNA-sequences that are processed by proteins may be used to transcriptionally couple the at least two guide RNAs.

In accordance with a more preferred embodiment of the first aspect of the invention, the transcriptional coupling is achieved by using (i) self-splicing RNAs, preferably hepatitis delta virus (HDV) and Hammerhead ribozymes (HH), or (ii) RNA-sequences that are processed by proteins, preferably by tRNA-sequences. The use of HDV/HH is illustrated in Example 5.

Self-splicing RNAs (also known as ribozymes or ribonucleic acid enzymes) are RNA molecules that are capable of catalyzing specific biochemical reactions, similar to the action of protein enzymes.

These RNA molecules act catalytic or autocatalytic and are capable of cleaving e.g. other RNAs at specific target sites but they have also been found to catalyze the aminotransferase activity of the ribosome. Selection of appropriate target sites and corresponding ribozymes are known in the art. Examples of well-characterized small self-cleaving RNAs are the hammerhead, hairpin, hepatitis delta virus, and in vitro-selected lead-dependent ribozymes. The organization of these small catalysts is in contrast to that of larger ribozymes, such as the group I intron.

Hepatitis delta virus (HDV) is a small (about 1,700-nucleotide (nt)) single-stranded RNA virus first isolated from human hepatocytes infected with hepatitis B virus. HDV harbors two structurally related self-cleaving ribozymes in its genome, one in the genomic and one in the complementary, antigenomic strand. Like other small self-cleaving ribozymes, these RNAs catalyze a transesterification reaction, promoting a nucleophilic attack by a 2′ hydroxyl on the adjacent phosphate and yield both a 2′-3′ cyclic phosphate and a liberated 5′ hydroxyl.

The hammerhead ribozyme (so named because diagrams of its nucleotide sequence look like a hammer) is the smallest natural ribozyme discovered so far. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

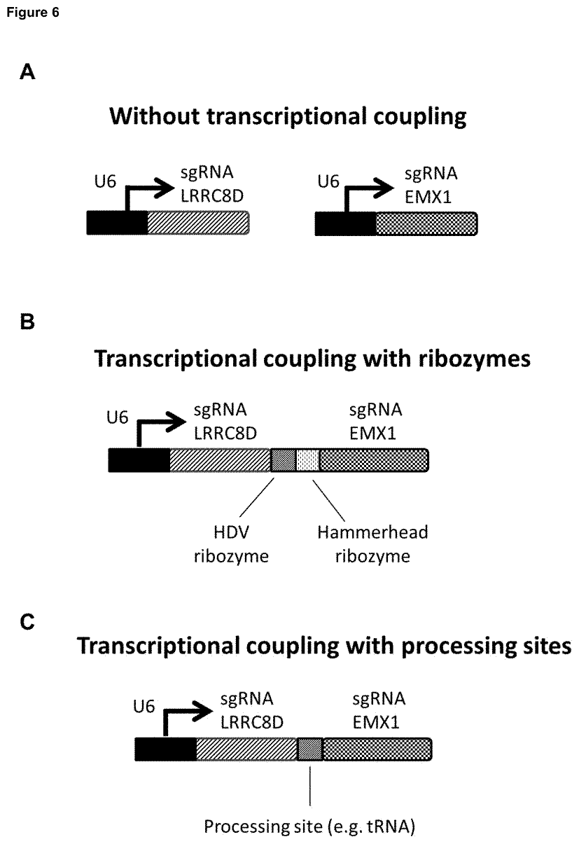

Transcriptional coupling using self-splicing RNAs, such as HDV and hammerhead ribozymes can be achieved by using a single promoter driving the expression of two or more gRNAs that are intervened by self-splicing ribozymes. HDV and hammerhead ribozymes can be used to generate compact gRNA-expression cassette and/or to circumvent the need of RNA polymerase III-dependent promoters for the expression of gRNAs, in particular in eukaryotic cells^{18,19}. In the method of the present invention, for example, the HDV ribozyme may be used to ensure the processing of the 3′-end of the first gRNA, and a hammerhead ribozyme to ensure the processing of the 5′-end of the second gRNA (FIG. 6).

Instead of ribozymes, regulatory sequences that act as cleavage site for RNA-processing enzymes can also be used for transcriptional coupling, i.e. RNA-sequences that are processed by proteins, preferably by tRNA-sequences. For example sequences derived from tRNAs for tRNA processing proteins or sequences for processing by Drosha or Csy4 protein ^{20,22.} Moreover, the concept of transcriptional coupling is illustrated by FIG. 6.

A tRNA-sequence is an adaptor molecule composed of RNA, typically 76 to 90 nucleotides in length, that serves as the physical link between the mRNA and the amino acid sequence of proteins. tRNA does this by carrying an amino acid to the protein synthetic machinery of a cell (ribosome) as directed by a three-nucleotide sequence (codon) in a messenger RNA (mRNA). As such, tRNAs are a necessary component of translation, the biological synthesis of new proteins in accordance with the genetic code. For example, more than 12.000 tRNA genes from 577 species and 623 tRNA sequences from 104 species are available via the database tRNAdb (http://trna.bioinf.uni-leipzig.de/DataOutput/).

In accordance with a more preferred embodiment of the first aspect of the invention, the method further comprises (c) isolating one or more cells, wherein the gene of interest is genome edited.

Means and methods for this isolation step are known in the art. Non-limiting examples are single-cell dilution, laser capture microdissection, manual or automated cell picking, FACS and MACS.

In single-cell dilution a solution comprising cells is diluted in more or more steps until a solution with only a single cell is obtained. Laser capture microdissection is a method for isolating specific cells of interest from microscopic regions of tissue, cells or organisms. A laser is coupled into a microscope and focuses onto on a selected cell within a cell population. By movement of the laser by optics or the stage the focus follows a trajectory which is predefined by the user. This trajectory with the selected cell, also called element, is then cut out and separated from the adjacent cells. Manual cell picking is a simple, convenient, and efficient method for isolating single cells. Manual cell picking micromanipulators consist of an inverted microscope combined with micro-pipettes that are movable through motorized mechanical stages. Cell picking can also be implemented into an automated device. Fluorescence Activated Cell Sorting (FACS), a specialized type of flow cytometry with sorting capacity, is the most sophisticated and user-friendly technique for characterizing and defining different cell types in a heterogeneous cell population based on size, granularity, and fluorescence. FACS allows simultaneous quantitative and qualitative multi-parametric analyses of single cells. Magnetic-Activated Cell Sorting (MACS) is another commonly used passive separation technique to isolate different types of cells depending on their cluster of differentiation. It has been reported that MACS is capable of isolating specific cell populations with a purity >90% purification.

The present invention relates in a second aspect to an isolated cell obtained by the method of the invention.

The definitions and preferred embodiments provided herein in connection with the first aspects of the invention are equally applicable to the second aspect of the invention.

For instance, also the isolated cell may be a chordate cell, preferably a mammalian and most preferably a human cell, such as a keratinocyte cell.

The present invention relates in a third aspect to a composition comprising the cell of the invention.

The definitions and preferred embodiments provided herein in connection with the first aspect of the invention are equally applicable to the third aspect the invention.

The term “composition”, as used in accordance with the present invention, relates to a composition which comprises at least one cell, generally a plurality of the cells of the invention. It may, optionally, comprise further ingredients and in particular ingredients which are capable to keep the cell alive. Such ingredients are essential nutrients (amino acids, carbohydrates, vitamins, minerals), growth factors, hormones, and or gases (CO₂, O₂).

The composition may be an industrial composition, a diagnostic composition or a pharmaceutical composition.

An industrial composition is intended to be used in industry, including agriculture. For instance, cells wherein a particular enzyme as the gene of interest has been introduced may be used in chemical production, biofuels, food & beverage, animal feeds, cosmetic products and consumer products.

A diagnostic composition is intended to be used in the diagnosis or a disease or condition. For instance, cells wherein a particular fluorescent protein as the gene of interest has been introduced may be used in diagnosis since they can be detected within an organism or a tissue sample.

In accordance with a preferred embodiment of the third aspect of the invention, the composition is a pharmaceutical composition.

By the method of the present invention it is possible to correct disease-causing mutations in patient-derived pluripotent stem cells and then to create isogenic cell lines to differentiate to any cell type of interest for disease research. Generating these isogenic lines makes it possible to generate cells that can be used to treat the disease in the patient.

In accordance with the present invention, the term “pharmaceutical composition” relates to a composition for administration to a patient, preferably a human patient. The pharmaceutical composition of the invention comprises the cells recited above. It may, optionally, comprise further molecules capable of altering the characteristics of the cells of the invention thereby, for example, stabilizing, modulating and/or activating their function. The composition is preferably in liquid form, e.g. (a) solution(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1×10⁴ to 1×10⁸ cells per day. However, a more preferred dosage might be in the range of 1×10⁵ to 1×10⁷ cells and most preferably 5×10⁸ to 5×10⁶ cells per day.

The length of treatment needed to observe changes and the interval following treatment for responses to occur vary depending on the desired effect. The particular amounts may be determined by conventional tests which are well known to the person skilled in the art.

The present invention relates in a fourth aspect to a vector comprising in expressible form (i) optionally a CRISPR nuclease, (ii) a guide RNA for knocking-out the LRRC8A, LRRC8B, LRRC8C, LRRC8D or LRRC8E gene or a guide RNA for knocking-out the innexin gene, and (iii) a guide RNA editing the gene of interest.

The definitions and preferred embodiments provided herein in connection with the first to third aspect of the invention are equally applicable to the fourth aspect the invention. Hence, in case the vector is intended to be used for genome editing via HDR the vector further comprises in expressible form (iv) a repair template for the LRRC8A, LRRC8B, LRRC8C, LRRC8D or LRRC8E gene or a repair template for the innexin gene, and/or (v) a repair template for the gene of interest. On the other hand, the above compounds (i) to (iii) suffice to render the vector suitable for NHEJ editing.

In particular and as discussed, single vectors wherein both the CRISPR endonuclease and the required at least two gRNAs can be introduced are commercially available.

The present invention relates in a fifth aspect to a kit comprising (a) one or more nucleic acid molecules encoding in expressible form (i) a CRISPR nuclease, (ii) a guide RNA for knocking-out LRRC8A, LRRC8B, LRRC8C, LRRC8D or LRRC8E or a guide RNA for knocking-out the innexin gene, and (iii) a guide RNA for editing the gene of interest, or (b) (i) a CRISPR nuclease, and one or more nucleic acid molecules encoding in expressible form (ii) a guide RNA for knocking-out LRRC8A, LRRC8B, LRRC8C, LRRC8D or LRRC8E or a guide RNA for knocking-out an innexin gene, and (iii) a guide RNA for editing the gene of interest.

The definitions and preferred embodiments provided herein in connection with the first to fourth aspect of the invention are equally applicable to the fourth aspect the invention. For instance, in the kit (i) optionally a CRISPR nuclease, (ii) the guide RNA for knocking-out LRRC8A, LRRC8B, LRRC8C, LRRC8D or LRRC8E or the guide RNA for knocking-out the innexin gene, and (iii) the guide RNA for editing the gene of interest may be present in the form of the vector and/or the cell of the invention. Also the one or more nucleic acid molecules of the kit may further encode in expressible form (iv) a repair template for the LRRC8A, LRRC8B, LRRC8C, LRRC8D or LRRC8E gene or a repair template for the innexin gene, and/or (v) a repair template for the gene of interest in case the kit is intended to be used for genome editing via HDR. On the other hand, the above compounds (i) to (iii) suffice to render the kit suitable for NHEJ editing. Furthermore the kit may optionally further comprise blasticidin or a derivative thereof.

The kit of the fifth aspect of the invention implements a/the means required for conducting the method of the invention in the format of a kit. For this reason the definitions and preferred embodiments provided herein above in connection with the first to fourth aspect of the invention are equally applicable to the kit of the thirteenth aspect of the invention.

The various components of the kit may be packaged into one or more containers such as one or more vials. The vials may, in addition to the components, comprise preservatives or buffers for storage. The kit may comprise instructions how to use the kit, which preferably inform how to use the components of the kit for genome editing the gene of interest.

In accordance with a preferred embodiment of all the foregoing aspects of the invention, the method, cell, vector or kit of any preceding claim, wherein the guide RNA of (ii) is for knocking-out the LRRC8D gene or the LRRC8A gene, preferably the LRRC8D gene.

The LRRC8D gene has been knocked-out as described in Example 4. It was found that the LRRC8D gene knock-out renders the cells resistant to blasticidin and thus provides proof-of-principle for the claimed method. Moreover, it is known from the prior art that LRRC8A is the major component of the ion channel being encoded by the LRRC8 genes. It is shown in Example 2 that LRRC8A knock-out cells are blasticidin resistant.

In accordance with a further preferred embodiment of all the foregoing aspects of the invention, the method, cell, vector or kit the CRISPR nuclease is a Class II CRISPR-Cas nuclease, and is preferably Cas9, Cpf1, CasX, CasY or C2c1/2/3.

CRISPR-Cas systems are generally classified into Classes I, II and III CRISPR-Cas systems. The CRISPR nuclease is preferably a Class II CRISPR-Cas nuclease. The Class II system has been most extensively studied. The Class II CRISPR mechanism is unique compared to other CRISPR systems, as only one CRISPR nuclease is required for gene silencing and is responsible for the destruction of the target DNA.

Preferred examples of CRISPR nucleases are Cas9, Cpf1, CasX, CasY and C2c1/2/3. Cas9 was the first identified CRISPR nuclease being suitable for genome editing. Since the development of Cas9 as a genome engineering tool in 2012-2013, various improvements and alternatives of CRISPR-Cas systems, including engineered Cas9 variants, Cas9 homologs, and novel Cas proteins other than Cas9 became available. These variations enable flexible genome engineering with high efficiency and specificity, orthogonal genetic control at multiple gene loci, gene knockdown, or fluorescence imaging of transcripts mediated by RNA targeting, and beyond (see for review, Nakade et al., Bioengineered. 2017; 8(3): 265-273). For example, highly specific SpCas9 variants, named eSpCas922 and SpCas9-HF are now available. One kind of endonuclease from the type-V CRISPR-Cas systems, called Cpf1, has been discovered and characterized. Cpf1 system can be used for genome engineering similar to Cas9. In addition, 53 class-2 CRISPR-Cas candidates were identified and categorized into 3 groups by the context characteristics; C2c1, C2c2 and C2c3. C2c1 and C2c3 were later grouped in type V, and C2c2 was grouped in the new type VI. Among them, C2c2 nucleases have an especially unique feature that its target molecule is not the double-strand DNA but the single-strand RNA; thus possibly contributing gene knockdown applications. CasX and CasY were recently identified in bacteria (Burstein et al., Nature. 2017 Feb 9; 542(7640): 237-241).

In accordance with another preferred embodiment of all the foregoing aspects of the invention, the guide RNA for editing the gene of interest is for knocking-out or knocking-in the gene of interest.

As discussed above, if a gene of interest due to the appliance of the method of the invention has a complete loss of function (wholly inactivated) this is called a gene knock-out. On the other hand, introducing a gene of interest into a cell by the method of the invention resulting in adding a new function or effect to a cell is called a gene knock-in. Both options are envisioned in accordance with the present invention.

In accordance with a further preferred embodiment of all the foregoing aspects of the invention, the guide RNA for knocking-out the LRRC8A, LRRC8B, LRRC8C, LRRC8D or LRRC8E gene targets a non-coding region of the LRRC8A, LRRC8B, LRRC8C, LRRC8D or LRRC8E gene; or the guide RNA for knocking-out an innexin gene targets a non-coding region of the innexin gene.

The non-coding region is preferably an intron and more preferably the intron preceding the translation start codon ATG. The corresponding intron in the LRRC8A, LRRC8B, LRRC8C, LRRC8D or LRRC8E gene including the final ATP codon are shown in SEQ ID NOs 6 to 11, respectively. The non-coding region is most preferably selected from SEQ ID NOs 6 to 11. As is commonly known, an intron is any nucleotide sequence within a gene that is removed by RNA splicing during maturation of the final RNA product.

Moreover, in the preferred embodiment preferably in addition a homologous recombination template to knock-out the LRRC8A, LRRC8B, LRRC8C, LRRC8D or LRRC8E gene or the innexin gene upon homologous recombination within the target cell is provided.

As discussed herein above genome editing uses the cell's own repair pathways, including the NHEJ or HDR pathway. The latter comprises a homologous recombination (HR) event in the target cell. In order to distinguish between target cells, wherein NHEJ took place and those wherein HR took place a guide RNA according to the above preferred embodiment can be used. This is because in the case of NHEJ after the CRISPR nuclease cut the target nucleic acid in the non-coding region, the two ends of the break are simply rejoined. Since the mutations introduced by the cut are in a non-coding region the gene stays functional in the case the ends of the break are rejoined by NHEJ. On the other hand, in the case of HDR the break is repaired by HR which comprises the introduction of a homologous recombination template at the site of the break. As will be further detailed herein below, via the introduction of the homologous recombination template into the gene, the gene can be knocked-out (i.e. completely loses its function or is wholly inactivated). This knock-out only can be observed in cells upon HDR and not upon NHEJ. This strategy is illustrated in Examples 6 to 8.

In accordance with a yet further preferred embodiment of all the foregoing aspects of the invention, in addition one or more nucleic acid molecules is/are provided as a first and second homologous recombination template, wherein (i) the first homologous recombination template is to knock-out the LRRC8A, LRRC8B, LRRC8C, LRRC8D or LRRC8E gene or the innexin gene upon homologous recombination repair, and (ii) the second homologous recombination template is to edit the gene of interest upon homologous recombination repair.

The one or more nucleic acid molecules is/are preferably single-stranded nucleic acid(s) (in particular single-stranded DNA) and/or vector(s).

In case of single-stranded nucleic acid(s) the first and second homologous recombination templates are preferably provided as two separate single-stranded nucleic acids, since longer single-stranded nucleic acids have a higher tendency of missfolding and/or degradation inside cells.

In the case of vector(s) the first and second homologous recombination template are either encoded by (i.e present on) one vector or on two separate vectors. In case a homologous recombination template is encoded by a vector, the vector generally comprises a flanking region on each site of the homologous recombination template within the vector that can be cut by the CRISPR nuclease. This ensures that the homologous recombination template becomes available for HDR as a single-stranded DNA within the target cell.

The use and design of homologous recombination templates to be used in genome editing via the CRISPR-Cas system is well established. As discussed herein above, in HDR a homologous sequence is utilized as a template for the regeneration of a missing DNA sequence at the break point introduced by the CRISPR nuclease and this sequence is designated “homologous recombination template” or “HDR donor template” herein.

The homologous recombination template needs to have sufficient homology to the regions flanking the cut site. Hence, a homologous recombination template comprises two homology arms on each site of the cut site. The length of the homology arms and the type of template (e.g. either a single stranded DNA oligo or a template being encoded by a plasmid) depend on the type and size of the precise modification to be made and the skilled person can design the homologous recombination template accordingly. Also web-based tools for designing and then ordering homologous recombination templates are available (for example, via https://dharmacon.horizondiscovery.com/gene-editing/crispr-cas9/edit-r-hdr-donor-designer-plasmid/).

Homology arm lengths are often about 30 nt to about 50 nt but can also be as long as about 60 nt to about 70 nt and even longer. For unmodified DNA templates, asymmetric arms showed modest improvement over symmetric arms consistent with the literature (Richardson et al., Nat Biotechnol 34, 339-344 (2016)). However, when phosphorothioated templates were used, symmetric arms resulted in the best knock-in efficiency. For most sequences tested, phosphorothioate-modified DNA templates outperformed unmodified DNA templates. This is consistent with recent publications (Renaud et al., Cell Rep 14, 2263-2272 (2016)). Hence, the homologous recombination templates herein are preferably homologous recombination templates.

The design of a homologous recombination template which results upon HDR in a gene knock-out is illustrated herein below in Examples 6 and 7. For instance, one or both arms of the homologous recombination template may comprise mutations which result in the gene to be knocked-out upon HDR. Such a mutation may, for example, introduce one or more point mutations, a frame shift or mutate the translation start codon ATG, for example, from ATG to ATT. In this respect a mutation of the stop codon is preferred.

In accordance with a more preferred embodiment of all the foregoing aspects of the invention, the second homologous recombination template is to (i) knock-in a gene or a nucleic acid sequence encoding a protein, peptide or RNA molecule, (ii) replace the gene of interest by another gene, or (iii) correct a mutation in the gene of interest.

How the second homologous recombination template is to edit the gene of interest upon homologous recombination is not particularly limited. However, the genomic editing results preferably in one of the above genome edits according to (i) to (iii). A gene knock-in either introduces a gene into the cell which is already present in the cell in order to increase its expression or a new gene thereby providing the cell with a new functionality. The protein or peptide being encoded by the nucleic acid sequence may comprise or be, for example, an epitope that can be bound by an antibody or a binding fragment thereof. The RNA being encoded by the nucleic acid sequence may be a non-coding RNA, such as miRNA, siRNA, piRNA, IncRNA (e.g. XIST), snoRNA, snRNA, exRNA or scaRNA. The gene of interest may be replaced by another gene, for example, in case the gene of interest does any harm to the cells or in case the phenotype of the gene replacement is to be characterized. The correction of a mutation in the gene of interest is of particular interest for therapeutic approaches, in order to correct for mutations causing a particular disease.

Unless otherwise defined, all technical and scientific terms used herein have the same meaning as commonly understood by one of ordinary skill in the art to which this invention belongs. In case of conflict, the patent specification, including definitions, will prevail.

Regarding the embodiments characterised in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

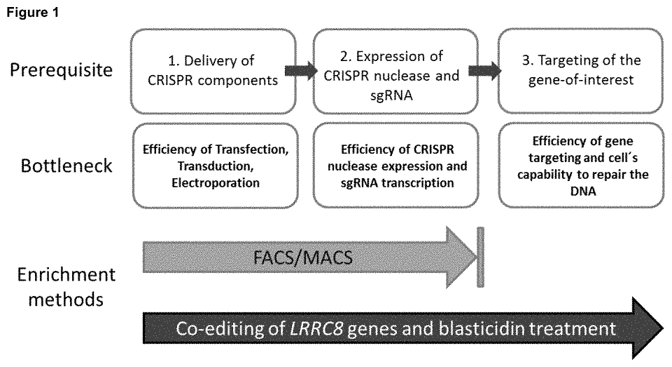

FIG. 1: Genome editing workflow, the bottlenecks of each single step and the enrichment methods compared to the present invention based on the co-editing of LRRC8 genes followed by blasticidin treatment.

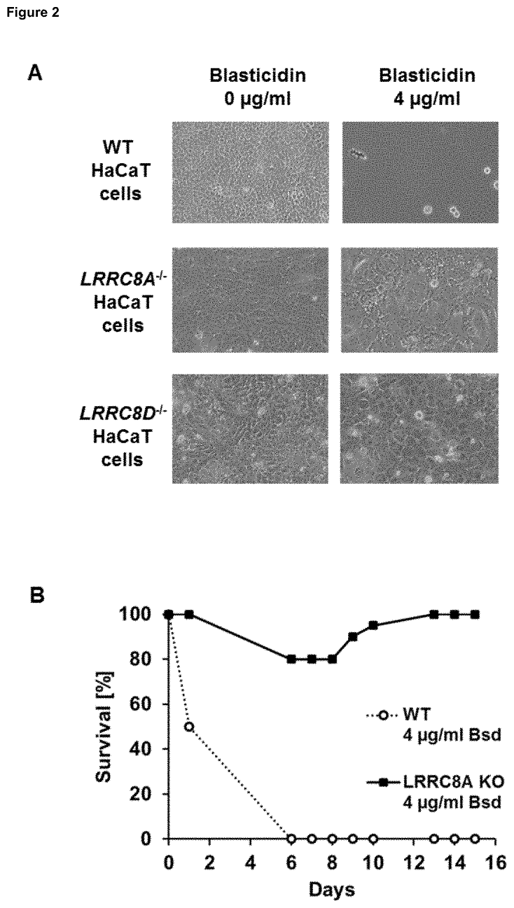

FIG. 2: (A) Microscopic analysis of cytotoxic effect of blasticidin on HaCaT wildtype keratinocytes (VVT) (upper panel), HaCaT knock-out cells devoid of LRRC8A (LRRC8A^{-/-} KO) and HaCaT knock-out cells devoid of LRRC8D (LRRC8D^{-/-} KO). (B) Graphical display of survival rate of HaCaT wildtype vs. LRRC8A knock-out cells in presence of blasticidin.

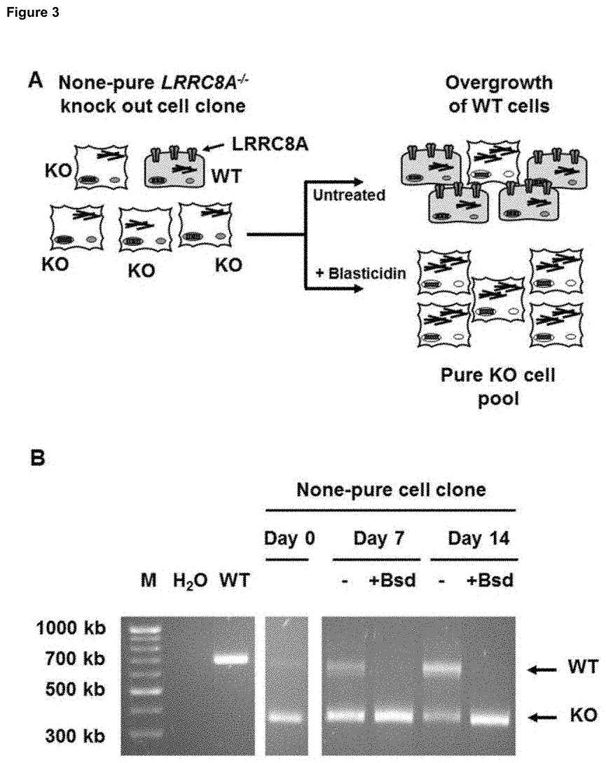

FIG. 3: (A) Schematic drawing which shows the general principal to remove unwanted wildtype cells from an heterogeneous LRRC8A^{-/-} cell clone by using blasticidin. (B) PCR analysis of genomic DNA isolated from non-treated and blasticidin-treated (one pulse 4 μg/mlblasticidin) LRRC8A knock-out cell clone after 7 and 14 days culturing in blasticidin-free medium. Arrows indicate the non-deleted, intact LRRC8A gene of wildtype cells (VVT) and the gene deletion fragment of the LRRC8A knock-out cells (KO). Blasticidin-treatment prevented the overgrowth of unwanted wildtype cells yielding a pure LRRC8A knock-out cell population.

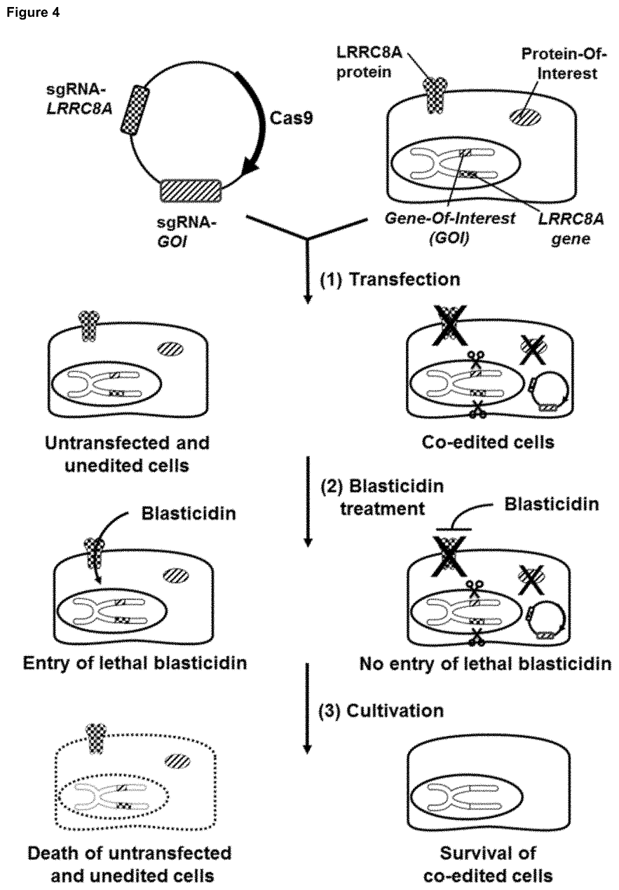

FIG. 4: Schematic drawing of the general procedure to enrich genome-edited cells by co-editing the gene-of-interest (GO!) with the LRRC8A gene followed by blasticidin treatment. For detailed explanation see Example 3.

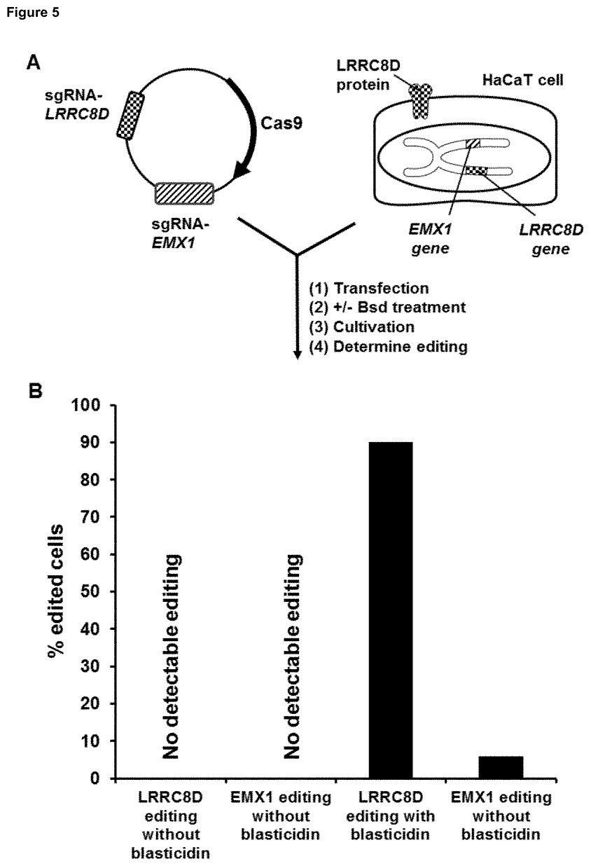

FIG. 5: (A) Schematic drawing of the procedures (step 1-4) and expression plasmids, which were used to perform co-editing of the EMX1 (as a typical example for a gene-of-interest in genome editing research) and the blasticidin (Bsd) resistance-mediating LRRC8D gene in HaCaT-cells. (B) Bar diagram that shows the percentage of edited cells (as determined by ICE analysis) in untreated vs. blasticidin-treated cells. Clearly, co-editing EMX1 and LRRC8D followed by blasticidin-treatment yielded higher percentage of edited cells in comparison to not-blasticidin-treated cells, which showed no gene editing at all.

FIG. 6: (A) Schematic drawing of the two sgRNA-expression cassettes each transcribed by two U6 promoters. (B) Schematic drawing of a single sgRNA-expression cassette with two sgRNAs separated by HDV and Hammerhead sequences. A single transcript will be generated at which the HDV will process the 3′-end of the first sgRNA and the Hammerhead the 5′-end of the second sgRNA. After the processing two native sgRNA will be liberated. (C) The same as in (B) but instead of ribozyme sequences the two sgRNAs are separated by processing sequence, which will be recognized in the cell by specific RNA-processing enzymes.

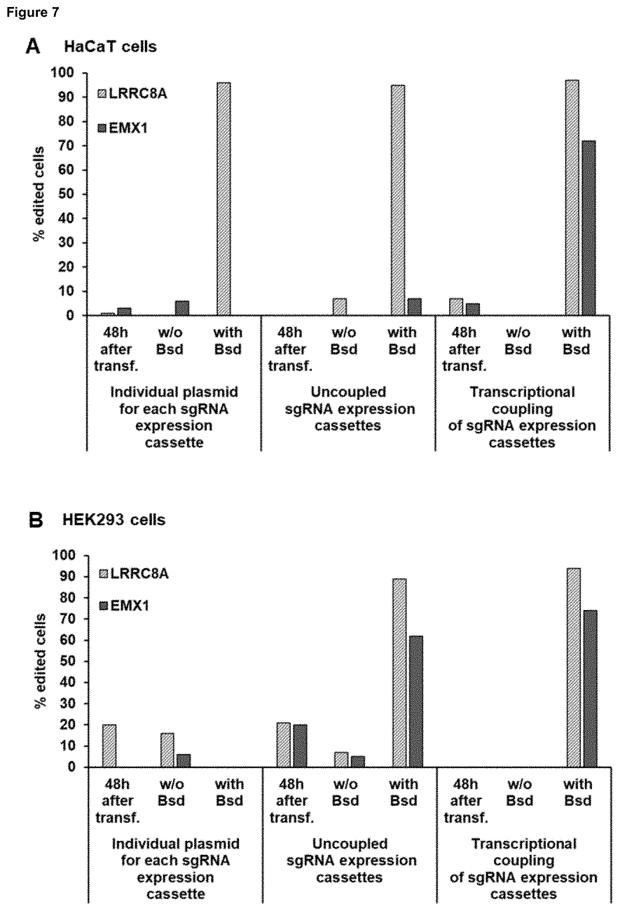

FIG. 7: Bar diagrams that show the percentage of edited cells (as determined by ICE analysis) with respect to editing of EMX1 as gene of interest and to editing of LRRC8A as blasticidin-resistance-mediating gene after 48 h post-transfection and after several days of cultivation without (w/o Bsd) or with blasticidin treatment (with Bsd) in HaCaT cells (A) and in HEK293 cells (B). Differently coupled gene expression cassettes for EMX1 and LRRC8A sgRNAs were used for transfection: single plasmids for each sgRNA, two independent, uncoupled gene expression cassettes on the same plasmid or transcriptionally coupled by ribozymes HH/HDV on the same plasmid. Clearly, co-editing EMX1 and LRRC8A followed by blasticidin-treatment yielded higher percentage of edited cells in comparison to not-blasticidin-treated cells. Moreover, transcriptional coupling of both sgRNAs could even further increase the percentage of edited cells that were edited at the gene of interest EMX1.

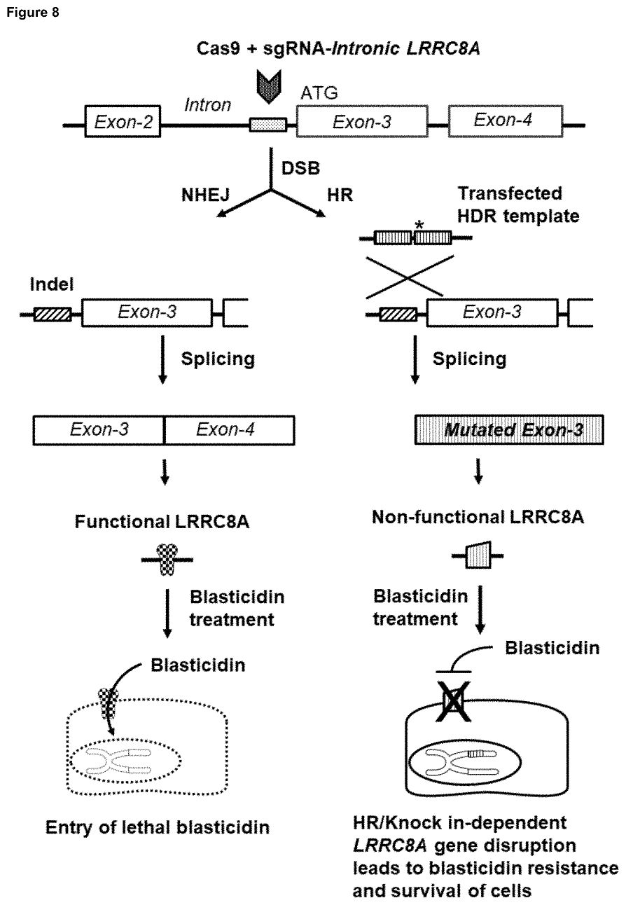

FIG. 8: Schematic drawing of the general procedure to enrich genome-edited knock-in cells by combining blasticidin treatment with HR-dependent editing of the blasticidin-resistance-mediating LRRC8A gene. For detailed explanation see Example 6.

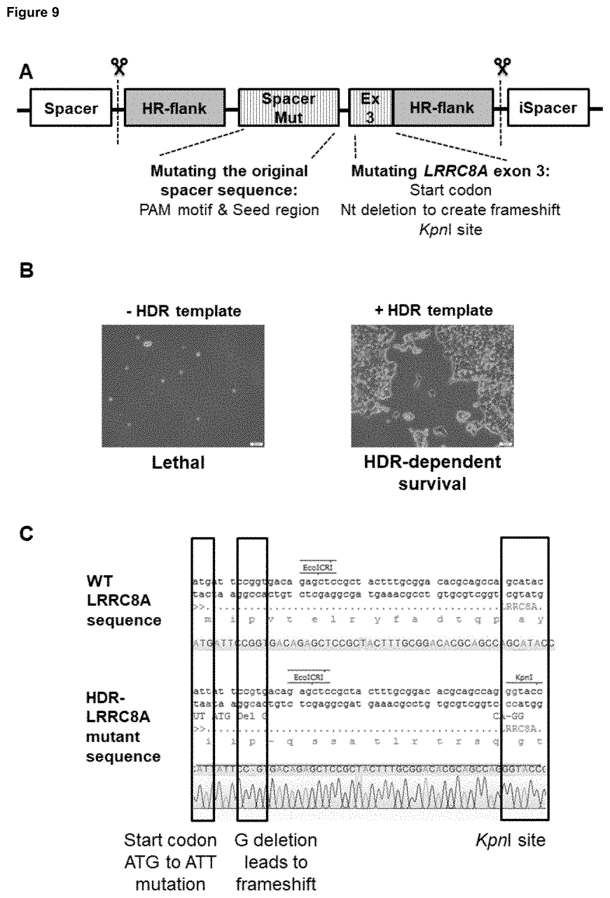

FIG. 9: (A) Schematic drawing (not to scale) of HDR donor template used for insertion at the LRRC8A locus after sgRNA/Cas9-induced DSB to induce HR-dependent LRRC8A gene disruption. Detailed description of the single features is provided in Example 7. (B) Microscopic analysis of cytotoxic effect of blasticidin treatment on HEK293 cells. HEK293 cells were transfected with (i) plasmid carrying gene expression cassettes for Cas9 and for sgRNA targeting intronic LRRC8A region and with or without (ii) plasmid carrying the HDR donor template depicted in (A). Only cells which were transfected with HDR donor template survived blasticidin treatment indicating successful integration of the HDR donor template, which led to LRRC8A gene disruption and resistance of the knock-in cells to blasticidin. (C) Comparison of Sanger DNA sequencing results of PCR-amplified genomic DNA from wildtype HEK293 cells and from HEK293 cell pool, which survived blasticidin treatment (see B) confirms successful integration of the HDR donor plasmid. The modified genomic sequence contains the mutated start codon (ATT instead of ATG), lacks one nucleotide (Guanin) and contains the Kpnl restriction enzyme site. Hence, enrichment of knock-in cells can be achieved by combining blasticidin treatment with HR-dependent LRRC8A gene disruption.

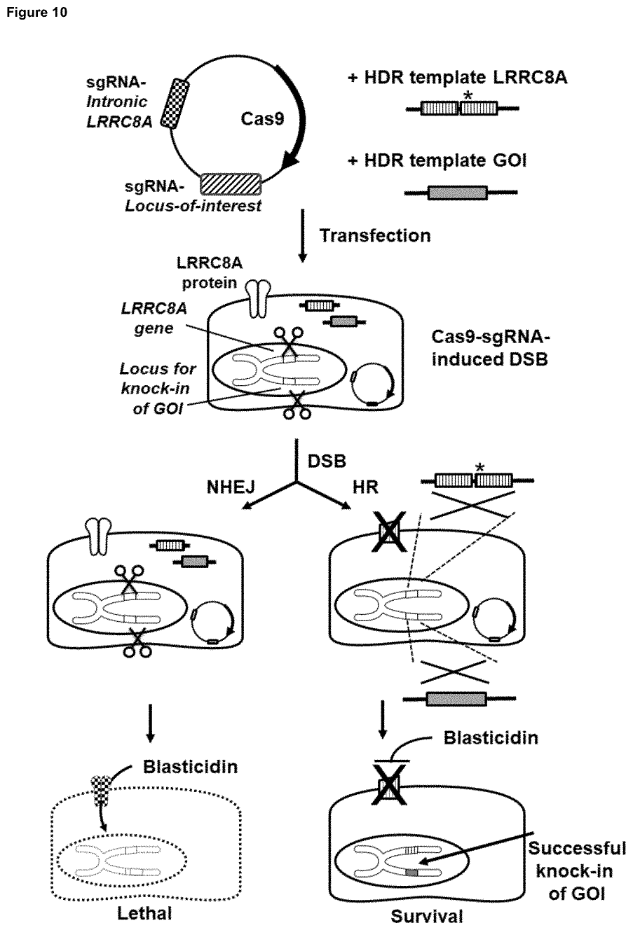

FIG. 10: Schematic drawing of the general procedure to universally enrich knock-in cells by HR-dependent co-editing of the locus of interest and LRRC8A. For detailed explanation see Example 8.

The Examples illustrate the invention.

### EXAMPLES

### Example 1

### Knock-Out of the Endogenous LRRC8A Gene Creates Cells that are Resistant to Blasticidin.

In order to determine the tolerance against the antibiotic blasticidin, HaCaT wildtype keratinocytes (VVT; Boukamp, et al.1988, The Journal of Cell Biology, 106(3):761-771.) as well as HaCaT-LRRC8A^{-/-} knock-out (KO) cells were treated with different concentrations of blasticidin and growth was determined microscopically for several days (FIG. 2). At a concentration of 4 μg/ml blasticidin, HaCaT-WT cells were not able to grow anymore and died (FIG. 2A). In contrast, surprisingly the HaCaT-LRRC8A-KO cells survived the treatment with blasticidin (FIG. 2A) and could be cultivated in the presence of blasticidin (FIG. 2B). As previously shown for KBM-7cells¹⁰, the LRRC8D subunit is also required for blasticidin uptake in HaCaT cells (FIG. 2A). This suggests that LRRC8 complexes mediate blasticidin transport in a wide-variety of cell types. Hence this unexpected finding offers a new and easy way to confer blasticidin resistance to any desired cell type, simply by disrupting the function of LRRC8A, LRRC8D or any other LRRC8 subunit.

### Example 2

### Removal of Wildtype Cells from a Mixed LRRC8A^{-/-} Cell Pool by Treating Cells with Blasticidin

Creating single cell clones is one of the most challenging tasks in all fields of genome engineering. In particular non-edited wildtype cells can impose a great problem since in general wildtype cells have superior growth advantages compared to genome edited cells and are likely to overgrow the desired cells clones over time. Therefore it was of interest to analyze whether the LRRC8A-mediated blasticidin resistance could be a very easy method to get rid of non-edited wildtype cells. To mimic this issue, a non-pure LRRC8A^{-/-} HaCaT cell clone, which contained the desired 300 bp LRRC8A genomic deletion as well as few undesired HaCaT wildtype cells, was chosen and either treated or not treated with a single blasticidin pulse (4 μg/ml blasticidin applied for 24 h) (FIG. 3A). Then cultivation medium was changed and cells were cultivated in absence of blasticidin. Genomic DNA was isolated and PCR was performed to visualize the genomic deletion of LRRC8A in knock-out cells and/or the non-deleted, intact LRRC8A gene of wildtype cells (FIG. 3B). As expected, when the non-pure cell clone was untreated, wildtype cells were able to overgrow the edited cells, as can be seen by the increasing PCR product signal that corresponds to wildtype cells and the decreasing PCR product signal that corresponds to knock-out cells (FIG. 3B). Strikingly, treatment with blasticidin prevented this effect: No

PCR product that corresponds to wildtype cells was detected anymore; instead only the PCR product of the pure knock-out cell clone was detectable (FIG. 3B). Hence, blasticidin treatment can successfully be used to very conveniently get rid of wildtype cells from a non-pure LRRC8A knock-out cell clone without any technical equipment (FIG. 3A). This also proves that disruption of the LRRC8A-mediated blasticidin resistance can not only be used to create blasticidin-resistant cells but that it is also suited to get rid of unwanted wildtype cells during genome engineering.

### Example 3

### General Procedure to Enrich Genome-Edited Cells by Combining Blasticidin Treatment with Co-Editing of the Gene-Of-Interest GOI and the Blasticidin-Resistance-Mediating LRRC8A Gene

In the next step it was envisioned to use these new findings and create a universal biotechnological tool for genome engineering that would easily allow enriching only successfully edited cells, while wildtype and non-edited cells would not survive blasticidin-treatment. The idea of the invention is illustrated in FIG. 4.
(1) First, the coding sequences of the sgRNA targeting the LRRC8A gene as well as the sgRNA targeting any desired gene-of-interest are cloned into an expression plasmid, which also contains the coding sequence for the Cas9 nuclease. The expression plasmid is used for transfection of any-desired cell type, which endogenously expresses the GOI and LRRC8A. After transfection of the expression plasmid, Cas9 nuclease will create DNA double-strand breaks at the positions that are defined by the sgRNAs, which will be repaired by the cell-inherent error-prone NHEJ system leading to missense mutations and gene knock-out of the GOI as well as LRRC8A. Since sgRNAs of the GOI and LRRC8A are coupled and editing generally occurs for both sgRNAs with the same chance, all cells that have successfully edited the GOI will also have successfully edited LRRC8A. This means that these cells will not form functional GOI as well as functional LRRC8A thereby creating cells, which are resistant to blasticidin. In contrast, un-transfected, non-edited wildtype cells will still express functional GOI and LRRC8A.
(2) In the second step, the heterogeneous cell pool will be treated with blasticidin. Co-edited cells will survive blasticidin treatment, because they do not only lack the GOI but also LRRC8A, which is required for blasticidin uptake. In contrast, un-transfected, non-edited wildtype cells still express functional LRRC8A, which allows blasticidin to enter the cell leading to cell death.
(3) Finally, only cells that have the desired genomic modification of both the GOI and LRRC8A survived blasticidin treatment and can be easily propagated.

In cases where editing efficiencies of both sgRNAs are not equal also single-edited cells containing either only LRRC8A or GOI genomic modification could occur. Single-edited GOI-KO cells will not survive blasticidin treatment since they still contain functional LRRC8A. Single-edited LRRC8A-KO cells, however, will survive blasticidin treatment. Therefore, the selection procedure may not exclusively yield co-edited cells but also single-edited LRRC8A-KO cells. In order to prevent the formation of single-edited LRRC8A-KO cells, the present invention includes methods to adjust editing efficiencies of the sgRNAs: (i) By using a LRRC8A-sgRNA, which is less efficient than the sgRNA of the GOI or (ii) by transfecting less LRRC8A-sgRNA expressing plasmids compared to the GOI expressing plasmids or (iii) by transcriptionally coupling expression of the GOI-sgRNA and the LRRC8A-sgRNA, for example by using self-splicing RNAs or tRNAs, the editing of LRRC8A becomes the rate limiting factor. Consequently, editing of the GOI will happen more frequently than editing of LRRC8A, which will hamper the creation of single-edited LRRC8A cells.

### Example 4

### Co-Editing of LRRC8D and EMX1 as Gene-of-Interest for Enrichment of CRISPR-Modified Cells

The functionality of the above-described co-editing system was tested by using EMX1 (Empty Spiracles Homeobox 1) as gene-of-interest and LRRC8D as blasticidin resistance-mediating gene. EMX1 is a commonly used reference gene in CRISPR-based research²³. Instead of LRRC8A, another member of the LRRC8 gene family, LRRC8D, was used. First, the coding sequences of two sgRNAs targeting the LRRC8D and EMX1 gene were cloned into an expression plasmid containing the coding sequence for the Cas9 nuclease (FIGS. 4 and 5A). Then HaCaT cells were transfected with the expression plasmids and cells were either treated or not treated with blasticidin (FIG. 5A). After several days of cultivation, genomic DNA was extracted and used in PCR to amplify the genomic-region of the LRRC8D and EMX gene. The PCR products were analyzed by Sanger sequencing and subsequent ICE (Inference of CRISPR Edits)²⁴ analysis to determine the genetic modifications occurring in the treated and non-treated HaCaT cells (FIG. 5B). Strikingly, when the transfected HaCaT cell pool was not treated with blasticidin, no editing event could be detected; neither in the EMX1 nor in the LRRC8D gene. In contrast, the treatment of transfected HaCaT cells with blasticidin resulted in 90% of editing percentage of the LRRC8D gene and 6% of the co-edited EMX1 gene (FIG. 5B).

These results suggest that cells which have been successfully co-edited were overgrown by wildtype, untransfected or unedited HaCaT cells. By applying blasticidin, it was possible to eliminate these unwanted cells thereby enriching the co-edited cells.

In summary, this clearly proves the principle functionality of the above-presented co-editing strategy based on LRRC8-knockout and blasticidin treatment.

### Example 5

### Transcriptional Coupling to Increase Frequency of Co-Editing

To further increase the editing rate of the gene of interest, the two single sgRNA cassettes of the gene of interest and the LRRC8A or LRRC8D selection gene (FIG. 6A) can be transcriptionally coupled, for example by coupling the sgRNAs via ribozyme structures (FIG. 6B) or by other processing sites such as tRNAs (FIG. 6C). In order to prove the functionality of this approach, the sgRNA of LRRC8A was coupled via riboyzmes HDV/HH to the sgRNA of the gene of interest EMX1 (FIG. 6B). Then HaCaT cells were transfected with the corresponding expression plasmids and cells were treated and analyzed as described in Example 4.

The initial editing rates for EMX1 and LRRC8A were determined 48 h post-transfection and were very low (<5%) (FIG. 7A). Treatment with blasticidin led to drastic increase of LRRC8A editing (>95%), which was equally high independent of the sgRNA expression construct showing efficient enrichment of LRRC8A-KO cells using blasticidin (FIG. 7A). When sgRNA expression cassette of EMX1 was independent from that of LRRC8A, EMX1 editing was increased from 3 to 7%. Strikingly, by coupling the two sgRNA cassettes of EMX1 and LRRC8A via ribozymes, EMX1 editing could drastically be increased from 7% to >70% (FIG. 7A). This clearly proves that the above-described strategy to increase low editing events by transcriptional coupling is functioning.

To confirm the suitability of the co-editing strategy of the present invention also for a different cell type, the same experimental setup was applied for HEK293 cells (FIG. 7B). Again, initial editing of the gene of interest EMX1 was also very low in HEK293 cells. By applying the co-editing strategy as presented above for HaCaT cells, it was also possible to increase the editing rates to approx. 60% for EMX1 and approx. 90% for LRRC8A in HEK293 cells (using uncoupled sgRNA expression constructs) (FIG. 7B).

Again, by transcriptional coupling it was possible to further increase editing rates for EMX1 from 62% to 74% (FIG. 7B).

Taken together, these results clearly show that transcriptional coupling is suitable to further increase editing frequency of the gene-of-interest; albeit it is not always necessary when editing rates with the uncoupled expression cassettes are already high (as shown here for HEK293 cells). In cases when initial editing rates of the genes of interest are low (as shown here for EMX1 in HaCaT cells), it is a highly advantageous to increase the enrichment of co-edited cells.

### Example 6

### General Procedure to Enrich Genome-Edited Knock-In Cells by Combining Blasticidin Treatment with HR-Dependent Editing of the Blasticidin-Resistance-Mediating LRRC8A Gene

After having shown that the co-editing strategy of this invention works for enrichment of knock-out cells, the strategy was further modified to also allow specific enrichment of knock-in cells. Such methods are of huge importance since in almost all somatic tissue culture cells creating knock-in cells is naturally more difficult than knock-out cells. The reason is that NHEJ (non-homologous end joining) is the major DNA-repair mechanism when CRISPR/Cas9 is applied, which ultimately leads to small nucleotide insertions or deletions (so-called indel mutations) and knock-out of the gene. In contrast to knocking out genes, the insertion of genes or DNA sequences of interest via CRISPR/Cas9 requires cells that are favoring the alternative DNA-repair mechanism based on HR (homologous recombination) which is a rare event in somatic tissue culture cells. The strategy described herein allows specific enrichment and survival of only those cells that have undergone HR after Cas9/sgRNA-mediated DNA cleavage, while cells that performed NHEJ are killed. The strategy is based on LRRC8A editing in a HR-dependent manner, which leads to LRRC8A gene disruption and subsequent blasticidin resistance and survival cells (FIG. 8).

In contrast to the knock-out strategy, where the sgRNA is directing the Cas9 nuclease to the coding sequence (coding sequence begins with the translational start site ATG in exon-3) of LRRC8A, the knock-in strategy is based on using a sgRNA targeting Cas9 to the intronic, non-coding region of the LRRC8A gene located just upstream of exon-3 (see FIG. 8). In addition, a homology-directed repair(HDR) donor template is co-transfected. Upon Cas9-induced DNA cleavage at this intronic region, cells will either use NHEJ or HR to repair the DSB.

In case of NHEJ, the HDR donor template will not be incorporated but instead indels will be created. However, they will not lead to a mutation of the LRRC8A coding sequence since the indels are appearing in the intronic and not in the coding LRRC8A sequence. As a consequence, splicing of exon-3 and exon-4 occurs without any perturbations creating functional LRRC8A. Due to functional LRRC8 channels, blasticidin can enter the cell, which will be lethal. Consequently, not only untransfected and non-edited wild-type cells will not survive blasticidin treatment but also cells that have performed NHEJ after Cas9/sgRNA-induced DSB.

In case of HR, the HDR donor template will be inserted at the intronic site of LRRC8A. The HDR donor template is designed in that way that the HDR flanks are matching to LRRC8A genomic sequence. But in addition, the right arm of the HDR template, which reaches into the 5′ coding sequence of LRRC8A (exon-3) is designed to carry mutations (indicated as *, FIG. 8; details see FIG. 9 A), which will lead to modified, non-functional gene sequence of LRRC8A (upon integration of the HDR donor template). After splicing of the LRRC8A gene, a mutated exon-3 is created, which in turn creates a non-functional LRRC8A ion channel subunit. Due to non-functional LRRC8 ion channels, blasticidin cannot enter the cell. Finally, only cells that have performed HR after Cas9/sgRNA-induced DSB will survive blasticidin treatment.

In summary, LRRC8A gene disruption and subsequent blasticidin resistance is only occurring in those cells, which have incorporated the mutated LRRC8A-HDR donor template allowing the enrichment of HR-capable cells via blasticidin. In contrast, cells that have performed NHEJ (or cells that were not edited or not transfected) will still carry a functional LRRC8A gene and ion channel and hence they will be sensitive to blasticidin treatment.

### Example 7

### Enrichment of Genome-Edited Knock-In Cells by Combining Blasticidin Treatment with HR-Dependent LRRC8A Gene Disruption

Next, it was tested whether the above-described strategy (Example 6, FIG. 8) can indeed be employed to enrich HR-capable cells (or conversely to get rid of cells which have performed NHEJ). First, a HDR donor template was designed (FIG. 9A), which creates a modified, non-functional LRRC8A sequence after successful insertion via HR. The HDR donor template is composed of left and right HR flaks, which are complementary to intronic and exon-3 LRRC8A sequence, respectively. The exon-3 sequence used in the HDR donor template carries two modifications to create non-functional LRRC8A: (i) Mutation of the translational start site ATG to ATT and (ii) deletion of one nucleotide (Guanin) to create LRRC8A frameshift. To also test whether foreign or recombinant DNA sequences can be inserted into the LRRC8A locus as well, the restriction site for the Kpnl restriction enzyme was included into the HDR donor template. To prevent the repeated cutting of the Cas9 enzyme (which would prevent successful HDR donor template insertion), the spacer sequence of the sgRNA (which is used to induce the DSB at the intronic region of LRRC8A) was modified by mutating the PAM motif and the seed region. The HDR donor template was cloned into a plasmid and additionally contained two flanking spacer sequences, which get cleaved by the Cas9 nuclease within the cell nucleus to release the HDR donor template from the plasmid to allow HR. The Cas9 nuclease and the sgRNA targeting the above-mentioned intronic site of LRRC8A were cloned into a separate expression vector (similar to plasmid shown in FIG. 5A but without the second sgRNA gene expression cassette).

Next, HEK293 cells were transfected with (i) expression plasmid carrying Cas9 and sgRNA expression cassette to induce DSB at the intronic site upstream of the first coding exon-3 of LRRC8A (see also FIG. 8) and (ii) additional plasmid carrying the HDR donor template (FIG. 9A). A control transfection without HDR donor template was performed in parallel. 72 h after transfection, HEK293 cells were treated with blastidicin and cultivated for several days. Importantly, no cell growth was observed when the HDR template was omitted (FIG. 9B) indicating that cells which were not able to perform HR (due to the lack of HDR donor template), i.e. cells which instead performed NHEJ, are sensitive to blasticidin. In contrast, cell growth was observed when HDR donor template was included in the transfection reaction (FIG. 9B). This strongly suggests that knock-in of the mutated LRRC8A-HDR donor template was successful, which in turn led to the proposed HR-dependent cell survival and enrichment of HR-capable cells.

To ensure that HR-dependent cell survival was indeed due to insertion of the mutated LRRC8A-HDR template (FIG. 9A), genomic DNA of the blasticidin-resistant HEK293 cells was extracted and PCR to amplify the exon-3 region of LRRC8A was performed followed by Sanger sequencing. Strikingly,

Sanger sequencing revealed successful integration of the entire HDR donor template at the predicted site of the targeting sgRNA. The engineered genomic sequence now contains the mutated ATG translational start codon (ATT) and lacks the nucleotide Guanin. It also now harbors the recombinant Kpnl site. These modifications altogether lead to the desired mutated, non-functional LRRC8A gene. These sequencing results confirm that HR-dependent insertion of a modified DNA sequence into the LRRC8A locus can be used to create non-functional LRRC8A gene, which in turn allows selection of corresponding blasticidin-resistant cells via blasticidin treatment.

In summary, this shows that the general procedure described in this invention is suitable to enrich genome-edited knock-in cells by combining blasticidin treatment with HR-dependent editing of the blasticidin-resistance-mediating LRRC8A gene.

### Example 8

### General Procedure to Universally Enrich Knock-In Cells by HR-Dependent Co-Editing of the Locus of Interest and LRRC8A

Next, the strategy was further modified to enable universal enrichment of knock-in cells that carry any desired DNA sequence at any desired locus in the genome. It is based on HR-dependent survival of edited cells, which have co-edited the locus of interest and the blasticidin-resistance-mediating LRRC8A gene, whereas wiltype, non-edited as well as cells which have performed NHEJ would not survive blasticidin-treatment. The idea of the invention is illustrated in FIG. 10.

To achieve this, a second HDR donor template for the gene or DNA sequence of interest, which is intended to be inserted, has to be designed in addition to the mutated HDR donor template for LRRC8A. In order to target the desired location in the genome, a corresponding sgRNA for the locus of interest has to be designed and cloned into sgRNA expression plasmid, which also contains the sgRNA expression cassette for the sgRNA targeting intronic LRRC8A sequence and for the Cas9 nuclease. To ensure equal expression and editing, both sgRNAs can be transcriptionally coupled, for example via ribozymes (as successfully applied in Example 5, FIG. 6 and FIG. 7).

After transfection, Cas9-sgRNA-induced DSB will appear at the LRRC8A locus as well as at the locus of interest. As described in detail above, when cells have performed NHEJ at the Cas9-induced break site, the LRRC8A coding sequence will not be modified and hence subsequent blasticidin treatment will be lethal for cells that have performed NHEJ. In contrast, only cells that have performed HR will survive blasticidin treatment, because HR will lead to insertion of the mutated LRRC8A-HDR donor template (mutations indicated as * in FIG. 10) and hence will create non-functional LRRC8A. By enriching specifically HR-capable cells the chance for successful integration of also the second HDR donor template at the second locus is drastically increased.

In conclusion, simultaneous use of (i) one sgRNA targeting any locus of interest and one sgRNA targeting LRRC8A intronic region and (ii) HDR donor templates for the DNA of interest to be inserted and for the LRRC8 gene (followed by blasticidin selection) will result in the enrichment of HR-competent cells that are able to insert the DNA of interest at any user-defined location in the genome.

### REFERENCES

1. Doudna J A, Charpentier E. Genome editing. The new frontier of genome engineering with CRISPR-Cas9. Science. 2014;346(6213):1258096.
2. Hsu P D, Lander E S, Zhang F. Development and applications of CRISPR-Cas9 for genome engineering. Cell. 2014;157(6):1262-1278.
3. Duda K, Lonowski L A, Kofoed-Nielsen M, et al. High-efficiency genome editing via 2A-coupled co-expression of fluorescent proteins and zinc finger nucleases or CRISPR/Cas9 nickase pairs. Nucleic Acids Res. 2014;42(10):e84.
4. Abascal F, Zardoya R. LRRC8 proteins share a common ancestor with pannexins, and may form hexameric channels involved in cell-cell communication. Bioessays. 2012;34(7):551-560.
5. Jinek M, Chylinski K, Fonfara I, Hauer M, Doudna J A, Charpentier E. A programmable dual-RNA-guided DNA endonuclease in adaptive bacterial immunity. Science. 2012;337(6096):816-821
6. Ran F A, Hsu P D, Wright J, Agarwala V, Scott D A, Zhang F. Genome engineering using the CRISPR-Cas9 system. Nat Protoc. 2013;8(11):2281-2308.
7. Hille F, Richter H, Wong S P, Bratovic M, Ressel S, Charpentier E. The Biology of CRISPR-Cas: Backward and Forward. Cell. 2018;172(6):1239-1259.
8. Qiu Z, Dubin A E, Mathur J, et al. SWELL1, a plasma membrane protein, is an essential component of volume-regulated anion channel. Cell. 2014;157(2):447-458.
9. Voss F K, Ullrich F, Munch J, et al. Identification of LRRC8 heteromers as an essential component of the volume-regulated anion channel VRAC. Science. 2014;344(6184):634-638.
10. Lee C C, Freinkman E, Sabatini D M, Ploegh H L. The protein synthesis inhibitor blasticidin s enters mammalian cells via leucine-rich repeat-containing protein 8D. J Biol Chem. 2014;289(24):17124-17131.
11. Yoshinari T, Sugita-Konishi Y, Ohnishi T, Terajima J. Inhibitory Activities of Blasticidin S Derivatives on Aflatoxin Production by Aspergillus Flavus. Toxins (Basel). 2017;9(6).
12. Agudelo D, Duringer A, Bozoyan L, et al. Marker-free coselection for CRISPR-driven genome editing in human cells. Nat Methods. 2017;14(6):615-620.
13. Liao S, Tammaro M, Yan H. Enriching CRISPR-Cas9 targeted cells by co-targeting the HPRT gene. Nucleic Acids Res. 2015;43(20):e134.
14. Kim H, Ishidate T, Ghanta K S, et al. A co-CRISPR strategy for efficient genome editing in Caenorhabditis elegans. Genetics. 2014;197(4):1069-1080.
15. Kang T H, Park Y, Bader J S, Friedmann T. The housekeeping gene hypoxanthine guanine phosphoribosyltransferase (HPRT) regulates multiple developmental and metabolic pathways of murine embryonic stem cell neuronal differentiation. PLoS One. 2013;8(10):e74967.
16. Guibinga G H, Hsu S, Friedmann T. Deficiency of the housekeeping gene hypoxanthine-guanine phosphoribosyltransferase (HPRT) dysregulates neurogenesis. Mol Ther. 2010;18(1):54-62.
17. Song S, Friedmann T. Tissue-specific aberrations of gene expression in HPRT-deficient mice: functional complexity in a monogenic disease? Mol Ther. 2007;15(8):1432-1443.
18. Gao Y, Zhao Y. Self-processing of ribozyme-flanked RNAs into guide RNAs in vitro and in vivo for CRISPR-mediated genome editing. J lntegr Plant Biol. 2014;56(4):343-349.
19. Yoshioka S, Fujii W, Ogawa T, Sugiura K, Naito K. Development of a mono-promoter-driven CRISPR/Cas9 system in mammalian cells. Sci Rep. 2015;5:18341.
20. Xu L, Zhao L, Gao Y, Xu J, Han R. Empower multiplex cell and tissue-specific CRISPR-mediated gene manipulation with self-cleaving ribozymes and tRNA. Nucleic Acids Res. 2017;45(5):e28.
21. Yan Q, Xu K, Xing J, et al. Multiplex CRISPR/Cas9-based genome engineering enhanced by Drosha-mediated sgRNA-shRNA structure. Sci Rep. 2016;6:38970.
22. Nissim L, Perli S D, Fridkin A, Perez-Pinera P, Lu T K. Multiplexed and programmable regulation of gene networks with an integrated RNA and CRISPR/Cas toolkit in human cells. Mol Cell. 2014;54(4):698-710.
23. Cong L, Ran FA, Cox D, et al. Multiplex genome engineering using CRISPR/Cas systems. Science. 2013;339(6121):819-823.
24. Hsiau T, Maures T, Waite K, et al. Inference of CRISPR Edits from Sanger Trace Data. bioRxiv. 2018.

1. A method for enriching cells within a cell population, wherein a gene of interest is genome edited, said method comprising
(a) introducing into the cells within the cell population one or more nucleic acid molecules, said one or more nucleic acid molecules encoding in expressible form
(i) a CRISPR nuclease,
(ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and
(iii) a guide RNA for editing the gene of interest; or
(a′) introducing into the cells within the cell population
(i) a CRISPR nuclease,
and one or more nucleic acid molecules, said one or more nucleic acid molecules encoding in expressible form
(ii) a guide RNA for knocking-out the leucine-rich repeat-containing protein 8 A (LRRC8A), LRRC8B, LRRC8C, LRRC8D or LRRC8E gene or a guide RNA for knocking-out an innexin gene, and
(iii) a guide RNA for editing the gene of interest; or
(a″) introducting into the cells within the cell population
(i) a ribonucleoprotein complex (RNP) comprising or consisting of a CRISPR nuclease protein in complex with a guide RNA for knocking-out leucine-rich repeat-containing protein 8 A (LRRC8A), LRRC8B, LRRC8C, LRRC8D or LRRC8E or a guide RNA for knocking-out an innexin gene, and
(ii) a RNP comprising or consisting of the CRISPR nuclease in complex with a guide RNA for editing the gene of interest; and
(b) treating the cells obtained after (a) or (a′) or (a″) with blasticidin or a derivative thereof,
thereby (i) selecting cells wherein the LRRC8A, LRRC8B, LRRC8C, LRRC8D or LRRC8E gene or the innexin gene is knocked-out, and (ii) enriching cells, wherein the gene of interest is genome edited.

2. The method of claim 1, wherein the expression of the gRNA for editing the gene of interest is transcriptionally coupled to the expression of the gRNA for knocking-out the LRRC8A, LRRC8B, LRRC8C, LRRC8D or LRRC8E gene or the guide RNA for knocking-out an innexin gene.

3. The method of claim 2, wherein the transcriptional coupling is achieved by using
(i) self-splicing RNAs, preferably HDV and Hammerhead ribozymes, or
(ii) RNA-sequences that are processed by proteins, preferably by tRNA-sequences.

4. The method of claim 1, wherein the method further comprises
(c) isolating one or more cells, wherein the gene of interest is genome edited.

5. An isolated cell obtained by the method of claim 4.

6. A composition comprising the cell of claim 5.

7. The composition of claim 6, wherein the composition is a pharmaceutical composition.

8. A vector comprising in expressible form
(i) optionally a CRISPR nuclease,
(ii) a guide RNA for knocking-out the LRRC8A, LRRC8B, LRRC8C, LRRC8D or LRRC8E gene or a guide RNA for knocking-out an innexin gene, and
(iii) a guide RNA editing the gene of interest.

9. A kit comprising
(a) one or more nucleic acid molecules encoding in expressible form
(i) a CRISPR nuclease,
(ii) a guide RNA for knocking-out LRRC8A, LRRC8B, LRRC8C, LRRC8D or LRRC8E or a guide RNA for knocking-out an innexin gene, and
(iii) a guide RNA for editing the gene of interest, or
(b) (i) a CRISPR nuclease, and one or more nucleic acid molecules encoding in expressible form (ii) a guide RNA for knocking-out LRRC8A, LRRC8B, LRRC8C, LRRC8D or LRRC8E or a guide RNA for knocking-out an innexin gene, and (iii) a guide RNA for editing the gene of interest.

10. The method of claim 1,
wherein the guide RNA of (ii) is for knocking-out the LRRC8D gene or the LRRC8A gene, preferably the LRRC8D gene.

11. The method of claim 1,
wherein the CRISPR nuclease is a Class II CRISPR-Cas nuclease, and is preferably Cas9, Cpf1, CasX, CasY or C2c1/2/3.

12. The method of claim 1,
wherein the guide RNA for editing the gene of interest is for knocking-out or knocking-in the gene of interest.

13. The method of claim 1,
wherein
the guide RNA for knocking-out the LRRC8A, LRRC8B, LRRC8C, LRRC8D or LRRC8E gene targets a non-coding region of the LRRC8A, LRRC8B, LRRC8C, LRRC8D or LRRC8E gene; or the guide RNA for knocking-out an innexin gene targets a non-coding region of the innexin gene.

14. The method of claim 1,
wherein in addition one or more nucleic acid molecules is/are provided as a first and second homologous recombination template, wherein
(i) the first homologous recombination template is to knock-out the LRRC8A, LRRC8B, LRRC8C, LRRC8D or LRRC8E gene or the innexin gene upon homologous recombination repair, and
(ii) the second homologous recombination template is to edit the gene of interest upon homologous recombination repair.

15. The method of claim 14,
wheren the second homologous recombination template is to
(i) knock-in a gene or a nucleic acid sequence encoding a protein, peptide or RNA molecule,
(ii) replace the gene of interest by another gene, or
(iii) correct a mutation in the gene of interest.
